(19) United States
(12) Patent Application Publication
(10) Pub. No.: US 2021/0115003 A1
(43) Pub. Date: Apr. 22, 2021

(54) NOVEL FULLY SYNTHETIC AND SEMISYNTHETIC PLEUROMUTILIN DERIVATIVES AS NEW ANTIBIOTICS AND THEIR PREPARATION

(71) Applicant: YALE UNIVERSITY, NEW HAVEN, CT (US)

(72) Inventors: Seth Herzon, Madison, CT (US); Xiaoshen MA, Boston, MA (US); Olivia Goethe, New Haven, CT (US); Abigail Heuer, New Haven, CT (US); Zhixun Wang, New Haven, CT (US)

(73) Assignee: YALE UNIVERSITY, NEW HAVEN, CT (US)

(21) Appl. No.: 17/040,181

(22) PCT Filed: Mar. 29, 2019
(86) PCT No.: PCT/US2019/024766

§ 371 Date: Sep. 22, 2020

Related U.S. Application Data
(60) Provisional application No. 62/649,759, filed on Mar. 29, 2018.

(51) Int. Cl. C07D 295/185; C07F 7/18

(52) U.S. Cl. CPC C07D 295/185; A61K 45/06; C07F 7/1804

(57) ABSTRACT

The present invention is directed to novel pleuromutilin antibiotic compounds, intermediates which are useful for making these novel antibiotic compounds, methods of synthesizing these compounds and related methods and pharmaceutical compositions for treating pathogens» especially bacterial infections, including gram negative bacteria.

### RELATED APPLICATIONS

The present application claims priority from provisional application No. U.S. 62/649,759, filed 29 Mar. 2018, the contents of which application is incorporated by reference in its entirety herein.

### FIELD OF THE INVENTION

The present invention is directed to novel pleuromutilin antibiotic compounds, intermediates which are useful for making these novel antibiotic compounds and related methods of preparation of these compounds. These compounds may be used as pharmaceutical agents or as intermediates in the synthesis of pharmacologically active compounds useful for treating pathogens, especially bacterial infections, including gram negative bacteria.

### BACKGROUND AND OVERVIEW OF THE INVENTION

(+)-Pleuromutilin (1) is a diterpene antibiotic¹ that inhibits protein synthesis by binding to the peptidyl transferase center (PTC) of the bacterial ribosome (Scheme 1).² Kilogram quantities of (+)-pleuromutilin (1) are accessible by fermentation. Several large pharmaceutical companies optimized the potency, metabolic stability, and spectrum of activity of 1 by semisynthesis.³ The large majority of these analogs were prepared by tosylation of the C22 hydroxyl group (1→2, Scheme 1), followed by displacement with thiol-based nucleophiles. Tiamulin (3) and valnemulin (4) are two C14 derivatives in veterinary use since the 1990s. Retapamulin (5) was approved for human use in 2007 (as a topical ointment) for the treatment for skin infections.⁴ Lefamulin (6) recently passed a Phase III clinical trial for the treatment of community-acquired pneumonia.⁵ Slow rates of resistance development and minimal cross-resistance with other ribosome-binding antibiotics are defining features of this class.^{3c,3g} The structures of tiamulin (3),^{2a} retapamulin (5),^{2b} and two additional semisynthetic derviatives^{2b} bound to the large ribosomal subunit of D. radiodurans have been determined. Each molecule binds the peptidyl transferase center (PTC) with the glycolic acid residue directed into the P-site and the hydrophobic tricyclic core positioned in the A-site. The key hydrogen bonding contacts involve the glycolic acid ester and G2061, and a weak interaction between the C11 hydroxyl group and G2505. The tricyclic core is largely devoid of polar interactions with the PTC. FIG. 1. Shows structures of (+)-pleuromutilin (1), the semisynthetic C14 derivatives 3-6, and the 12-epi-mutilin derivative 8.

Most pleuromutilins possessing the native tricyclic architecture have selective activity against Gram-positive pathogens. In 1986, Heinz Berner and colleagues, working at the Sandoz Research Institute, discovered a process to epimerize the C12 quaternary position of 2 by an unusual retroallylation-allylation pathway to provide 12-epi-pleuromutilin 22-O-tosylate (7).⁶ Recently, researchers at Nabriva explored functionalization of the pseudoequatorial alkene formed in this isomerization. An oxidative cleavage-reductive amination sequence followed by C22 functionalization provided 12-epi-mutilin derivatives such as 8. These derivatives have extended spectra of activities.⁷ They possess MIC values in the 0.125-8 μg/mL range against Gram-negative and drug resistant strains such as carbapenem-resistant Enterobacteriaceae (CRE),^{7h} Klebsiella pneumoniae,^{7d,7e} and Citrobacter freundii.^{7e} This improvement in activity is due in part to decreased resistance from AcrAB-TolC efflux.^{7b}

Collectively, these reports provide a strong case for further development of this class of compounds. Because alterations to the tricyclic skeleton are underexplored, the present inventors targeted derivatives with modified ring sizes, exocyclic substituents at sites other than C12 and C14, and atomic substitution. As the first step of this research program, the inventors developed a fully-synthetic route to (+)-pleuromutilin (1) and 12-epi-mutilin (11) that proceeds by the convergent union of the eneimide 9 with the C11-C13 synthon 10 (FIG. 2).⁸ FIG. 2 shows the convergent fragment coupling en route to 12-epi-mutilin (11).

Many different annulation reagents and cyclization strategies can be envisioned to access pleuromutilins with non-natural skeletons. To guide synthetic planning, the present inventors sought to rapidly evaluate substituent effects at sites on the periphery of the tricyclic skeleton. To achieve this, they focused on identifying methods to functionalize the C—H bonds of the C15, C16, C17, and C18 methyl substituents of (+)-pleuromutilin (1). They hypothesized that these might be artifacts of the biosynthesis, which proceeds from geranylgeranyl diphosphate,^{1e-h} and may not be fully optimized for binding to the ribosome. These efforts were inspired by recent successes in the controlled, site-selective modification of complex natural products.⁹

Other researchers have examined direct functionalization of (+)-pleuromutilin (1) or its derivatives. These studies include microbial oxidation of C7 and C8,¹⁰ vinylic hydrogen-deuterium exchange at C20,¹¹ silver-catalyzed C13-H amination,¹² and iron-catalyzed C7-H oxidation.¹³ To our knowledge, only a single study describes methyl group oxidation and involves a manganese-catalyzed C16-H amination,¹⁴ usig a non-natural C7-hydroxyl group to direct the oxidation. The antimicrobial activity of this derivative was not evaluated, to our knowledge.

### BRIEF DESCRIPTION OF THE INVENTION

The present invention is directed to the synthesis of pleuromutilin compounds and derivatives. The present invention has two general components. In a first general embodiment, the present invention is directed to methods for modifying pleuromutilin to access novel derivatives that have not yet been prepared. In one embodiment, these modifications proceed by hydroxyl-directed iridium or ruthenium catalyzed C—H silylation/C—H functionalization to provide a cyclosilane (the hydrosilane forms a cyclic ring (cyclosilane) between a hydroxyl group modified to be substituted with a dialkyl- or diphenyl hydrosilane and an adjacent methyl group). The cyclosilane intermediate is then subjected to Tamao-Fleming oxidation to provide the corresponding alcohol groups. This embodiment establishes a method to modify the methyl substituents of pleuromutilins to hydroxyl groups which were not previously accessible. This approach provides a platform with readily derivatized (hydroxyl) functional groups for the production of new antibiotics by semisynthesis. In a second general embodiment, the present invention is directed to novel strategies for the construction of synthetic pleuromutilins by total synthesis. The design of these strategies was informed by the known interactions of pleuromutilins with the bacterial ribosome and the growing working knowledge of the chemistry that is useful for accessing pleuromutilin-like structures.

Thus, in one embodiment, the present invention is directed to a method of introducing a hydroxyl group onto a methyl group of a pleuromutilin compound (pleuromutilin or a pleuromutilin derivative which is or has been reduced or derivatized to reduce or remove the vinyl group at C₁₉-C₂₀ and is otherwise appropriately protected) wherein the pleuromutilin compound contains a hydroxyl group adjacent to the methyl group to be hydroxylated, the method comprising introducing a dialkyl- or diphenylhydrosilyl group (preferably, diphenylhydrosilyl) onto the adjacent hydroxyl group to form a hydrosilane group, conducting a dehydrogenative C—H silylation reaction catalyzed by iridium ruthenium on the hydrosilane group to form a cyclosilane with the methyl group and thereafter conducting a Tamao-Fleming oxidation on the cyclosilane group to provide alcohol groups on the two carbon atoms which formed the cyclosilane. It is noted that the pleuromutilin compound must be appropriately protected with protecting groups prior to introducing the hydrosilane group and/or prior to conducting the dehydrogenative C—H silylation reaction to form the cyclosilane group. In preferred aspects, the hydrosilane group is introduced using a dialkylchlorohydrosilane (often a C₁-C₄ alkyl, more often a dimethyl or diethylchlorohydrosilane, or a diphenylchlorohydrosilane) in a weak base (e.g. triethylamine, pyridine or other base) in solvent to provide the hydrosilane on the free hydroxyl group adjacent to the methyl group to be hydroxylated. Preferably, the dehydrogenative C—H silylation reaction to form the cyclosilane on the adjacent methyl group is carried out using an iridium catalyst (e.g. methoxy(cyclooctadiene)iridium(I) dimer [Ir(OCH₃)(COD)]₂ in the presence of norbornene and 3,4,78-teramethyl-1,10-phenantholine/Me₄phen in solvent (e.g. THF) preferably at elevated temperature (e.g. 100-125° C. or more). Once the cyclosilane is formed, it is subjected to Tamao-Fleming oxidation conditions using desilylation agent such as hydrogen fluoride, tetra-n-butylammonium fluoride (TBAF) and an oxidizing agent such as hydrogen peroxide, meta-chloroperbenzoic acid or a related oxidizing agent in weak base such as potassium bicarbonate, among others). Note that the desilylation agent may be used before, after or in conjunction with oxidizing agent. This produces a compound containing two hydroxyl groups where the cyclosilane was previously substituted. The dihydroxyl group containing compound can thereafter be deprotected or further derivatized at functional groups within the compound.

In preferred methods according to the present invention, the starting pleuromutilin derivative is compound 12 (FIG. 3), compound S18 (similar to as compound 20 in which the vinyl group has been reduced) (see FIG. 5 and supporting information set forth in the examples section of the present application), compound 25 (FIG. 7), compound 30 (FIG. 8, protecting group shown is BOM, but can be any other protecting group stable to the conditions which follow), compound 38 (which is protected at the C11 hydroxyl group, such as with a trifluoroacetate group as in FIG. 10, Scheme 10), compound 43, 44 or 45 (Scheme 11, FIG. 11), compound 46 (which is reduced), compound 47 (FIG. 12), compound 54 (FIG. 13, or compound with a protecting group at the C11 hydroxyl group and at the C16 hydroxyl group), compound 59 (FIG. 14) which can be hydrogenated, esterified and reduced to form compound 62, compound 19 (FIG. 18) or compound S10 (FIG. 19). Using these compounds as indicated in the attached Scheme 3, FIG. 3, Scheme 5, FIG. 5, Scheme 7, FIG. 7, Scheme 8, FIG. 8, Scheme 10, FIG. 10, Scheme 12, FIG. 12 (compound 46 which is reduced to compound 47), Scheme 13, FIG. 13, Scheme 18, FIG. 18 and Scheme 19, FIG. 19, the C18 methyl group, C17 methyl group, C20 methyl group, C16 methyl group or C15 methyl group may be derivatized readily to a hydroxyl group, which can be further functionalized.

In an additional embodiment, the present invention is directed to one or more of the compounds which is disclosed herein, including the compounds which are presented in attached examples section. Preferred compounds of the present invention include one or more of the following compounds: 12, 13, 14a, 14b, 15a and 15b of FIG. 3; compounds 20, S18, 21, 22a, 22b, 23a and 23b of FIG. 5; compound 24 of FIG. 6; compounds 25, 26, 27 and 28 of FIG. 7; compounds 30, 31 and 32 of FIG. 8; compounds 33, 34, 35 and 36 of FIG. 9, compound 37 of FIG. 9A; compounds 38, 39, 40, 41 and 42 of FIG. 10; compounds 43, 44 and 45 of FIG. 11; compounds 46, 47 and 49 of FIG. 12; compounds 50, 51, 52 and 53 of FIGS. 13A and 13B; compounds 54, 55 and 56 of FIG. 13C; compounds 57, 58A, 58B, 58C and 58D (and their non-salts or alternative salts) of FIG. 13D, Table 1, compounds 59, 60a, 60b, 61 and 62 of FIG. 14, compounds entry 5, 6 or 7 of FIG. 15, Table S1, compounds 14a, S3a and 15a of FIG. 16, compound 31, 41, 42, S5, 37 or 32 of FIG. 17, compound 19, S6, S7 or S8 of FIG. 18, Compound 20, S9, 510, S11 or S12 of FIG. 19, compound S13, S14 or S15 of FIG. 20, compound 14A (where R is methyl or phenyl) or pharmaceutically acceptable salts, stereoisomers, solvates and polymorphs thereof. Preferred compounds according to the present invention include compound 14a (FIG. 3), compounds 22a, 23a and 23b (FIG. 5) compounds 40 and 41 (FIG. 10) and compound 48 (FIG. 12). Any one or more of the compounds disclosed herein may be used as active antimicrobial agents and/or intermediates/starting materials in the synthesis of compounds exhibiting antimicrobial and other bioactive properties.

In one embodiment, the present invention is directed to methods for promoting a C18 oxidation of (+) pleuromutilin employing the hydroxyl-directed iridium-catalyzed C—H silylation developed by Hartwig and co-workers (see, Simmons, et al., Nature 2012, 483, 70 and Li, et al., J. Am. Chm. Soc., 2014, 136, 6586. In this method, as presented in attached FIG. 3, scheme III, the vinyl group at C19 of (+)-pleuromutilin is reduced to produce the corresponding methyl group at C19 (compound 12) after protection of the hydroxylmethyl ester (C22) in this case with a protecting group, in this case a silyl protecting agent (tert.-butyl diphenyl silyl or TBDPS) and reducing the vinyl group at C19-C20. The C18 methyl group of pleuromutilin derivative (12) is oxidized/converted to the corresponding alcohol compound (15a) by first silylating the alcohol group at C11 (i.e, on a carbon atom adjacent to the carbon atom to which the methyl group to be oxidized is bonded) using a silylating reagent containing a C—H group (e.g., dialkylchlorohydrosilane, diarylchlorohydrosilane, dialkylchlorohydrosilane or diarylchlorohydrosilane, among other silylating agents) in the presence of a weak base such as trimethylamine or similar base) to form a dialkylhydrosilane (alkyl is C₁-C₄, preferably C₁ or C₂) or diarylhydrosilane (aryl is preferably phenyl or substituted phenyl) on the alcohol group as in compound 13 (dimethyl hydrosilane at C11 shown in compound 13). Compound 13, which contains the hydrosilane group on the oxygen at C11 is subjected to a dehydrogenative C—H silylation reaction catalyzed by iridium (preferably a iridium catalyst such as methoxy(cyclooctadiene)iridium(I) dimer [Ir(OCH₃)(COD)]₂ in the presence of norbornene and 3,4,78-teramethyl-1,10-phenantholine, Me₄phen in solvent (e.g. THF) preferably at elevated temperature (e.g. 120° C.); alternatively, in certain embodiments, a ruthenium catalyst may also be used) to produce the cyclosilane (compound 14a or 14b of FIG. 3 or corresponding dialkyl or diphenyloxysilane). The silyl group of compound 14a or 14b is subjected to a silyl deprotecting agent (e.g. tetra-n-butyl ammonium fluoride, TBAF) and the resulting intermediate oxidized (preferably with hydrogen peroxide in weak base such as potassium bicarbonate) to afford the corresponding alcohol substituted methyl group in compound 15 a or 15b. These compounds may be derivatized further to produce numerous derivatives of (+)-pleuromutilin which possess antimicrobial activity.

Compound 15a may be oxidized to the corresponding keto compound 17 (R is ethyl or methyl) using Tosyl chloride followed by Dess-Marin Periodinane (DMP) in two steps as set forth in FIG. 4 hereof in high yield (at least 50%, more often 60-65% or more in two steps from the starting material compound 15a).

In another embodiment, the present invention is directed to providing C17 oxidation products of (+)-pleuromutilin. The synthesis of the C17 alcohol 23a of FIG. 5, Scheme 5 is prepared from compound 19, by epimerizing the vinyl group at C12 using diethylzinc (ZnEt₂) over several cycles to produce the vinyl-epimerized compound 20 which is exposed to reduction conditions (e.g. Pd/C/hydrogen) followed by hydrosilylation in weak base (dialkyl or diphenylchlorohydrosilane in triethylamine) to provide the hydrosilylated intermediate 21. Intermediate 21 is then exposed to iridium catalyzed C—H functionalization as described above (norbornene and 3,4,78-teramethyl-1,10-phenantholine, Me₄phen in solvent (e.g. THF) preferably at elevated temperature (e.g. 120° C.)) to provide compounds 22a and 22b which are subjected to oxidation conditions (e.g. hydrogen peroxide in weak base), followed by removal of the silyl groups with a silyl removing agent (e.g. TBAF) to provide compound 23a in high yield (81%). Compound 23a can be readily converted to the corresponding aldehyde at C17 using Tosyl chloride followed by Dess-Marin Periodinane (DMP) in two steps to afford compound 24 in high yield (60+% from 23a).

Trialcohol (C11, C17, C18 OH) compound 28 may be prepared from compound 15a (FIG. 7, scheme 7) by first protecting the hydroxy ester group at carbon 22 with a silyl protecting group (e.g. tert-butyldiphenylsilyl/TBDPS) followed by selective silylation at the C18 hydroxyl group (e.g. triethylsilyl/TES) to afford the di-silylprotected intermediate compound 25, which is hydrosilylated in weak base (dialkyl or diphenylchlorohydrosilane in triethylamine) to provide the diprotected hydrosilane compound 26. Compound 26 is then exposed to iridium catalyzed C—H functionalization as described above (norbornene and 3,4,78-teramethyl-1,10-phenantholine, Me₄phen in solvent (e.g. THF) preferably at elevated temperature (e.g. 120° C.)), followed by oxidation (hydrogen peroxide/weak base) to provide compound 27 which is subjected to silyl removal of the tetrabutyldiphenyl silyl group using hydrogen fluoride in pyridine to afford compound 28.

In another embodiment, the present invention is directed to the conversion of a methyl group at C16 of (+)-pleuromutilin to a hydroxyl group to produce compound 32 (Scheme 8, FIG. 8) which can be converted into compounds 35 and 36 (Scheme 9, FIG. 9) in which the C16 methyl group has been converted to an alcohol (compound 35) or a hydroxyester (compound 36). Pursuant to Scheme 8, FIG. 8, (+)-pleuromutilin is first subjected to protection of the two free hydroxyl groups with a benzylmethylether (benzyloxymethyl/BOM) using benzyl chloromethyl ether in the presence of sodium iodide, N,N-diisopropylethylamine and dimethoxyethane (DME) at elevated temperature (e.g. 60-65° C.) to produce the diBOM protected compound 29 in near quantitative yield. Compound 29 was then exposed to sodium hydroxide to remove the ester, followed by reduction of the C12 vinyl group using tris(2,2,6,6-tetramethyl1-3,5-heptanedionate) manganese III (Mn(dpm)3 in isopropanol at about ambient temperature (e.g. 24° C.) for several hours to provide compound 30 in 79-80% yield over two steps. Compound 30 is then hydrosilylated in weak base (dialkyl or diphenylchlorohydrosilane in triethylamine) to provide the hydrosilylated intermediate (not shown) which is subjected to iridium catalyzed C—H functionalization as described above (norbornene and 3,4,78-teramethyl-1,10-phenantholine, Me₄phen in solvent (e.g. THF) preferably at elevated temperature (e.g. 120° C.)) to produce compound 31 adequate yield. Compound 31 is then subjected to silyl group removal using TBAF and oxidation (e.g. m-chloroperbenzoic acid to produce compound 32, which has had a hydroxyl group introduced at C16.

Compound 32 can be converted to compound 33 (introducing a benzyloxymethyl group as shown in Scheme 9, FIG. 9 using sodium hydride, followed by benzylchloromethyl ether and then tetrabutylammonium iodide to provide compound 33. Compound 33 (which contains two BOM protecting groups) is then converted to compound 34 which contains benzylprotected hydroxyl ester at C14 using benzyloxyacetic acid, followed by 1-ethyl-3-(3-dimethylaminopropyl)carbodiimide hydrochloride (EDCl) and dimethylaminopyridine (DMAP) in high yield. Compound 34 is subjected to benzyl deprotection conditions (e.g. reduction in (PdOH)₂/C, H₂ at 800 psi) to deprotect the benzyl protecting groups (BOM and benzyloxyacetate) and provide compound 35, which can be transesterified (acyl group migration from the C14 position to the C16 position in chloroform or trifluoroacetic acid in methylene chloride) to provide compound 36. Alternatively, compound 36 can be prepared by esterifying the hydroxyl group at C16 of compound 32 (using benzyloxyacetic acid, followed by 1-ethyl-3-(3-dimethylaminopropyl)carbodiimide hydrochloride (EDCl) and dimethylaminopyridine (DMAP) as described above, followed by removing the benzyl protecting groups using reducing conditions (e.g. reduction in (PdOH)₂/C, H₂ at 800 psi). FIG. 9A shows the X-ray structure of 16-hydroxy-19, 23-0-dihydromutilin (compound 37). The C14 and C16 carbon atoms are labeled in FIG. 9. Attached to C14 and C16 in the compound at the two hydroxyl groups are the hydroxyacetate groups (compounds 35 and 36). All other atoms are carbon atoms. Hydrogen atoms have been omitted for the sake of clarity.

In an alternative route to the C16 silylation product 31 which is presented in FIG. 8, Scheme 8, (+)-pleuromutilin is subjected to removal of the hydroxyacyl group from C14 (using for example, sodium hydroxide) in a first step, followed by reduction of the vinyl group on C13 (using e.g. Pd/C in pressurized hydrogen) to produce compound 38 in high yield (>90%) over two steps. See FIG. 10. Scheme 10. Compound 38 is converted to the corresponding hydrosilylated compound 39 by protecting the C11 hydroxyl group (e.g. trifluoroacetic anhydride (TFAA) in triethylamine and then hydrosilylating the C14 hydroxyl group (dialkyl or diphenylchlorohydrosilane in trimethylamine) to provide compound 39. Compound 39 is then subjected to iridium catalyzed C—H functionalization as described above (norbornene and 3,4,78-teramethyl-1,10-phenantholine, Me₄phen in solvent (e.g. THF) preferably at elevated temperature (e.g. 120-125° C.)) to provide compound 40 in more than 50% yield. The protecting group (TFA) on C11 of compound 40 is removed with base (e.g. using sodium hyderoxide solution) to provide compound 41, which is reprotected with a benzyloxymethyl(BOM) or acetyl (Ac) group to provide compound 31 (R is BOM) or 42 (R is Ac).

Compound 32 may also be derivatized to produce a C16 carboxylic acid (compound 45) as set forth in Scheme 11, FIG. 11. The hydroxyl group at C16 of Compound 32 is silyl protected (triethylsilyl protection shown, other protecting groups, including other silyl groups can be used) using triethylsilylchloride/weak base and the hydroxyl group at C14 is protected with a methyloxylmethylether group (MOM)(MOMCl, sodium iodide in weak base (DIPEA) to provide compound 43. Compound 43 is de-silylated using a fluoride deprotecting agent (e.g., tetra-n-butyl ammonium fluoride) to provide compound 44, which is then converted to the C16 carboxylic acid compound 45 using Dess-Martin periodinane (DMP) in pyridine/solvent to provide a C16 aldehyde intermediate followed by oxidation to the corresponding C16 carboxylic acid (using for example, sodium chlorite, 2-methyl-2-butene and sodium phosphate monobasic) to afford the diprotected (MOM) compound 45.

(+)-Pleuromutilin may also be derivatized to compound 49 (containing a C16 hydroxyl group, a C11 keto group and a C12 ethyl group) as per Scheme 12, FIG. 12 by first converting (+)-pleuromutilin to the C11 keto compound 46 (compound 46 also introduces a methyl ether group by conversion of the keto group at C3) using sulfuric acid/trimethylorthoformate in solvent. Compound 46 is converted to compound 47 in two steps by de-esterifying the hydroxymethylester at C14 using basic conditions (NaOH) and reducing the vinyl group at C12 under reducing conditions (e.g., Pd/H₂ under pressure). Compound 47 is then hydrosilylated at the C14 hydroxyl group (diphenylhydrosilane in weak base) and the hydrosilylated intermediate is subjected to iridium catalyzed ([Ir(OCH₃)(COD)]₂) C—H functionalization as described herein above (norbornene and 3,4,78-teramethyl-1,10-phenantholine, Me₄phen in solvent (e.g. THF) preferably at elevated temperature (e.g. 120-125° C.)) to provide cyclosilane compound 48 which is then exposed to a silyl deprotection agent (TBAF), followed by oxidizing conditions (e.g., meta-chloroperbenzoic acid (m-CPBA)) to provide compound 49, which contains a methoxy group at C3, a keto group at C11 and a hydroxyl group at C16.

Further derivitization of pleuromutilin compounds can be seen in Scheme 13, FIG. 13C. Compound 32 can be readily converted to the acetyl protected compound 54 in acetic anhydride and weak base to afford compound 54, which can be hydrosilylated at the C14 hydroxyl group using the usual conditions (dialkyl or diphenylchlorohydrosilane in weak base) to provide compound 55 which can be subjected to iridium catalyzed C—H functionalization on an adjacent methyl group (C15), followed by de-silylation (TBAF) and oxidation (m-CPBA) to afford compound 56.

FIG. 13A, Table 1, shows the introduction of various diamine functionalities at the C17 position of pleuromutilin derivative 57. As indicated in FIG. 13D, compound 57 is first reacted with Dess-Martin periodinane to provide the corresponding aldehyde (not shown, compound S40 in the examples section) which is then reacted with a diamine (as disclosed in compounds 58A-D) followed by reduction and silyl deprotection (HF/pyridine) and trifluoroacetic acid to provide the corresponding C17 diamine compounds 58A-D as their trifluoroacetate ammonium salts 58A-D.

Scheme 14, FIG. 14 shows the derivatization of a hydroxyl methyl group at C6 of compound 32 to provide a C6-normethyl pleuromutilin derivative (62). In this scheme, compound 32 is subjected to Dess-Martin periodinane/weak base to provide the corresponding aldehyde (59) which is subjected to rhodium-mediated decarbonylation (using RhCl(PPh₃)₃) to yield the protected C6 normethyl compound (60b) (lactone compound 60a was also produced). Compound 60b is then esterified with benzyloxyacetic acid in EDCl/DMAP to provide the benzyl protected ester at C14 to provide compound 61 which is subjected to reducing conditions (Pd(OH)₂/C in H₂ at high pressure) to remove the remaining protecting groups (benzyl and BOM) to provide compound 62.

To demonstrate the necessity of saturating the vinyl functionality before the iridium-catalyzed C—H functionalization process, the inventors also prepared silane S6 from 19 under a silylation procedure similar to that described in FIG. 15, Table S1, entry 6 (FIG. 18, Scheme 18). The iridium-catalyzed C—H functionalization process afforded a complex mixture of unidentified compounds which afforded the desired product S7 and the undesired diketone S8 in 14% and 8% yields after TBDPS-deprotection followed Tamao-Fleming oxidation (FIG. 18, Scheme 18). The formation S8 stemmed from the Markovnikov-hydrosilylation of the C19-C20 alkene occurred under the C—H functionalization conditions.

The C16 functionalization could also be achieved with 12-epi-mutilin derivative. 12-epi-Pleuromutilin derivative 20 underwent smooth BOM protection affording S9 in 93% yield (FIG. 19, Scheme 19). Hydrolysis of the glycolic ester fragment followed by saturation of C19-C20 alkene afforded S10 in 93% yield over two steps. C14 silylation with HSiPh₂Cl followed by iridium-catalyzed C—H functionalization afforded silacycle S11, which was converted to the diol S12 in 35% yield over three steps (FIG. 19, Scheme 19).

Selective protection of the primary alcohol of compound S12 with BOMCl followed by esterification between the secondary alcohol and benzyloxyacetic acid afforded the fully protected 12-epi-16-hydroxypleuromutilin derivative S14 (FIG. 20, Scheme 20). After global deprotection with reductive conditions, only the glycolic ester migration product S15 was observed in near quantitative yield. The migration product S15 could also be synthesized from compound S12 with a two-step sequence in 75% overall yield (FIG. 20, Scheme 20).

Thus, the description of the synthetic methods provided herein provides a general method for allowing the skilled practitioner to introduce functional groups on pleuromutilin or related derivatives and to provide pleuromutilin derivatives which were previously unknown or could not be readily made, thus opening a whole new class of antimicrobial compositions.

In embodiments, the present invention is directed to a method for synthesizing a compound according to the chemical structure 14A

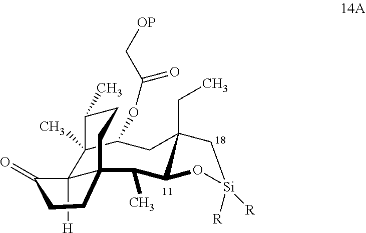

where R is C₁-C₃ alkyl (often methyl) or phenyl and P is a protecting group, preferably a silyl protecting group especially a TBDPS (tert.-Butyldiphenylsilyl) group, from compound 12A

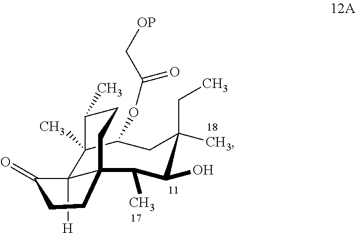

comprising reacting compound 12A with HSi(R)₂Cl, where P is a protecting group, preferably a silyl protecting group especially a TBDPS (tert.-Butyldiphenylsilyl) group and R is C₁-C₃ alkyl or phenyl (often methyl) group in a weak base (preferably triethylamine) to produce compound 13A

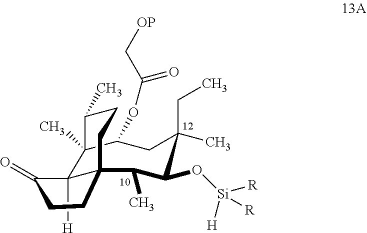

where P and R are the same as above, which is reacted in a dehydrogenative C—H silylation reaction catalyzed by iridium or ruthenium catalyst on the hydrosilane group to form cyclosilane compound 14A.

In embodiments, R is methyl to form compound 14a at a ratio of 4:1 compound 14a to 14b (FIG. 3). In alternative embodiments, R is phenyl. In embodiments, the dehydrogenative C—H silylation reaction is conducted using Methoxy(cyclooctadiene)iridium(I) dimer, 3,4,7,8-tetramethyl-1,10-phenthroline (Me₄phen) and norbornene in an appropriate solvent (e.g., THF) at elevated temperature (e.g. 100-150° C.).

In embodiments, the present invention is directed a method for synthesizing a compound according to the chemical structure 22A:

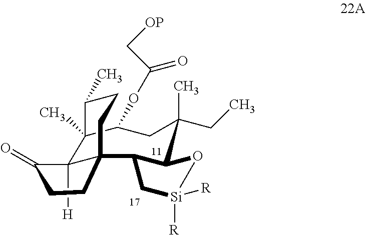

where R is a C₁-C₃ alkyl or phenyl group and P is a protecting group (preferably a silyl protecting group such as OTBDPS (tert.-buyldimethylsilyl group)), comprising reacting compound 20A according to the chemical structure:

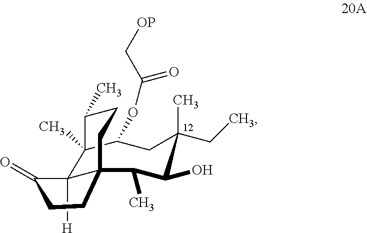

comprising reacting compound 20A with HSi(R)₂Cl, where P is a protecting group, preferably a silyl protecting group especially a TBDPS (tert.-Butyldiphenylsilyl) group and R is C₁-C₃ alkyl or phenyl (often methyl) group in a weak base (preferably triethylamine) to produce compound 21A.

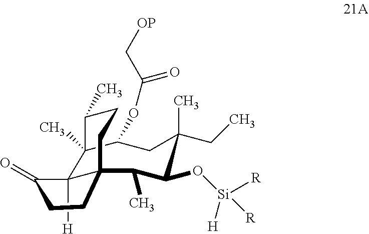

A where P and R are the same as described above, which is reacted in a dehydrogenative C—H silylation reaction catalyzed by iridium or ruthenium catalyst on the hydrosilane group to form cyclosilane compound 22A.

In embodiments, R is methyl to form compound 22a at a ratio of 4:1 compound 22a to 22b (FIG. 5, examples). In alternative embodiments, R is phenyl. In embodiments, the dehydrogenative C—H silylation reaction is conducted using Methoxy(cyclooctadiene)iridium(I) dimer, 3,4,7,8-tetramethyl-1,10-phenthroline (Me₄phen) and norbornene in an appropriate solvent (e.g., THF) at elevated temperature (e.g. 100-150° C.).

In embodiments, the present invention is directed a method for synthesizing a compound according to the chemical structure 31A:

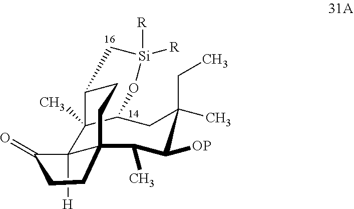

where P is a protecting group and R is a C₁-C₃ alkyl group or a phenyl group, comprising reacting compound 30P

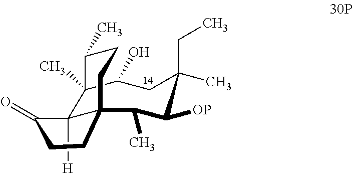

with HSi(R)₂Cl, where P is a protecting group, preferably a benzyloxylmethyl acetal (BOM) group and R is a C₁-C₃ alkyl or phenyl (often methyl) group in a weak base (preferably triethylamine) to produce compound 30B

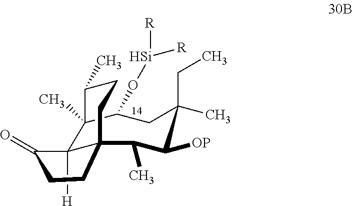

which is reacted in a dehydrogenative C—H silylation reaction catalyzed by iridium or ruthenium catalyst on the hydrosilane group to form cyclosilane compound 31A.

In embodiments, R is phenyl group and P is a benzyloxymethylacetal (BOM) group to form compound 31 (FIG. 8, examples). In alternative embodiments, R is methyl. In embodiments, the dehydrogenative C—H silylation reaction is conducted using Methoxy(cyclooctadiene)iridium(I) dimer, 3,4,7,8-tetramethyl-1,10-phenthroline (Me₄phen) and norbornene in an appropriate solvent (e.g., THF) at elevated temperature (e.g. 100-150° C.).

In embodiments, the present invention is directed a method for synthesizing a compound according to the chemical structure 48B:

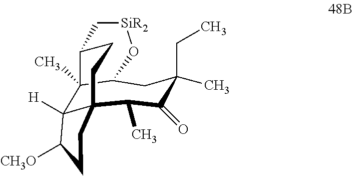

where R is a C₁-C₃ alkyl group or a phenyl group, comprising reacting compound 47

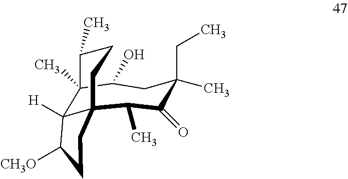

with HSi(RhCl, where R is a C₁-C₃ alkyl or phenyl (often phenyl) group in a weak base (preferably triethylamine) to produce compound 47B

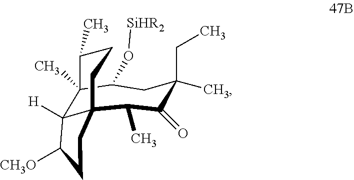

which is reacted in a dehydrogenative C—H silylation reaction catalyzed by iridium or ruthenium catalyst on the hydrosilane group to form cyclosilane compound 48B.

In embodiments, R is a phenyl group as in compound 48 (FIG. 12, examples). In alternative embodiments, R is methyl. In embodiments, the dehydrogenative C—H silylation reaction is conducted using Methoxy(cyclooctadiene)iridium(I) dimer, 3,4,7,8-tetramethyl-1,10-phenthroline (Me₄phen) and norbornene in an appropriate solvent (e.g., THF) at elevated temperature (e.g. 100-150° C.).

In further embodiments, the invention is directed to compound 12A, 13A, 14A or 14a. In further embodiments, the invention is directed to compound 20A, 21A, 22A or 22a. Instill other embodiments, the invention is directed to compound 30B, 30P, 31A or 31. In additional embodiments, the invention is directed to compound 47B, 48B or 48.

In more particular embodiments, the present invention is directed to a method for synthesizing a compound according to the chemical structure 14A

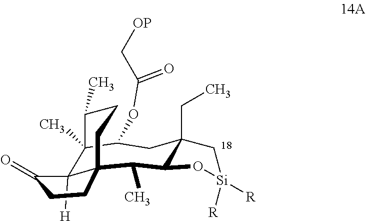

where R is C₁-C₃ alkyl (often methyl) or phenyl and P is a protecting group, preferably a silyl protecting group especially a TBDPS (tert.-Butyldiphenylsilyl) group, from compound 12A

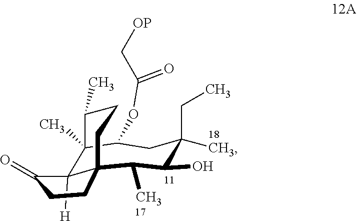

comprising reacting compound 12A with HSi(R)₂Cl, where P is a protecting group, preferably a silyl protecting group especially a TBDPS (tert.-Butyldiphenylsilyl) group and R is C₁-C₃ alkyl or phenyl (often methyl) group in a weak base (preferably triethylamine) to produce compound 13A

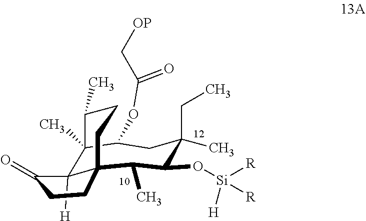

where P and R are the same as above, which is reacted in a dehydrogenative C—H silylation reaction catalyzed by iridium or ruthenium catalyst on the hydrosilane group to form cyclosilane compound 14A.

In embodiments, R is methyl to form compound 14a at a ratio of 4:1 compound 14a to 14b (FIG. 3). In alternative embodiments, R is phenyl. In embodiments, the dehydrogenative C—H silylation reaction is conducted using Methoxy(cyclooctadiene)iridium(I) dimer, 3,4,7,8-tetramethyl-1,10-phenthroline (Me₄phen) and norbornene in an appropriate solvent (e.g., THF) at elevated temperature (e.g. 100-150° C.).

In embodiments, the present invention is directed a method for synthesizing a compound according to the chemical structure 22A:

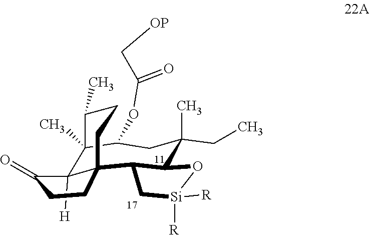

where R is a C₁-C₃ alkyl or phenyl group and P is a protecting group (preferably a silyl protecting group such as OTBDPS (tert.-buyldimethylsilyl group)), comprising reacting compound 20A according to the chemical structure:

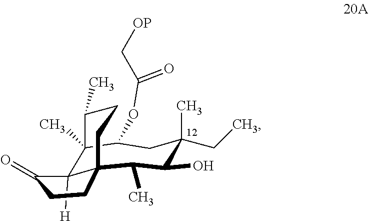

comprising reacting compound 20A with HSi(R)₂Cl, where P is a protecting group, preferably a silyl protecting group especially a TBDPS (tert.-Butyldiphenylsilyl) group and R is C₁-C₃ alkyl or phenyl (often methyl) group in a weak base (preferably triethylamine) to produce compound 21A.

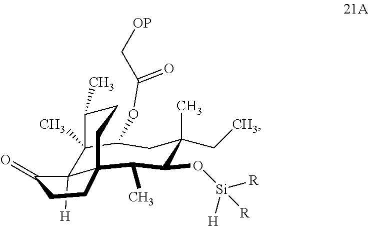

where P and R are the same as described above, which is reacted in a dehydrogenative C—H silylation reaction catalyzed by iridium or ruthenium catalyst on the hydrosilane group to form cyclosilane compound 22A.

In embodiments, R is methyl to form compound 22a at a ratio of 4:1 compound 22a to 22b (FIG. 5, examples). In alternative embodiments, R is phenyl. In embodiments, the dehydrogenative C—H silylation reaction is conducted using Methoxy(cyclooctadiene)iridium(I) dimer, 3,4,7,8-tetramethyl-1,10-phenthroline (Me₄phen) and norbornene in an appropriate solvent (e.g., THF) at elevated temperature (e.g. 100-150° C.).

In embodiments, the present invention is directed a method for synthesizing a compound according to the chemical structure 31A:

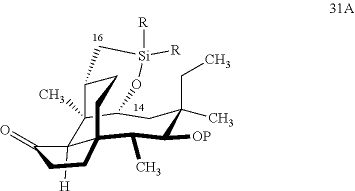

where P is a protecting group and R is a C₁-C₃ alkyl group or a phenyl group, comprising reacting compound 30P

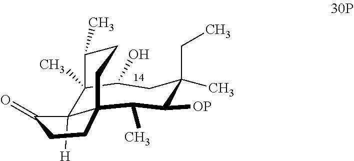

with HSi(R)₂Cl, where P is a protecting group, preferably a benzyloxylmethyl acetal (BOM) group and R is a C₁-C₃ alkyl or phenyl (often methyl) group in a weak base (preferably triethylamine) to produce compound 30B

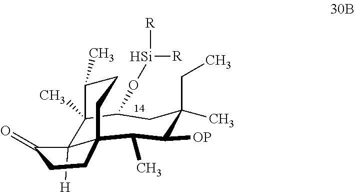

which is reacted in a dehydrogenative C—H silylation reaction catalyzed by iridium or ruthenium catalyst on the hydrosilane group to form cyclosilane compound 31A.

In embodiments, R is phenyl group and P is a benzyloxymethylacetal (BOM) group to form compound 31 (FIG. 8, examples). In alternative embodiments, R is methyl. In embodiments, the dehydrogenative C—H silylation reaction is conducted using Methoxy(cyclooctadiene)iridium(I) dimer, 3,4,7,8-tetramethyl-1,10-phenthroline (Me₄phen) and norbornene in an appropriate solvent (e.g., THF) at elevated temperature (e.g. 100-150° C.).

In embodiments, the present invention is directed a method for synthesizing a compound according to the chemical structure 48B:

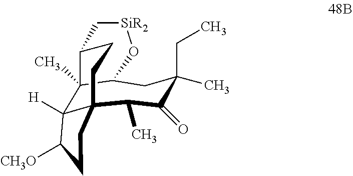

where R is a C₁-C₃ alkyl group or a phenyl group, comprising reacting compound 47

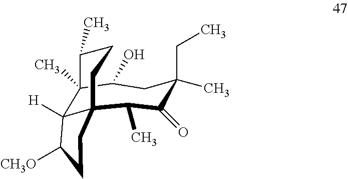

with HSi(R)₂Cl, where R is a C₁-C₃ alkyl or phenyl (often phenyl) group in a weak base (preferably triethylamine) to produce compound 47B

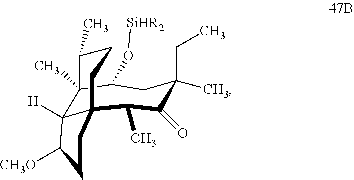

which is reacted in a dehydrogenative C—H silylation reaction catalyzed by iridium or ruthenium catalyst on the hydrosilane group to form cyclosilane compound 48B.

In embodiments, R is a phenyl group as in compound 48 (FIG. 12, examples). In alternative embodiments, R is methyl. In embodiments, the dehydrogenative C—H silylation reaction is conducted using Methoxy(cyclooctadiene)iridium(I) dimer, 3,4,7,8-tetramethyl-1,10-phenthroline (Me₄phen) and norbornene in an appropriate solvent (e.g., THF) at elevated temperature (e.g. 100-150° C.).

In further embodiments, the invention is directed to compound 12A, 13A, 14A or 14a. In further embodiments, the invention is directed to compound 20A, 21A, 22A or 22a. In still other embodiments, the invention is directed to compound 3013, 30P, 31A or 31. In additional embodiments, the invention is directed to compound 47B, 48B or 48.

In embodiments, the present invention is directed to pharmaceutical compositions comprising an antimicrobial (often an antibacterial) effective amount of a compound according to the present invention in combination with a pharmaceutically acceptable carrier, additive or excipient. Compositions according to the present invention may optionally comprise an effective amount of at least one additional bioactive agent, often an alternative antimicrobial agent.

In alternative embodiments, the present invention is directed methods of treating a microbial infection in a patient need comprising administering an effective amount of a compound or pharmaceutical composition to a Often, the microbial infection is a bacterial infection, including a drug resistant or multiple drug resistant bacterial infection, including drug resistant S. aureus (e.g. MRSA) infections.

Various embodiments and aspects of the present invention will be further described in the sections which follow.

### BRIEF DESCRIPTION OF THE FIGURES

FIG. 1 shows Scheme 1 which is directed to chemical structures of (+)-pleuromutilin (1), the C22 sulfonate 2, the semisynthetic C14 derivatives 34, and the 12-epi-mutilins 7 and 8.

FIG. 2 shows Scheme 2 which is directed to convergent fragment coupling en route to 12-epi-mutilin (11).

FIG. 3 shows Scheme 3 which is directed to A. Synthesis of the C18 oxidation product 15a. B. Shows a ball and stick representation of the X-ray structure of 19,20-dihydropleuromutilin (16). The C17-C10-C11-O11 and O11-C11-C12-C18 dihedral angles are 0.95° and 49.9°, respectively. The C17, C10, C11, C12, and C18 atoms are shown in blue, all other carbon atoms are shown in grey. Oxygen atoms are shown in red. Hydrogen atoms are omitted for clarity.

FIG. 4 shows Scheme 4 which is directed to the chemical synthesis of the aldehyde 17.

FIG. 5 shows Scheme 5 which is directed to the chemical synthesis of the C17 oxidation product 23a.

FIG. 6 shows Scheme 6 directed to the chemical synthesis of the aldehyde 24 from compound 23a in two steps.

FIG. 7 shows Scheme 7 which is directed to the chemical synthesis of the C17, C18 dioxidized product 28 through a series of chemical synthetic steps.

FIG. 8 shows Scheme 8 which is directed to the chemical synthesis of the C16-oxidized derivative 32 using the C14 hydroxyl substituent as a directing group.

FIG. 9 shows Scheme 9 which is directed to the chemical synthesis of the C16-oxidized derivatives 35 and 36.

FIG. 9A shows a Ball and stick representation of the X-ray structure of 16-hydroxy-19,20-dihydromutilin (37). The C14 and C16 carbon atoms are shown in blue. All other carbon atoms are shown in grey. Hydrogen atoms have been omitted for clarity.

FIG. 10 shows Scheme 10 which is directed to an alternative chemical synthetic route to the C16 silylation product 31.

FIG. 11 shows Scheme 11 which is directed to the chemical synthesis of the identified C6 carboxylic acid 45.

FIG. 12 shows Scheme 12 which is directed to C16 functionalization via 4-cpi-pleuromutilin (46).

FIG. 13 shows Scheme 13 which is directed to A. C3 silyl ether derivatives and B. C16 silyl ether derivatives employed in attempted C15 oxidation. C. Successful use of the C14 hydroxyl to direct C15 oxidation.

FIG. 13D shows Table 1, which is directed to the installation of diamine substituents at the C17 position. The figure indicates the isolated yield for each compound over 4 steps. Detailed synthetic conditions are found in the examples section of the present application.

FIG. 14 shows Scheme 2 which is directed to the chemical synthesis of C6-normethyl-19,20-dihydropleuromutilin (62).

FIG. 15 shows Table S1 which is directed to the optimization of the silane installation. ^{a}Detailed reaction conditions are found in the examples section of the present application. ^{b}Conversion is determined by the yield of the recovered starting material after flash column chromatography. ^{c}Isolated yields after purification by flash-column chromatography. ^{d}Reaction conducted on 4.2-g scale.

FIG. 16 shows Table S2 which is directed to the optimization of the Tamao-Fleming oxidation for compound 14a. ^{a}Reaction conducted on 100-mg scales unless otherwise noted. ^{b}For detailed reaction conditions, see Supporting Information. ^{c}Conversion determined by the yield of recovered starting material after column chromatography. ^{d}Isolated yields after purification by flash-column chromatography. ^{e}Complex mixture. ^{f}Yields for two steps. ^{g}Reaction conducted on 4.4-g scale.

FIG. 17 shows Table 3S which is directed to the optimization of the Tamao-Fleming oxidation of compounds 31, 41 and 42 as identified in the scheme. ^{a}For detailed reaction conditions, see Supporting Information. ^{b}Conversion determined by the yield of recovered starting material after column chromatography. ^{c}Isolated yields after purification by flash-column chromatography. ^{d}Complex mixture. ^{e}Yields for two steps.

FIG. 18 shows Scheme 18 which is directed to a C—H functionalization sequence without saturating the C₁₉-C₂₀ alkene.

FIG. 19 shows Scheme 18 which is directed to C16 functionalization with a 12-epi-mutilin derivative.

FIG. 20 shows Scheme 18 which is directed to installation of a glycolic ester fragment.

### DETAILED DESCRIPTION OF THE INVENTION

The following terms shall be used throughout the specification to describe the present invention. Where a term is not specifically defined herein, that term shall be understood to be used in a manner consistent with its use by those of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges that may independently be included in the smaller ranges are also encompassed within the invention, subject to any specifically excluded limit in the stated range. Where the stated range includes one or both of the limits, ranges excluding either both of those included limits are also included in the invention. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal, especially including a domesticated animal and preferably a human, to whom treatment, including prophylactic treatment (prophylaxis), with the compounds or compositions according to the present invention is provided. For treatment of those infections, conditions or disease states which are specific for a specific animal such as a human patient, the term patient refers to that specific animal. In most instances, the patient or subject of the present invention is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state, especially a bacterial infection including a drug resistant bacterial infection including a MRSA infection within the context of its use or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diastereomers, individual optical isomers/enantiomers or racemic mixtures and geometric isomers), pharmaceutically acceptable salts and prodrug forms. The term compound herein refers to stable compounds. Within its use in context, the term compound may refer to a single compound or a mixture of compounds as otherwise described herein. It is understood that the choice of substituents or bonds within a Markush or other group of substituents or bonds is provided to form a stable compound from those choices within that Markush or other group. The symbol [symbol] used alone or in the symbol [symbol] in a compound according to the present invention is used to represent an optional bond. Note that no more than one optional bond exists in a compound according to the present invention.

The term “adjacent” is used to describe the relationship (distance) in a pleuromutilin compound between a hydroxyl group and a methyl group to be functionalized with a hydroxyl group. In the present invention an important feature is the ability to hydrosilylate a hydroxyl group and catalyze (using an iridium or ruthenium, preferably iridium catalyst as described here) the formation of a cyclosilane with a methyl group in proximity to the hydrosilane group. Once obtained, the cyclosilane group is subjected to Tamao-Fleming oxidation (i.e., fluoride desilylation and oxidation) to provide the original free hydroxyl group and to introduce a free hydroxyl group on the adjacent methyl group. It is noted that the hydroxyl and methyl group which form the cyclosilane do not have to be substituted on vicinal carbon atoms in order to participate in the hydrosilylation and cyclosilylation reactions, but these groups have to be positioned within a compound in proximity to allow the formation of a cyclosilane group between the hydroxyl group and the methyl group to be functionalized.

The term “pharmaceutically acceptable” as used herein means that the compound or composition is suitable for administration to a subject to achieve the treatments described herein, without unduly deleterious side effects in light of the severity of the disease and necessity of the treatment.

The term “independently” is used herein to indicate that the variable, which is independently applied, varies independently from application to application.

The term “non-existent” or “absent” refers to the fact that a substituent is absent and the group to which such substituent is attached forms an additional bond with an adjacent atom or group.

The terms “treat”, “treating”, and “treatment”, etc., as used herein within context, also refers to any action providing a benefit to a patient at risk for any of the disease states or conditions (bacterial pathogens, especially MRSA infections) which can be treated pursuant to the present invention (e.g., inhibit, reduce the severity, cure, etc.). Treatment, as used herein, principally encompasses therapeutic treatment, but may also encompass both prophylactic and therapeutic treatment, depending on the context of the treatment. The term “prophylactic” when used in context, means to reduce the likelihood of an occurrence or in some cases, reduce the severity of an occurrence within the context of the treatment of a disease state or condition otherwise described herein.

The term “prevention” is used within context to mean “reducing the likelihood” of a condition or disease state from occurring as a consequence of administration or concurrent administration of one or more compounds or compositions according to the present invention, alone or in combination with another agent. Thus, the term prevention is used within the context of a qualitative measure and it is understood that the use of a compound according to the present invention to reduce the likelihood of an occurrence of a condition or disease state as otherwise described herein will not be absolute, but will reflect the ability of the compound to reduce the likelihood of the occurrence within a population of patients or subjects in need of such prevention.

The ter “gram negative bacteria” is used to describe any number of bacteria which are characterized in that they do not retain crystal violet stain used in the gram staining method of bacterial differentiation. These bacteria are further characterized by their cell walls, which are composed of a thing layer of peptidoglycans sandwiched between an outer membrane and an inner cytoplasmic cell membrane. Examplary gram negative bacteria include, for example, Escherichia sp., (Escherichia coli), as well as a larger number of pathogenic bacteria, including Salmonella sp. Shigella sp., Helicobacter sp. (e.g. H. pylori), Acetic acid bacteria, Legionella sp., Cyanobacteria sp., Neisseria sp. (Neisseria gonorrhaeae), Acinetobacter baumanii, Fusobacterium sp., Haemophilus sp. (Haemophilus influenzae), Klebsiella sp., Leptospiria, Nitrobacter sp., Proteus sp., Rickettsia sp., Serratia sp., Thiobacter sp., Treponema sp., Vibrio sp., and Yersinia sp., among others. Compounds according to the present invention are particularly useful for the treatment of gram negative bacterial infections, especially infections caused by the gram negative bacteria se forth above. In certain embodiments, the infection to be treated is caused by Staphylococcus aureus, especially MRSA, which is a gram positive bacteria.

The term “gram positive bacteria” is used to describe any number of bacteria which are characterized in that they do retain crystal violet stain used in the gram staining method of bacterial differentiation. These bacteria are further characterized by their cell walls, which are composed of a thick layer of peptidoglycans sandwiched underneath an outer membrane. Gram positive bacteria have no inner cytoplasmic cell membrane such as in the case of the gram negative bacteria. Exemplary gram positive bacteria include Actinomyces sp., Bacillus sp., especially Bacillus anthracis (anthrax), Clostridium sp., especially Clostridium tetani, Clostridium perfringens and Clostridium botulinum, Corynebacterium sp., Enterococcus sp., Gardnerella sp., Lactobacillus sp., Listeria sp., Mycobacterium sp., especially Mycobacterium tuberculosis, Nocardia sp., Propionibacterium sp., Staphylococcus sp., especially Staphylococcus aureus, Streptococcus sp., especially Streptococcus pneumonia, and Streptomyces sp., among others.

The term “bacterial infection” or infection is used to describe any disease state and/or condition in a patient or subject which is caused by a bacteria, especially including one or more of the bacteria which are described herein. The term “microbial infection” is used to describe a disease state and/or condition in a patient or subject which is caused by a microbe such as a bacteria, virus, fungus or protozoa.

The term “additional antibiotic” or “alternative antibiotic” is used to describe an agent which may be used to treat a bacterial infection which is other than the antibiotic agents pursuant to the present invention and may be used in cotherapy with compounds according to the present invention. Additional antibiotics which may be combined in therapy with antibiotic compounds according to the present invention include:

Aminoglycosides including amikacin, gentamycin, kanamycin, neomycin, netilmicin, tobramycin, paromomycin, streptomycin, spectinomycin;

Ansamycins, including geldanamycin, herbimycin and rifazimin;

Carbacephems, including, loracarbef, ertapenem, doripenem, imipenem/cilastatin and meropenem;

Cephalosporins, including cefadroxil, cefazolin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, cetriaxxone, cefepime, ceftaroline fosamil and ceftobiprole;

Glycopeptides, including teicoplanin, vancomycin, telavancin, dalbavancin and orivitavancin;

Lincosamides, including clindamycin and lincomycin;

Lipopeptides, including daptomycin;

Macrolides, including azithromycin, clarithromycin, dirithromycin, erythromycin, roxithromycin, troleandomycin, telithromycin and spiramycin;

Monobactams, including aztreonam:

Nitrofurans, including furazolidone and nitrofurantoin;

Oxazollidinones, including linezolid, posizolid, radezolid and torezolid;

Penicillins, including amoxicillin, ampicillin, azlocillin, carbenicillin, cloxacillin, dicloxacillin, flucloxacillin, mezlicillin, methicillin, nafcillin, oxacillin, penicillin G, penicillin V, piperacillin, temocillin, ticarcillin, amoxiciflin/clavulanate, ampcillin/sulbactam, piperacillin/tazobactam and ticarcillin/clavulanate;

Polypeptides, including bacitracin, colistin and polymixin B;

Quinolones/Fluoroquinolines, including ciprofloxacin, enoxacin, gatifloxacin, gemifloxacin, levofloxacin, lomefloxecin, moxifloxacin, naldixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfasalazine, sulfisoxazole, Trimethoprim-sulfamethoxazole and sulfonamidochysoidine;

Tetracyclines, including demeclocycline, doxycycline, minocycline, oxytetracycline and tetracycline;

Anti-Mycobacterial agents, including clofazimine, dapsone, capreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupiocin, platensimycin, quinupristin/dalfopristin, thiamphenicol, tigecycline, tinidazole and trimethoprim.

The term “MRSA” as used herein describes any strain of Staphylococcus aureus that has antibiotic resistance, including resistance to methicillin, nafcillin, oxacillin. Staphylococcus aureus (S. aureus) is a gram-positive bacterium that is frequently found in the human respiratory tract and on the human skin. Although S. aureus is not usually pathogenic, it is known to cause skin infections (e.g., boils), respiratory disease (e.g., pneumonia), bloodstream infections, bone infections (osteomyelitis), endocarditis and food poisoning. The bacterial strains that often produce infections generate protein toxins and also express cell-surface proteins that apparently bind and inactivate antibodies. MRSA is responsible for a number of very difficult-to-treat infections in humans. The resistance does render MRSA infections far more difficult to treat. MRSA is often labeled as being community acquired MRSA (“CA-MRSA”) and hospital acquired MRSA (“HA-MRSA”). MSSA (methicillin sensitive Staphylococcus aureus) refers to a strain of Staphylococcus aureus that exhibits sensitivity to methicillin.

The term “additional bioactive agent” including an “additional antibiotic” an “additional anti-Staph aureus agent”, including an “additional anti-MRSA agent” is used to describe a drug or other bioactive agent which itself is useful in the treatment of bacterial infections, including Staphylococcus aureus infections, especially including MRSA and is other than an antibiotic useful in the treatment of bacterial infections, especially gram negative bacterial infections, including Staphylococcus aureus, especially including MRSA infections as described herein.

These additional bioactive agents may be used to treat disease states and conditions which are commonly found in patients who also have Staphylococcus aureus infections, especially MRSA infections. These additional bioactive agents, include additional antibiotics, essential oils and alternative therapies which may be useful for the treatment of bacterial pathogens. In particular, antibiotics and other bioactive agents, including essential oils may be included in compositions and co-administered along with the antibiotics according to the present invention.

Preferred bioactive agents for the treatment of MRSA include, for example, oritavancin (Orbactiv), dalvavancin (Dalvance), tedizolid phosphate, (Sivextro), clindamycin, linezolid (Zyvox), mupirocin (Bactroban), trimethoprim, sulfamethoxazole, trimethoprim-sulfamethoxazole (Septra or Bactrim), tetracyclines (e.g., doxycycline, minocycline), vancomycin, daptomycin, fluoroquinolines (e.g. ciprofloxacin, levofloxacin), macrolides (e.g. erythromycin, clarithromycin, azithromycine) or mixtures thereof. In addition to antibiotics, alternative therapies may be used in combination with the antiobiotics pursuant to the present invention and include the use of manuka honey and/or essential oils such as tea tree oil, oregano oil, thyme, clove, cinnamon, cinnamon bark, Eucalyptus, rosemary, lemongrass, geranium, lavender, nutmeg and mixtures thereof.

Antibiotics which are useful in the treatment of Staphylococcus aureus infections (both MSSA and MRSA) depend upon the tissue where the infection is found and whether the Staphylococcus aureus infection is MSSA or MRSA. In general, antibiotics which are found useful in the treatment of general MSSA infections include, for example, f-lactams, oxacillin, nafcillin and cefazolin, which are often used. For general MRSA infections, vancomycin, daptomycin, linezolid, Quinupristin/dalfopristin, Cotrimoxazole, Ceftaroline and Telavancin are more often used.

For treatment of Staphylococcus aureus infections of the heart or its valves (Endocarditis) oxacillin, cefazolin, nafcillin or gentamycin are used for methicillin sensitive strains (MSSA). For MRSA infections of the heart or its valves, useful antibiotics include ciprofloxacin, rifampin, vancomycin and daptomycin as preferred agents.

For Staphylococcus aureus infections of soft tissues and skin the primary treatment using antibiotics for MSSA includes Cephalexin, Dicloxacillin, Clindamycin and Amoxicillin/clavulanate. For MRSA infections, the preferred antibiotics include Cotrimoxazole, Clindamycin, tetracyclines, Doxycycline, Minocycline and Linezolide, although others may be used.

For skin infections local application of antibiotics like Mupirocin 2% ointment are generally prescribed.

For lung infections or pneumonia—for MRSA cases Linezolid, Vancomycin and Clindamycin are preferred.

For bone and joint infections—for MSSA oxacillin, cefazolin, nafcillin and gentamycin are often used. For MRSA infections, Linezolid, Vancomycin, Clindamycin, Daptomycin and Coptrimoxazole are often used.

For infections of the brain and meninges infection (meningitis)—for MSSA oxacillin, cefazolin, nafcillin, and gentamycin are preferred. For MRSA infections, Linezolid, Vancomycin, Clindamycin, Daptomycin and Cotrimoxazole may be used.

For Toxic Shock Syndrome—for MSSA oxacillin, nafcillin and clindamycin are often used. For MRSA infections Linezolid, Vancomycin and Clindamycin are often used.

Each of the above antibiotics may be combined in methods of the present invention for treating bacterial pathogens, especially Staphylococcus aureus infections (MSSA or MRSA). In addition, one or more of these antibiotics may be combined with one or GPER modulators in pharmaceutical compositions for the treatment of bacterial pathogens, especially Staphylococcus aureus infections (MSSA or MRSA).

“Hydrocarbon” or “hydrocarbyl” refers to any monovalent (or divalent in the case of alkylene groups) radical containing carbon and hydrogen, which may be straight, branch-chained or cyclic in nature. Hydrocarbons include linear, branched and cyclic hydrocarbons, including alkyl groups, alkylene groups, saturated and unsaturated hydrocarbon groups including aromatic groups both substituted and unsubstituted, alkene groups (containing double bonds between two carbon atoms) and alkyne groups (containing triple bonds between two carbon atoms). In certain instances, the terms substituted alkyl and alkylene are sometimes used synonymously.

“Alkyl” refers to a fully saturated monovalent radical containing carbon and hydrogen, and which may be cyclic, branched or a straight chain containing from 1 to 12 carbon atoms (C₁-C₁₂ alkyl) and are optionally substituted. Examples of alkyl groups are methyl, ethyl, n-butyl, n-hexyl, n-heptyl, n-octyl, n-nonyl, n-decyl, isopropyl, 2-methyl-propyl, cyclopropyl, cyclopropylmethyl, cyclobutyl, cyclopentyl, cyclopentylethyl, cyclohexylethyl and cyclohexyl. Preferred alkyl groups are C₁-C₆ alkyl groups. “Alkylene” refers to a fully saturated hydrocarbon which is divalent (may be linear, branched or cyclic) and which is optionally substituted. Preferred alkylene groups are C₁-C₆ alkylene groups. Other terms used to indicate substituent groups in compounds according to the present invention are as conventionally used in the art.

The term “aryl” or “aromatic”, in context, refers to a substituted or unsubstituted monovalent aromatic radical having a single ring (e.g., benzene or phenyl) or fused rings (naphthyl, phenanthryl, anthracenyl). Preferably the aryl or aromatic group is a phenyl group, often an unsubstituted phenyl group, especially when used in hydrosilylating agents to produce cyclosilane intermediates. Other examples of aryl groups, in context, may include heterocyclic aromatic ring systems “heteroaryl” groups having one or more nitrogen, oxygen, or sulfur atoms in the ring (5- or 6-membered heterocyclic rings) such as imidazole, furyl, pyrrole, pyridyl, furanyl, thiene, thiazole, pyridine, pyrimidine, pyrazine, triazine, triazole, oxazole, among others, which may be substituted or unsubstituted as otherwise described herein.

The term “substituted” shall mean substituted at a carbon or nitrogen position within a molecule or moiety within context, a hydroxyl, carboxyl, cyano (C≡N), nitro (NO₂), halogen (preferably, 1, 2 or 3 halogens, especially on an alkyl, especially a methyl group such as a trifluoromethyl), alkyl group (preferably, C₁-C₂, more preferably, C₁-C₆), alkoxy group (preferably, C₁-C₆ alkyl or aryl, including phenyl and substituted phenyl), a C₁-C₆ thioether, ester (both oxycarbonyl esters and carboxy ester, preferably, C₁-C₆ alkyl or aryl esters) including alkylene ester (such that attachment is on the alkylene group, rather than at the ester function which is preferably substituted with a C₁-C₆ alkyl or aryl group), thioester (preferably, C₁-C₆ alkyl or aryl), halogen (preferably, F or Cl), nitro or amine (including a five- or six-membered cyclic alkylene amine, further including a C₁-C₆ alkyl amine or C₁-C₆ dialkyl amine which alkyl groups may be substituted with one or two hydroxyl groups), amido, which is preferably substituted with one or two C₁-C₆ alkyl groups (including a carboxamide which is substituted with one or two C₁-C₆ alkyl groups), alkanol (preferably, C₁-C₆ alkyl or aryl), or alkanoic acid (preferably, C₁-C₆ alkyl or aryl) or a thiol (preferably, C₁-C₆ alkyl or aryl), or thioalkanoic acid (preferably, C₁-C₆ alkyl or aryl). Preferably, the term “substituted” shall mean within its context of use alkyl, alkoxy, halogen (preferably F), ester, keto, nitro, cyano and amine (especially including mono- or di-C₁-C₆ alkyl substituted amines which may be optionally substituted with one or two hydroxyl groups). Any substitutable position in a compound according to the present invention may be substituted in the present invention, but often no more than 3, more preferably no more than 2 substituents (in some instances only 1 or no substituents) is present on a ring. Preferably, the term “unsubstituted” shall mean substituted with one or more H atoms.

The term “protecting group” or “blocking group” refers to a group which is introduced into a molecule by chemical modification of a functional group to obtain chemoselectivity in a subsequent chemical reaction. It plays an important role in providing precursors to chemical components which provide compounds according to the present invention. Protecting groups may be used to protect functional groups on hydroxyl groups or other functional groups in order to facilitate selective hydrosilylation and C—H dehydrogenation to form cyclosilane groups. Typical protecting groups are used on alcohol groups, amine groups, carbonyl groups, carboxylic acid groups, phosphate groups and alkyne groups among others. The use of blocking groups is well known in the art. Protecting groups are used to prevent or limit a functional group in a molecular entity or compound from taking place in an undesired reaction and are generally removed from a compound or molecular entity using selective conditions which otherwise don't effect or impact the compound or molecular entity other than to remove the protecting group.

Exemplary alcohol/hydroxyl protecting groups include acetyl (removed by acid or base), trifluoroacetyl (TFA), benzoyl (removed by acid or base), benzyl (removed by hydrogenolysis, β-methoxyethoxymethyl ether (MEM, removed by acid), dimethoxytrityl [bis-(4-methoxyphenyl)phenylmethyl] (DMT, removed by weak acid), methoxymethyl ether (MOM, removed by acid), Benzyloxymethyl (BOM, removed by acid or reducing conditions), methoxytrityl [(4-methoxyphenyl)diphenylmethyl], (MMT, Removed by acid and hydrogenolysis), β-methoxylbenzyl ether (PMB, removed by acid, hydrogenolysis, or oxidation), methylthiomethyl ether (removed by acid), pivaloyl (Piv, removed by acid, base or reductant agents), methanesulfonyl (Mesyl) and toluenesulfonyl (Tosyl). More stable than other acyl protecting groups, tetrahydropyranyl (THP, removed by acid), tetrahydrofuran (THF, removed by acid), trityl (triphenyl methyl, (Tr, removed by acid), silyl ether (e.g. trimethylsilyl or TMS, triethylsily; or TES; tert-butyldimethylsilyl or TBDMS, tert-butyldiphenylsilyl or TBDPS, tri-iso-propylsilyloxymethyl or TOM, and triisopropylsilyl or TIPS, all removed by acid or fluoride ion such as such as NaF, TBAF (tetra-n-butylammonium fluoride, HF-Py, or HF-NEt₃); methyl ethers (removed by TMSI in DCM, MeCN or chloroform or by BBr₃ in DCM) or ethoxyethlyl ethers (removed by strong acid).

Exemplary amine-protecting groups include carbobenzyloxy (Cbz group, removed by hydrogenolysis), p-Methoxylbenzyl carbon (Moz or MeOZ group, removed by hydrogenolysis), tert-butyloxycarbonyl (BOC group, removed by concentrated strong acid or by heating at elevated temperatures), 9-Fluorenylmethyloxycarbonyl (FMOC group, removed by weak base, such as piperidine or pyridine), acyl group (acetyl, benzoyl, pivaloyl, by treatment with base), benzyl (Bn groups, removed by hydrogenolysis), carbamate, removed by acid and mild heating, p-methoxybenzyl (PMB, removed by hydrogenolysis), 3,4-dimethoxybenzyl (DMPM, removed by hydrogenolysis), p-methoxyphenyl (PMP group, removed by ammonium cerium IV nitrate or CAN); tosyl (Ts group removed by concentrated acid and reducing agents, other sulfonamides, Mesyl, Nosyl & Nps groups, removed by samarium iodide, tributyl tin hydride.

Exemplary carbonyl protecting groups include acyclical and cyclical acetals and ketals (removed by acid), acylals (removed by Lewis acids) and dithianes (removed by metal salts or oxidizing agents).

Exemplary carboxylic acid protecting groups include methyl esters (removed by acid or base), benzyl esters (removed by hydrogenolysis), tert-butyl esters (removed by acid, base and reductants), esters of 2,6-disubstituted phenols (e.g. 2,6-dimethylphenol, 2,6-diisopropylphenol, 2,6-di-tert-butylphenol, removed at room temperature by DBU-catalyzed methanolysis under high-pressure conditions, silyl esters (removed by acid, base and organometallic reagents), orthoesters (removed by mild aqueous acid), oxazoline (removed by strong hot acid (pH<1, T>100° C.) or strong hot alkali (pH>12, T>100° C.)).

Exemplary phosphate group protecting groups including cyanoethyl(removed by weak base) and methyl (removed by strong nucleophiles, e.g. thiophenol/TEA).

Exemplary terminal alkyne protecting groups include propargyl alcohols and silyl groups.

The term “pharmaceutically acceptable salt” or “salt” is used throughout the specification to describe a salt form of one or more of the compositions herein which are presented to increase the solubility of the compound in saline for parenteral delivery or in the gastric juices of the patient's gastrointestinal tract in order to promote dissolution and the bioavailability of the compounds. Pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Sodium and potassium salts may be preferred as neutralization salts of carboxylic acids and free acid phosphate containing compositions according to the present invention. The term “salt” shall mean any salt consistent with the use of the compounds according to the present invention. In the case where the compounds are used in pharmaceutical indications, including the treatment of prostate cancer, including metastatic prostate cancer, the term “salt” shall mean a pharmaceutically acceptable salt, consistent with the use of the compounds as pharmaceutical agents.

The term “coadministration” shall mean that at least two compounds or compositions are administered to the patient at the same time, such that effective amounts or concentrations of each of the two or more compounds may be found in the patient at a given point in time. Although compounds according to the present invention may be co-administered to a patient at the same time, the term embraces both administration of two or more agents at the same time or at different times, provided that effective concentrations of all coadministered compounds or compositions are found in the subject at a given time. Compounds according to the present invention may be administered with one or more additional bioactive agents, especially including an additional antibiotic for purposes of treating bacterial, especially gram negative bacteria and certain types of gram positive bacteria.

Pharmaceutical compositions comprising combinations of an effective amount of at least one compound disclosed herein, often a according to the present invention and one or additional compounds as otherwise described herein, all in effective amounts, in combination with a pharmaceutically effective amount of a carrier, additive or excipient, represents a further aspect of the present invention. These may be used in combination with at least one additional, optional bioactive agents, especially antibiotics as otherwise disclosed herein.

The compositions of the present invention may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir, among others. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally (including via intubation through the mouth or nose into the stomach), intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially to treat skin bacterial infections or other diseases which occur in or on the skin. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition of the instant invention that may be combined with the carrier materials to produce a single dosage form will vary depending upon the host and disease treated, the particular mode of administration. Preferably, the compositions should be formulated to contain between about 0.05 milligram to about 750 milligrams or more, more preferably about 1 milligram to about 600 milligrams, and even more preferably about 10 milligrams to about 500 milligrams of active ingredient, alone or in combination with at least one additional compound which may be used to treat a pathogen, especially a bacterial (often a gram-negative bacterial) infection or a secondary effect or condition thereof.

Methods of treating patients or subjects in need for a particular disease state or condition as otherwise described herein, especially a pathogen, especially a bacterial infection, in particular, a gram-negative bacterial infection, comprise administration of an effective amount of a pharmaceutical composition comprising therapeutic amounts of one or more of the novel compounds described herein and optionally at least one additional bioactive (e.g. additional antibiotic) agent according to the present invention. The amount of active ingredient(s) used in the methods of treatment of the instant invention that may be combined with the carrier materials to produce a single dosage form will vary depending upon the host treated, the particular mode of administration. For example, the compositions could be formulated so that a therapeutically effective dose of between about 0.001, 0.01, 0.1, 1, 5, 10, 15, 20, 25, 30, 35, 40, 45, 50, 55, 60, 65, 70, 75, 80, 85, 90 or 100 mg/kg of patient/day or in some embodiments, greater than 100, 110, 120, 130, 140, 150, 160, 170, 180, 190 or 200 mg/kg of the novel compounds can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

A patient or subject (e.g. a human) suffering from a bacterial infection can be treated by administering to the patient (subject) an effective amount of a compound according to the present invention including pharmaceutically acceptable salts, solvates or polymorphs, thereof optionally in a pharmaceutically acceptable carrier or diluent, either alone, or in combination with other known antibiotic or pharmaceutical agents, preferably agents which can assist in treating the bacterial infection or ameliorate the secondary effects and conditions associated with the infection. This treatment can also be administered in conjunction with other conventional therapies known in the art.

The present compounds, alone or in combination with other agents as described herein, can be administered by any appropriate route, for example, orally, parenterally, intravenously, intradermally, subcutaneously, or topically, in liquid, cream, gel, or solid form, or by aerosol form.

The active compound is included in the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 ng/kg to 300 mg/kg, preferably 0.1 to 100 mg/kg per day, more generally 0.5 to about 25 mg per kilogram body weight of the recipient/patient per day. A typical topical dosage will range from about 0.01-3% wt/wt in a suitable carrier.

The compound is conveniently administered in any suitable unit dosage form, including but not limited to one containing less than 1 mg, 1 mg to 3000 mg, preferably 5 to 500 mg of active ingredient per unit dosage form. An oral dosage of about 25-250 mg is often convenient.

The active ingredient is preferably administered to achieve peak plasma concentrations of the active compound of about 0.00001-30 mM, preferably about 0.1-30 μM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as other anticancer agents, antibiotics, antifungals, antiinflammatories, or antiviral compounds. In certain preferred aspects of the invention, one or more chimeric antibody-recruiting compound according to the present invention is coadministered with another anticancer agent and/or another bioactive agent, as otherwise described herein.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compounds are prepared with carriers that will protect the compound against rapid elimination from the body, such as a controlled and/or sustained release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions or cholestosomes may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s)(such as stearoyl phosphatidyl ethanolamine, stearoyl phosphatidyl choline, arachadoyl phosphatidyl choline, and cholesterol) in an inorganic solvent that is then evaporated, leaving behind a thin fACM of dried lipid on the surface of the container. An aqueous solution of the active compound are then introduced into the container. The container is then swirled by hand to free lipid material from the sides of the container and to disperse lipid aggregates, thereby forming the liposomal suspension.

### Chemistry

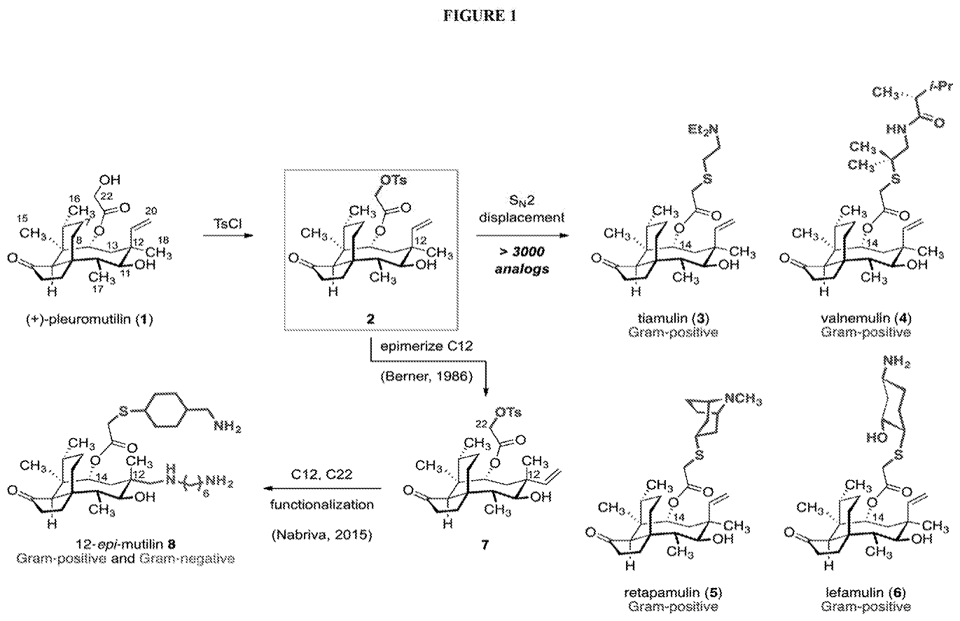

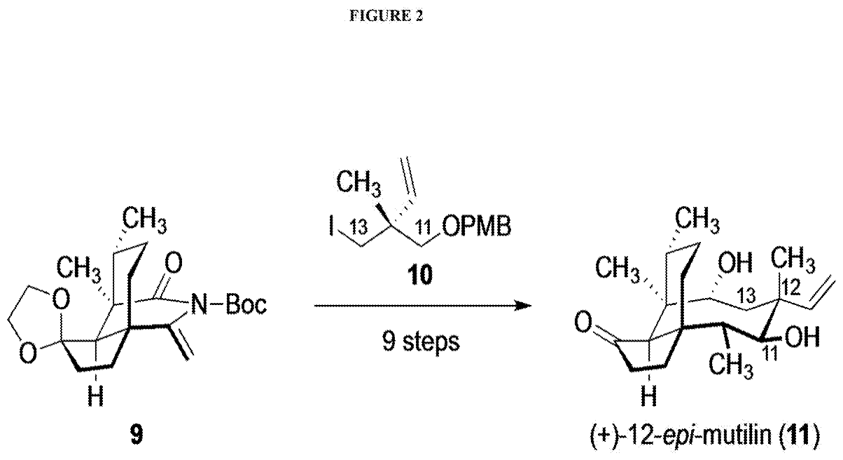

Compounds according to the present invention are synthesized according to the Schemes which are presented in the attached Figures as described in the Brief Description of the Figures section. FIGS. 1-14 and 18-20 set forth specific synthetic chemical schemes of compounds which find use in the present invention. The following examples provide detail of the various reactions which are used or may be used to produce compounds according to the present invention. Those compounds which are not specifically exemplified herein may be synthesized by analogy readily from the detailed description of the synthetic methodologies which are presented in the example section which follows and following the general synthetic chemical teachings which are set forth herein.

### EXAMPLES

General Experimental Procedures. All reactions were performed in single-neck, flame-dried, round-bottomed flasks fitted with rubber septa under a positive pressure of argon, unless otherwise noted. Air- and moisture-sensitive liquids were transferred via syringe or stainless steel cannula, or were handled in a nitrogen-filled drybox (working oxygen level <5 ppm). Organic solutions were concentrated by rotary evaporation at 30-33° C. Intermediates were purified using a Biotage Isolera system, employing polypropylene cartridges preloaded with silica gel (60 Å, 40-63 μm particle size, purchased from Silicycle, Quebec City, Canada) or neutral aluminium oxide (60 Å, 50-200 μm particle size, purchased from Acros Organics, New Jersey, USA). Samples were eluted using a flow rate of 12-50 mL/min, with detection by UV (254 nm). Analytical thin-layered chromatography (TLC) was performed using glass plates pre-coated with silica gel (0.25 mm, 60 Å pore size) impregnated with a fluorescent indicator (254 nm). TLC plates were visualized by exposure to ultraviolet light (UV) and/or submersion in aqueous ceric ammonium molybdate solution (CAM), acidic p-anisaldehyde solution (PAA), or aqueous potassium permanganate solution (KMnO₄), followed by brief heating on a hot plate (120° C., 10-15 s).
Materials. Commercial solvents and reagents were used as received with the following exceptions. Toluene were purified according to the method of Pangborn et al.¹ Dichloromethane was purified according to the method of Pangborn et al,¹ degassed by three freeze-pump-thaw cycles, and stored under an atmosphere of argon over 4 Å molecular sieves before use. 1,2-Dichloroethane, acetone, chloroform, and pyridine were distilled from calcium hydride under an atmosphere of nitrogen immediately before use. Commercial anhydrous N,N-dimethylformamide (Sigma-Aldrich Corporation, St. Louis, Mo.) was degassed by three freeze-pump-thaw cycles and stored over activated 4A MS under an atmosphere of nitrogen before use. Tetrahydrofuran was distilled from sodium-benzophenone under an atmosphere of nitrogen immediately before use. Triethylamine and N,N-diisopropylethylamine was distilled from calcium hydride under an atmosphere of nitrogen immediately before use. Methanol was distilled from magnesium under an atmosphere of nitrogen immediately before use.
Instrumentation. Instrumentation. Proton nuclear magnetic resonance spectra (¹H NMR) were recorded at 400, 500, or 600 MHz at 24° C., unless otherwise noted. Chemical shifts are expressed in parts per million (ppm, δ scale) downfield from tetramethylsilane and are referenced to residual protium in the NMR solvent (CHCl₃, δ 7.26; CD₂HOD, δ 3.30; CDHCl₂, δ 5.33; C₆HD₅, δ 7.16). Data are represented as follows: chemical shift, multiplicity (s=singlet, d=doublet, t=triplet, q=quartet, sep=septet, m=multiplet and/or multiple resonances, br=broad), integration, coupling constant in Hertz, and assignment. Proton-decoupled carbon nuclear magnetic resonance spectra (¹³C NMR) were recorded at 100, 125, or 150 MHz at 24° C., unless otherwise noted. Chemical shifts are expressed in parts per million (ppm, δ scale) downfield from tetramethylsilane and are referenced to the carbon resonances of the solvent (CDCl₃, δ 77.0; CD₃OD, δ 49.0; CD₂Cl₂, δ 54.0; C₆D_{b}, δ 128.1). Distortionless enhancement by polarization transfer spectra [DEPT (135)] were recorded at 100, 125, or 150 MHz at 24° C., unless otherwise noted. ¹³C NMR and DEPT (135) data are combined and represented as follows: chemical shift, carbon type [obtained from DEPT (135) experiments]. Proton-decoupled fluorine nuclear magnetic resonance spectra (¹⁹F NMR) were recorded at 375 MHz or 470 MHz at 24° C., unless otherwise noted. Chemical shifts are expressed in parts per million (ppm, scale) downfield from fluorotrichloromethane. Attenuated total reflectance Fourier transform infrared spectra (ATR-FTIR) were obtained using a Thermo Electron Corporation Nicolet 6700 FTIR spectrometer referenced to a polystyrene standard. Data are represented as follows: frequency of absorption (cm⁻¹), intensity of absorption (s=strong, m=medium, w=weak, br=broad). High-resolution mass spectrometry (HRMS) were obtained on a Waters UPLC/HRMS instrument equipped with a dual API/ESI high-resolution mass spectrometry detector and photodiode array detector. Unless otherwise noted, samples were eluted over a reverse-phase C18 column (1.7 μm particle size, 2.1×50 mm) with a linear gradient of 5% acetonitrile-water containing 0.1% formic acid→95% acetonitrile-water containing 0.1% formic acid over 1.6 min, followed by 100% acetonitrile containing 0.1% formic acid for 1 min, at a flow rate of 600 μL/min.

### Synthetic Procedures.

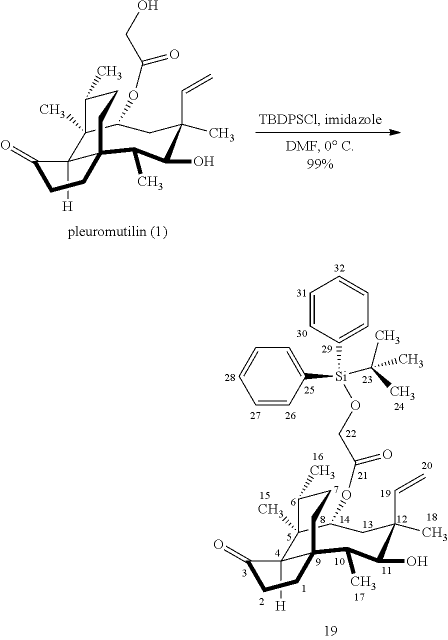

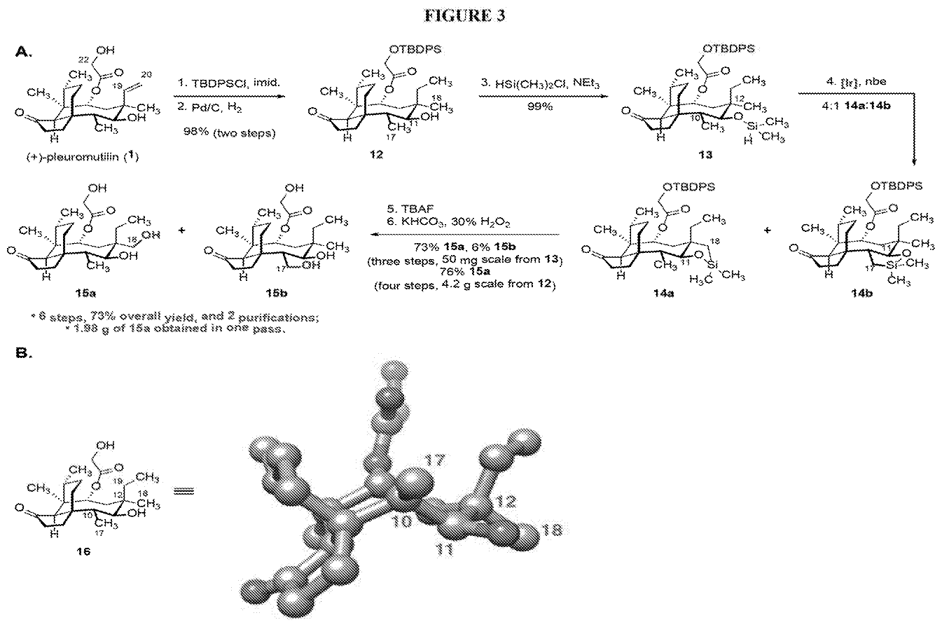

### Synthesis of O-tert-butyldiphenylsilylpleuromutilin (19, FIG. 3, Scheme 3)

tert-Butyl(chloro)diphenylsilane (3.43 mL, 13.2 mmol, 1.10 equiv) was added dropwise via syringe to a solution of pleuromutilin (1, 4.54 g, 12.0 mmol, 1 equiv) and imidazole (1.63 g, 24.0 mmol, 2.00 equiv) in N,N-dimethylformamide (90 mL) at 0° C. The reaction mixture was stirred for 50 min at 0° C. The product mixture was transferred to a separatory funnel that had been charged with ether (200 mL). The layers that formed were separated and the organic layer was washed with water (3×25 mL). The organic layer was dried over sodium sulfate. The dried solution was filtered and the filtrate was concentrated. The residue obtained was purified by automated flash-column chromatography (eluting with hexanes initially, grading to 50% ethyl acetate-hexanes, linear gradient) to afford O-tert-butyldiphenylsilylpleuromutilin (19) as an amorphous white solid (7.40 g, 99%).

R_{f}=0.48 (5% ether-dichloromethane; UV, CAM, PAA). ¹H NMR (400 MHz, CD₂Cl₂) δ 7.68-7.65 (m, 4H, 2×H₂₇, 2×H₃₁), 7.56-7.36 (m, 6H, 2×H₂₆, 1×H₂₈, 2×H₃₀, 1×H₃₂), 6.53 (dd, J=17.6, 11.2 Hz, 1H, H₁₉), 5.78 (d, J=8.4 Hz, 1H, H₁₄), 5.35 (d, J=11.2 Hz, 1H, 1×H₂₀), 5.22 (d, J=17.6 Hz, 1H, 1×H₂₀), 4.14 (dd, J=22.8, 6.0 Hz, 2H, H₂₂), 3.35 (dd, J=10.0, 3.6 Hz, 1H, H₁₁), 2.39-2.32 (m, 1H, H₁₀), 2.26-2.00 (m, 5H, 2×H₂, 1×H₄, 1×H₁₃, OH), 1.81-1.75 (m, 1H, 1×H₈), 1.67-1.52 (m, 3H, 1×H₁, 1×H₆, 1×H₇), 1.50-1.44 (m, 2H, 1×H₁, 1×H₇), 1.36 (s, 3H, H₁₃), 1.25-1.19 (m, 1H, 1×H₈), 1.16-1.12 (m, 4H, 1×H₃, 3×H₁₈), 1.07 (s, 9H, H₂₄), 0.87 (d, J=6.8 Hz, 3H, H₁₇), 0.60 (d, J=6.8 Hz, 3H, H₁₆). ¹³C NMR (100 MHz, CD₂Cl₂) δ 317.4 (C), 170.3 (C), 140.1 (CH), 136.1 (CH), 133.4 (C), 130.4 (CH), 128.3 (CH), 128.3 (CH), 117.8 (CH₂), 75.1 (CH), 69.0 (CH), 63.4 (CH₂), 58.6 (CH), 46.0 (C), 45.3 (C), 44.5 (CH₂), 42.4 (C), 37.4 (CH), 36.6 (CH), 35.0 (CH₂), 30.9 (CH₂), 27.5 (CH₂), 27.0 (CH₃), 26.7 (CH₃), 25.4 (CH₂), 19.6 (C), 17.0 (CH), 15.2 (CH₃), 11.8 (CH₃). IR (ATR-FTIR), cm⁻¹: 2932 (w), 1735 (m), 1462 (w), 1140 (m), 113 (s), 1015 (w), 824 (m), 701 (s). HRMS-ESI (m/z): [M+Na]⁺ calcd for C₃₈H₅₂NaO₅Si, 639.3482; found, 639.3486. [α]_{D}²⁵=+27° (c=1.0, CHCl₃).

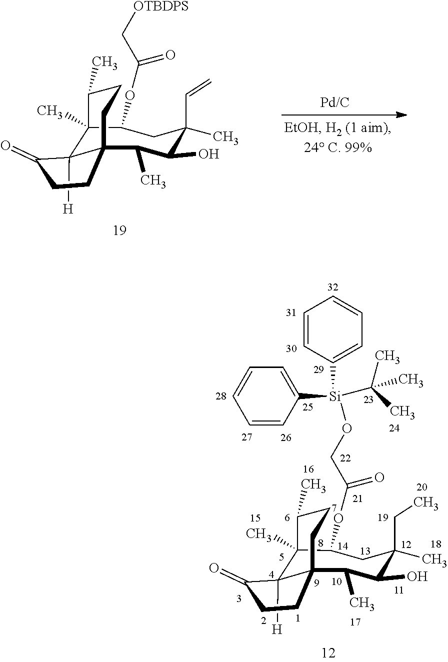

### Synthesis of O-tert-butyldiphenylsilyl-19,20-dihydropleuromutilin (12, FIG. 3, Scheme 3)

Palladium on carbon (5 wt. % loading, 1.28 g, 600 μmol, 0.05 equiv) was added to a solution of O-tert-butyldiphenylsilylpleuromutilin (19, 7.40 g, 12.0 mmol, 1 equiv) in ethanol (75 mL) at 24° C. The reaction vessel was evacuated and re-filled using a balloon of dihydrogen. This process was repeated four times. The reaction mixture was stirred for 12 h at 24° C. The product mixture was filtered through a short column of celite and the short column was rinsed with dichloromethane (250 mL). The filtrates were combined and the combined filtrates were concentrated to afford O-tert-butyldiphenylsilyl-19,20-dihydropleuromutilin (12) as an amorphous white solid (7.43 g, 99%).

R_{f}=0.54 (20% ethyl acetate-hexanes; UV, CAM, PAA). ¹H NMR (400 MHz, CDCl₃) δ 7.68-7.66 (m, 4H, 2×H₂₇, 2×H₃₁), 7.45-7.35 (m, 6H, 2×H₂₆, 1×H₂₃, 2×H₁₀, 1×H₃₂), 5.68 (d, J=8.4 Hz, 1H, H₆), 4.15 (dd, J=23.2, 6.8 Hz, 2H, H₂₂), 3.40 (t, J=5.8 Hz, 1H, H₁₁), 2.48-2.41 (m, 1H, H₁₀), 2.25-2.08 (m, 2H, H₂), 2.15 (s, 1H, H₄), 1.84-1.75 (m, 2H, 1×H₈, 1×H₁₃), 1.69-1.52 (m, 6H, 1×H₁, 1×H₆, 1×H₇, 1×H₁₃, 1×H₁₉, 1×OH), 1.49-1.43 (m, 1H, 1×H₇), 1.40 (s, 3H, H₁₅), 1.35-1.29 (m, 1H, 1×H₁), 1.15-1.10 (m, 1H, 1×H₈), 1.07 (s, 9H, H₂₄), 0.97-0.93 (m, 6H, 3×H₁₇, 3×H₁₈), 0.90-0.83 (m, 1H, 1×H₁₉), 0.76 (t, J=7.4 Hz, 3H, H₂₀), 0.63 (d, J=6.4 Hz, 3H, H₁₆). ¹³C NMR (100 MHz, CDCl₃) δ 217.2 (C), 170.0 (C), 135.5 (CH), 132.8 (C), 129.9 (CH), 127.8 (CH), 76.6 (CH), 68.7 (CH), 62.8 (CH₂), 58.6 (CH), 45.8 (C), 41.8 (C), 41.1 (CH₂), 40.9 (C), 36.7 (CH), 34.5 (CH), 34.3 (CH₂), 30.3 (CH₂), 26.8 (CH₂), 26.6 (CH₃), 26.3 (CH₃), 24.9 (CH₂), 20.8 (CH₂), 19.2 (C), 16.5 (CH₃), 14.9 (CH₃), 11.0 (CH₃), 8.3 (CH₃). IR (ATR-FTIR), cm⁻¹: 2933. (w), 2860 (w), 1734 (m), 1462 (w), 1428 (w), 1215 (w), 1141 (m), 1113 (s), 1008 (w), 824 (m), 702 (s), 505 (s). HRMS-ESI (m/z): [M+Na]⁺ calcd for C₃₈H₅₄NaO₅Si, 641.3638; found, 641.3631. [α]=+32° (c=1.0, CHCl₃).

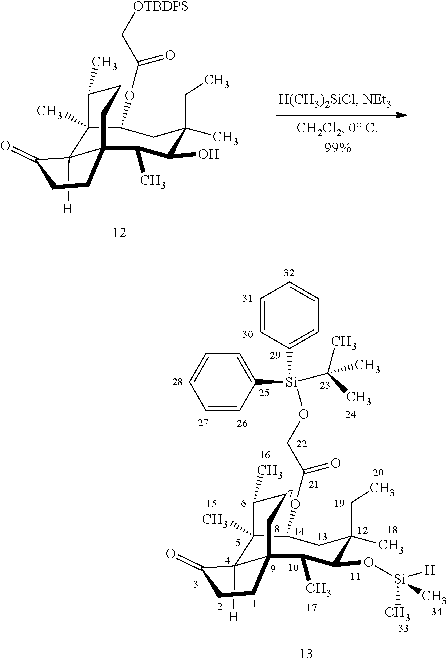

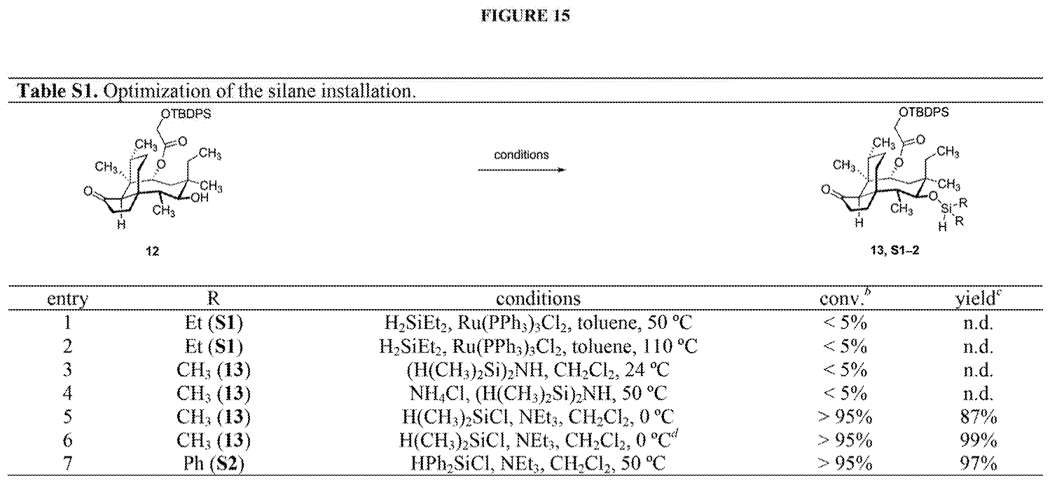

### Synthesis of Silane 13 (FIG. 3, Scheme 3 and FIG. 15. Table S1, Entry 6)

Dimethylchorosilane (1.50 mL, 13.5 mmol, 2.00 equiv) was added dropwise via syringe to a solution of O-tert-butyldiphenylsilyl-19,20-dihydropleuromutilin [12, 4.17 g, 6.74 mmol, 1 equiv, dried by azeotropic distillation with benzene (50 mL)] and trietylamine (3.75 mL, 27.0 mmol, 4.00 equiv) in dichloromethane (42 mL) at 0° C. The reaction mixture was stirred for 30 min at 0° C. The product mixture was diluted sequentially with pentane (50 mL) and aqueous potassium phosphate buffer solution (pH 7, 0.10 M, 15 mL) at 0° C. The diluted mixture was transferred to a separatory funnel and the layers formed were separated. The aqueous layer was extracted with dichloromethane (3×50 mL). The organic layers were combined and the combined organic layers were dried over sodium sulfate. The dried solution was filtered and the filtrate was concentrated to dryness to afford silane 13 as an amorphous white solid (4.57 g, 99%). The silane 13 prepared this way was analytically pure and was used in the next step without further purification.

R_{f}=0.57 (10% ether-hexanes; UV, CAM, PAA). ¹H NMR (400 MHz, C₆D₆) δ 7.77-7.73 (m, 4H, 2×H₂₇, 2×H₃₁), 7.21-7.16 (m, 6H, 2×H₂₆, 1×H₂₈, 2×H₃₀, 1×H₃₂), 5.67 (d, J=8.0 Hz, 1H, H₁₄), 4.80 (sep, J=2.8 Hz, 1H, Si—H), 4.16 (s, 2H, H₂₂), 3.22 (d, J=6.0 Hz, 1H, H₁₁), 2.41-2.34 (m, 1H, H₁₀), 1.96-1.89 (m, 1H, 1×H₉), 1.85-1.80 (m, 2H, H₂), 1.78-1.72 (m, 2H, 1×H₄, 1×H₁₉), 1.70-1.63 (m, 1H, H₆), 1.61 (s, 3H, H₁₈), 1.57-1.45 (m, 2H, 1×H₇, 1×H₁₃), 1.41-1.24 (m, 3H, 1×H₁, 1×H₈, 1×H₁₃), 1.16 (s, 9H, H₂₄), 1.10-0.99 (m, 2H, 1×H₁, 1×H₇), 0.86-0.77 (m, 6H, 3×H₁₈, 3×H₂₀), 0.77-0.72 (m, 4H, 1×H₈, 3×H₁₇), 0.66 (d, J=7.2 Hz, 3H, H₁₆), 0.12 (app d, 6H, 3×H₃₃, 3×H₃₄). ¹³C NMR (100 MHz, C₆D₆) δ 214.8 (C), 169.4 (C), 135.7 (CH), 133.0 (C), 129.8 (CH), 127.8 (CH), 127.8 (CH), 80.1 (CH), 68.4 (CH), 62.9 (CH₂), 58.0 (CH), 45.0 (C), 41.9 (C), 41.4 (C), 40.9 (CH₂), 36.6 (CH), 35.0 (CH), 34.0 (CH₂), 30.2 (CH₂), 27.1 (CH₂), 26.8 (CH), 26.5 (CH), 25.0 (CH₂), 21.1 (CH₂), 19.1 (C), 16.4 (CH₃), 14.8 (CH₃), 11.9 (CH₃), 8.3 (CH₃), −0.82 (CH₃), −0.84 (CH₃). IR (ATR-FTIR), cm⁻¹: 2969 (w), 1738 (s), 1366 (m), 1218 (m), 1143 (m), 1115 (m), 912 (m). HRMS-ESI (m/z): [M-Si(CH₃)₂+Na]⁺ calcd for C₃₈H₅₄NaO₅Si, 641.3638; found, 641.3643. [α]_{D}²⁵=+34° (c=1.0, CHCl₃).

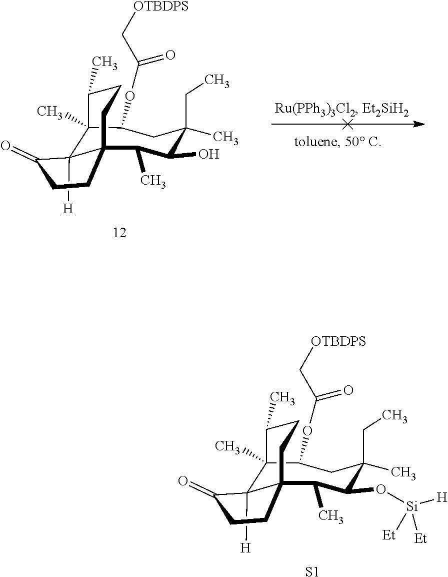

### Attempted Synthesis of Silane S1 (FIG. 15, Table S1, Entry 1)

A solution of diethylsilane (2.5 μL, 19.4 mmol, 1.20 equiv) in toluene (50 μL) was added dropwise via syringe to a solution of 0-tert-butyldiphenylsilyl-19,20-dihydropleuromutilin [12, 10.0 mg, 16.2 μmol, 1 equiv, dried by azeotropic distillation with benzene (500 μL)] and tris(triphenylphosphine)ruthenium(II) dichloride (0.300 mg, 0.300 μmol, 2.00 mol %) in toluene (100 μL) at 24° C. in the glovebox. The reaction vessel was sealed and the sealed vessel was removed from the glovebox. The reaction vessel was placed in an oil bath that had been previously heated to 50° C. The reaction mixture was stirred and heated for 12 h at 50° C. The product mixture was concentrated to dryness and the residue obtained was purified by automated flash-column chromatography (eluting with hexanes initially, grading to 50% ethyl acetate-hexanes, linear gradient) to afford the recovered starting material 12 as an amorphous white solid (9.9 mg, 99%).

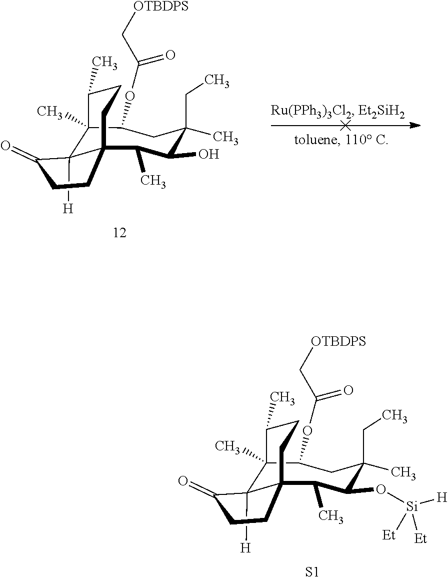

### Attempted Synthesis of Silane S1 (FIG. 15, Table S1, Entry 2)

A solution of diethylsilane (2.5 μL, 19.4 mmol, 1.20 equiv) in toluene (50 μL) was added dropwise via syringe to a solution of O-tert-butyldiphenylsilyl-19,20-dihydropleuromutilin [12, 10.0 mg, 16.2 μmol, 1 equiv, dried by azeotropic distillation with benzene (5001 μL)] and tris(triphenylphosphine)ruthenium(II) dichloride (0.300 mg, 0.300 μmol, 2.00 mol %) in toluene (100 μL) at 24° C. in the glovebox. The reaction vessel was sealed and the sealed vessel was removed from the glovebox. The reaction vessel was placed in an oil bath that had been previously heated to 110° C. The reaction mixture was stirred and heated for 12 h at 110° C. The product mixture was concentrated to dryness and the residue obtained was purified by automated flash-column chromatography (eluting with hexanes initially, grading to 50% ethyl acetate-hexanes, linear gradient) to afford the recovered starting material 12 as an amorphous white solid (10.0 mg, >99%).

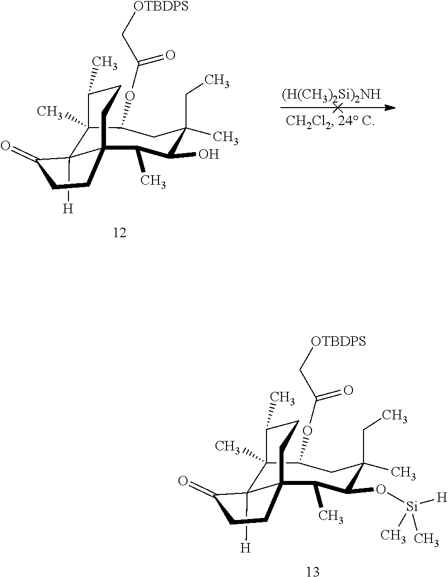

### Attempted Synthesis of Silane 13 (FIG. 15, Table S1, Entry 3)

Bis(dimethylsilyl)amine (4.6 μL, 26.0 mmol, 2.00 equiv) was added dropwise via syringe to a solution of O-tert-butyldiphenylsilyl-19,20-dihydropleuromutilin [12, 8.1 mg, 13.0 μmol, 1 equiv, dried by azeotropic distillation with benzene (300 μL)] in dichloromethane (200 μL) at 24° C. The reaction mixture was stirred for 12 h at 24° C. The product mixture was concentrated to dryness and the residue obtained was purified by automated flash-column chromatography (eluting with hexanes initially, grading to 50% ethyl acetate-hexanes, linear gradient) to afford the recovered starting material 12 as an amorphous white solid (8.1 mg, >99%).

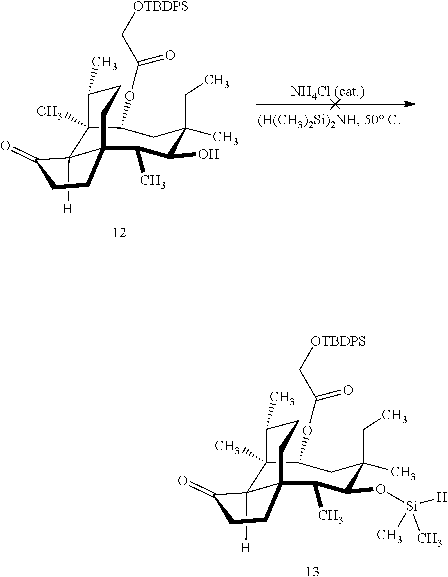

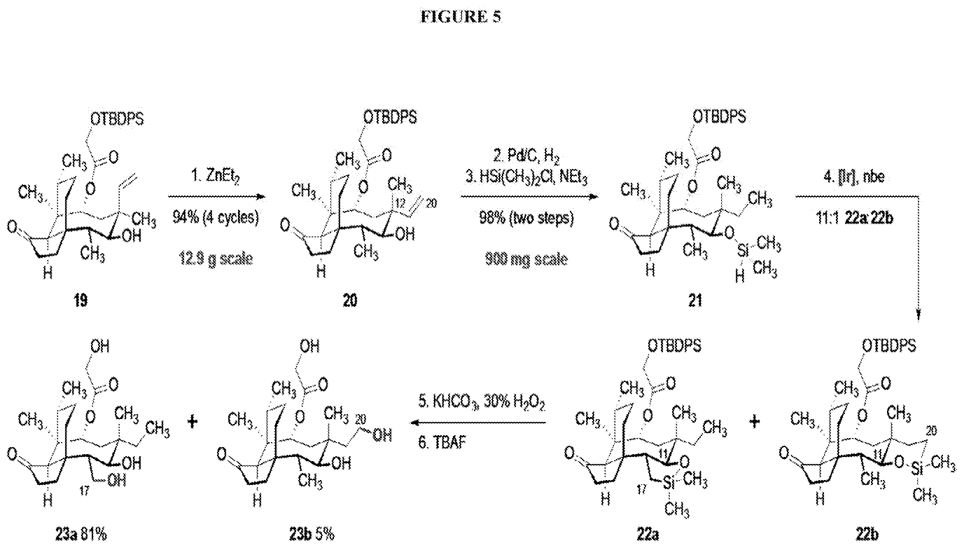

### Attempted Synthesis of Silane 13 (FIG. 5, Table S1, Entry 4)

A catalytic amount of ammonium chloride was added to a solution of O-tert-butyldiphenylsilyl-19,20-dihydropleuromutilin [12, 8.1 mg, 13.0 μmol, 1 equiv, dried by azeotropic distillation with benzene (300 μL)] in bis(dimethylsilyl)amine (200 μL) at 24° C. The reaction vessel was placed in an oil bath that had been previously heated to 50° C. The reaction mixture was stirred and heated for 12 h at 50° C. The product mixture was concentrated to dryness and the residue obtained was purified by automated flash-column chromatography (eluting with hexanes initially, grading to 50% ethyl acetate-hexanes, linear gradient) to afford the recovered starting material 12 as an amorphous white solid (8.1 mg, >99%).

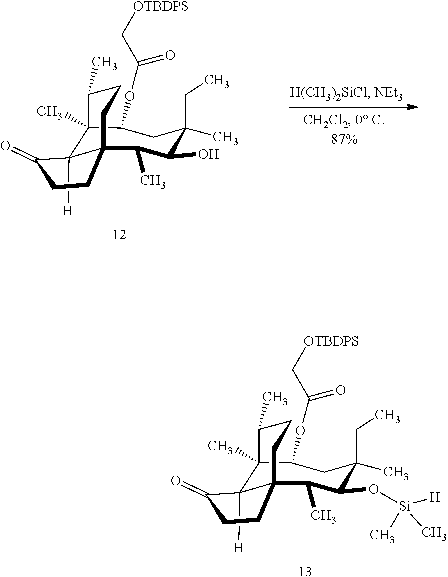

### Synthesis of Silane 13 (FIG. 15, Table S1, Entry 6)

Dimethylchlorosilane (5.8 μL, 52.0 mmol, 2.00 equiv) was added dropwise via syringe to a solution of O-tert-butyldiphenylsilyl-19,20-dihydropleuromutilin [12, 16.1 mg, 26.0 μmol, 1 equiv, dried by azeotropic distillation with benzene (500 μL)] and triethylamine (14.5 μL, 104 μmol, 4.00 equiv) in dichloromethane (300 μL) at 0° C. The reaction mixture was stirred for 30 min at 0° C. The product mixture was diluted sequentially with pentane (1.0 mL) and an aqueous potassium phosphate buffer solution (pH 7, 0.10 M, 1.0 mL) at 0° C. The diluted mixture was transferred to a separatory funnel and the layers formed were separated. The aqueous layer was extracted with dichloromethane (3×5.0 mL). The organic layers were combined and the combined organic layers were dried over sodium sulfate. The dried solution was filtered and the filtrate was concentrated to dryness. The residue obtained was purified by automated flash-column chromatography (eluting with hexanes initially, grading to 25% ether-hexanes, linear gradient) to afford silane 13 as an amorphous white solid (15.4 mg, 87%).

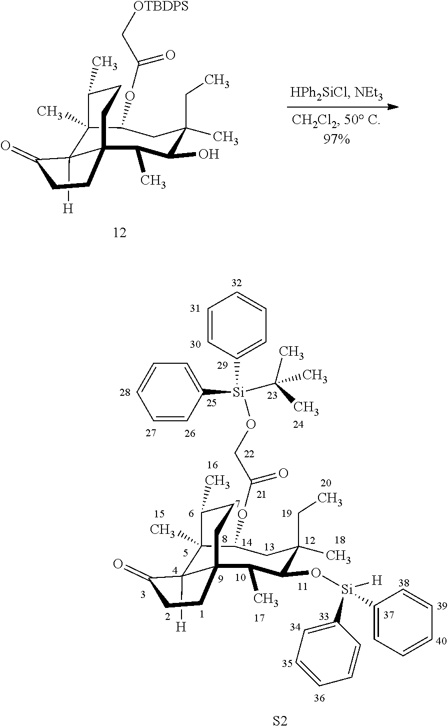

### Synthesis of Silane S2 (FIG. 15, Table S1, Entry 7)

A 10-mL round-bottomed flask fused to a Teflon-coated valve was charged with O-tert-butyldiphenylsilyl-19,20-dihydropleuromutilin (12, 50.0 mg, 80.8 μmol, 1 equiv), Benzene (1.0 mL) was added and the solution was concentrated to dryness. This process was repeated twice. The reaction vessel was evacuated and refilled using a balloon of argon. This process was repeated two times. Dichloromethane (300 μL), triethylamine (45.0 μL, 323 μmol, 4.00 equiv), and (chloro)diphenylsilane (25.0 μL, 121 μmol, 2.00 equiv, 95% purity) were added sequentially to the reaction vessel. The reaction vessel was sealed and the sealed vessel was placed in an oil bath that had been previous heated to 50° C. The reaction was stirred and heated for 3 h at 50° C. The reaction vessel was allowed to cool over 30 min to 24° C. The product mixture was diluted sequentially with pentane (1.0 mL) and an aqueous potassium phosphate buffer solution (pH 7, 0.10 M, 1.0 mL). The diluted mixture was transferred to a separatory funnel and the layers formed were separated. The aqueous layer was extracted with dichloromethane (3×5.0 mL). The organic layers were combined and the combined organic layers were dried over sodium sulfate. The dried solution was filtered and the filtrate was concentrated to dryness. The residue obtained was purified by automated flash-column chromatography (eluting with hexanes initially, grading to 25% ether-hexanes, linear gradient) to afford silane S2 as an amorphous white solid (64.0 mg, 97%).

R_{f}=0.39 (10% ethyl acetate-hexanes; UV, CAM, PAA). ¹H NMR (400 MHz, C₆D₆) δ 7.80-7.77 (m, 4H, 2×H₂₇, 2×H₃₁), 7.75-7.72 (m, 2H, H₁₅), 7.68-7.66 (m, 2H, H₃₉), 7.25-7.14 (m, 12H, 2×H₂₆, 1×H₂₈, 2×H₃₀, 1×H₃₂, 2×H₃₄, 1×H₃₆, 2×H₃₈, 1×H₄₀), 5.82 (d, J=8.0 Hz, 1H, H₁₄), 5.72 (s, 1H, Si—H), 4.19 (s, 2H, H₂₂), 3.56 (d, J=5.6 Hz, 1H, H₁₁), 2.53-2.50 (m, 1H, H₁₀), 2.13-1.98 (m, 2H, H₉), 1.85-1.80 (m, 2H, H₂), 1.77 (s, 1H, H₄), 1.71-1.64 (m, 1H, H₆), 1.56 (s, 3H, H₁₅), 1.54-1.48 (m, 2H, 1×H₇, 1×H₁₃), 1.40-1.35 (m, 1H, 1×H₈), 1.23-1.19 (m, 1H, 1×H₁, 9×H₂₄), 1.11-1.07 (m, 1H, 1×H₇), 1.03-0.97 (m, 1H, 1×H₁), 0.95-0.89 (m, 6H, 3×H₁, 3×H₂₀), 0.87 (d, J=6.8 Hz, 3H, H₁₇), 0.84-0.71 (m, 2H, 1×H₁, 1×H₈), 0.68 (d, J=7.2 Hz, 3H, H₆). ¹³C NMR (100 MHz, C₆D₆) δ 215.1 (C), 169.8 (C), 136.1 (CH), 136.1 (CH), 135.6 (CH), 135.5 (CH), 135.1 (C), 134.7 (C), 134.5 (C), 133.5 (CH), 130.8 (CH), 130.7 (CH), 130.5 (C), 130.2 (CH), 128.6 (CH), 128.4 (CH), 128.2 (CH), 128.2 (CH), 80.1 (CH), 68.8 (CH), 63.3 (CH₂), 58.3 (CH), 45.3 (C), 42.4 (C), 42.2 (C), 41.3 (CH₂), 37.0 (CH), 35.7 (CH), 34.5 (CH₂), 30.7 (CH₂), 28.0 (CH₃), 27.2 (CH₂), 27.0 (CH₃), 25.1 (CH₂), 21.7 (CH₂), 19.5 (C), 16.8 (CH₃), 15.1 (CH₃), 12.7 (CH₃), 8.8 (CH₃). IR (ATR-FTIR), cm⁻¹: 2933 (w), 1736 (m), 1428 (m), 1214 (w), 1112 (s), 1053 (m), 863 (s), 808 (s), 734 (m), 699 (s), 498 (s). HRMS-ESI (m/z): [M+K]⁺ calcd for C₅₀H₆₄KO₅Si₂, 839.3929; found, 839.3955. [α]_{D}²⁵=+32° (c=1.0, CHCl₃).

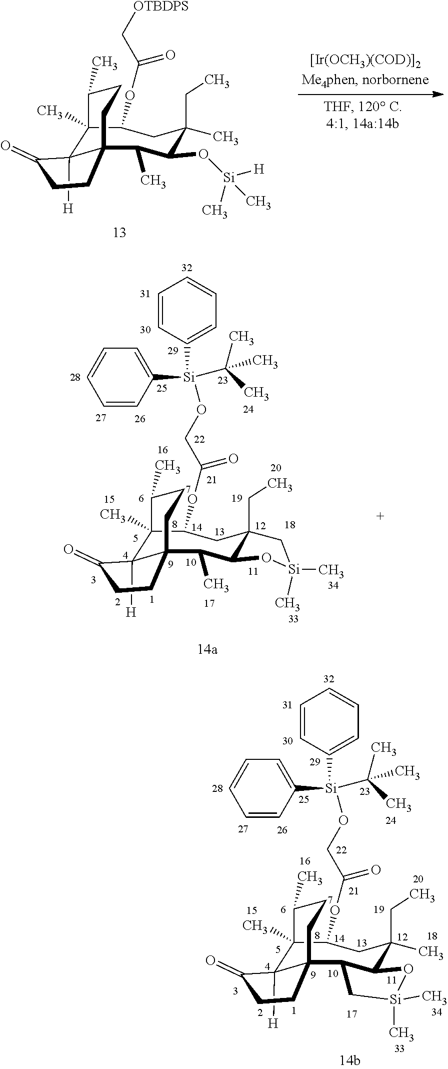

### Synthesis of Silacycles 14a and 14b (FIG. 3, Scheme 3)

This experiment was adapted from the work of Hartwig and co-workers.² A 50-mL pressure tube with a Teflon-coated valve was charged with 3,4,7,8-tetramethyl-1,10-phenanthroline (199 mg, 843 μmol, 12.5 mol %) and norbornene (952 mg, 10.1 mmol, 1.50 equiv) in the glovebox. A 100-mL pear-shaped flask was charged with silane 13 [4.57 g, 6.74 mmol, 1 equiv, dried by azeotropic distillation with benzene (3×50 mL)]. The vessel containing the silane was evacuated and refilled using a balloon of argon. This process was repeated two times. Tetrahydrofuran (10 mL) was transferred into the vessel containing the silane and the resulting solution was added to the vessel containing the ligand and norbornene in the glovebox. The vessel containing the silane was rinsed with tetrahydrofuran (3×2.0 mL) and the combined rinses were transferred to the reaction vessel. Methoxy(cyclooctadiene)iridium(I) dimer (233 mg, 337 μmol, 5.0 mol %) was added to an oven-dried 20-mL vial. Tetrahydrofuran (2.0 mL) was added into the vial containing the catalyst and the resulting solution was transferred dropwise via syringe to the reaction vessel in the glovebox. The vial containing the catalyst was rinsed with tetrahydrofuran (3×1.0 mL) and the combined rinses were transferred into the reaction vessel. The reaction vessel was sealed and the reaction mixture was stirred for 1 h at 24° C. in the glovebox. The sealed reaction vessel was then removed from the glovebox and placed in an oil bath that had been preheated to 120° C. The reaction mixture was stirred and heated for 2 h at 120° C. The reaction vessel was allowed to cool over 30 min to 24° C. and the cooled product mixture was concentrated to dryness. The residue obtained was filtered through a pad of silica gel (2.5×4.5 cm). The filter cake was washed with a mixture of ether and hexanes (1:1, v/v, 500 mL). The filtrate were combined and the combined filtrates were concentrated to dryness. The residue obtained contained a mixture of C11-C18-silacycle 14a and C11-C17-silacycle 14b (4.56 g, 99%) and was used in the next step without further purification. ¹H NMR study of the unpurified mixture revealed an approximate 4:1 mixture of 14a:14b. An analytically pure sample of 14a and 14b were obtained for characterization by automated flash-column chromatography (eluting with hexanes initially, grading to 25% ethyl acetate-hexanes, linear gradient).

C11-C18-silacycle 14a: Amorphous white solid. R_{f}=0.51 (10% ethyl acetate-hexanes; UV, CAM). ¹H NMR (500 MHz, CD₂Cl₂) δ 7.68-7.66 (m, 4H, 2×H₂₇, 2×H₃₁), 7.45-7.37 (m, 6H, 2×H₂₆, 1×H₂₈, 2×H₃₀, 1×H₃₂), 5.61 (d, J=8.0 Hz, 1H, H₁₄) 4.15 (dd, J=27.0, 16.5 Hz, 2H, H₂), 3.49 (d, J=6.5 Hz, 1H, H₁), 2.40-2.34 (m, 1H, H₁₀), 2.17-2.08 (m, 3H, 2×H₂₁ 1×H₄), 2.04-2.00 (m, 1H, 1×H₁₉), 1.75 (d, J=14.5 Hz, 1H, 1×H₈), 1.68-1.60 (m, 2H, 1×H₇, 1×H₁₃), 1.60-1.55 (m, 1H, 1×H₁), 1.55-1.52 (m, 2H, 1×H₇, 1×H₁₃), 1.52-1.38 (m, 3H, 1×H₁, 1×H₇, 1×H₁₉), 1.36 (s, 3H, H₁₈), 1.17-1.11 (m, 2H, 1×H₁₅, 1×H₁₈), 1.07 (s, 9H, H₂₄), 0.92 (d, J=9.0 Hz, 3H, H₁₇), 0.89-0.86 (m, 1H, 1×H₁₈), 0.71 (t, J=7.3 Hz, 3H, H₂₀), 0.91 (d, J=6.0 Hz, 3H, H₁₆), 0.23 (s, 3H, H₃₃), 0.16 (s, 3H, H₃₄). ¹³C NMR (125 MHz, CD₂Cl₂) δ 218.0 (C), 170.3 (C), 136.1 (CH), 133.5 (C), 133.5 (C), 130.4 (CH), 128.3 (CH), 128.3 (CH), 82.2 (CH), 69.5 (CH), 63.5 (CH₂), 59.1 (CH), 47.3 (C), 46.3 (C), 42.5 (C), 41.9 (CH₂), 37.5 (CH), 35.0 (CH₂), 33.7 (CH), 30.6 (CH₂), 27.6 (CH₂), 27.0 (CH₃), 26.2 (CH₂), 25.6 (CH₂), 19.9 (CH₂), 19.7 (C), 16.9 (CH₃), 15.3 (CH₃), 12.1 (CH₃), 8.7 (CH₃), 0.53 (CH₃), 0.47 (CH₃). IR (ATR-FTIR), cm⁻¹: 2933 (w), 1736 (m), 1428 (m), 1112 (s), 1053 (m), 863 (m), 808 (s), 734 (s), 699 (s), 498 (s). HRMS-ESI (m/z): [M+K]⁺ calcd for C₄₀H₃KO₅Si₂, 713.3460; found, 713.3488. [α]_{D}²⁵=+28° (c=0.5, CHCl₃).

C11-C17-silacycle 14b: Amorphous white solid. R_{f}=0.48 (10% ethyl acetate-hexanes; UV, CAM). ¹H NMR (400 MHz, CD₂Cl₂) δ 7.69-7.66 (m, 4H, 2×H₂₇, 2×H₃₁), 7.46-7.36 (m, 6H, 2×H₂₆, 1×H₂₈, 2×H₃₀, 1×H₃₂), 5.68 (d, J=8.0 Hz, 1H, H₁₄), 4.17 (dd, J=20.8, 4.4 Hz, 2H, H₂), 3.73 (d, J=5.2 Hz, 1H, H₁₁), 2.86-2.80 (m, 1H, H₁₀), 2.23-2.09 (m, 3H, 2×H₂, 1×H₄), 1.76-1.50 (m, 7H, 2×H₁, 1×H₆, 1×H₇, 1×H₈, 1×H₁₃, 1×H₁₉), 1.47-1.40 (m, 1H, 1×H₁₉), 1.36 (s, 3H, H₁₅), 1.29-1.25 (m, 1H, 1×H₁₃), 1.23-1.20 (m, 1H, 1×H₈), 1.18-1.12 (m, 1H, 1×H₇), 1.07 (s, 9H, H₂₄), 1.00-0.96 (m, 1H, 1×H₁₇), 0.92 (s, 3H, H₁₈), 0.79-0.73 (m, 1H, 1×H₁₇), 0.69 (t, J=7.4 Hz, 3H, H₂₀), 0.62 (d, J=7.2 Hz, 3H, H₁₆), 0.24 (s, 3H, H₃₃), 0.17 (s, 3H, H₃₄). ¹³C NMR (100 MHz, CD₂Cl₂) δ 217.1 (C), 170.5 (C), 136.1 (CH), 133.5 (C), 1334 (C), 130.4 (CH), 128.3 (CH), 128.3 (CH), 87.3 (CH), 69.2 (CH), 63.5 (CH₂), 59.6 (CH), 46.1 (C), 42.4 (C), 41.2 (C), 40.4 (CH₂), 39.5 (CH), 37.2 (CH), 34.8 (CH₂), 31.9 (CH₂), 27.4 (CH₂), 27.0 (CH₂), 25.7 (2×CH₃), 21.2 (CH₂), 19.6 (C), 16.8 (CH₃), 12.1 (CH₃), 13.5 (CH₂), 8.5 (CH₃), 0.85 (CH₃), 0.79 (CH₃). IR (ATR-FTIR), cm⁻¹: 2958 (w), 1738 (m), 1462 (w), 1251 (w), 1114 (s), 1056 (m), 971 (w), 873 (m), 814 (m), 702 (s), 613 (m), 504 (s). HRMS-ESI (m/z): [M+K]⁺ calcd for C₄₀H₄₈KO₅Si₂, 713.3460; found, 713.3444. [α]_{D}²⁵=+50 (c=0.5, CHCl₃).

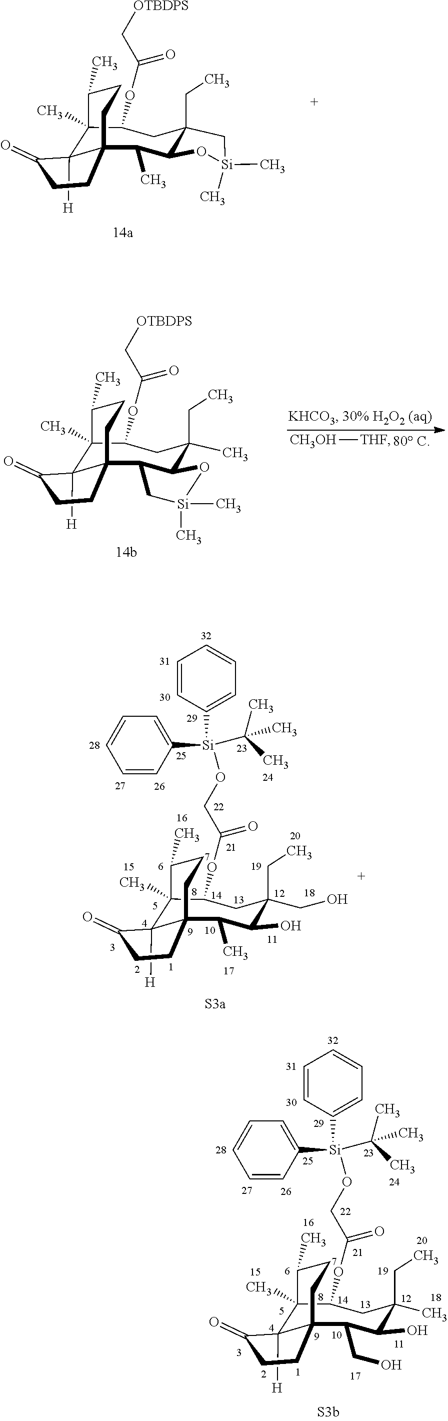

### Tamao-Fleming Oxidation of a Mixture of 14a and 14b (Scheme 3)

Tetrahydrofuran (150 μL) and an aqueous hydrogen peroxide solution (30% w/w, 168 μL, 1.48 mmol, 20.0 equiv) were added sequentially to a suspension of the unpurified mixture of the two silacycles 14a and 14b (50.0 mg, 74.1 μmol, 1 equiv) and potassium bicarbonate (44.4 mg, 444 μmol, 6.00 equiv) in methanol (150 μL) at 24° C. in a 4-mL vial. The vial was sealed with a Teflon-lined cap and the sealed vial was placed in an oil bat that had been preheated to 80° C. The reaction mixture was stirred and heated for 3 h at 80° C. The product mixture was diluted sequentially with dichloromethane (2.0 mL) and saturated aqueous sodium thiosulfate (1.0 mL). The diluted product mixture was transferred to a separatory funnel and the layers that formed were separated. The aqueous layer was extracted with dichloromethane (3×5 mL). The organic layers were combined and the combined organic layers were dried over sodium sulfate. The dried solution was filtered and the filtrate was concentrated to dryness. The residue obtained contained a mixture of diols S3a and S3b (47.1 mg, 99%) and was used in the next step without further purification. An analytically pure sample of S3a and S3b were obtained for characterization by automated flash-column chromatography (eluting with dichloromethane initially, grading to 100% ether-dichloromethane, linear gradient; then eluting with 10% methanol-dichloromethane).

Diol S3a: Amorphous white solid. R_{f}=0.42 (40% ethyl acetate-hexanes; UV, CAM). ¹H NMR (400 MHz, CD₂C12) δ 7.68-7.64 (m, 4H, 2×H₂₇, 2×H₃₁), 7.45-7.35 (m, 6H, 2×H₂₆, 1×H₂₈, 2×H₃₀, 1×H₃₂), 5.70 (d, J=8.4 Hz, 1H, H₁₄), 4.16 (dd, J=24.8, 8.4 Hz, 2H, H₂), 3.83 (d, J=6.4 Hz, 1H, H₁₁), 3.51 (d, J 11.2 Hz, 1H, 1×H₁₁), 3.39 (d, J=11.2 Hz, 1H, 1×H₁₈), 3.00-3.65 (br m, 2H, 2×OH), 2.47-2.40 (m, 1H, H₁₀), 2.22-2.08 (m, 2H, H₂), 2.06 (s, 1H, H₄), 1.86-1.75 (m, 3H, 1×H₈, 2×H₁₉), 1.68-1.62 (m, 1H, 1×H₁₃), 1.60-1.53 (m, 3H, 1×H₁, 1×H₆, 1×H₇), 1.48-1.41 (m, 1H, H₁), 1.36-1.30 (m, 5H, 1×H₇, 1×H₁, 3×H₁), 1.14-1.04 (m, 10H, 1×H₈, 9×H₂₄), 0.92 (d, J=7.2 Hz, 3H, H₁₇), 0.74 (t, J=7.4 Hz, 3H, H₂), 0.60 (d, J=6.4 Hz, 3H, H₁₆). ¹³C NMR (100 MHz, CD₂Cl₂) δ 217.2 (C), 169.9 (C), 135.5 (CH), 132.9 (C), 132.8 (C), 129.9 (CH), 127.8 (CH), 75.5 (CH), 70.4 (CH₂), 67.9 (CH), 62.9 (CH₂), 58.3 (CH), 45.4 (C), 43.8 (C), 41.9 (C), 36.8 (CH), 35.4 (CH₂), 34.5 (CH), 34.4 (CH₂), 30.2 (CH₂), 26.9 (CH₂), 26.4 (CH₃), 25.0 (CH₂), 19.1 (C), 16.9 (CH₂), 16.4 (CH₃), 14.6 (CH₃), 10.7 (CH₃), 7.5 (CH₃). IR (ATR-FTIR), cm⁻¹: 3265 (br w), 2927 (w), 1759 (m), 1738 (w), 1462 (w), 1134 (s), 1112 (s), 826 (m), 702 (s), 613 (s), 507 (s), 491 (s). HRMS-ESI (m/z): [M+H]⁺ calcd for C₃₈H₅₅NaO₆Si, 635.3768; found, 635.3768. [α]_{D}²⁵=+32° (c=0.33, CHCl₃).

Diol S3b: Amorphous white solid. R_{f}=0.33 (40% ethyl acetate-hexanes; UV, CAM). ¹H NMR (400 MHz, CD₂Cl₂) δ 7.68-7.66 (m, 4H, 2×H₂₇, 2×H₃₁), 7.47-7.38 (m, 6H, 2×H₂₆, 1×H₂₈, 2×H₃₀, 1×H₃₂), 5.64 (d, J=8.0 Hz, 1H, H₄), 4.18 (dd, J=26.4, 10.0 Hz, 2H, H₂₂), 3.91-3.88 (m, 1H, 1×H₁₇), 3.82-3.79 (m, 1H, 1×H₁₇), 3.60-3.57 (m, 1H, H₁), 3.24 (d, J=7.2 Hz, 1H, C11-OH), 2.62 (t, J=5.6 Hz, 1H, C17-OH), 2.43 (t, J=6.4 Hz, 1H, H₁₀), 2.26-2.14 (m, 2H, 2×H₂), 2.08 (s, 1H, 1×H₄) 1.89-1.82 (m, 2H, 1×H₈, 1×H₁₉), 1.80-1.65 (m, 3H, 1×H₁, 1×H₇, 1×H₁), 1.54-1.44 (m, 1H, 1×H₁₉), 1.42-1.39 (m, 2H, 1×H₁, 1×H₇), 1.37 (s, 3H, H₁), 1.27-1.25 (m, 1H, 1×H₁₃), 1.20-1.12 (m, 1H, 1×H₈), 1.08 (3, 9H, H₂), 0.94 (s, 3H, H₁₈), 0.72 (t, J=7.4 Hz, 3H, H₂₀), 0.63 (d, J=6.4 Hz, 3H, H₁₆). ¹³C NMR (100 MHz, CD₂Cl₂) δ 216.3 (C), 169.9 (C), 135.5 (CH), 132.9 (C), 129.8 (CH), 127.8 (CH), 127.7 (CH), 77.8 (CH), 68.4 (CH), 62.9 (CH₂), 61.4 (CH₂), 58.6 (CH), 44.1 (C), 42.7 (CH), 41.9 (C), 40.5 (C), 4.2 (CH₂), 36.7 (CH), 34.4 (CH₂), 30.4 (CH₂), 26.9 (CH₂), 26.4 (CH₃), 26.0 (CH₃), 25.6 (CH₂), 20.9 (CH₂), 19.1 (C), 16.3 (CH₃), 14.6 (CH₃), 7.9 (CH₃). IR (ATR-FTIR), cm⁻¹: 2932 (w), 1735 (m), 1461 (w), 1284 (w), 1142 (m), 1113 (s), 1038 (m), 1013 (m), 823 (m), 701 (s), 504 (s). HRMS-ESI (m/z): [M+H]⁺ calcd for C₃₈H₅₅O₆Si, 635.3768; found, 635.3772. [α]_{D}²⁵=+31° (c=0.33, CHCl₃).

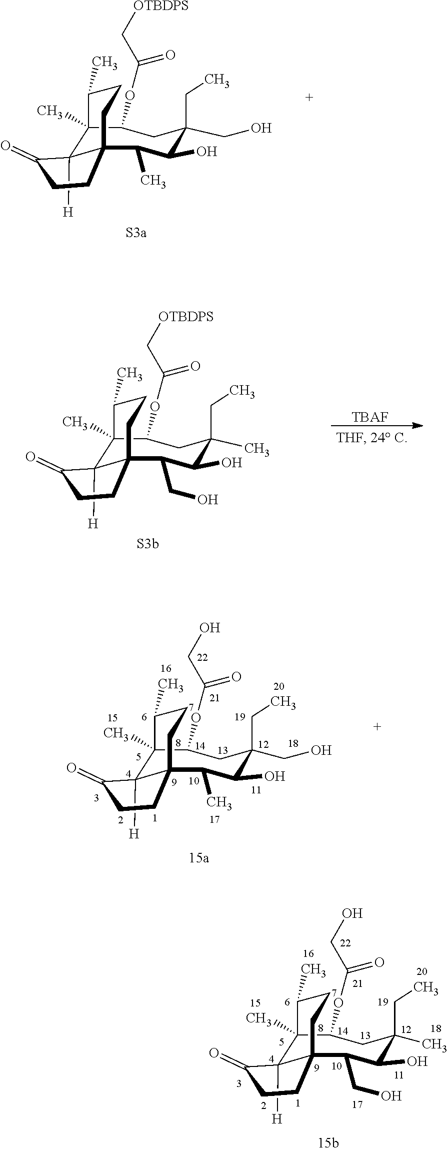

### Silyldeprotection of a Mixture of S3a and S3b (FIG. 3, Scheme 3)

A solution of tetrabutylammonium fluoride in tetrahydrofuran (1.00 M, 148 μL, 148 μmol, 2.00 equiv) was added dropwise via syringe to a solution of the unpurified mixture of the diols S3a and S3b (47.1 mg, 74.1 μmol, 1 equiv) in tetrahydrofuran (1.5 mL) at 24° C. The reaction mixture was stirred for 2 h at 24° C. The product mixture was diluted sequentially with dichloromethane (3.0 mL) and saturated aqueous sodium bicarbonate (2.0 mL). The diluted product mixture was transferred to a separatory funnel and the layers that formed were separated. The aqueous layer was extracted with ethyl acetate (3×10 mL). The organic layers were combined and the combined organic layers were dried over sodium sulfate. The dried solution was filtered and the filtrate was concentrated to dryness. The residue obtained was purified by automated flash-column chromatography (eluting with hexanes initially, grading to 100% ethyl acetate-hexanes, linear gradient) to afford separately 18-hydroxy-19,20-dihydropleuromutilin (15a) as an amorphous white solid (21.4 mg, 73%) and 17-hydroxy-19,20-dihydropleuromutilin (15b) as an amorphous white solid (1.8 mg, 6%).

18-Hydroxy-19,20-dihydropleuromutilin (15a): R_{f}=0.33 (75% ethyl acetate-hexanes; PAA, CAM). ¹H NMR (400 MHz, CD₂Cl₂) δ 5.77 (d, J=8.4 Hz, 1H, H₁₄), 4.04 (dd, J=31.2, 14.4 Hz, 2H, H₂₂), 3.87 (d, J=6.4 Hz, 1H, H₁₁), 3.63 (br s, 1H, C8-OH), 3.56 (d, J=10.8 Hz, 1H, 1×H₁₈), 3.43 (d, J=10.8 Hz, 1H, 1×H₁₈), 2.69 (br s, H, C11-OH), 2.47-2.40 (m, 1H, H₁₀), 2.29-2.13 (m, 2H, H₂) 2.11 (s, 1H, H₄), 1.87-1.74 (m, 4H, 1×H₈, 1×H₁₃, 2×H₁₉), 1.65-1.54 (m, 3H, 1×H₁, 1×H₆, 1×H₇), 1.51-1.42 (m, 1H, 1×H₁), 1.42-1.36 (m, 4H, 1×H₇, 3×H₅), 1.20-1.07 (m, 2H, 1×H₈, 1×H₁₃), 0.95 (d, J=7.2 Hz, 3H, H₁₇), 0.77 (t, J=7.4 Hz, 3H, H₂₀), 0.69 (d, J=6.4 Hz, 3H, H₁₆). ¹³C NMR (100 MHz, CD₂Cl₂) δ 216.7 (C), 172.3 (C), 75.2 (CH), 70.3 (CH₂), 69.1 (CH), 61.3 (CH₂), 58.1 (CH), 45.4 (C), 43.9 (C), 41.9 (C), 36.6 (CH), 35.4 (CH₂), 34.4 (CH), 34.3 (CH₂), 30.2 (CH₂), 26.8 (CH₂), 24.9 (CH₂), 17.0 (CH₂), 16.2 (CH₃), 14.4 (CH₃), 10.6 (CH₃), 7.4 (CH₃). IR (ATR-FTIR), cm⁻¹: 3373 (m), 2944 (m), 1728 (s), 1461 (w), 1385 (w), 1233 (m), 1098 (m), 911 (m), 731 (s). HRMS-ESI (m/z): [M+H]⁺ calcd for C₂₂H₃₇O₆, 397.2590; found, 397.2603. [α]_{D}²⁵=+33° (c=1.0, CHCl₃).

17-Hydroxy-19,20-dihydropleuromutilin (15b): R_{f}=0.11 (75% ethyl acetate-hexanes; PAA, CAM). ¹H NMR (400 MHz, CDCl₃) δ 5.71 (d, J=7.6 Hz, 1H, H₁₄), 4.05 (t, J=16 Hz, 2H, H₂₂), 3.94 (t, J=10.0 Hz, 1H, 1×H₁₇), 3.81 (d, J=10.4 Hz, 1H, 1×H₇), 3.61 (d, J=6.4 Hz, 1H, H₁₁), 3.10 (br s, H, C11-OH), 2.48-2.40 (m, 1H, H₁₀), 2.29-2.13 (m, 2H, H₂), 2.07 (s, 1H, H₄), 1.93-1.83 (m, 1H, 1×H₁₉), 1.79-1.72 (m, 2H, 1×H₈, 1×H₁₃), 1.70-1.59 (m, 3H, 1×H₁, 1×H₆, 1×H₇), 1.54-1.46 (m, 1H, 1×H₁₉), 1.44-1.36 (m, 5H, 1×H₁, 1×H₇, 3×H₁₅), 1.26-1.22 (m, 1H, 1×H₁₃), 1.21-1.13 (m, 1H, 1×H₈), 0.97 (s, 3H, H₁₈), 0.87 (br m, 1H, C17-OH), 1.26-1.22 (m, 6H, 1×H₁₆, 1×H₂₀). ¹³C NMR (100 MHz, CDCl₃) δ 216.3 (C), 172.2 (C), 78.01 (CH), 69.7 (CH), 61.6 (CH₂), 61.3 (CH₂), 58.7 (CH), 44.1 (C), 42.7 (CH), 41.9 (C), 40.6 (C), 40.2 (CH), 36.5 (CH₂), 34.4 (CH₂), 30.4 (CH₂), 26.8 (CH₂), 26.3 (CH), 25.7 (CH₂), 20.9 (CH₂), 16.4 (CH₃), 14.7 (CH), 8.0 (CH₃). IR (ATR-FTIR), cm⁻¹: 3385 (br w), 2930 (s), 2870 (w), 1734 (s), 1458 (m), 1376 (w), 1282 (w), 1232 (m), 1157 (w), 1097 (m), 1038 (w), 1008 (w), 736 (w). HRMS-ESI (m/z): [M+H]⁺ calcd for C₂₂H₃₇O₆, 397.2590; found, 397.2591.

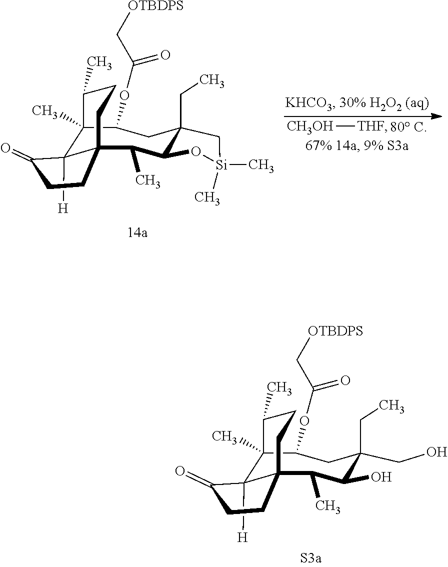

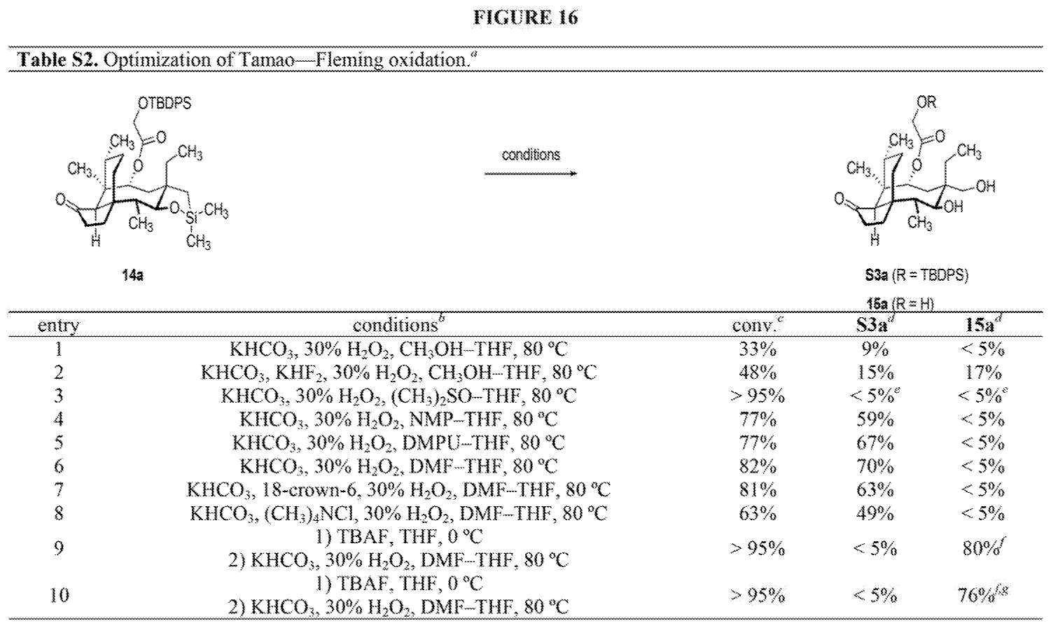

### Tamao-Fleming Oxidation of a Mixture of 14a and 14b (FIG. 16, Table S2, Entry 1)

Tetrahydrofuran (277 μL) and an aqueous hydrogen peroxide solution (30% w/w, 336 μL, 2.96 mmol, 20.0 equiv) were added sequentially to a suspension of the unpurified mixture of the two silacycles 14a and 14b (100.0 mg, 148 μmol, 1 equiv) and potassium bicarbonate (88.9 mg, 889 μmol, 6.00 equiv) in methanol (277 μL) at 24° C. in a 4-mL pressure tube with a Teflon-coated valve. The tube was sealed and the sealed tube was placed in an oil bat that had been preheated to 80° C. The reaction mixture was stirred and heated for 3 h at 80° C. The product mixture was diluted sequentially with dichloromethane (2.0 mL) and saturated aqueous sodium thiosulfate (1.0 mL). The diluted product mixture was transferred to a separatory funnel and the layers that formed were separated. The aqueous layer was extracted with dichloromethane (3×5.0 mL). The organic layers were combined and the combined organic layers were dried over sodium sulfate. The dried solution was filtered and the filtrate was concentrated to dryness. The residue obtained was purified by automated flash-column chromatography (eluting with hexanes initially, grading to 100% ethyl acetate-hexanes, linear gradient) to afford separately the silacycle 14a as an amorphous white solid (66.9 mg, 67%) and the diol S3a as an amorphous white solid (8.4 mg, 9%).

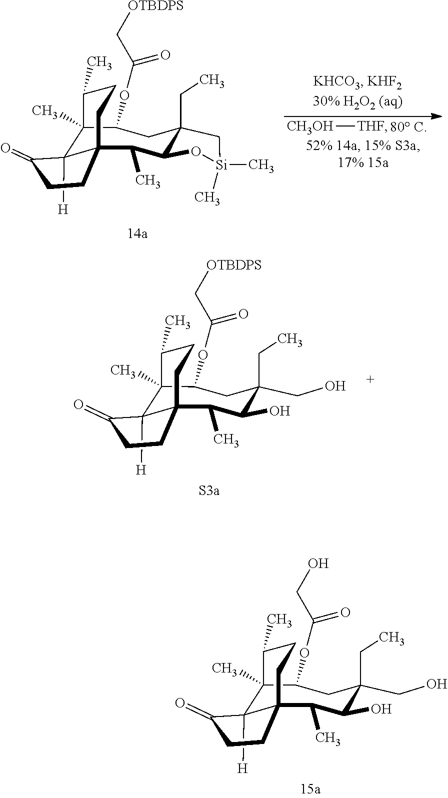

### Tamao-Fleming Oxidation of a Mixture of 14a and 14b (FIG. 16, Table 52, Entry 2)

Tetrahydrofuran (277 μL) and an aqueous hydrogen peroxide solution (30% w/w, 336 μL, 2.96 mmol, 20.0 equiv) were added sequentially to a suspension of the unpurified mixture of the two silacycles 14a and 14b (100.0 mg, 148 μmol, 1 equiv), potassium bifluoride (23.1 mg, 111 μmol, 2.00 equiv), and potassium bicarbonate (88.9 mg, 889 μmol, 6.00 equiv) in methanol (277 μL) at 24° C. in a 4-mL pressure tube with a Teflon-coated valve. The tube was sealed and the sealed tube was placed in an oil bat that had been preheated to 80° C. The reaction mixture was stirred and heated for 3 h at 80° C. The product mixture was diluted sequentially with dichloromethane (2.0 mL), saturated aqueous sodium thiosulfate (1.0 mL), and saturated aqueous sodium bicarbonate (500 μL). The diluted product mixture was transferred to a separatory funnel and the layers that formed were separated. The aqueous layer was extracted with dichloromethane (3×5.0 mL). The organic layers were combined and the combined organic layers were dried over sodium sulfate. The dried solution was filtered and the filtrate was concentrated to dryness. The residue obtained was purified by automated flash-column chromatography (eluting with hexanes initially, grading to 100% ethyl acetate-hexanes, linear gradient) to afford separately the silacycle 14a as an amorphous white solid (51.7 mg, 52%), the diol S3a as an amorphous white solid (14.3 mg, 15%), and 18-hydroxyl-19,20-dihydropleuromutilin as an amorphous white solid (15a, 9.7 mg, 17%).

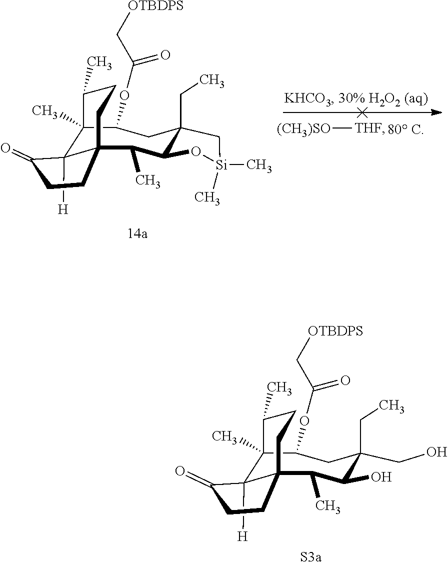

### Tamao-Fleming Oxidation of a Mixture of 14a and 14b FIG. 16, Table S2, Entry 3)

Dimethylsulfoxide (277 μL) and an aqueous hydrogen peroxide solution (30% w/w, 336 μL, 2.96 mmol, 20.0 equiv) were added sequentially to a suspension of the unpurified mixture of the two silacycles 14a and 14b (100.0 mg, 148 μmol, 1 equiv) and potassium bicarbonate (88.9 mg, 889 μmol, 6.00 equiv) in tetrahydrofuran (277 μL) at 24° C. in a 4-mL pressure tube with a Teflon-coated valve. The tube was sealed and the sealed tube was placed in an oil bat that had been preheated to 80° C. The reaction mixture was stirred and heated for 3 h at 80° C. The product mixture was diluted sequentially with dichloromethane (2.0 mL) and saturated aqueous sodium thiosulfate (1.0 mL). The diluted product mixture was transferred to a separatory funnel and the layers that formed were separated. The aqueous layer was extracted with dichloromethane (3×5.0 mL). The organic layers were combined and the combined organic layers were dried over sodium sulfate. The dried solution was filtered and the filtrate was concentrated to dryness. ¹H NMR analysis of the unpurified mixture showed complex decompositions.

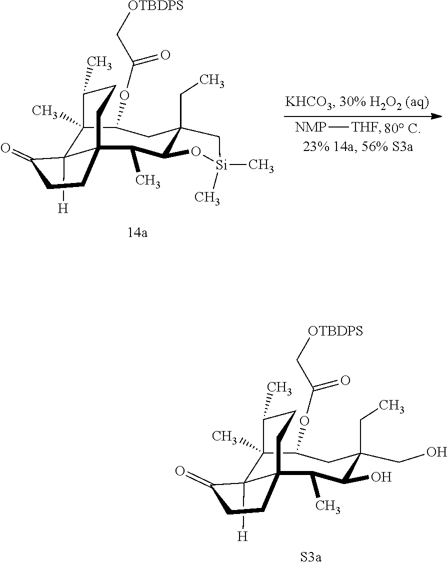

### Tamao-Fleming Oxidation of a Mixture of 14a and 14b (Table S2, Entry 4)

N-Methylpyrrolidone (277 μL) and an aqueous hydrogen peroxide solution (30% w/w, 336 μL, 2.96 mmol, 20.0 equiv) were added sequentially to a suspension of the unpurified mixture of the two silacycles 14a and 14b (100.0 mg, 148 μmol, 1 equiv) and potassium bicarbonate (118 mg, 1.18 mmol, 8.00 equiv) in tetrahydrofuran (277 μL) at 24° C. in a 4-mL pressure tube with a Teflon-coated valve. The tube was sealed and the sealed tube was placed in an oil bat that had been preheated to 80° C. The reaction mixture was stirred and heated for 3 h at 80° C. The product mixture was diluted sequentially with dichloromethane (2.0 mL) and saturated aqueous sodium thiosulfate (1.0 mL). The diluted product mixture was transferred to a separatory funnel and the layers that formed were separated. The aqueous layer was extracted with dichloromethane (3×5.0 mL). The organic layers were combined and the combined organic layers were dried over sodium sulfate. The dried solution was filtered and the filtrate was concentrated to dryness. The residue obtained was purified by automated flash-column chromatography (eluting with hexanes initially, grading to 100% ethyl acetate-hexanes, linear gradient) to afford separately the silacycle 14a as an amorphous white solid (22.5 mg, 23%) and the diol S3a as an amorphous white solid (52.3 mg, 56%).

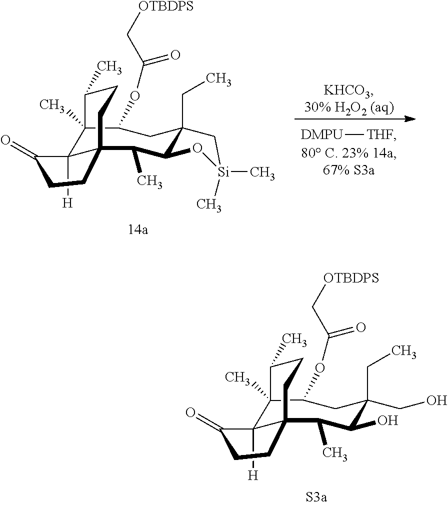

### Tamao-Fleming Oxidation of a Mixture of 14a and 14b (FIG. 16, Table S2, Entry 5)

1,3-Dimethyl-3,4,5,6-tetrahydro-2-pyrimidinone (277 μL) and an aqueous hydrogen peroxide solution (30% w/w, 336 μL, 2.96 mmol, 20.0 equiv) were added sequentially to a suspension of the unpurified mixture of the two silacycles 14a and 14b (100.0 mg, 148 μmol, 1 equiv) and potassium bicarbonate (118 mg, 1.18 mmol, 8.00 equiv) in tetrahydrofuran (277 μL) at 24° C. in a 4-mL pressure tube with a Teflon-coated valve. The tube was sealed and the sealed tube was placed in an oil bat that had been preheated to 80° C. The reaction mixture was stirred and heated for 3 h at 80° C. The product mixture was diluted sequentially with dichloromethane (2.0 mL) and saturated aqueous sodium thiosulfate (1.0 mL). The diluted product mixture was transferred to a separatory funnel and the layers that formed were separated. The aqueous layer was extracted with dichloromethane (3×5.0 mL). The organic layers were combined and the combined organic layers were dried over sodium sulfate. The dried solution was filtered and the filtrate was concentrated to dryness. The residue obtained was purified by automated flash-column chromatography (eluting with hexanes initially, grading to 100% ethyl acetate-hexanes, linear gradient) to afford separately the silacycle 14a as an amorphous white solid (22.6 mg, 23%) and the diol S3a as an amorphous white solid (63.7 mg, 67%).

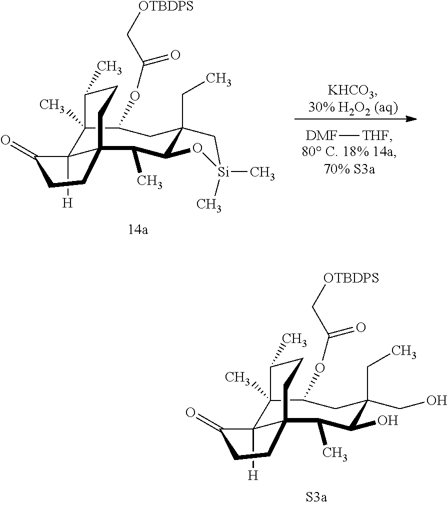

### Tamao-Fleming Oxidation of a Mixture of 14a and 14b (FIG. 16, Table S2, Entry 6)

N,N-Dimethylformamide (277 μL) and an aqueous hydrogen peroxide solution (30% w/w, 336 μL, 2.96 mmol, 20.0 equiv) were added sequentially to a suspension of the unpurified mixture of the two silacycles 14A and 14B (100.0 mg, 148 μmol, 1 equiv) and potassium bicarbonate (118 mg, 1.18 mmol, 8.00 equiv) in tetrahydrofuran (277 μL) at 24° C. in a 4-mL pressure tube with a Teflon-coated valve. The tube was sealed and the sealed tube was placed in an oil bat that had been preheated to 80° C. The reaction mixture was stirred and heated for 3 h at 80° C. The product mixture was diluted sequentially with dichloromethane (2.0 mL) and saturated aqueous sodium thiosulfate (1.0 mL). The diluted product mixture was transferred to a separatory funnel and the layers that formed were separated. The aqueous layer was extracted with dichloromethane (3×5.0 mL). The organic layers were combined and the combined organic layers were washed with water (5×1.0 mL). The washed organic layer was dried over sodium sulfate. The dried solution was filtered and the filtrate was concentrated to dryness. The residue obtained was purified by automated flash-column chromatography (eluting with hexanes initially, grading to 100% ethyl acetate-hexanes, linear gradient) to afford separately the silacycle 14A as an amorphous white solid (18.0 mg, 18%) and the diol S3A as an amorphous white solid (65.3 mg, 70%).

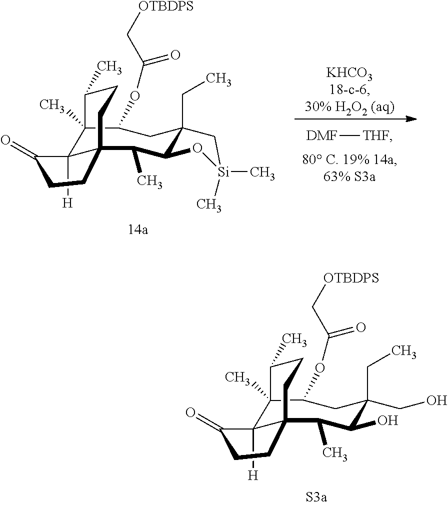

### Tamao-Fleming Oxidation of a Mixture of 14a and 14b (FIG. 16, Table S2, Entry 7)

N,N-Dimethylformamide (277 μL) and an aqueous hydrogen peroxide solution (30% w/w, 336 μL, 2.96 mmol, 20.0 equiv) were added sequentially to a suspension of the unpurified mixture of the two silacycles 14a and 14a (100.0 mg, 148 μmol, 1 equiv), 18-crown-6 (19.6 mg, 74.1 μmol, 0.500 equiv), and potassium bicarbonate (118 mg, 1.18 mmol, 8.00 equiv) in tetrahydrofuran (277 μL) at 24° C. in a 4-mL pressure tube with a Teflon-coated valve. The tube was sealed and the sealed tube was placed in an oil bat that had been preheated to 80° C. The reaction mixture was stirred and heated for 3 h at 80° C. The product mixture was diluted sequentially with dichloromethane (2.0 mL) and saturated aqueous sodium thiosulfate (1.0 mL). The diluted product mixture was transferred to a separatory funnel and the layers that formed were separated. The aqueous layer was extracted with dichloromethane (3×5.0 mL). The organic layers were combined and the combined organic layers were washed with water (5×1.0 mL). The washed organic layer was dried over sodium sulfate. The dried solution was filtered and the filtrate was concentrated to dryness. The residue obtained was purified by automated flash-column chromatography (eluting with hexanes initially, grading to 100% ethyl acetate-hexanes, linear gradient) to afford separately the silacycle 14a as an amorphous white solid (18.8 mg, 19%) and the diol S3a as an amorphous white solid (59.6 mg, 63%).

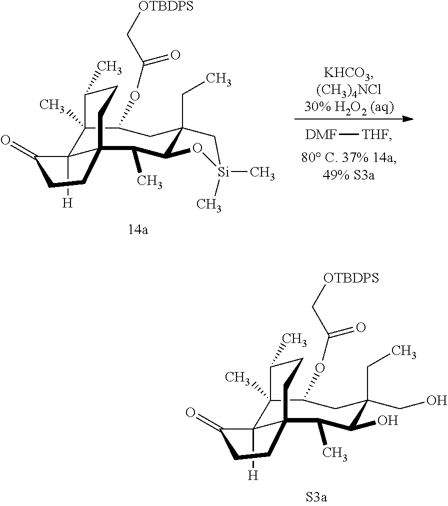

### Tamao-Fleming Oxidation of a Mixture of 14a and 14b (FIG. 16, Table S2, Entry 8)

N,N-Dimethylformamide (277 μL) and an aqueous hydrogen peroxide solution (30% w/w, 336 μL, 2.96 mmol, 20.0 equiv) were added sequentially to a suspension of the unpurified mixture of the two silacycles 14a and 14b (100.0 mg, 148 μmol, 1 equiv), tetramethylammonium chloride (20.6 mg, 74.1 μmol, 0.500 equiv), and potassium bicarbonate (118 mg, 1.18 mmol, 8.00 equiv) in tetrahydrofuran (277 μL) at 24° C. in a 4-mL pressure tube with a Teflon-coated valve. The tube was sealed and the sealed tube was placed in an oil bat that had been preheated to 80° C. The reaction mixture was stirred and heated for 3 h at 80° C. The product mixture was diluted sequentially with dichloromethane (2.0 mL) and saturated aqueous sodium thiosulfate (1.0 mL). The diluted product mixture was transferred to a separatory funnel and the layers that formed were separated. The aqueous layer was extracted with dichloromethane (3×5.0 mL). The organic layers were combined and the combined organic layers were washed with water (5×1.0 mL). The washed organic layer was dried over sodium sulfate. The dried solution was filtered and the filtrate was concentrated to dryness. The residue obtained was purified by automated flash-column chromatography (eluting with hexanes initially, grading to 100% ethyl acetate-hexanes, linear gradient) to afford separately the silacycle 14a as an amorphous white solid (37.2 mg, 37%) and the diol S3a as an amorphous white solid (46.3 mg, 49%).

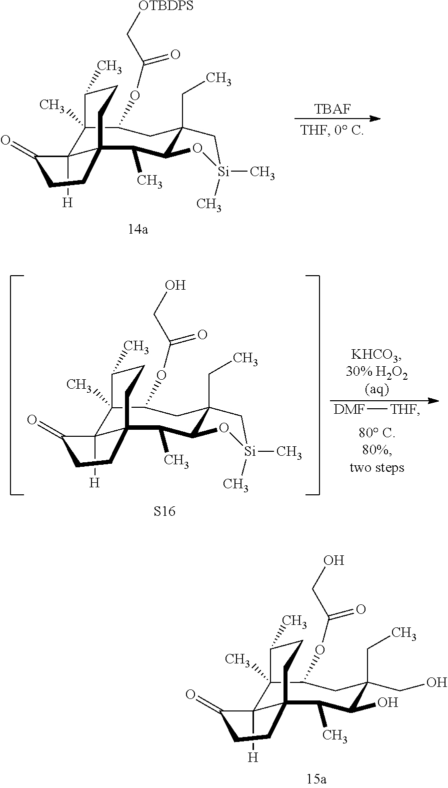

### Tamao-Fleming Oxidation of a Mixture of 14a and 14b (Table S2, Entry 9)

A solution of tetrabutylammonium fluoride in tetrahydrofuran (1.00 M, 156 μL, 156 μmol, 1.05 equiv) was added dropwise via syringe to a solution of the unpurified mixture of the two silacycles 14a and 14b (100.0 mg, 148 μmol, 1 equiv) in tetrahydrofuran (1.0 mL) at 0° C. The reaction was stirred at 0° C. for 25 min. The reaction was diluted sequentially with pentane (1.5 mL) and an aqueous potassium phosphate buffer solution (pH 7, 0.10 M, 1.0 mL). The diluted mixture was transferred to a separatory funnel that had been charged with a mixture of ethyl acetate and hexanes (1:1, v/v, 50 mL). The layers that formed were separated and the organic layer obtained was washed with water (3×5.0 mL). The washed organic layer was dried over sodium sulfate. The dried solution was filtered and the filtrate was concentrated to dryness. The residue obtained containing the highly unstable primaryl alcohol intermediate S16 was used immediately in the next step without purification.

N,N-Dimethylformamide (667 μL) and an aqueous hydrogen peroxide solution (30% w/w, 336 μL, 2.96 mmol, 20.0 equiv) were added sequentially to a suspension of the unpurified intermediate S16 (148 μmol, 1 equiv) and potassium bicarbonate (326 mg, 3.26 mmol, 22.0 equiv) in tetrahydrofuran (333 μL) at 24° C. in a 4-mL pressure tube with a Teflon-coated valve. The tube was sealed and the sealed tube was placed in an oil bat that had been preheated to 80° C. The reaction mixture was stirred and heated for 3 h at 80° C. The product mixture was diluted sequentially with dichloromethane (2.0 mL) and saturated aqueous sodium thiosulfate (1.0 mL). The diluted product mixture was transferred to a separatory funnel and the layers that formed were separated. The aqueous layer was extracted with dichloromethane (3×5.0 mL). The organic layers were combined and the combined organic layers were washed with water (5×1.0 mL). The washed organic layer was dried over sodium sulfate. The dried solution was filtered and the filtrate was concentrated to dryness. The residue obtained was purified by automated flash-column chromatography (eluting with hexanes initially, grading to 100% ethyl acetate-hexanes, linear gradient) to afford 18-hydroxy-19,20-dihydropleuromutilin 15a as an amorphous white solid (47.1 mg, 80%, two steps).

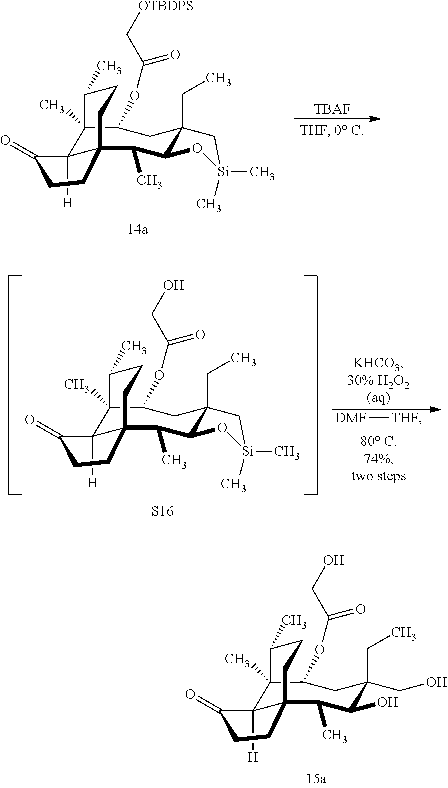

### Tamao-Fleming Oxidation of a Mixture of 14a and 14b (FIG. 3, Scheme 3 and FIG. 16, Table S2, Entry 10)

A solution of tetrabutylammonium fluoride in tetrahydrofuran (1.00 M, 6.91 mL, 6.91 mmol, 1.05 equiv) was added dropwise via syringe to a solution of the unpurified mixture of the two silacycles 14a and 14b (6.58 μmol, 1 equiv) in tetrahydrofuran (45 mL) at 0° C. The reaction was stirred for 30 min at 0° C. The reaction was diluted sequentially with pentane (45 mL) and an aqueous potassium phosphate buffer solution (pH 7, 0.10 M, 20 mL). The diluted mixture was transferred to a separatory funnel that had been charged with a mixture of ethyl acetate and hexanes (1:1, v/v, 300 mL). The layers that formed were separated and the organic layer obtained was washed with water (3×25 mL). The washed organic layer was dried over sodium sulfate. The dried solution was filtered and the filtrate was concentrated to dryness. The residue obtained containing the highly unstable primaryl alcohol intermediate S16 was used immediately in the next step without purification.

N,N-Dimethylformamide (28 mL) and an aqueous hydrogen peroxide solution (30% w/w, 14.9 mL, 145 mmol, 20.0 equiv) were added sequentially to a suspension of the unpurified intermediate S16 (6.58 mmol, 1 equiv) and potassium bicarbonate (14.5 g, 145 mmol, 22.0 equiv) in tetrahydrofuran (14 mL) at 24° C. in a 1-L round-bottomed flask equipped with a reflux condenser. The reaction vessel was placed in an oil bat that had been preheated to 80° C. and the reaction mixture was stirred and heated for 3 h at 80° C. The product mixture was diluted sequentially with dichloromethane (200 mL) and saturated aqueous sodium thiosulfate (50 mL). The diluted product mixture was transferred to a separatory funnel and the layers that formed were separated. The aqueous layer was extracted with dichloromethane (3×100 mL). The organic layers were combined and the combined organic layers were washed with water (10×20 mL). The washed organic layer was dried over sodium sulfate. The dried solution was filtered and the filtrate was concentrated to dryness. The residue obtained was purified by automated flash-column chromatography (eluting with hexanes initially, grading to 100% ethyl acetate-hexanes, linear gradient) to afford 18-hydroxy-19,20-dihydropleuromutilin 15a as an amorphous white solid (1.98 g, 74%, two steps).

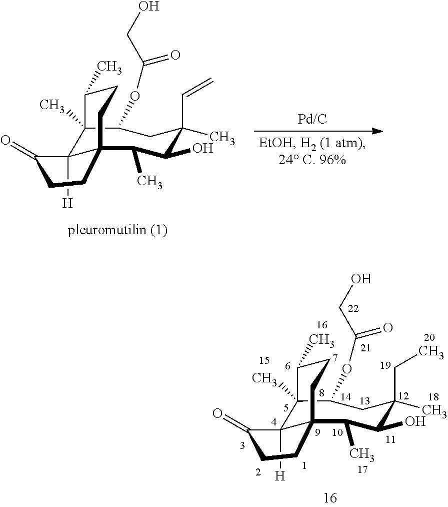

### Synthesis of 19,20-dihydropleuromutilin (16, FIG. 3, Scheme 3)

Palladium on carbon (5 wt. % loading, 338 mg, 159 μmol, 0.05 equiv) was added to a solution of pleuromutilin (1, 1.20 g, 3.17 mmol, 1 equiv) in ethanol (15 mL) at 24° C. The reaction vessel was evacuated and re-filled using a balloon of dihydrogen. This process was repeated four times. The reaction mixture was stirred for 12 h at 24° C. The product mixture was filtered through a short column of celite and the short column was rinsed with dichloromethane (200 mL). The filtrates were combined and the combined filtrates were concentrated to afford 19,20-dihydropleuromutilin (16) as an amorphous white solid (1.15 g, 96%).

19,20-dihydropleuromutilin (16): R_{f}=0.34 (50% ethyl acetate-hexanes; CAM, PAA). ¹H NMR (400 MHz, CDCl₃) 5.68 (d, J=8.0 Hz, 1H, H₁₄), 4.02 (q, J=16.0 Hz, 2H, H₂₂), 3.90 (d, J=6.0 Hz, 1H, H₁₁), 2.79 (br s, 1H, C22-OH), 2.41-2.33 (m, 1H, H₁₀), 2.28-2.12 (m, 2H, H₂), 2.08 (s, 1H, H₄), 1.80-1.66 (m, 4H, 1×H₈, 1×H₁₃, 1×H₁₉, 1×C11-OH), 1.65-1.49 (m, 4H, 1×H₁, 1×H₆, 1×H₇, 1×H₉), 1.45 (dt, J=12.4, 3.8 Hz, 1H, 1×H₁), 1.41-1.33 (m, 4H, 1×H₇, 3×H₁₈), 1.32-1.26 (m, 1H, 1×H₁₃), 1.09 (td, J=14.0, 4.8 Hz, 1H, 1×H₈), 0.95-0.87 (m, 6H, 3×H₁₇, 3×H₁₈), 0.72 (t, J=7.4 Hz, 3H, H₂O), 0.65 (d, J=6.8 Hz, 3H, H₁₆). ¹³C NMR (100 MHz, CDCl₃) δ 217.1 (C), 172.2 (C), 76.3 (CH), 69.9 (CH), 61.2 (CH₂), 58.3 (CH), 45.4 (C), 41.8 (C), 40.9 (C), 40.8 (CH₂), 36.5 (CH), 34.3 (CH₂), 34.3 (CH), 30.1 (CH₂), 26.7 (CH₂), 26.2 (CH₃), 24.8 (CH₂), 20.5 (CH₂), 16.4 (CH₃), 14.7 (CH₃), 11.0 (CH₃), 8.1 (CH₃). IR (ATR-FTIR), cm⁻¹: 3485 (br w), 2937 (w), 2879 (w), 1727 (s), 1460 (w), 1375 (w), 1283 (w), 1232 (m), 1157 (w), 1096 (m), 1046 (w), 1007 (w), 990 (w), 909 (s), 729 (s), 647 (w). HRMS-ESI (m/z): [M+H]⁺ calcd for C₂₂H₃₇O₅, 381.2641; found, 381.2640. [α]_{D}²⁵=+27° (c=1.0, CHCl₃).

A portion of 16 was further purified by recrystallization from methanol to afford a sample of 16.H₂O for X-ray analysis.

16.H₂O: mp 140-142° C.

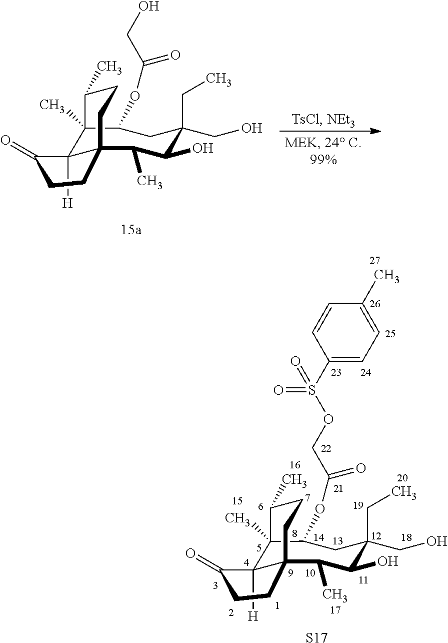

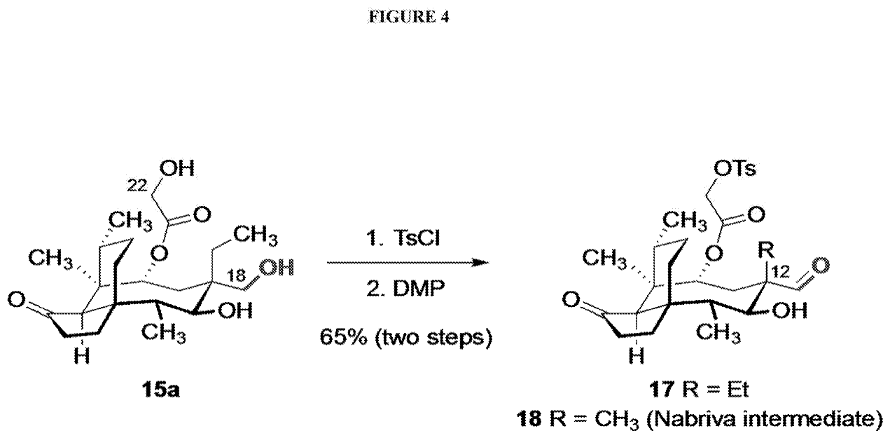

### Synthesis of O-(p-tolylsulfonyl)-18-hydroxy-19,20-dihydropleuromutilin (S17, FIG. 4, Scheme 4)

Triethylamine (76.7 μL, 550 μmol, 1.10 equiv) was added dropwise via syringe to a solution of 18-hydroxy-19,20-dihydropleuromutilin [15a, 198 mg, 500 μmol, 1 equiv, dried by azeotropic distillation with benzene (2.0 mL)] and p-tolylsulfonyl chloride (105 mg, 550 μmol, 1.10 equiv) in methyl ethyl ketone (9.0 mL) at 24° C. The reaction mixture was stirred at 24° C. for 12 h. The product mixture was diluted with saturated aqueous sodium bicarbonate solution (2.0 mL). The diluted mixture was transferred to a separatory funnel and the layers that formed were separated. The aqueous layer obtained was extracted with dichloromethane (3×5.0 mL). The organic layers were combined and the combined organic layer was dried over sodium sulfate. The dried solution was filtered and the filtrate was concentrated to dryness. The residue obtained was purified by automated flash-column chromatography (eluting with hexanes initially, grading to 40% acetone-hexanes, linear gradient) to afford O-(p-tolylsulfonyl)-18-hydroxy-19,20-dihydropleuromutilin S17 as an amorphous white solid (274 mg, 99%).

R_{f}=0.56 (50% acetone-dichloromethane; UV, CAM). ¹H NMR (400 MHz, CDCl₃) δ 7.82 (d, J 8.0 Hz, 2H, H₂₄), 7.35 (d, J 8.0 Hz, 2H, H₂₅), 5.70 (d, J=8.0 Hz, 1H, H₁₄), 4.49 (s, 2H, H₂₂), 3.85 (d, J=6.4 Hz, 1H, H₁₁), 3.57 (d, J=10.8 Hz, 1H, 1×H₁₈), 3.42 (d, J=11.2 Hz, 1H, 1×H₁₈), 2.45 (s, 3H, H₂₇), 2.42-2.35 (m, 1H, 1×H₁₉), 2.29-2.13 (m, 2H, H₂), 2.06 (s, 1H, H₄), 1.80-1.66 (m, 4H, 1×H₈, 1×H₁₃, 2×H₁₉), 1.63-1.54 (m, 2H, 1×H₁, 1×H₆), 1.52-1.41 (m, 2H, 1×H₁, 1×H₇), 1.40-1.32 (m, 4H, 1×H₇, 3×H₁), 1.14-1.06 (m, 2H, 1×H₈, 1×H₁₃), 0.94 (d, J=6.8 Hz, 3H, H₁₇), 0.73 (t, J=7.4 Hz, 3H, H₂₀), 0.60 (d, J=6.8 Hz, 3H, H₁₆). ¹³C NMR (100 MHz, CDCl₃) δ 216.6 (C), 165.1 (C), 145.3 (C), 132.6 (C), 129.9 (CH), 128.0 (CH), 75.1 (CH), 70.4 (CH₂), 69.7 (CH), 64.9 (CH₂), 58.2 (CH), 45.4 (C), 43.9 (C), 41.9 (C), 36.4 (CH), 35.2 (CH₂), 34.3 (CH), 34.2 (CH₂), 30.1 (CH₂), 26.7 (CH₂), 25.0 (CH₂), 21.6 (CH₃), 17.0 (CH₂), 16.4 (CH₃), 14.7 (CH₃), 10.7 (CH₃), 7.4 (CH₃). IR (ATR-FTIR), cm⁻¹: 3333 (br w), 2942 (w), 2881 (w), 1732 (m), 1598 (w), 1448 (w), 1371 (m), 1291 (w), 1219 (w), 1176 (s), 1119 (in), 1096 (m), 1037 (s), 952 (in), 910 (w), 816 (m), 773 (m), 663 (s), 552 (s). HRMS-ESI (m/z): [M+Na]⁺ calcd for C₂₉H₄O₈S, 551.2679; found, 551.2681. [α]_{D}²⁵=+25° (c=1.0, CHCl₃).

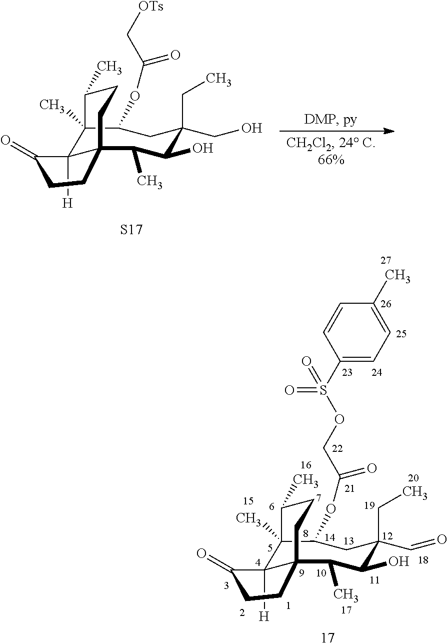

### Synthesis of O-(p-tolylsulfonyl)-18-oxo-19,20-dihydropleuromutilin (17. FIG. 4, Scheme 4)

Six equal portions of Dess-Martin periodinane (16.9 mg, 39.9 μmol, 1.10 equiv) was added over 1 h to a solution of O-(p-tolysulfonyl)-18-hydroxy-19,20-dihydropleuromutilin S17 (20.0 mg, 36.3 μmol, 1 equiv) and pyridine (29.4 μL, 363 μmol, 10.0 equiv) in dichloromethane (400 μL) at 24° C. The resulting mixture was stirred for 30 min at 24° C. The product mixture was diluted sequentially with ether (1.0 mL), a saturated aqueous sodium bicarbonate solution (500 μL) and a saturated aqueous sodium thiosulfate solution (500 μL). The resulting mixture was stirred for 5 min at 24° C. The resulting mixture was transferred to a separatory funnel and the layers that formed were separated. The aqueous layer obtained was extracted with dichloromethane (3×5.0 mL). The organic layers were combined and the combined organic layer was dried over sodium sulfate. The dried solution was filtered and the filtrate was concentrated to dryness. The residue obtained was purified by automated flash-column chromatography (eluting with hexanes initially, grading to 40% ethyl acetate-hexanes, linear gradient) to afford 0-(p-tolylsulfonyl)-18-oxo-19,20-dihydropleuromutilin (17) as an amorphous white solid (13.1 mg, 66%).

R_{f}=0.46 (33% ethyl acetate-hexanes; UV, CAM). ¹H NMR (400 MHz, CD₂Cl₂) δ 9.68 (s, 1H, H₁₈), 7.79 (d, J=8.0 Hz, 2H, H₂₄), 7.39 (d, J=8.0 Hz, 2H, H₂₅), 5.90 (d, J=9.2 Hz, 1H, H₁₄), 4.56-4.47 (m, 2H, H₂₂), 3.36 (dd, J=13.2, 6.4 Hz, 1H, H₁₁), 2.46 (s, 3H, H₂₇) 2.33-2.22 (m, 2H, H₂), 2.17-2.06 (m, 3H, 1×H₄, 1×H₁₀, 1×H₁₃), 1.68-1.52 (m, 4H, 1×H₁, 1×H₈, 2×H₁₉), 1.48-1.40 (m, 5H, 1×H₁, 1×H₆, 3×H₁₅), 1.32-1.17 (m, 3H, 2×H₇, 1×H₁₃), 1.14 (d, J=6.8 Hz, 3H, H₁₇), 0.90-0.85 (m, 1H, 1×H₈), 0.80 (t, J=7.4 Hz, 3H, H₂₀), 0.69 (d, J=6.8 Hz, 3H, H₁). ¹³C NMR (100 MHz, CDCl₃) δ 216.9 (C), 215.8 (C), 201.7 (CH), 165.9 (C), 146.4 (C), 133.0 (C), 130.6 (CH), 128.6 (CH), 69.9 (CH), 65.8 (CH₂) 64.7 (CH), 59.2 (CH₂), 46.0 (C), 44.3 (C), 42.6 (C), 37.6 (CH), 34.9 (CH₂), 32.7 (CH), 30.3 (CH₂), 27.2 (CH₃), 24.9 (CH₂), 24.2 (CH₂), 22.0 (CH), 17.2 (CH₂) 15.2 (CH), 12.9 (CH), 8.8 (CH). IR (ATR-FTIR), cm⁻¹: 2925 (m), 1735 (s), 1686 (m), 1454 (w), 1373 (m), 1289 (w), 1218 (w), 1190 (m), 1177 (s), 1110 (w), 1095 (w), 1046 (s), 816 (w), 779 (w), 664 (w), 554 (w). HRMS-ESI (m/z): [M+Na]⁺ calcd for C₂₉H₄₁O₈S, 549.2522; found, 549.2522. [α]_{D}²⁵=+24° (c=0.25, CHCl₃).

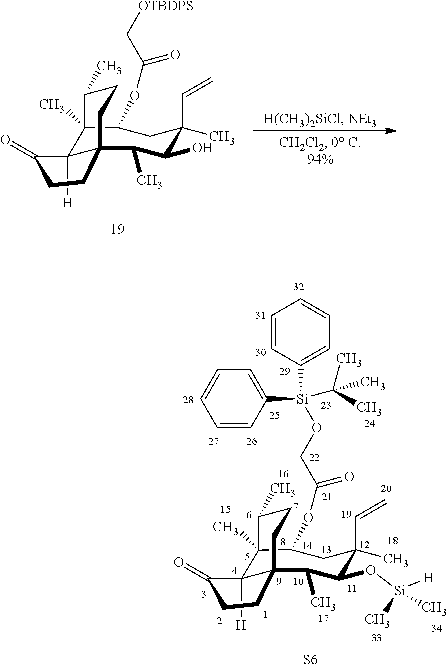

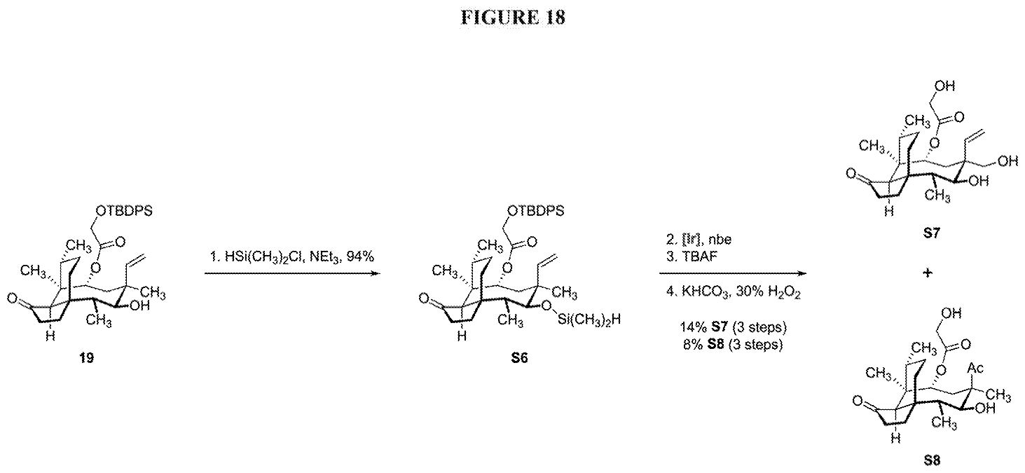

### Synthesis of Silane S6 (FIG. 18, Scheme S1)

Dimethylchlorosilane (18.0 μL, 162 μmol, 2.00 equiv) was added dropwise via syringe to a solution of O-tert-butyldiphenylsilylpleuromutilin [19, 50 mg, 81.1 μmol, 1 equiv, dried by azeotropic distillation with benzene (500 μL)] and triethylamine (45.2 μL, 324 μmol, 4.00 equiv) in dichloromethane (500 μL) at 0° C. The reaction mixture was stirred at 0° C. for 30 min. The product mixture was diluted sequentially with pentane (1.0 mL) and aqueous potassium phosphate buffer solution (pH 7, 0.10 M, 1.0 mL). The diluted mixture was transferred to a separatory funnel and the layers formed were separated. The aqueous layer was extracted with dichloromethane (3×5 mL). The organic layers were combined and the combined organic layers were dried over sodium sulfate. The dried solution was filtered and the filtrate was concentrated to dryness to afford silane S6 as an amorphous white solid (51.2 mg, 94%). The silane S6 prepared this way was analytically pure and was used in the next step without further purification.

R_{f}=0.60 (15% ethyl acetate-hexanes; UV, CAM). ¹H NMR (400 MHz, C₆D₆) δ 7.82-7.77 (m, 4H, 2×H₂₇, 2×H₃₁), 7.25-7.21 (m, 6H, 2×H₂₆, 1×H₂₈, 2×H₃₀, 1×H₃₂), 6.58 (dd, J=17.6, 11.2 Hz, 1H, H₁₉), 5.87 (d, J=8.01 Hz, 1H, H₁₄), 5.37-5.29 (m, 2H, H₂₀), 4.84 (sep, J=2.8 Hz, 1H, Si—H), 4.18 (s, 2H, H₂₂), 3.29 (d, J=6.0 Hz, 1H, H₁₁), 2.41-2.34 (m, 1H, H₁₀), 1.89-1.85 (m, 2H, H₂), 1.82-1.75 (m, 2H, 1×H₄, 1×H₁₃), 1.74-1.68 (m, 1H, H₁), 1.65 (s, 3H, H₁₅), 1.58-1.51 (m, 1H, 1×H₇), 1.44-1.28 (m, 3H, 1×H₆, 1×H₈, 1×H₁₃), 1.07 (s, 9H, H₂₄), 1.14-1.02 (m, 5H, 1×H₁, 1×H₇, 3×H₁₈), 0.92-0.75 (m, 4H, 1×H₈, 3×H₁₇), 0.70 (d, J=6.8 Hz, 3H, H₁₆), 0.17-0.14 (m, 6H, 3×H₃₃, 3×H₃₄). ¹³C NMR (100 MHz, C₆D₆) δ 214.8 (C), 169.1 (C), 139.9 (CH), 135.7 (CH), 133.0 (C), 133.0 (C), 129.8 (CH), 128.2 (CH), 127.8 (CH), 128.8 (CH), 127.5 (CH), 116.3 (CH₂), 78.9 (CH), 68.9 (CH), 62.9 (CH₂), 58.0 (CH), 45.0 (C), 44.6 (CH₂), 44.5 (C), 42.0 (C), 37.0 (CH), 36.6 (CH), 34.0 (CH), 30.1 (CH₂), 29.2 (CH₃), 26.6 (CH₂), 26.5 (CH₃), 26.1 (CH₂), 19.1 (C), 16.2 (CH₃), 14.8 (CH₃), 12.0 (CH₃), −0.93 (CH₃), −1.00 (CH₃). IR (ATR-FTIR), cm⁻¹: 2955 (w), 2861 (w), 1755 (w), 1734 (m), 1457 (w), 1252 (w), 1134 (m), 1113 (s), 1053 (m), 910 (s), 702 (s), 613 (m), 499 (s). HRMS-ESI (m/z): [M-Si(CH₃)₂+Na]⁺ calcd for C₃₈H₅₂NaO₅Si, 639.3482; found, 639.3486. [α]_{D}²⁵=+30° (c=0.20, CHCl₃).

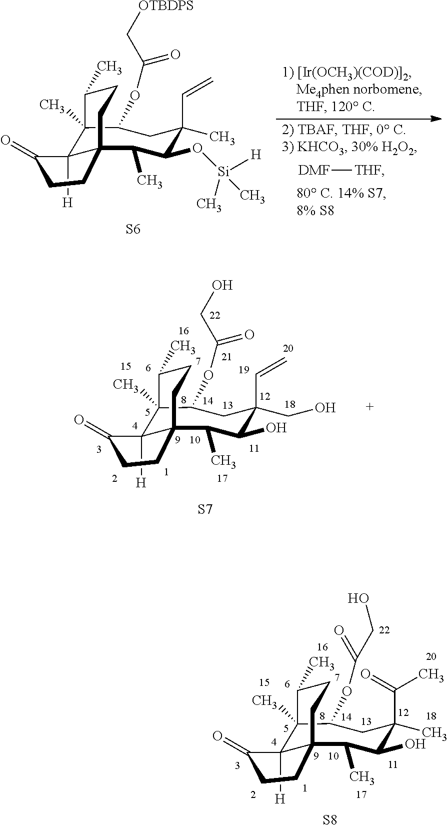

### Synthesis of 18-hydroxypleuromutilin (S7) and 19-oxo-20-hydropleuromutilin (S8)

This experiment was adapted from the work of Hartwig and co-workers.² A 4-mL pressure tube with a Teflon-coated valve was charged with 3,4,7,8-tetramethyl-1,10-phenanthroline (2.3 mg, 9.9 μmol, 12.5 mol %) and norbornene (10.7 mg, 114 μmol, 1.50 equiv) in the glovebox. A 4-mL vial was charged with silane S6 [51.2 mg, 75.9 μmol, 1 equiv, dried by azeotropic distillation with benzene (3×500 μL)]. The vessel containing the silane was evacuated and refilled using a balloon of argon. This process was repeated two times. Tetrahydrofuran (50 μL) was transferred into the vessel containing the silane and the resulting solution was added to the vessel containing the ligand and norbornene in the glovebox. The vessel containing the silane was rinsed with tetrahydrofuran (3×25 μL) and the combined rinses were transferred to the reaction vessel.

Methoxy(cyclooctadiene)iridium(I) dimer (2.4 mg, 3.8 μmol, 5.0 mol %) was added to an oven-dried 4-mL vial. Tetrahydrofuran (70 μL) was added into the vial containing the catalyst and the resulting solution was transferred dropwise via syringe to the reaction vessel in the glovebox. The vial containing the catalyst was rinsed with tetrahydrofuran (3×20 μL) and the combined rinses were transferred into the reaction vessel. The reaction vessel was sealed and the reaction mixture was stirred for 1 h at 24° C. in the glovebox. The sealed reaction vessel was then removed from the glovebox and placed in an oil bath that had been preheated to 120° C. The reaction mixture was stirred and heated for 2 h at 120° C. The reaction vessel was allowed to cool over 30 min to 24° C. and the cooled product mixture was concentrated to dryness. The residue obtained was filtered through a pad of silica gel (2.5×1.0 cm). The filter cake was washed with a mixture of ether and hexanes (1:1, v/v, 500 mL). The filtrate were combined and the combined filtrates were concentrated to dryness. The residue obtained contained was used in the next step without further purification.

A solution of tetrabutylammonium fluoride in tetrahydrofuran (1.00 M, 79.9 μL, 79.7 mmol, 1.05 equiv) was added dropwise via syringe to a solution of the unpurified mixture (nominally 79.7 μmol, 1 equiv) in tetrahydrofuran (500 μL) at 0° C. The reaction was stirred for 30 min at 0° C. The reaction was diluted sequentially with pentane (500 μL) and an aqueous potassium phosphate buffer solution (pH 7, 0.10 M, 500 μL). The diluted mixture was transferred to a separatory funnel that had been charged with a mixture of ethyl acetate and hexanes (1:1, v/v, 10 mL). The layers that formed were separated and the organic layer obtained was washed with water (3×2.0 mL). The washed organic layer was dried over sodium sulfate. The dried solution was filtered and the filtrate was concentrated to dryness. The residue obtained was used immediately in the next step without purification.

N,N-Dimethylformamide (4001 μL) and an aqueous hydrogen peroxide solution (30% w/w, 180 μL, 1.76 mmol, 20.0 equiv) were added sequentially to a suspension of the unpurified mixture (nominally 79.7 μmol, 1 equiv) and potassium bicarbonate (175 mg, 1.75 mmol, 22.0 equiv) in tetrahydrofuran (200 L) at 24° C. in a 4-mL vial. The vial was sealed with a Teflon-lined cap. The sealed vial was placed in an oil bat that had been preheated to 80° C. and the reaction mixture was stirred and heated for 3 h at 80° C. The product mixture was diluted sequentially with dichloromethane (1.0 mL) and saturated aqueous sodium thiosulfate (1.0 mL). The diluted product mixture was transferred to a separatory funnel and the layers that formed were separated. The aqueous layer was extracted with dichloromethane (3×5.0 mL). The organic layers were combined and the combined organic layers were washed with water (5×1.0 mL). The washed organic layer was dried over sodium sulfate. The dried solution was filtered and the filtrate was concentrated to dryness. The residue obtained was purified by automated flash-column chromatography (eluting with hexanes initially, grading to 100% ethyl acetate-hexanes, linear gradient) to afford separately 18-hydroxypleuromutilin (S7) as an amorphous white solid (4.1 mg, 14%, three steps) and 19-oxo-20-hydropleuromutilin (S8) as an amorphous white solid (2.3 mg, 8%).

18-Hydroxypleuromutilin (S7): R_{f}=0.11 (66% ethyl acetate-hexanes; PAA, CAM). ¹H NMR (500 MHz, CD₂Cl₂) 6.23 (dd, J=14.4, 9.2 Hz, 1H, H₁₉), 5.75 (d, J=6.8 Hz, 1H, H₁₄), 5.40 (d, J=9.2 Hz, 1H, 1×H₂₀), 5.25 (d, J=14.4 Hz, 1H, 1×H₂₀), 4.02 (td, J=11.6, 3.6 Hz, 2H, H₂₂), 3.87-3.84 (m, 1H, H₁₁), 3.76 (d, J=8.8 Hz, 1H, 1×H₁₈), 3.48 (d, J=8.8 Hz, 1H, 1×H₁₈), 2.36-2.10 (m, 6H, 2×H₂, 1×H₄, 1×H₁₀, 1×H₁, 1×C18-OH), 2.05-1.97 (br m, 1H, C22-OH), 1.80-1.76 (m, 1H, 1×H₈), 1.67-1.58 (m, 3H, 1×H₆, 1×H₇, 1×C11-OH), 1.52-1.46 (m, 2H, 1×H₁, 1×H₇), 1.43 (s, 3H, H₁₅), 1.40-1.32 (m, 2H, 1×H₁, 1×H₁₃), 1.17-1.10 (m, 1H, 1×H₈), 0.95 (d, =5.6 Hz, 3H, H₁), 0.69 (d, J=5.6 Hz, 3H, H₁₆). ¹³C NMR (125 MHz, CD₂Cl₂) δ 217.2 (C), 172.6 (C), 137.7 (CH), 118.8 (CH₂), 72.3 (CH), 70.4 (CH), 70.2 (CH₂), 61.9 (CH₂), 58.6 (CH), 49.1 (C), 46.1 (C), 42.6 (C), 40.0 (CH₂), 37.2 (CH), 36.6 (CH), 34.9 (CH₂), 30.8 (CH₂), 27.4 (CH₂), 25.6 (CH₂), 16.7 (CH₃), 15.1 (CH₃), 11.6 (CH₃). IR (ATR-FTIR), cm⁻¹: 3414 (br m), 2939 (m), 2883 (w), 1729 (s), 1456 (w), 1374 (w), 1233 (m), 1096 (m), 1037 (m), 725 (w). HRMS-ESI (m/z): [M+Na]⁺ calcd for C₂₂H₃₄NaO₆, 417.2253; found, 417.2249.

19-Oxo-20-hydropleuromutilin (S8): R_{f}=0.34 (66% ethyl acetate-hexanes; PAA, CAM). ¹H NMR (400 MHz, CD₂Cl₂) δ 5.56 (d, J=8.0 Hz, 1H, H₁₄), 4.13-4.00 (m, 2H, H₂₂), 3.22 (dd, J=12.0, 6.4 Hz, 1H, H₁₁), 2.58 (d, J=12.0 Hz, 1H, C11-OH), 2.43-2.29 (m, 2H, 1×H₁, 1×H₁₀), 2.24-2.10 (m, 4H, 2×H₂, 1×H₄, 1×H₁₃), 2.05 (s, 3H, H₂₀), 1.90-1.84 (m, 1H, 1×H₁₃), 1.80 (dt, J=19.2, 4.4 Hz, 1H, 1×H₈), 1.67-1.58 (m, 3H, 1×H₆, 1×H₇, 1×C22-OH), 1.51-1.44 (m, 2H, 1×H₁, 1×H₇), 1.42 (s, 3H, H₁₅), 1.34 (s, 3H, H₁₈), 1.17-1.12 (m, 1H, 1×H₈), 1.09 (d, J=6.8 Hz, 3H, H₁₇), 0.67 (d, J=6.8 Hz, 3H, H₁₆). ¹³C NMR (125 MHz, CD₂Cl₂) δ 217.1 (C), 215.4 (C), 173.2 (C), 76.2 (CH), 71.0 (CH), 61.9 (CH₂), 58.7 (CH), 57.3 (C), 46.1 (C), 42.8 (C), 42.5 (CH₂), 38.8 (CH), 37.0 (CH), 34.8 (CH₂), 30.9 (CH₂), 27.3 (CH₃), 26.9 (CH₂), 26.3 (CH₃), 25.3 (CH₂), 16.8 (CH₃), 14.9 (CH₃), 11.6 (CH₃). IR (ATR-FTIR) cm⁻¹: 3391 (br w), 2931 (m) 1731 (s), 1691 (m) 1456 (m), 1222 (m), 1094 (s), 1016 (m), 736 (m). HRMS-ESI (m/z): [M+Na]⁺ calcd for C₂₂H₃₄NaO₆, 417.2253; found, 417.2248.

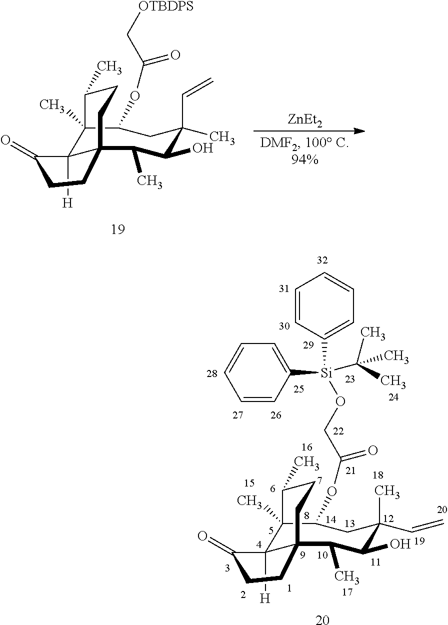

### Synthesis of Silane 20 (FIG. 5, Scheme 5)

A 500-mL round-bottomed flask fused to a Teflon-coated valve was charged with O-tert-butyldiphenylsilylpleuromutilin (19, 12.3 g, 20.0 mmol, 1 equiv). Benzene (50 mL) was added and the solution was concentrated to dryness. This process was repeated twice. Deoxygenated N,N-dimethylformamide (180 mL) was added to the reaction vessel and the vessel was sealed. The sealed vessel was transferred to the glovebox. A solution of diethylzinc (1.0 M, 21.0 mL, 21.0 mmol, 1.05 equiv) in toluene was added dropwise under vigorous stirring at 24° C. The reaction vessel was removed from the glovebox and placed in an oil bath that had been previously heated to 100° C. The reaction mixture was stirred and heated for 2 h at 100° C. The product mixture was allowed to cool to 0° C. with an ice bath over 30 min. A saturated aqueous ammonium chloride solution (50 mL) was added dropwise via syringe to the product mixture. The resulting mixture was stirred for 10 min at 0° C. The diluted mixture was transferred to a separatory funnel that had been previously charged with ethyl acetate (200 mL) and water (20 mL) and the layers were separated. The layers that formed were separated and the aqueous layer was extracted with ethyl acetate (3×100 mL). The organic layers were combined and the combined organic layers were washed with water (5×25 mL). The organic layer was dried over sodium sulfate. The dried solution was filtered and the filtrate was concentrated. The residue obtained was purified by automated flash-column chromatography (eluting with dichloromethane initially, grading to 5% ether-dichloromethane, linear gradient) to afford separately 0-tert-butyldiphenylsilyl-12-epi-pleuromutilin (20, combined with future fractions) and O-tert-butyldiphenylsilylpleuromutilin (19, 5.07 g).

The recovered O-tert-butyldiphenylsilylpleuromutilin (19, 5.07 g, 8.21 mmol, 1 equiv) was subjected to the same epimerization procedure with a solution of diethylzinc (8.62 mL, 8.62 mmol, 1.05 equiv) and N,N-dimethylformamide (70 mL). The resulting product mixture was purified by automated flash-column chromatography (eluting with dichloromethane initially, grading to 5% ether-dichloromethane, linear gradient) to afford separately O-tert-butyldiphenylsilyl-12-epi-pleuromutilin (20, combined with future fractions) and O-tert-butyldiphenylsilylpleuromutilin (19, 2.08 g).

The recovered O-tert-butyldiphenylsilylpleuromutilin (19, 2.08 g, 3.37 mmol, 1 equiv) was subjected to the same epimerization procedure with a solution of diethylzinc (3.54 mL, 3.54 mmol, 1.05 equiv) and N,N-dimethylformamide (30 mL). The resulting product mixture was purified by automated flash-column chromatography (eluting with dichloromethane initially, grading to 5% ether-dichloromethane, linear gradient) to afford separately O-tert-butyldiphenylsilyl-12-epi-pleuromutilin (20, combined with future fractions) and O-tert-butyldiphenylsilylpleuromutilin (19, 1.12 g).

The recovered O-tert-butyldiphenylsilylpleuromutilin (19, 1.12 g, 1.82 mmol, 1 equiv) was subjected to the same epimerization procedure with a solution of diethylzinc (1.91 mL, 1.91 mmol, 1.05 equiv) and N,N-dimethylformamide (15 mL). The resulting product mixture was purified by automated flash-column chromatography (eluting with dichloromethane initially, grading to 5% ether-dichloromethane, linear gradient) to afford separately O-tert-butyldiphenylsilyl-12-epi-pleuromutilin (20, combined with future fractions) and O-tert-butyldiphenylsilylpleuromutilin (19, 592 mg).

The recovered O-tert-butyldiphenylsilylpleuromutilin (19, 592 mg, 960 μmol, 1 equiv) was subjected to the same epimerization procedure with a solution of diethylzinc (1.01 mL, 1.01 mmol, 1.05 equiv) and N,N-dimethylformamide (9.0 mL). The resulting product mixture was purified by automated flash-column chromatography (eluting with dichloromethane initially, grading to 5% ether-dichloromethane, linear gradient) to afford separately O-tert-butyldiphenylsilyl-12-epi-pleuromutilin (20) as an amorphous white solid (11.8 g, 94% after four recycles).

O-tert-Butyldiphenylsilyl-12-epi-pleuromutilin (20): R_{f}=0.51 (5% ether-dichloromethane; UV, PAA, CAM). ¹H NMR (500 MHz, CDCl₃) δ 7.69-7.67 (m, 4H, 2×H₂₇, 2×H₃₁), 7.44-7.37 (m, 6H, 2×H₂₆₁×H₂₈, 2×H₃₀, 1×H₃₂), 5.73 (dd, J=17.0, 8.4 Hz, 1H, H₁₉), 5.67 (d, J=6.4 Hz, 1H, H₁₄), 5.24-5.20 (m, 2H, H₂₀), 4.15 (dd, J=18.4, 5.2 Hz, 2H, H₂₂), 3.44 (d, J=4.0 Hz, 1H, H₁₁), 2.45-2.39 (m, 1H, H₁₀), 2.28-2.15 (m, 2H, H₂), 2.09 (s, 1H, H₄), 2.00 (dd, J=12.4, 6.8 Hz, 1H, 1×H₁₃), 1.80 (dt, J=11.6, 2.0 Hz, 1H, 1×H₈), 1.68-1.47 (m, 5H, 1×H₁, 1×H₆, 2×H₇, 1×OH), 1.40-1.35 (m, 4H, 1×H₁, 3×H₁₅), 1.26 (s, 3H, H₁₈), 1.15-1.08 (m, 10H, 1×H₈, 9×H₂₄), 1.01-0.96 (m, 4H, 1×H₁₃, 3×H₁₇), 0.62 (d, J=5.2 Hz, 3H, H₁₆). ¹³C NMR (125 MHz, CDCl₃) δ 217.1 (C), 169.8 (C), 147.1 (CH), 135.5 (CH), 132.8 (C), 132.7 (C), 129.8 (CH), 128.3 (CH), 127.7 (CH), 115.0 (CH₂), 72.0 (CH), 68.6 (CH), 62.8 (CH₂), 58.3 (CH), 7834 (C), 45.2 (C), 43.6 (CH₂), 41.8 (C), 36.7 (CH), 34.5 (CH₂), 34.3 (CH), 30.1 (CH₂), 26.9 (CH₂), 26.6 (CH₃), 25.0 (CH₂), 19.1 (C), 16.6 (CH₃), 14.9 (CH₃), 14.3 (CH₃), 10.7 (CH₃). IR (ATR-FTIR), cm⁻¹: 2932 (w), 2862 (w), 1734 (m), 1472 (w), 1135 (m), 1113 (s), 1032 (w), 907 (m), 824 (w), 729 (s), 701 (s), 504 (m). HRMS-ESI (m/z): [M+Na]⁺ calcd for C₃₈H₅₂NaO₅Si, 639.3482; found, 639.3486. [α]_{D}²⁵=+34° (c=1.0, CHCl₃).

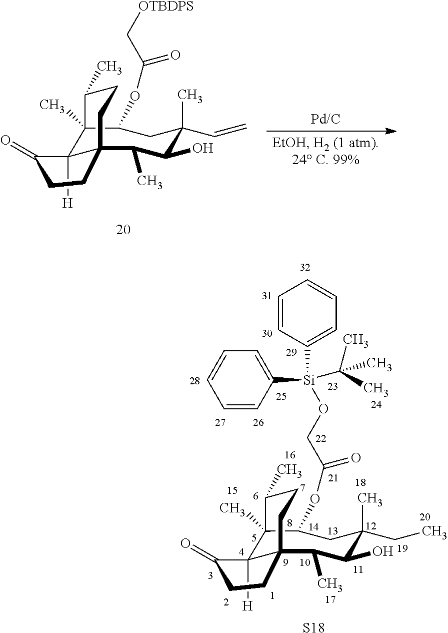

### Synthesis of O-tert-butyldiphenylsilyl-12-epi-19,20-dihydropleuromutilin (S18, FIG. 5, Scheme 5)

Palladium on carbon (5 wt. % loading, 156 mg, 73.0 μmol, 0.05 equiv) was added to a solution of O-tert-butyldiphenylsilyl-12-epi-pleuromutilin (20, 900 mg, 1.46 mmol, 1 equiv) ethanol (10 mL) at 24° C. The reaction vessel was evacuated and re-filled using a balloon of dihydrogen. This process was repeated four times. The reaction mixture was stirred for 12 h at 24° C. The product mixture was filtered through a short column of celite and the short column was rinsed with dichloromethane (50 mL). The filtrates were combined and the combined filtrates were concentrated to afford O-tert-butyldiphenylsilyl-12-epi-19,20-dihydropleuromutilin (S18) as an amorphous white solid (904 mg, 99%).

R_{f}=0.54 (20% ethyl acetate-hexanes; UV, PAA, CAM). ¹H NMR (400 MHz, CDCl₃) δ 7.69-7.66 (m, 4H, 2×H₂₇, 2×H₃₁), 7.45-7.34 (m, 6H, 2×H₂₆, 1×2H₂₈, 2×H₃₀, 1×H₃₂), 5.62 (d, J=8.4 Hz, 1H, H₁₄), 4.14 (dd, J=24.2, 7.2 Hz, 2H, H₂₂), 3.49 (t, J=6.0 Hz, 1H, H₁₁), 2.42-2.35 (m, 1H, H₁₀), 2.29-2.13 (m, 2H, H₂), 2.04-1.95 (m, 2H, 1×H₄, 1×H₁₃), 1.80 (dt, J=14.4, 2.0 Hz, 1H, 1×H₈), 1.65-1.43 (m, 6H, 2×H₁, 1×He, 1×H₇, 1×H₁₉, 1×OH), 1.37 (s, 3H, H₁₅), 1.35-1.24 (m, 2H, 1×H₇, 1×H₁₉), 1.14-1.10 (m, 1H, 1×H₈), 1.08 (s, 9H, 9×H₂₄), 1.04 (s, 3H, H₁₈), 0.93 (d, J=7.2 Hz, 3H, H₁₇), 0.88-0.84 (m, 4H, 1×H₁₃, 3×H₂₀), 0.60 (d, J=6.4 Hz, 3H, H₁₆). ¹³C NMR (100 MHz, CDCl₃) δ 217.2 (C), 169.8 (C), 135.5 (CH), 132.8 (C), 132.7 (C), 129.9 (CH), 127.8 (CH), 72.0 (CH), 69.0 (CH), 62.8 (CH₂), 58.2 (CH), 45.5 (C), 41.9 (CH₂), 41.7 (C), 40.2 (C), 36.7 (CH), 34.7 (CH₂), 34.5 (1×CH₂, 1×CH), 30.3 (CH₂), 26.9 (CH₂), 26.7 (CH₃), 25.0 (CH₂), 19.2 (C), 17.8 (CH₃), 16.7 (CH), 14.9 (CH₃), 10.9 (CH₃), 7.9 (CH₃). IR (ATR-FTIR), cm⁻¹: 2956 (w), 2860 (w), 1734 (m), 1463 (w), 1217 (w), 1138 (m), 1113 (s), 966 (w), 910 (m), 824 (m), 732 (s), 702 (s), 505 (s). HRMS-ESI (m/z): [M+Na]⁺ calcd for C₃₈H₅₄NaO₅Si, 641.3638; found, 641.3635. [α]_{D}²⁵=+32° (c=1.0, CHCl₃).

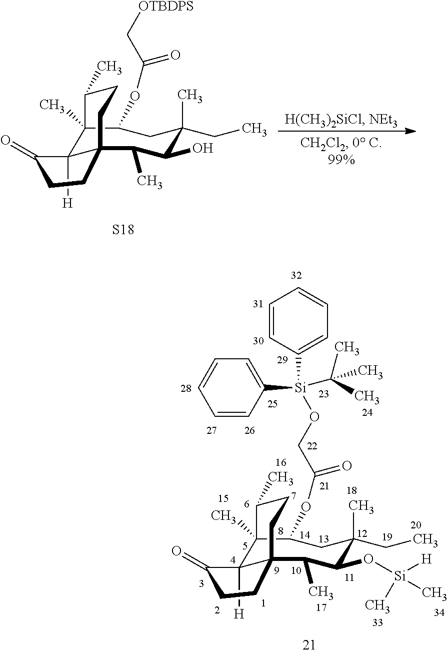

### Synthesis of Silane 2 (FIG. 5, Scheme 5)

Dimethylchlorosilane (324 μL, 2.92 mmol, 2.00 equiv) was added dropwise via syringe to a solution of O-tert-butyldiphenylsilyl-12-epi-19,20-dihydropleuromutilin [S18, 904 mg, 1.46 mmol, 1 equiv, dried by azeotropic distillation with benzene (5.0 mL)] and triethylamine (814 μL, 5.84 mmol, 4.00 equiv) in dichloromethane (8.0 mL) at 0° C. The reaction mixture was stirred for 30 min at 0° C. The product mixture was diluted sequentially with pentane (10 mL) and aqueous potassium phosphate buffer solution (pH 7, 0.10 M, 5.0 mL). The diluted mixture was transferred to a separatory funnel and the layers that formed were separated. The aqueous layer was extracted with dichloromethane (3×10 mL). The organic layers were combined and the combined organic layers were dried over sodium sulfate. The dried solution was filtered and the filtrate was concentrated to dryness to afford silane 21 as an amorphous white solid (991 mg, 99%). The silane 21 prepared this way was analytically pure and was used in the next step without further purification.

R_{f}=0.63 (20% ether-hexanes; UV, PAA, CAM). ¹H NMR (400 MHz, C₆D₆) δ 7.75-7.72 (m, 4H, 2×H₂₇, 2×H₃₁), 7.19-7.16 (m, 6H, 2×H₂₆, 1×H₂₈, 2×H₃₀, 1×H₃₂), 5.76 (d, J=8.8 Hz, 1H, H₁₄), 4.81 (sep, J=2.7 Hz, 1H, Si—H), 4.14 (s, 2H, H₂₂), 3.40 (d, J=6.0 Hz, 1H, H₁₁), 2.41-2.34 (m, 1H, H₁₀), 1.87-1.72 (m, 4H, 2×H₂, 1×H₄, 1×H₁₃), 1.68-1.59 (m, 4H, 1×H₆, 3×H₁₅), 1.50-1.30 (m, 4H, 1×H₁, 1×H₇, 1×H₈, 1×H₁₉), 1.27-1.22 (m, 1H, 1×H₁), 1.19 (s, 3H, H₁₈), 1.15 (s, 9H, 9×H₂₄), 1.09-0.97 (m, 3H, 1×H₇, 1×H₈, 1×H₁₉), 0.81-0.74 (m, 4H, 1×H₁, 3×H₁₇), 0.71 (t, J=7.8 Hz, 3H, H₂₀), 0.63 (d, J=7.2 Hz, 3H, H₁₆), 0.13-0.11 (m, 6H, 3×H₃₃, 3×H₃₄). ¹³C NMR (100 MHz, C₆D₆) δ 214.7 (C), 169.2 (C), 135.7 (CH), 135.6 (CH), 133.1 (C), 133.0 (C), 129.8 (CH), 128.2 (CH), 127.8 (CH), 77.4 (CH), 68.9 (CH), 62.8 (CH₂), 57.8 (CH), 45.0 (C), 41.9 (C), 41.2 (C), 41.0 (CH₂), 36.6 (CH), 35.5 (CH), 34.4 (CH₂), 34.1 (CH₂), 30.3 (CH₂), 26.8 (CH₂), 26.5 (CH₃), 25.1 (CH₂), 19.1 (C), 16.8 (CH₃), 16.6 (CH₃), 14.9 (CH₃), 11.9 (CH), 7.9 (CH₃), −0.64 (CH₃), −0.77 (CH₃). IR (ATR-FTIR), cm⁻¹: 2959 (w), 2860 (w), 1737 (m), 1463 (w), 1252 (w), 1215 (w), 1133 (m), 1113 (m), 1077 (w), 1054 (m), 910 (m), 824 (m), 701 (s), 613 (w), 498 (s). HRMS-ESI (m/z): [M-Si(CH₃)₂+Na]⁺ calcd for C₃₈H₅₄NaO₅Si, 641.3438; found, 641.3443. [α]_{D}²⁵=+32° (c=1.0, CHCl₃).

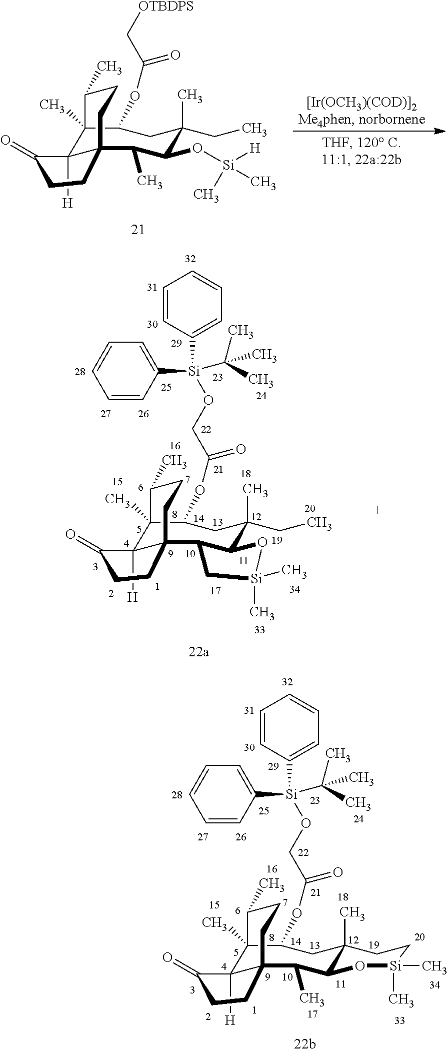

### Synthesis of Silacycles 22a and 22b (FIG. 5, Scheme 5)

This experiment was adapted from the work of Hartwig and co-workers.² A 25-mL pressure tube with a Teflon-coated valve was charged with 3,4,7,8-tetramethyl-1,10-phenanthroline (33.4 mg, 141 μmol, 12.5 mol %) and norbornene (160 mg, 1.70 mmol, 1.50 equiv) in the glovebox. A 20-mL vial was charged with silane 21 [766 mg, 1.13 mmol, 1 equiv, dried by azeotropic distillation with benzene (3×5.0 mL)]. The vessel containing the silane was evacuated and refilled using a balloon of argon. This process was repeated two times. Tetrahydrofuran (1.0 mL) was transferred into the vessel containing the silane and the resulting solution was added to the vessel containing the ligand and norbornene in the glovebox. The vessel containing the silane was rinsed with tetrahydrofuran (3×200 μL) and the combined rinses were transferred to the reaction vessel.

Methoxy(cyclooctadiene)iridium(I) dimer (37.5 mg, 56.6 μmol, 5.0 mol %) was added to an oven-dried 4-mL vial. Tetrahydrofuran (1.0 mL) was added into the vial containing the catalyst and the resulting solution was transferred dropwise via syringe to the reaction vessel in the glovebox. The vial containing the catalyst was rinsed with tetrahydrofuran (3×300 μL) and the combined rinses were transferred into the reaction vessel. The reaction vessel was sealed and the reaction mixture was stirred for 1 h at 24° C. in the glovebox. The sealed reaction vessel was then removed from the glovebox and placed in an oil bath that had been preheated to 120° C. The reaction mixture was stirred and heated for 2 h at 120° C. The reaction vessel was allowed to cool over 30 min to 24° C. and the cooled product mixture was concentrated to dryness. The residue obtained was filtered through a pad of silica gel (2.5×2.5 cm). The filter cake was washed with a mixture of ether and hexanes (1:1, v/v, 100 mL). The filtrate were combined and the combined filtrates were concentrated to dryness. The residue obtained contained a mixture of C11-C17-silacycle 22a and C11-C20-silacycle 22b (763 mg, 99%) and was used in the next step without further purification. ¹H NMR study of the unpurified mixture revealed an approximate 11:1 mixture of 22a:22b. An analytically pure sample of 22a and 22b were obtained for characterization by automated flash-column chromatography (eluting with hexanes initially, grading to 15% ethyl acetate-hexanes, linear gradient).

C11-C17-silacycle 22a: Amorphous white solid. R_{f}=0.55 (20% ether-hexanes; UV, PAA, CAM). ¹H NMR (400 MHz, C₆D₆) δ 7.75-7.73 (m, 4H, 2×H₂₇, 2×H₃₁), 7.19-7.17 (m, 6H, 2×H₂₆, 1×H₂₈, 2×H₃₀, 1×H₃₂), 5.76 (d, J=8.0 Hz, 1H, H₁₄), 4.14 (s, 2H, H₂₂), 3.71 (d, J=5.6 Hz, 1H, H₁₁), 2.71-2.65 (m, 1H, H₁₀), 1.91-1.66 (m, 6H, 2×H₂, 1×H₄, 1×H_{f}, 1×H₁₃, 1×H₁₉), 1.62-1.59 (m, 4H, 3×H₁₅, 1×H₁₉), 1.58-1.54 (m, 2H, H₇), 1.33 (dt, J=13.2, 2.0 Hz, 1×H₈), 1.18 (s, 9H, 9×H₂₄), 1.19 (s, 3H, H₁₈), 1.07-0.94 (m, 3H, 2×H₁, 1×H₁₃), 0.87-0.78 (m, 4H, 1×H₈, 3×H₂₀), 0.66 (d, J=7.2 Hz, 3H, H₁₆), 0.52 (dd, J=15.6, 12.0 Hz, 1H, 1×H₁₇), 0.52 (dd, J=12.0, 6.4 Hz, 1H, 1×H₁₇), 0.09 (s, 3H, H₃₃), 0.04 (s, 3H, H₃₃). ¹³C NMR (100 MHz, C₆D₆) δ 214.3 (C), 169.3 (C), 135.7 (CH), 133.1 (C), 133.0 (C), 129.8 (CH), 128.2 (CH), 127.8 (CH), 82.6 (CH), 68.9 (CH), 67.8 (CH₂), 58.4 (CH), 45.1 (C), 41.7 (CH₂), 41.6 (C), 40.1 (CH), 39.4 (C), 36.5 (CH), 34.9 (CH₂), 33.8 (CH₂), 31.1 (CH₂), 26.9 (CH₂), 26.5 (CH₃), 24.9 (CH₂), 19.1 (C), 18.5 (CH₃), 16.6 (CH₃), 14.9 (CH₃), 12.5 (CH₂), 7.9 (CH₃), −0.29 (CH₃), −2.5 (CH₃). IR (ATR-FTIR), cm⁻¹: 2958 (w), 2931 (w), 2859 (w), 1738 (m), 1463 (w), 1252 (w), 1215 (w), 1141 (m), 1113 (s), 1056 (m), 863 (m), 824 (m), 702 (s), 613 (m), 498 (s). HRMS-ESI (m/z): [M+Na]⁺ calcd for C₄₀H₅₉NaO₅Si₂, 697.3720; found, 697.3719. [α]_{D}²⁵=+27° (c=1.0, CHCl₃).

C11-C20-silacycle 22b: Amorphous white solid. R_{f}=0.63 (20% ether-hexanes; UV, PAA, CAM). ¹H NMR (500 MHz, C₆D₆) δ 7.82-7.79 (m, 4H, 2×H₂₇, 2×H₃₁), 7.25-7.21 (m, 6H, 2×H₂, 1×H₂₈, 2×H₃₀, 1×H₃₂), 5.83 (d, J=8.5 Hz, 1H, H₁₄), 4.20 (s, 2H, H₂₂), 3.47 (d, J=6.0 Hz, 1H, H₁₁), 2.37-2.31 (m, 1H, H₁₀), 1.92-1.80 (m, 3H, 2×H₂, 1×H₄), 1.78-1.68 (m, 1H, 1×H₆), 1.66 (s, 3H, H₁₈), 1.53-1.43 (m, 3H, 1×H₇, 1×H₁, 1×H₉), 1.41-1.32 (m, 2H, 1×H₈, 1×H₁₉), 1.30-1.27 (m, 1H, 1×H₁), 1.25 (s, 3H, H₁₈), 1.22 (s, 9H, 9×H₂₄), 1.15-1.01 (m, 2H, 1×H₁, 1×H₇), 0.89 (d, J=7.0 Hz, 3H, H₁₇), 0.86-0.82 (m, 1H, 1×H₈), 0.79 (dd, J=14.0, 4.0 Hz, 1H, 1×H₁₃), 0.73 (dd, J=14.5, 6.0 Hz, 1H, 1×H₂₀), 0.68 (d, J=7.0 Hz, 3H, H₁₆), 0.34 (dt, J=14.5, 3.5 Hz, 1H, 1×H₂₀), 0.10 (s, 3H, H₃₃), 0.04 (s, 3H, H₃₃). ¹³C NMR (125 MHz, C₆D₆) δ 214.9 (C), 169.2 (C), 135.7 (CH), 135.7 (CH), 133.1 (C), 133.0 (C), 129.8 (CH), 127.8 (CH), 76.1 (CH), 68.8 (CH), 62.8 (CH₂), 57.8 (CH), 47.0 (CH₂), 45.0 (C), 41.8 (C), 39.3 (CH₂), 38.9 (C), 36.6 (CH), 36.0 (CH), 34.0 (CH₂), 30.2 (CH₂), 26.8 (CH₂), 26.5 (CH₃), 24.6 (CH₂), 16.1 (C), 16.6 (CH₃), 15.0 (CH₃), 14.9 (CH₃), 10.9 (CH₃), 8.7 (CH₂), −0.94 (CH₃), −3.3 (CH₃). IR (ATR-FTIR), cm⁻¹: 2958 (w), 2931 (w), 2859 (w), 1738 (m), 1463 (w), 1252 (w), 1215 (w), 1141 (m), 1113 (s), 1056 (m), 863 (m), 824 (m), 702 (s), 613 (m), 498 (s). HRMS-ESI (m/z): [M+K]⁺ calcd for C₄₀H₅₈KO₅Si₂, 713.3460; found, 713.3450.

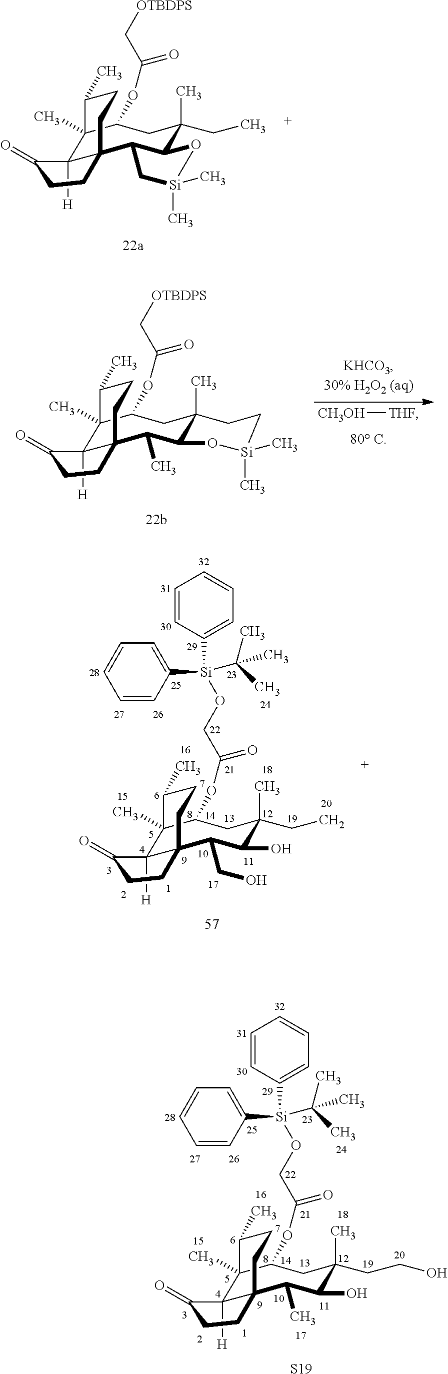

### Tamao-Fleming Oxidation of a Mixture of 57 and S19 (FIG. 5, Scheme 5)

Tetrahydrofuran (300 μL) and an aqueous hydrogen peroxide solution (30% w/w, 336 μL, 2.96 mmol, 20.0 equiv) were added sequentially to a suspension of the unpurified mixture of the two silacycles 22a and 22b (100 mg, 148 μmol, 1 equiv) and potassium bicarbonate (88.9 mg, 889 μmol, 6.00 equiv) in methanol (300 μL) at 24° C. in a 4-mL vial. The vial was sealed with a Teflon-lined cap and the sealed vial was placed in an oil bat that had been preheated to 80° C. The reaction mixture was stirred and heated for 3 h at 80° C. The product mixture was diluted sequentially with dichloromethane (2.0 mL) and saturated aqueous sodium thiosulfate (1.0 mL). The diluted product mixture was transferred to a separatory funnel and the layers that formed were separated. The aqueous layer was extracted with dichloromethane (3×5 mL). The organic layers were combined and the combined organic layers were dried over sodium sulfate. The dried solution was filtered and the filtrate was concentrated to dryness. The residue obtained contained a mixture of diols 57 and S19 (94.2 mg, 99%) and was used in the next step without further purification. An analytically pure sample of 57 and S19 were obtained for characterization by automated flash-column chromatography (eluting with hexanes initially, grading to 100% ethyl acetate-hexanes, linear gradient).

Diol 57: Amorphous white solid. R_{f}=0.33 (66% ethyl acetate-hexanes; UV, PAA, CAM). ¹H NMR (400 MHz, CDCl₃) δ 7.69-7.66 (m, 4H, 2×H₂₇, 2×H₃₁), 7.45-7.35 (m, 6H, 2×H₂₆, 1×H₂, 2×H₃₀, 1×H₃₂), 5.59 (d, J=8.4 Hz, 1H, H₁₄), 4.14 (dd, J=25.2, 8.1 Hz, 2H, H₂₂), 3.93 (td, J=9.8, 4.8 Hz, 1H, 1×H₁₇), 3.86-3.81 (br m, 1H, 1×H₁₇), 3.67 (t, J=7.0 Hz, 1H, H₁₁), 3.31 (d, J=7.6 Hz, 1H, C11-OH), 2.69 (t, J=5.6 Hz, 1H, C17-OH), 2.41 (td, J=6.8, 2.8 Hz, 1H, H₁₀), 2.28-2.11 (m, 2H, H₂), 1.99-1.95 (m, 2H, 1×H₄, 1×H₁₃), 1.82-1.73 (m, 2H, 1×H₁, 1×H₈), 1.68-1.62 (m, 1H, 1×H₇), 1.61-1.50 (m, 2H, 1×H₆, 1×H₁₉), 1.43-1.33 (m, 5H, 1×H₁, 3×H₁₅, 1×H₁₉), 1.19-1.11 (m, 2H, 1×H₇, 1×H₈), 1.10-1.05 (m, 12H, 3×H₁₈, 9×H₂₄), 0.88-0.84 (m, 4H, 1×H₁, 3×H₂), 0.66 (d, J=6.4 Hz, 3H, H₁₆). ¹³C NMR (100 MHz, C₆D₆) δ 216.6 (C), 169.9 (C), 135.6 (CH), 135.5 (CH), 132.8 (C), 132.7 (C), 129.9 (CH), 127.8 (CH), 73.6 (CH), 68.8 (CH), 62.8 (CH₂), 61.6 (CH₂), 58.4 (CH), 44.1 (C), 42.9 (CH), 41.9 (CH₂), 41.6 (C), 40.0 (C), 36.7 (CH), 34.5 (CH₂), 34.4 (CH₂), 30.5 (CH₂), 26.9 (CH₂), 26.7 (CH₃), 25.8 (CH₂), 19.2 (C), 18.5 (CH₃), 16.6 (CH₃), 14.9 (CH₃), 7.9 (CH₃). IR (ATR-FTIR), cm⁻¹: 3353 (br w), 2957 (w), 2860 (w), 1735 (m), 1462 (w), 1428 (w), 1216 (m), 1139 (s), 1113 (s), 1015 (w), 824 (m), 702 (s), 678 (s), 505 (m). HRMS-ESI (m/z): [M+H]⁺ calcd for C₃₈H₅₅O₆Si, 635.3768; found, 635.3766. [α]_{D}²⁵=+29° (c=0.50, CHCl₃).

Diol S19: Amorphous white solid. R_{f}=0.55 (75% ethyl acetate-hexanes; UV, PAA, CAM). ¹H NMR (400 MHz, CDCl₃) δ 7.68-7.66 (m, 4H, 2×H₂₇, 2×H₃₁), 7.43-7.34 (m, 6H, 2×H₂, 1×H₂₈, 2×H₃₀, 1×H₃₂), 5.63 (d, J=8.4 Hz, 1H, H₁₄), 4.13 (dd, J=21.6, 4.8 Hz, 2H, H₂₂), 3.83 (td, J=11.2, 2.4 Hz, 1H, 1×H₂₀), 3.79-3.74 (m, 1H, 1×H₂₀), 3.65 (d, J=6.0 Hz, 1H, H₁₁), 2.59 (br s, 1H, C11-OH), 2.38-2.31 (m, 1H, H₁₀), 2.27-2.17 (m, 2H, H₂), 2.15-2.08 (m, 1H, 1×H₁₃), 2.05 (s, 1H, H₄), 1.89 (ddd, J=14.4, 8.0, 3.2 Hz, 1H, 1×H₉), 1.77 (dt, J=14.4, 1.6 Hz, 1H, 1×H₈), 1.64-1.53 (m, 4H, 1×H₁, 1×H₆, 1×H₇, 1×C22-OH), 1.48-1.42 (m, 1H, 1×H₁), 1.41-1.26 (m, 5H, 1×H₇, 3×H₁₅, 1×H₁₉), 1.39-1.28 (m, 13H, 1×H₁₃, 3×H₁₈, 9×H₂₄), 0.94 (d, J=7.2 Hz, 3H, H₁₇), 0.78-0.74 (app d, 1H, 1×H₁₃), 0.59 (d, J=6.4 Hz, 3H, H₁₆). ¹³C NMR (100 MHz, CDCl₃) δ 217.4 (C), 169.8 (C), 135.6 (CH), 132.8 (C), 132.7 (C), 129.9 (CH), 127.8 (CH), 71.4 (CH), 68.9 (CH), 62.8 (CH₂), 58.7 (CH₂), 58.3 (CH), 45.6 (C), 44.9 (CH₂), 43.5 (CH₂), 41.8 (C), 40.8 (C), 36.7 (CH), 34.6 (CH), 34.5 (CH₂), 30.1 (CH₂), 26.9 (CH₂), 26.7 (CH₃), 24.9 (CH₂), 19.2 (C), 18.8 (CH₃), 16.7 (C₃), 15.0 (CH₃), 10.8 (CH₃). IR (ATR-FTIR), cm⁻¹: 2928 (w), 2862 (w), 1734 (m), 1464 (w), 1250 (w), 1188 (m), 1113 (s), 1056 (w), 1039 (m), 804 (m), 701 (s), 613 (m), 505 (s). HRMS-ESI (m/z): [M+H]⁺ calcd for C₃₈H₅₅O₆Si, 635.3768; found, 635.3755.

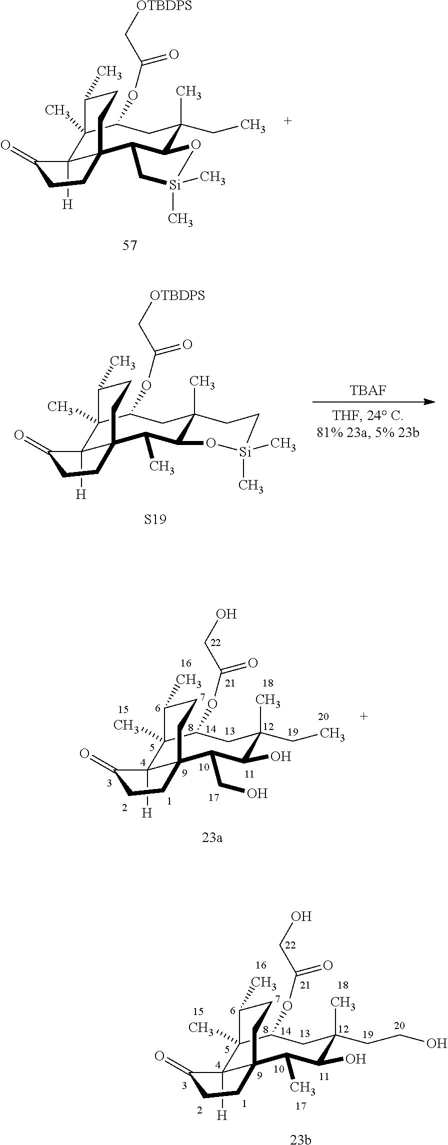

### Silyldeprotection of a Mixture of S7 and S19 (FIG. 5, Scheme 5)

A solution of tetrabutylammonium fluoride in tetrahydrofuran (1.00 M, 296 μL, 296 μmol, 2.00 equiv) was added dropwise via syringe to a solution of the unpurified mixture of the diols 57 and S19 (94.2 mg, 148 μmol, 1 equiv) in tetrahydrofuran (3.0 mL) at 24° C. The reaction mixture was stirred for 2 h at 24° C. The product mixture was diluted sequentially with dichloromethane (5.0 mL) and saturated aqueous sodium bicarbonate (3.0 mL). The diluted product mixture was transferred to a separatory funnel and the layers that formed were separated. The aqueous layer was extracted with ethyl acetate (3×10 mL). The organic layers were combined and the combined organic layers were dried over sodium sulfate. The dried solution was filtered and the filtrate was concentrated to dryness. The residue obtained was purified by automated flash-column chromatography (eluting with hexanes initially, grading to 100% ethyl acetate-hexanes, linear gradient) to afford separately 12-epi-17-hydroxy-19,20-dihydropleuromutilin (23a) as an amorphous white solid (47.5 mg, 81%) and 12-epi-19-hydro-20-hydroxypleuromutilin (23b) as an amorphous white solid (3.0 mg, 5%).

12-epi-17-Hydroxy-19,20-dihydropleuromutilin (23a): R_{f}=0.11 (75% ethyl acetate-hexanes; PAA, CAM). ¹H NMR (600 MHz, CDCl₃) δ 5.65 (d, J=8.4 Hz, 1H, H₄), 4.08 (d, J=17.4 Hz, 1H, 1×H₂), 4.02 (d, J=17.4 Hz, 1H, 1×H₂), 3.94 (td, J=10.2, 3.6 Hz, 1H, 1×H₁₇), 3.85-3.82 (br m, 1H, 1×H₁₇), 3.70 (t, J=7.0 Hz, 1H, H₁₁), 3.29 (d, J=7.2 Hz, 1H, C11-OH), 2.80 (t, J=5.4 Hz, 1H, C17-OH), 2.55 (br s, 1H, C22-OH), 2.40 (td, J=6.6, 3.0 Hz, 1H, H₁₀), 2.29-2.15 (m, 2H, H₂), 2.06 (dd, J=16.2, 8.4 Hz, 1×H₁₃), 1.99 (s, 1H, H₄), 1.83-1.67 (m, 3H, 1×H₈, 2×H₁₉), 1.66-1.59 (m, 2H, 1×H₁, 1×H₆), 1.57-1.51 (m, 1H, 1×H₇), 1.43-1.37 (m, 5H, 1×H₁, 1×H₇, 3×H s), 1.17 (td, J=13.8, 4.2 Hz, 1H, 1×H₈), 1.07 (s, 3H, H₁₈), 1.04 (app d, 1H, 1×H₁₃), 0.88 (t, J=7.5 Hz, 3H, H₂₀), 0.70 (d, J=6.0 Hz, 3H, H₁₆). ¹³C NMR (150 MHz, CDCl₃) δ 216.4 (C), 172.1 (C), 73.6 (CH), 70.2 (CH), 61.6 (CH₂), 61.3 (CH₂), 58.3 (CH), 44.0 (C), 42.9 (CH), 41.9 (C), 41.6 (CH₂), 40.1 (C), 36.6 (CH), 34.5 (CH₂), 34.4 (CH₂), 30.4 (CH₂), 26.9 (CH₂), 25.7 (CH₂), 18.3 (CH₃), 16.7 (CH₃), 14.8 (CH₃), 7.9 (CH₃). IR (ATR-FTIR), cm⁻¹: 3398 (br w), 2926 (w), 2883 (w), 1729 (m), 1458 (w), 1386 (w), 1231 (w), 1067 (m), 1015 (w), 908 (s), 726 (s), 648 (m). HRMS-ESI (m/z): [M+H]⁺ calcd for C₂₂H₃₇O₆, 397.2590; found, 397.2587. [α]_{D}²⁵=+33° (c=0.33, CHCl₃).

12-epi-19-Hydro-20-hydroxypleuromutilin (23b): R_{f}=0.37 (100% ethyl acetate-hexanes; PAA, CAM). ¹H NMR (500 MHz, CDCl₃) δ 5.70 (d, J=9.0 Hz, 1H, H₁₄), 4.08 (dd, J=29.0, 17.0 Hz, 2H, H₂₂), 3.86 (td, J=11.0, 2.5 Hz, 1H, 1×H₂₀), 3.80-3.76 (br m, 1H, 1×H₂₀), 3.68 (d, J=6.0 Hz, 1H, H₁), 2.40 (br s, 1H, C20-OH), 2.35-2.30 (m, 1H, H₁₀), 2.29-2.16 (m, 3H, 2×H₂, 1×H₁₃), 2.10 (s, 1H, H₄), 1.92 (ddd, J=15.0, 9.0, 3.0 Hz, 1H, 1×H₁₉), 1.80 (dt, J=14.5, 3.0 Hz, 1H, 1×H₈), 1.68-1.46 (m, 5H, 2×H₁, 1×H₆, 1×H₇, 1×C22-OH), 1.44 (s, 3H, H₁₅), 1.43-1.36 (m, 2H, 1×H₇, 1×H₁₉), 1.17-1.10 (m, 4H, 1×H₈, 3×H₁₈), 0.96 (d, J=7.0, 3H, H₁₇), 1.04 (app d, 1H, 1×H₁₃), 0.70 (d, J=6.0 Hz, 3H, H₁₆). ¹³C NMR (125 MHz, CDCl₃) δ 217.2 (C), 172.1 (C), 71.3 (CH), 70.4 (CH), 61.3 (CH₂), 58.7 (CH₂), 58.2 (CH), 45.7 (C), 44.9 (CH₂), 43.5 (CH₂), 41.9 (C), 40.9 (C), 36.6 (CH), 34.6 (CH), 34.3 (CH₂), 30.1 (CH₂), 26.9 (CH₂), 24.9 (CH₂), 18.6 (CH₃), 16.7 (CH₃), 14.9 (CH₃), 10.9 (CH₃). IR (ATR-FTIR), cm⁻¹: 3407 (br m), 2927 (m), 1730 (s), 1457 (w), 1384 (w), 1263 (m), 1215 (m), 1153 (w), 1098 (s), 1019 (m), 965 (m), 736 (m). HRMS-ESI (m/z): [M+Na]⁺ calcd for C₂₂H₃₇O₆, 397.2590; found, 397.2598.

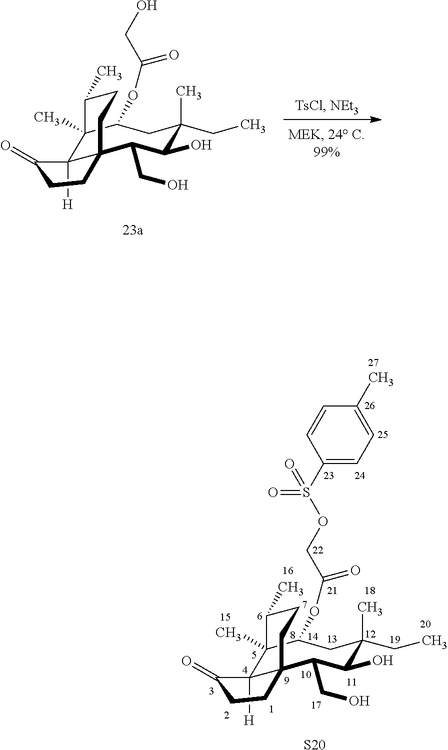

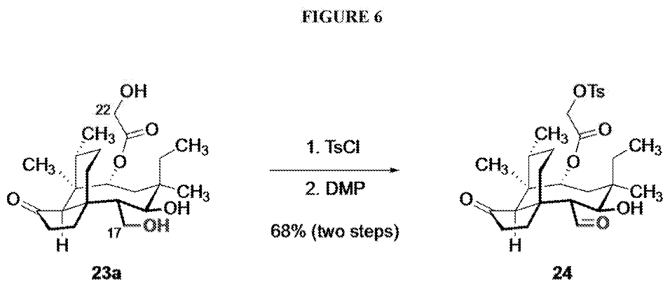

### Synthesis of O-(p-tolylsulfonyl)-12-epi-17-hydroxy-19,20-dihydropleuromutilin (S20, FIG. 6, Scheme 6)

A solution of triethylamine (9.4 μL, 67.4 μmol, 1.10 equiv) in methyl ethyl ketone (200 μL) was added dropwise via syringe to a solution of 12-epi-17-hydroxy-19,20-dihydropleuromutilin [23a, 24.3 mg, 500 μmol, 1 equiv, dried by azeotropic distillation with benzene (500 μL)] and p-tolylsulfonyl chloride (12.9 mg, 67.4 μmol, 1.10 equiv) in methyl ethyl ketone (300 μL) at 24° C. The reaction mixture was stirred for 12 h at 24° C. The reaction was diluted with saturated aqueous sodium bicarbonate solution (1.0 mL). The diluted mixture was transferred to a separatory funnel and the layers that formed were separated. The aqueous layer obtained was extracted with dichloromethane (3×5.0 mL). The organic layers were combined and the combined organic layer was dried over sodium sulfate. The dried solution was filtered and the filtrate was concentrated to dryness. The residue obtained was purified by automated flash-column chromatography (eluting with dichloromethane initially, grading to 50% ether-dichloromethane, linear gradient) to afford O-(p-tolylsulfonyl)-12-epi-17-hydroxy-19,20-dihydropleuromutilin S20 as an amorphous white solid (36.5 mg, 99%).

R_{f}=0.47 (50% ether-dichloromethane; UV, PAA, CAM). ¹H NMR (400 MHz, CDCl₃) δ 7.82 (d, J=8.4 Hz, 2H, H₂₄), 7.35 (d, J=8.4 Hz, 2H, H₂₅), 5.59 (d, J=9.0 Hz, 1H, H₁₄), 4.49 (s, 2H, H₂₂), 3.92 (td, J=9.2, 4.0 Hz, 1H, 1×H₁₇), 3.85-3.76 (br m, 1H, 1×H₁), 3.68 (t, J=6.8 Hz, 1H, H₁), 3.04 (d, J=7.2 Hz, 1H, C11-OH), 2.50 (t, J=5.2 Hz, 1H, C17-OH), 2.45 (s, 3H, H₂₇), 2.35 (td, J=7.8, 2.4 Hz, 1H, H₁₀), 2.25-2.14 (m, 2H, H₂), 2.02 (dd, J=16.4, 8.4 Hz, 1H, 1×H₁₃), 1.97 (s, 1H, H₄), 1.84-1.73 (m, 2H, 1×H₈, 1×H₁₉), 1.64-1.49 (m, 3H, 1×H₁, 1×H₆, 1×H₇), 1.44-1.36 (m, 6H, 1×H₁, 1×H₇, 3×H₁₅, 1×H₁₉), 1.18 (td, J=13.6, 3.6 Hz, 1H, 1×H₈), 1.07-0.97 (m, 4H, 1×H₁₃, 3×H₁₈), 0.88 (t, J=7.4 Hz, 3H, H₂₀), 0.63 (d, J=6.0 Hz, 3H, H₁₆). ¹³C NMR (100 MHz, CDCl₃) δ 216.2 (C), 164.8 (C), 145.3 (C), 132.6 (C), 129.9 (CH), 128.1 (CH), 73.5 (CH), 70.7 (CH), 65.1 (CH₂), 61.6 (CH₂), 58.2 (CH), 44.0 (C), 43.0 (CH), 41.9 (C), 41.4 (CH₂), 40.0 (C), 36.5 (CH), 34.4 (CH₂), 34.3 (CH₂), 30.4 (CH₂), 26.9 (CH₂), 25.7 (CH₂), 21.7 (CH₃), 18.3 (CH₃), 16.6 (CH), 14.8 (CH₃), 7.9 (CH₃). IR (ATR-FTIR), cm⁻¹: 3446 (br w), 2959 (m), 2882 (w), 1734 (m), 1598 (w), 1453 (w), 1370 (m), 1289 (w), 1225 (w), 1190 (m), 1177 (s), 1096 (w), 1042 (m), 816 (w), 777 (w), 664 (w), 554 (w). H RMS-ESI (m/z): [M+H]⁺ calcd for C₂H₄₃O₈S, 551.2679; found, 551.2678. [α]_{D}²⁵=+26° (c=0.25, CHCl₃).

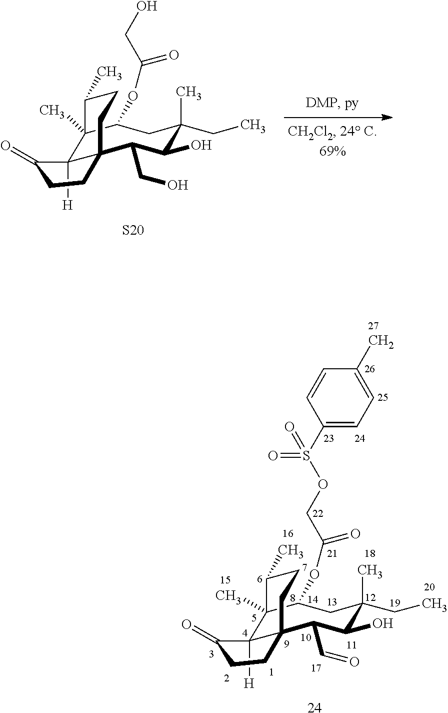

### Synthesis of O-(p-tolylsulfonyl)-12-epi-7-oxo-19,20-dihydropleuromutilin (24. FIG. 6, Scheme 6)

Six equal portions of Dess-Martin periodinane (25.4 mg, 59.9 μmol, 1.10 equiv) was added over 1 h to a solution of O-(p-tolylsulfonyl)-12-epi-17-hydroxy-19,20-dihydropleuromutilin S20 (30.0 mg, 54.5 μmol, 1 equiv) and pyridine (44/1 μL, 545 μmol, 10.0 equiv) in dichloromethane (400 μL) at 24° C. The resulting mixture was stirred for 30 min at 24° C. The product mixture was diluted sequentially with ether (1.0 mL), a saturated aqueous sodium bicarbonate solution (500 μL) and a saturated aqueous sodium thiosulfate solution (500 μL). The resulting mixture was stirred for 5 min at 24° C. The resulting mixture was transferred to a separatory funnel and the layers that formed were separated. The aqueous layer obtained was extracted with dichloromethane (3×5.0 mL). The organic layers were combined and the combined organic layer was dried over sodium sulfate. The dried solution was filtered and the filtrate was concentrated to dryness. The residue obtained was purified by automated flash-column chromatography (eluting with dichloromethane initially, grading to 50% ether-dichloromethane, linear gradient) to afford O-(p-tolylsulfonyl)-12-epi-17-oxo-19,20-dihydropleuromutilin (24) as an amorphous white solid (20.6 mg, 69%).

R_{f}=0.42 (20% ether-dichloromethane; UV, PAA, CAM). ¹H NMR (500 MHz, C₆D₆) δ 9.58 (d, J=4.5 Hz, 1H, H₁₇), 7.77 (d, J=8.0 Hz, 2H, H₂₄), 6.66 (d, J=8.4 Hz, 2H, H₂₅), 5.58 (d, J=9.0 Hz, 1H, H₁), 4.21 (td, J=14.0, 2.0 Hz, 2H, H₂₂), 3.50 (d, J=7.0 Hz, 1H, H₁), 3.00 (t, J=6.0 Hz, 1H, H₁₀), 2.23-2.18 (m, 1H, OH), 1.81-1.78 (m, 5H, 2×H₂, 3×H₂₇), 1.74 (dd, J=16.0, 9.0 Hz, 1H, 1×H₁₃), 1.68 (s, 1H, H₄), 1.66-1.54 (m, 5H, 1×H₁, 3×H₁₅, 1×H₁₉), 1.50-1.35 (m, 2H, 1×H₁, 1×H₁₉), 1.27-1.22 (m, 5H, 1×H₈), 1.18 (dt, J=12.5, 6.0 Hz, 1H, 1×H₇), 1.10 (s, 3H, H₁₈), 1.07-0.99 (m, 2H, 1×H₇, 1×H₁₃), 0.75 (td, J=14.0, 4.5 Hz, 1H, 1×H₈), 0.67 (t, J=7.5 Hz, 3H, H₂₀), 0.59 (d, J=7.0 Hz, 3H, H₁₆). ¹³C NMR (125 MHz, C₆D₆) δ 213.0 (C), 201.1 (CH), 165.2 (C), 144.8 (C), 133.9 (C), 129.9 (CH), 128.4 (CH), 73.0 (CH), 70.4 (CH), 65.0 (CH₂), 57.6 (CH), 55.1 (CH), 43.3 (C), 42.1 (C), 41.2 (C), 40.5 (CH₂), 36.5 (CH), 34.0 (CH₂), 33.4 (CH₂), 30.8 (CH₂), 26.6 (CH₂), 26.4 (CH₂), 21.2 (CH₃), 17.3 (CH), 16.7 (CH₃), 14.9 (CH₃), 7.9 (CH₃). IR (ATR-FTIR), cm⁻¹: 2936 (w), 1736 (m), 1717 (m), 1460 (w), 1368 (m), 1296 (w), 1224 9w), 1190 (w), 1177 (s), 1094 (w), 1043 (m), 968 (w), 816 (m), 664 (w), 554 (w). HRMS-ESI (m/z): [M+H]⁺ calcd for C₂₉H₄₁O₈S, 549.2522; found, 549.2526. [α]_{D}²⁵=+29° (c=0.10, CHCl₃).

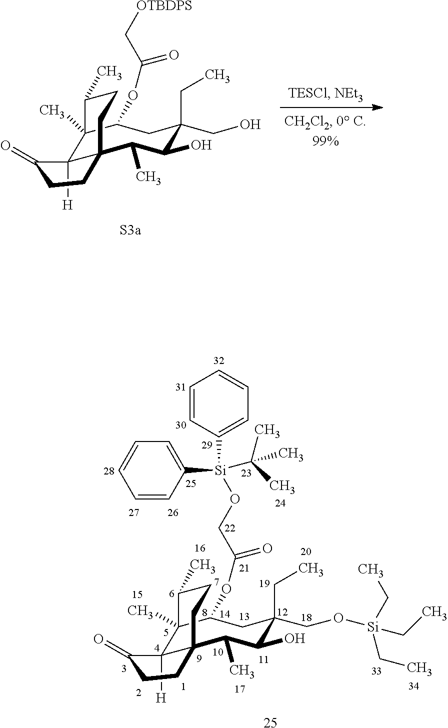

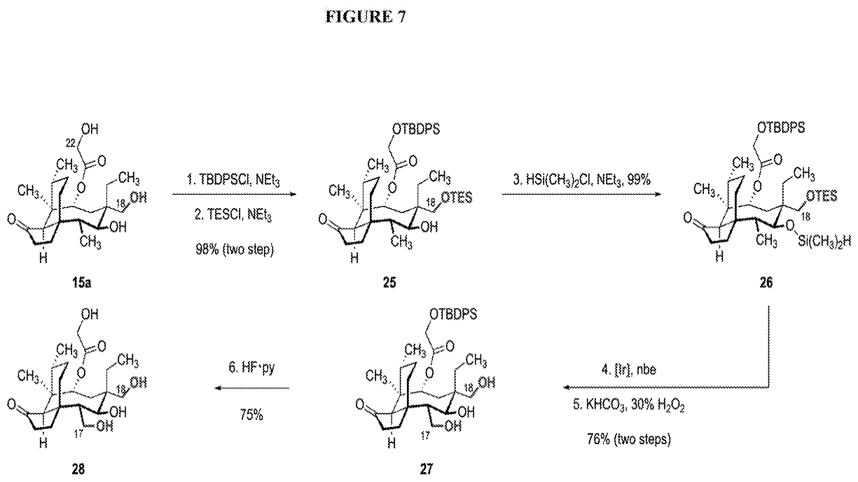

### Synthesis of bis(silyl)ether 25 (FIG. 7, Scheme 7)

Chlorotriethylsilane (42.5 μL, 253 μmol, 1.05 equiv) was added dropwise via syringe to a solution of O-tert-butyldiphenylsilyl-18-hydroxyl-19,20-dihydropleuromutilin [S3a, 153 mg, 241 μmol, 1 equiv, dried by azeotropic distillation with benzene (500 μL)] and triethylamine (67.2 μL, 482 mmol, 4.00 equiv) in dichloromethane (2.8 mL) at 0° C. The reaction mixture was stirred for 30 min at 0° C. The product mixture was diluted with an aqueous potassium phosphate buffer solution (pH 7, 0.10 M, 5.0 mL). The diluted mixture was transferred to a separatory funnel and the layers formed were separated. The aqueous layer was extracted with dichloromethane (3×15 mL). The organic layers were combined and the combined organic layers were dried over sodium sulfate. The dried solution was filtered and the filtrate was concentrated to dryness. The residue obtained was purified by automated flash-column chromatography (eluting with hexanes initially, grading to 20% ethyl acetate-hexanes, linear gradient) to afford the bis(silyl) ether 25 as an amorphous white solid (181 mg, 99%).

R_{f}=0.21 (10% ethyl acetate-hexanes; UV, PAA, CAM). ¹H NMR (400 MHz, CDCl₃) δ 7.69-7.66 (m, 4H, 2×H₂₇, 2×H₃₁), 7.46-7.36 (m, 6H, 2×H₂₆, 1×H₂, 2×H₃₀, 1×H₃₂), 5.73 (d, J=8.0 Hz, 1H, H₁₄), 4.16 (dd, J=30.0, 9.5 Hz, 2H, H₂₂), 3.85 (d, J=8.0 Hz, 1H, H₁₁), 3.61 (s, 1H, OH), 3.58 (d, J=12.5 Hz, 1H, 1×H₁₈), 3.37 (d, J=12.5 Hz, 1H, 1×H₁₈), 2.42-2.36 (m, 1H, H₁₀), 2.24-2.10 (m, 2H, H₂), 2.08 (s, 1H, H₄), 1.95-1.84 (m, 1H, 1×H₁₉), 1.82-1.75 (m, 2H, 1×H₁, 1×H₁₉), 1.70-1.62 (m, 2H, 1×H₁, 1×H₇), 1.59-1.54 (m, 2H, 1×H₆, 1×H₁₃), 1.45 (td, J=13.0, 4.0 Hz, 1H, 1×H₁), 1.38-1.31 (m, 4H, 1×H₇, 3×H₁₅), 1.10-1.04 (m, 10H, 1×H₈, 9×H₂₄), 1.02-0.95 (m, 10H, 1×H₁₃, 9×H₃₄), 0.92 (d, J=8.5 Hz, 3H, H₇), 0.71 (t, J=9.3 Hz, 3H, H₂₀), 0.68-0.59 (m, 9H, 3×H₁₆, 6×H₃₃). ¹³C NMR (150 MHz, CDCl₃) δ 217.7 (C), 1704 (C), 136.1 (CH), 133.5 (C), 133.4 (C), 130.4 (CH), 128.3 (CH), 128.3 (CH), 75.1 (CH), 70.9 (CH₂), 68.5 (CH), 63.5 (CH₂), 58.9 (CH), 46.0 (C), 444 (C), 42.5 (C), 37.3 (CH), 36.0 (CH₂), 35.1 (CH), 35.0 (CH), 30.8 (CH₂), 27.5 (CH₂), 27.0 (CH₃), 25.4 (CH₂), 19.6 (C), 17.5 (CH₂), 16.9 (CH₃), 15.2 (CH₃), 11.2 (CH₃), 8.0 (CH₃), 7.1 (CH₃), 4.7 (CH₂). IR (ATR-FTIR), cm⁻¹: 2954 (w), 2878 (w), 1735 (w) 1113 (s), 1006 (s), 965 (s), 806 (w), 701 (s). HRMS-ESI (m/z): [M+H]⁺ calcd for C₄₄H₆₉O₆Si, 749.4633; found, 749.4634. [α]_{D}²⁵=+30° (c=1.0, CHCl₃).

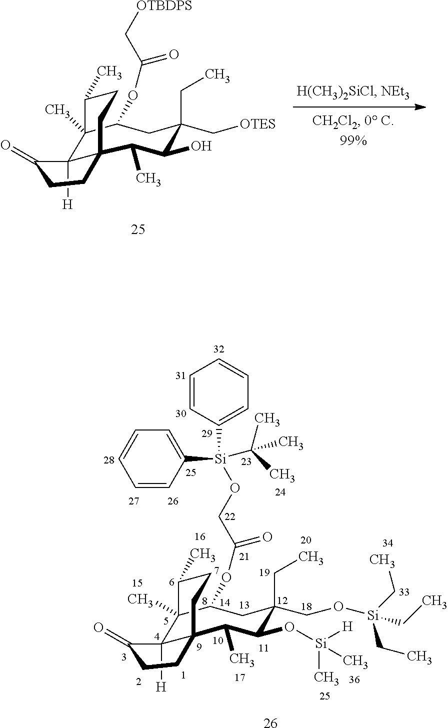

### Synthesis of Silane 26 (FIG. 7, Scheme 7)

Dimethylchorosilane (9.6 μL, 34.4 mmol, 2.00 equiv) was added dropwise via syringe to a solution of the bis(silyl) ether 25 [12.9 mg, 17.2 mmol, 1 equiv, dried by azeotropic distillation with benzene (200 μL)] and triethylamine (3.8 μL, 68.9 mmol, 4.00 equiv) in dichloromethane (200 mL) at 0° C. The reaction mixture was stirred for 30 min at 0° C. The product mixture was diluted sequentially with pentane (1.0 mL) and aqueous potassium phosphate buffer solution (pH 7, 0.10 M, 1.0 mL). The diluted mixture was transferred to a separatory funnel and the layers formed were separated. The aqueous layer was extracted with dichloromethane (3×10 mL). The organic layers were combined and the combined organic layers were dried over sodium sulfate. The dried solution was filtered and the filtrate was concentrated to dryness to afford silane 26 as an amorphous white solid (14.1 mg, 99%). The silane 26 prepared this way was analytically pure and was used in the next step without further purification.

R_{f}=0.66 (10% ethyl acetate-hexanes; UV, PAA, CAM). ¹H NMR (500 MHz, C₆D₆) δ 7.79-7.77 (m, 4H, 2×H₂₇, 2×H₃₁), 7.24-7.22 (m, 6H, 2×H₂₆, 1×H₂₈, 2×H₃₀, 1×H₃₂), 5.95 (d, J=8.5 Hz, 1H, H₁₄), 4.95 (sep, J=3.0 Hz, 1H, Si—H), 4.19 (s, 2H, H₂₂), 4.15 (d, J=6.5 Hz, 1H, H₁), 3.79 (d, J=11.0 Hz, 1H, 1×H₁₈), 3.32 (d, J=9.5 Hz, 1H, 1×H₁₈), 2.51-2.44 (m, 2H, 1×H₁, 1×H₁₃), 2.24 (s, 1H, H₄), 2.06-1.98 (m, 1H, 1×H₁₉), 1.97-1.87 (m, 3H, 2×H₂, 1×H₁₉), 1.82 (s, 3H, H₁₅), 1.80-1.72 (m, 1H, H₆), 1.69-1.54 (m, 2H, 1×H₁, 1×H₇), 1.52-1.46 (m, 1H, 1×H₈), 1.38 (app d, 1H, 1×H₁₃), 1.21 (s, 9H, H₂₄), 1.15-1.08 (m, 2H, 1×H₁, 3×H₇), 0.90 (t, J=8.0 Hz, 9H, H₃₄), 0.90-0.84 (m, 7H, 1×H₈, 3×H₁₇, 3×H₂₀), 0.70 (d, J=7.0 Hz, 3H, H₁₆), 0.60 (q, 6H, H₃₃), 0.27 (d, J=2.5 Hz, 3H, H₃₅), 0.24 (d, J=2.5 Hz, 3H, H₃₆). ¹³C NMR (125 MHz, C₆D₆) δ 215.2 (C), 170.0 (C), 136.1 (CH), 136.0 (CH), 133.5 (C), 133.4 (C), 130.2 (CH), 73.9 (CH), 68.9 (CH), 36.5 (CH₂), 63.3 (CH₂), 58.5 (CH), 45.9 (C), 45.8 (C), 42.6 (C), 37.1 (CH), 36.9 (CH₂), 35.0 (CH), 34.3 (CH₂), 30.8 (CH₂), 27.3 (CH₂), 26.9 (CH₃), 25.7 (CH₂), 20.1 (CH₂), 19.6 (C), 16.8 (CH₃), 15.6 (CH₃), 12.7 (CH₃), 8.3 (CH₃), 7.3 (CH₃), 5.0 (CH₂), −0.18 (CH₃), −0.38 (CH₃). IR (ATR-FTIR), cm⁻¹: 2955 (m), 2878 (w), 1739 (m), 1462 (w), 1249 (w), 1130 (s), 1113 (s), 910 (s), 814 (m), 702 (s), 506 (s). HRMS-ESI (m/z): [M+H]⁺ calcd for C₄₆H₇₅O₆Si₃, 807.4871; found, 807.4886. [α]_{D}²⁵=+24° (c=0.10, CHCl₃).

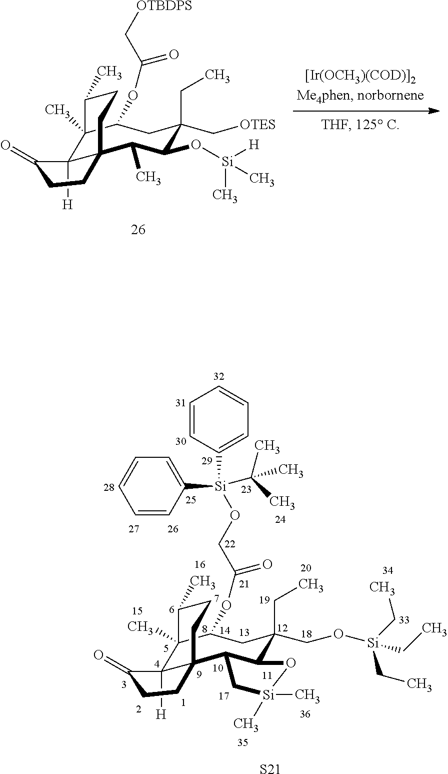

### Synthesis of Silacycle S21 (FIG. 7, Scheme 7)

This experiment was adapted from the work of Hartwig and co-workers.² A 4-mL pressure tube with a Teflon-coated valve was charged with 3,4,7,8-tetramethyl-1,10-phenanthroline (4.7 mg, 19.9 μmol, 12.5 mol %) and norbornene (21.6 mg, 230 μmol, 1.50 equiv) in the glovebox. A 4-mL vial was charged with silane 26 [115 mg, 153 μmol, 1 equiv, dried by azeotropic distillation with benzene (3×500 μL)]. The vessel containing the silane was evacuated and refilled using a balloon of argon. This process was repeated two times. Tetrahydrofuran (100 μL) was transferred into the vessel containing the silane and the resulting solution was added to the vessel containing the ligand and norbornene the glovebox. The vessel containing the silane was rinsed with tetrahydrofuran (3×50 μL) and the combined rinses were transferred to the reaction vessel.

Methoxy(cyclooctadiene)iridium(I) dimer (5.1 mg, 7.7 μmol, 5.0 mol %) was added to an oven-dried 4-mL vial. Tetrahydrofuran (200 μL) was added into the vial containing the catalyst and the resulting solution was transferred dropwise via syringe to the reaction vessel in the glovebox. The vial containing the catalyst was rinsed with tetrahydrofuran (3×40 μL) and the combined rinses were transferred into the reaction vessel. The reaction vessel was sealed and the reaction mixture was stirred for 1 h at 24° C. in the glovebox. The sealed reaction vessel was then removed from the glovebox and placed in an oil bath that had been preheated to 120° C. The reaction mixture was stirred and heated for 2 h at 120° C. The reaction vessel was allowed to cool over 30 min to 24° C. and the cooled product mixture was concentrated to dryness. The residue obtained was filtered through a pad of silica gel (2.5×2.5 cm). The filter cake was washed with a mixture of ether and hexanes (1:1, v/v, 100 mL). The filtrate were combined and the combined filtrates were concentrated to dryness. The residue obtained contained the silacycle S21 and was used in the next step without further purification. An analytically pure sample of S21 was obtained for characterization by automated flash-column chromatography (eluting with hexanes initially, grading to 15% ether-hexanes, linear gradient).

Amorphous white solid. R_{f}=0.66 (10% ethyl acetate-hexanes; UV, PAA, CAM). ¹H NMR (400 MHz, C₆D₆) δ 7.81-7.78 (m, 4H, 2×H₂₇, 2×H₃₁), 7.24-7.22 (m, 6H, 2×H₂₄, 1×H₂₈, 2×H₃₀, 1×H₃₂), 5.95 (d, J=8.4 Hz, 1H, H₁₄), 4.41 (d, J=7.2 Hz, 1H, H₁₁), 4.21 (s, 2H, H₂₂), 3.99 (d, J=11.5 Hz, 1H, 1×H₁₈), 3.42 (d, J=11.5 Hz, 1H, 1×H₁₈), 2.83-2.77 (m, 1H, 1×H₁₀), 2.59 (dd, J=16.4, 8.8 Hz, 1×H₁₃), 2.30 (s, 1H, H₄), 2.02-1.88 (m, 3H, 2×H₂, 1×H₁₉), 1.84-1.74 (m, 4H, 1×H₆, 3×H₁₈), 1.72-1.67 (m, 1H, 1×H₇), 1.64-1.55 (m, 1H, 1×H₁₉), 1.46-1.33 (m, 3H, 1×H₇, 1×H₇, 1×H₇), 1.28-1.24 (m, 1H, 1×H₁₃), 1.20 (s, 9H, H₂₄), 1.17-1.05 (m, 2H, 1×H₁, 1×H₈), 1.01 (t, J=8.0 Hz, 9H, H₃₄), 0.83 (t, J=8.0 Hz, 3H, H₂₀), 0.75 (d, J=8.0 Hz, 3H, H₁₆), 0.64-0.58 (m, 7H, 1×H₁₇, 6×H₃₃), 0.43 (dd, J=15.6, 5.6 Hz, 1H, 1×H₇), 0.13 (s, 3H, H₃₅), 0.10 (s, 3H, H₃₆). ¹³C NMR (100 MHz, C₆D₆) δ 214.7 (C), 170.2 (C), 136.1 (CH), 136.1 (CH), 133.5 (C), 130.2 (C), 128.6 (CH), 128.2 (CH), 127.2 (CH), 79.6 (CH), 68.4 (CH), 68.2 (CH₂), 63.2 (CH₂), 59.1 (CH), 45.9 (C), 45.0 (C), 42.5 (C), 38.8 (CH), 37.0 (CH), 35.1 (CH₂), 34.2 (CH₂), 31.7 (CH₂) 27.3 (CH₂), 27.0 (CH₃), 25.6 (CH₂), 19.8 (C), 19.6 (CH₂), 16.8 (CH₃), 15.5 (CH₃), 13.1 (CH₂), 8.2 (CH₃), 7.2 (CH₃), 5.0 (CH₂), 0.59 (CH₃), 0.54 (CH₃). IR (ATR-FTIR), cm⁻¹: 2953 (w), 2877 (w), 1739 (m), 1460 (w), 1428 (w), 1251 (w), 1212 (w), 1143 (m), 1113 (s), 1094 (s), 1041 (m), 1009 (m), 894 (m), 847 (m), 812 (s), 739 (s), 701 (s), 613 (m), 497 (s). HRMS-ESI (m/z): [M+H]⁺ calcd for C₄₆H₇₃O₆Si₃, 805.4715; found, 805.4742. [α]_{D}²⁵=+24° (c=0.10, CHCl₃).

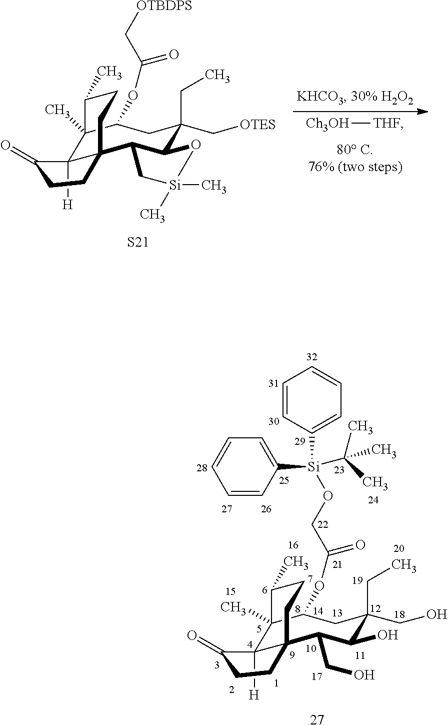

### Tamao-Fleming Oxidation of Silacycle S32 (FIG. 7, Scheme 7)

Tetrahydrofuran (900 μL) and an aqueous hydrogen peroxide solution (30% w/w, 141 μL, 1.24 mmol, 20.0 equiv) were added sequentially to a suspension of the unpurified silacycle S21 (50.0 mg, 62.1 μmol, 1 equiv) and potassium bicarbonate (37.3 mg, 373 μmol, 6.00 equiv) in methanol (900 μL) at 24° C. in a 4-mL vial. The vial was sealed with a Teflon-lined cap and the sealed vial was placed in an oil bat that had been preheated to 80° C. The reaction mixture was stirred and heated for 1 h at 80° C. The product mixture was diluted sequentially with dichloromethane (2.0 mL) and saturated aqueous sodium thiosulfate (1.0 mL). The diluted product mixture was transferred to a separatory funnel and the layers that formed were separated. The aqueous layer was extracted with dichloromethane (3×5 mL). The organic layers were combined and the combined organic layers were dried over sodium sulfate. The dried solution was filtered and the filtrate was concentrated to dryness. The residue obtained was purified by automated flash-column chromatography (eluting with hexanes initially, grading to 100% ethyl acetate-hexanes, linear gradient; then eluting with 2% methanol-ethyl acetate) to afford triol 27 as anamorphous white solid (30.8 mg, 76%).

R_{f}=0.20 (70% ethyl acetate-hexanes; UV, PAA, CAM). ¹H NMR (400 MHz, CDCl₃) δ 7.67-7.66 (m, 4H, 2×H₂₇, 2×H₃₁), 7.44-7.35 (m, 6H, 2×H₂₆, 1×H₂₈, 2×H₃₀, 1×H₃₂), 5.70 (d, J=7.6 Hz, 1H, H₁₄), 4.15 (dd, =25.2, 8.86 Hz, 2H, 1H₂₂), 4.03 (d, J=6.4 Hz, 11H, H₁₁), 3.91 (t, J=9.8 Hz, 1H, 1×H₁₇), 3.79 (dd, J=10.8, 2.8 Hz, 1H, 1×H₁₇), 3.56 (d, J=11.2 Hz, 1H, 1×H₅), 3.46 (d, J=11.2 Hz, 1H, 1×H₁₈), 2.96-2.90 (m, 1H, OH), 2.48 (td, J=10.0, 3.6 Hz, 1H, 1×H₁₀), 2.28-2.11 (m, 2H, H₂), 205-1.93 (m, 3H, 1×H₄, 1×H₁₃, 1×H₁₉), 1.84-1.72 (m, 4H, 1×H₁, 1×H₈, 1×H₁₉, 1×OH), 1.72-1.65 (m, 1H, H₆), 1.69-1.54 (m, 3H, 1×H₇, 1×H₁₃, 1×OH), 1.43-1.38 (m, 2H, 1×H₁, 1×H₇), 1.36 (s, 3H, H₁₅), 1.15 (td, J=13.2, 4.8 Hz, 1H, 1×H₈), 1.07 (s, 9H, H₂₄), 0.75 (t, J=7.4 Hz, 3H, H₂₀), 0.62 (t, J=6.8 Hz, 3H, H₁₆). ¹³C NMR (100 MHz, CDCl₃) δ 216.4 (C), 170.0 (C), 135.6 (CH), 135.5 (CH), 132.8 (C), 132.7 (C), 129.9 (CH), 127.9 (CH), 127.8 (CH), 77.7 (CH), 70.9 (CH₂), 67.5 (CH), 62.8 (CH₂), 61.3 (CH₂), 58.7 (CH), 44.0 (C), 43.4 (C), 42.6 (CH), 41.9 (C), 36.6 (CH), 35.0 (CH₂), 34.4 (CH₂), 30.5 (CH₂), 26.8 (CH₂), 26.7 (CH₃), 25.8 (CH₂), 19.2 (C), 17.1 (CH₂), 16.4 (CH₃), 14.8 (CH₃), 7.6 (CH₃). IR (ATR-FTR), cm⁻¹: 3370 (br w), 2734 (w), 2860 (w), 1736 (s), 1461 (w), 1428 (w), 1286 (w), 1209 (w), 1140 (s), 1113 (s), 1044 (s), 963 (w), 824 (w), 703 (s), 613 (w), 505 (s). HRMS-ESI (m/z): [M+H]⁺ calcd for C₃₈H₅₅O₇Si, 651.3717; found, 651.3718. [α]_{D}²⁵=+−33° (c=0.50, CHCl₃).

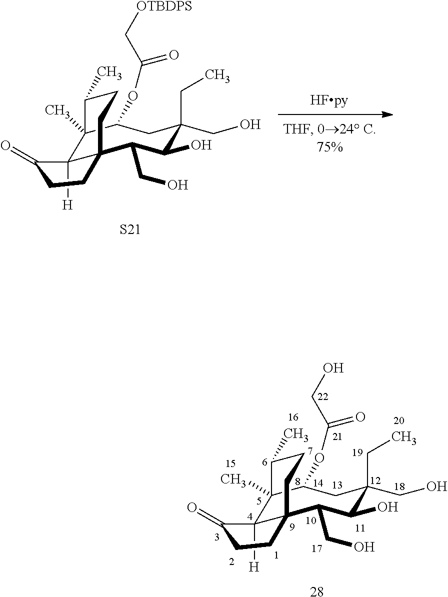

### Synthesis of 11,18-dihydroxy-19,20-dihydropleuromutilin (28, FIG. 7, Scheme 7)

Olah's reagent (5.0 μL, 192 μmol, 5.00 equiv) was added dropwise via syringe to a solution of the triol (27, 25.0 mg, 38.4 μmol, 1 equiv) in tetrahydrofuran (1.2 mL) at 0° C. The reaction mixture was stirred for 1 h at 0° C. The product mixture was diluted sequentially with dichloromethane (2.0 mL) and saturated aqueous sodium bicarbonate (5.0 mL). The diluted product mixture was transferred to a separatory funnel and the layers that formed were separated. The aqueous layer was extracted with ethyl acetate (3×15 mL). The organic layers were combined and the combined organic layers were dried over sodium sulfate. The dried solution was filtered and the filtrate was concentrated to dryness. The residue obtained was purified by automated flash-column chromatography (eluting with hexanes initially, grading to 100% ethyl acetate-hexanes, linear gradient; then eluting with ethyl acetate initially, grading to 10% methanol-ethyl acetate, linear gradient) to afford 11,18-dihydroxy-19,20-dihydropleuromutilin (28) as an amorphous white solid (11.9 mg, 75%).

R_{f}=0.20 (70% ethyl acetate-hexanes; UV, PAA, CAM). ¹H NMR (500 MHz, CD₃OD) δ 5.78 (d, J=8.0 Hz, 1H, H₄), 4.11 (d, J=7.0 Hz, 1H, H₁₁), 4.03 (t, J=17.5 Hz, 2H, H₂₂), 3.81 (t, J=10.3 Hz, 1H, 1×H₁₇), 3.73 (dd, J=11.0, 3.0 Hz, 1H, 1×H₁₇), 3.63 (d, J=10.5 Hz, 1H, 1×H₁₈), 3.37 (d, J=10.5 Hz, 1H, 1×H₁₈), 2.46 (td, J=10.0, 3.0 Hz, 1H, 1×H₁₀), 2.28 (s, 1H, H₄), 2.26-2.22 (m, 1H, 1×H₂), 2.18-2.10 (m, 1H, 1×H₂), 2.02 (dd, J=16.5, 8.0 Hz, 1H, 1×H₁₃), 1.76-1.57 (m, 3H, 1×H₁, 1×H₈, 1×H₁₉) 1.76-1.57 (m, 3H, 1×H₆, 1×H₇, 1×H₁₉), 1.44-1.37 (m, 5H, 1×H₁, 1×H₇, 3×H₁₅), 1.24 (app d, 1H, 1×H₁₃), 1.18 (td, J=14.5, 4.0 Hz, 1H, 1×H₈), 0.75-0.70 (m, 6H, 3×H₂₀, 3×H₁₆). ¹³C NMR (100 MHz, CDCl₃) δ 217.5 (C), 171.9 (C), 73.5 (CH), 68.1 (CH), 67.9 (CH₂), 60.6 (CH₂), 60.4 (CH₂), 58.1 (CH), 43.9 (C), 43.3 (C), 42.5 (CH), 41.8 (C), 36.6 (CH), 34.5 (CH₂), 33.8 (CH), 30.2 (CH₂), 26.7 (CH₂), 25.1 (CH₂), 18.0 (CH₂), 15.4 (CH₃), 13.9 (CH₃), 6.5 (CH₃). IR (ATR-FTIR), cm⁻¹: 3389 (br m), 2942 (m), 2882 (w), 1733 (s), 1456 (m), 1384 (w), 1285 (w), 1232 (m), 1091 (s), 1042 (s), 1017 (w), 952 (w). HRMS-ESI (m/z): [M+H]⁺ calcd for C₂₂H₃₇O₇, 413.2539; found, 413.2531. [α]_{D}²⁵=+31° (c=0.25, CH₃OH).

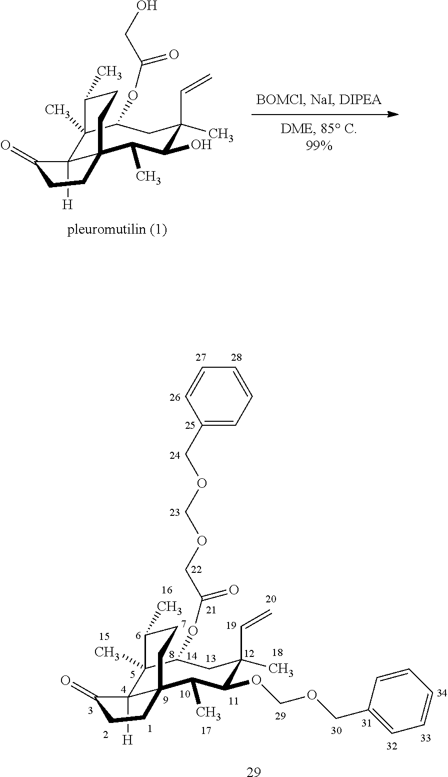

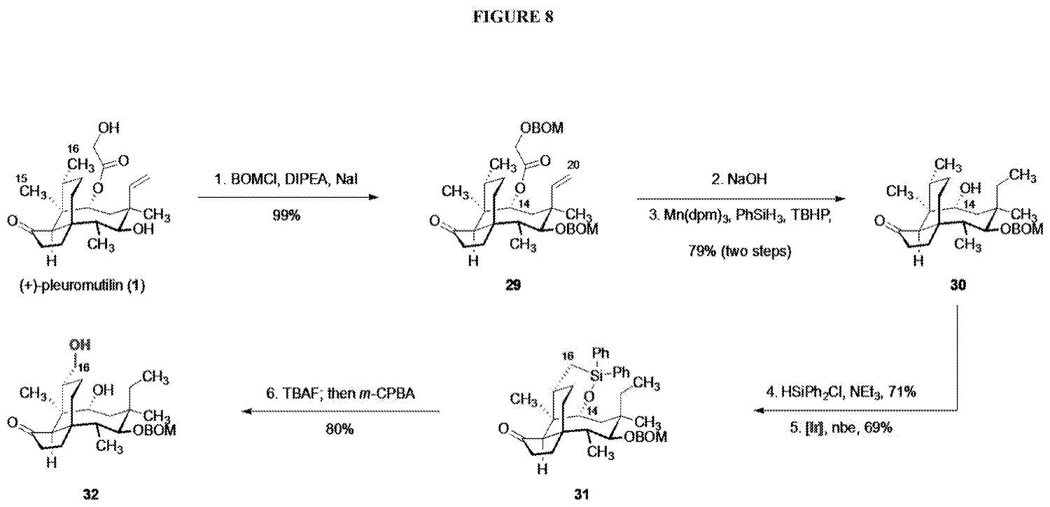

### Synthesis of 11,22-bis(benzyloxymethylenoxy)pleuromutilin 29 (FIG. 8, Scheme 8)

A 100-mL round-bottomed flask fused to a Teflon-coated valve was charged with pleuromutilin (1, 757 mg, 2.00 mmol, 1 equiv). Benzene (5.0 mL) was added and the solution was concentrated to dryness. This process was repeated twice. Sodium iodide (1.80 g, 12.0 mmol, 6.00 equiv) was added to the reaction vessel. The reaction vessel was evacuated and refilled using a balloon of argon. This process was repeated twice. 1,2-Dimethoxyethane (20 mL), N,N-diisopropylethylamine (2.79 mL, 16.0 mmol, 8.00 equiv), and benzyl chloromethyl ether (1.67 mL, 12.0 mmol, 6.00 equiv) was added sequentially via syringe to the reaction mixture at 24° C. The reaction vessel was sealed and the sealed vessel was placed in an oil bath that had been previously heated to 850°. The reaction mixture was stirred and heated for 3.5 h at 85° C. The product mixture was allowed to cool over 30 min to 0° C. with an ice bath. A saturated aqueous sodium bicarbonate solution (20 mL) was added dropwise via syringe to the product mixture. The resulting mixture was stirred for 10 mi at 0° C. The resulting mixture was transfeed to a separatory funnel that had been charged with dichloromethane (50 mL). The layers that formed were a separated and the aqueous layer was extracted with dichloromethane (3×20 mL). The organic layers were combined and the combined organic layers were dried over sodium sulfate. The dried solution was filtered and the filtrate was concentrated. The residue obtained was purified by automated flash-column chromatography (eluting with hexanes initially, grading to 50% ether-hexanes, linear gradient) to afford 11,22-bis(benzyloxymethylenoxy)pleuromutilin (29) as an amorphous white solid (1.24 g, 99%).

R_{f}=0.20 (70% ethyl acetate-hexanes; UV, PAA, CAM). ¹H NMR (400 MHz, CDCl₃) δ 7.37-7.27 (m, 10H, 2×H₂₆, 2×H₂₇, 1×H₂, 2×H₃₀, 2×H₃₁, 1×H₃₂), 6.34 (dd, J=17.5, 11.0 Hz, 1H, H₁₉), 5.76 (d, J=8.5 Hz, 1H, H₁₄), 5.28 (d, J=11.0 Hz, 1H, 1×H₂₀), 5.22 (d, J=17.5 Hz, 1H, 1×H₂₀), 4.84-4.78 (m, 4H, 2×H₂₃, 2×H₂₉), 4.68 (s, 2H, H₃₀), 4.64 (s, 2H, H₂₄), 4.15 (dd, J=24.5, 16.5 Hz, 2H, H₂₂), 3.37 (d, J=6.0 Hz, 1H, H₁₁), 2.47-2.42 (m, 1H, H₁₀), 2.27-2.14 (m, 2H, H₂), 2.09 (s, 1H, H₄), 2.03 (dd, J=16.0, 8.5 Hz, 1H, 1×H₁₃), 1.81-1.71 (m, 2H, 1×H₁, 1×H₈), 1.66-1.55 (m, 2H, 1×H₆, 1×H₇), 1.47-1.42 (m, 4H, 1×H₁, 3×H₁₅), 1.40-1.33 (m, 2H, 1×H₇, 1×H₁₃), 1.18 (s, 3H, H₁₈), 1.13 (td, J=14.0, 4.5 Hz, 1H, 1×H₈), 0.98 (d, J=7.0, 3H, H₁₇), 0.98 (d, J=6.5, 3H, H₁₆). ¹³C NMR (100 MHz, CDCl₃) δ 217.1 (C), 168 6 (C), 140.0 (CH), 137.8 (CH), 137.4 (C), 128.4 (CH), 128.4 (CH), 127.9 (CH), 127.8 (CH), 127.7 (CH), 127.6 (CH), 116.2 (CH₂), 96.9 (CH₂), 94.4 (CH₂), 83.6 (CH), 70.7 (CH₂), 69.8 (CH₂), 79.3 (CH), 65.0 (CH₂), 58.5 (CH), 45.4 (C), 45.1 (CH₂), 44.6 (C), 42.0 (C), 37.0 (C), 36.6 (CH), 34.6 (CH₂), 30.4 (CH₂), 28.7 (CH), 26.7 (CH₂), 25.1 (CH₂), 16.3 (CH₃), 14.8 (CH₃), 12.0 (CH₃). IR (ATR-FTIR), cm⁻¹: 2935 (w), 1733 (m), 1454 (w), 1375 (w), 1284 (w), 1210 (w), 1165 (w), 1114 (w), 1058 (s), 1025 (s), 952 (m), 914 (m), 735 (m), 697 (m). HRMS-ESI (m/z): [M+Na]⁺ calcd for C₃₈H₅₀NaO₇, 641.3457; found, 641.3450. [α]_{D}²⁵+26° (c=1.0, CHCl₃).

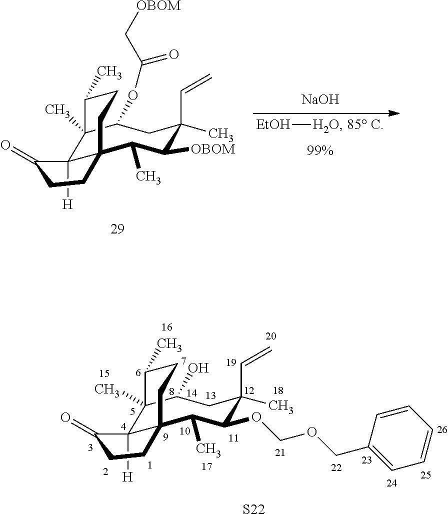

### Synthesis of 11-benzyloxymethylenoxymutilin (S22, FIG. 8, Scheme 8)

Water (1.42 mL) and an aqueous sodium hydroxide solution (50% w/w, 199 μL) were added dropwise via syringe to a solution of 1,22-bis(benzyloxymethylenoxy)pleuromutilin (29, 739 mg, 1.00 mmol, 1 equiv) in ethanol (2.27 mL) in a 25-mL round-bottomed flask fitted with a reflux condenser at 24° C. The reaction vessel was placed in an oil bath that had been previously heated to 85° C. The reaction mixture was stirred and heated for 3 h at 85° C. The resulting mixture was allowed to cool to 24° C. over 30 min. The product mixture was transferred to a separatory funnel that had been charged with dichloromethane (50 mL). The layers that formed were separated and the aqueous layer was extracted with dichloromethane (3×20 mL). The organic layers were combined and the combined organic layers were dried over sodium sulfate. The dried solution was filtered and the filtrate was concentrated. The residue obtained was purified by automated flash-column chromatography (eluting with hexanes initially, grading to 20% ethyl acetate-hexanes, linear gradient) to afford 11-benzyloxymethylenoxypleuromutilin (S22) as an amorphous white solid (459 mg, 99%).

R_{f}=0.34 (33% ether-hexanes; UV, PAA, CAM). ¹H NMR (600 MHz, CDCl₃) δ 7.37-7.28 (m, 5H, 2×H₂₄, 2×H₂₅, 1×H₂₆), 6.12 (dd, J=18.0, 11.4 Hz, 1H, H₁₉), 5.38 (d, J=18 Hz, 1H, H₂₀), 5.23 (d, J=11.4 Hz, 1H, 1×H₂₀), 4.80 (dd, J=18.6, 5.4 Hz, 2H, H₂₁), 4.70-4.65 (m, 2H, H₂₂), 4.31 (dd, J=7.8, 6.0 Hz, 1H, H₁₁), 3.33 (d, J=6.0 Hz, 1H, H₁₄), 2.25-2.12 (m, 3H, 2×H₂, 1×H₁₀), 2.02 (s, 1H, H₄), 1.87 (dd, J=16.2, 7.8 Hz, 1H, 1×H₁₃), 1.75-1.63 (m, 4H, 1×H₁, 1×H₆, 1×H₈, 1×H₁₃), 1.50-1.41 (m, 2H, 1×H₁, 1×H₇), 1.38-1.34 (m, 4H, 1×H₇, 3×H₁₅), 1.28 (d, J=5.4 Hz, OH), 1.15 (s, 3H, H₁₈), 1.12 (td, J=13.8, 4.8 Hz, 1H, 1×H₈), 0.96-0.94 (m, 6H, 3×H₁₆, 3×H₁₇). ¹³C NMR (150 MHz, CDCl₃) δ 217.9 (C), 140.8 (CH), 137.8 (C), 128.4 (CH), 128.3 (CH), 127.6 (CH), 114.7 (CH₂), 96.8 (CH), 83.4 (CH₂), 70.7 (CH₂), 66.7 (CH), 59.2 (CH), 46.1 (C), 45.3 (C), 44.3 (CH₂), 42.3 (C), 37.6 (CH), 36.8 (CH), 34.6 (CH₂), 30.4 (CH₂), 30.1 (CH₃), 27.1 (CH), 25.2 (CH₂), 18.2 (CH₃), 13.4 (CH₃), 12.0 (CH₃). IR (ATR-FTIR), cm⁻¹: 2929 (w), 2826 (w), 1732 (m), 1498 (w), 1455 (m), 1373 (w), 1163 (m), 1023 (s), 947 (m), 921 (w), 733 (m), 697 (m). HRMS-ESI (m/z): [M+H]⁺ calcd for C₂₈H₄₁O₄, 441.3005; found, 441.3003. [α]_{D}²⁵=+58° (c=0.50, CHCl₃).

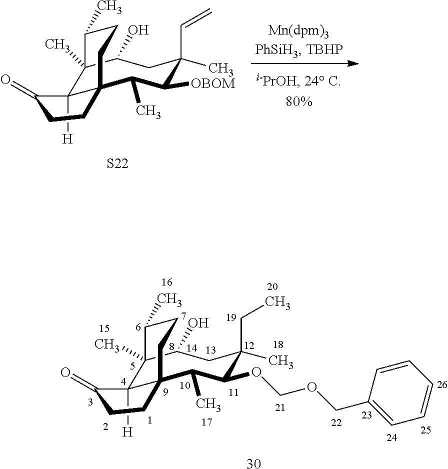

### Synthesis of 11-benzyloxymethylenoxy-19,20-dihydromutilin (30) via HAT hydrogenation (FIG. 8, Scheme 8)

This experiment was adapted from the work of Shenvi and co-workers.³ Phenylsilane (629 μL, 5.10 mmol, 6.00 equiv) and a solution of tert-butyl hydrogen peroxide (5.5 M, 309 μL, 1.70 mmol, 2.00 equiv) in nonane were added dropwise sequentially via syringe to a solution of 11-benzyloxymethylenoxymutilin (S22, 375 mg, 850 μmol, 1 equiv) and tris(2,2,6,6-tetramethyl-3,5-heptanedionato) manganese (1) (76.5 mg, 128 μmol, 0.150 equiv) in iso-propanol (2.0 mL) under argon at 24° C. The reaction exhibited exothermicity in the initiation stage. The resulting mixture was stirred for 4 h at 24° C. The product mixture was concentrated to dryness. The residue obtained was purified by automated flash-column chromatography (eluting with hexanes initially, grading to 20% ethyl acetate-hexanes, linear gradient) to afford 11-benzyloxymethylenoxy-19,20-dihydropleuromutilin (30) as an amorphous white solid (300 mg, 80%).

R_{f}=0.34 (33% ether-hexanes; UV, PAA, CAM). ¹H NMR (400 MHz, CDCl₃) δ 7.35-7.29 (m, 5H, 2×H₂₄, 2×H₂₃, 1×H₂₆), 4.79-4.72 (m, 2H, H₂₁), 4.67-4.64 (m, 2H, H₂₂), 4.27 (d, J=7.6 Hz, 1H, H₁₁), 3.27 (d, J=6.0 Hz, 1H, H₁₄), 2.41-2.35 (m, 1H, H₁₀), 2.28-2.10 (m, 2H, H₂), 2.03 (s, 1H, H₄), 1.77-1.36 (m, 10H, 2×H₁, 1×H₆, 2×H₇, 1×H₈, 2×H₃, 2×H₁₉), 1.31 (M, 3H, H₁₈), 1.13 (td, J=13.6, 4.0 Hz, 1×H₈), 1.02 (s, 3H, H₁₈), 0.97-0.92 (m, 9H, 3×Hic, 3×H₁, 3×H₂). ¹³C NMR (100 MHz, CDCl₃) δ 217.9 (C), 137.9 (C), 128.4 (CH), 128.6 (2×CH), 96.9 (CH₂), 85.2 (CH), 70.7 (CH₂), 66.5 (CH), 59.2 (CH), 45.3 (C), 43.4 (CH), 42.5 (C), 41.3 (C), 36.8 (CH), 35.0 (CH), 34.6 (CH₂), 30.6 (CH₂), 27.2 (CH₂), 27.1 (CH₃), 25.1 (CH₂), 22.0 (CH₂), 18.1 (CH₃), 13.3 (CH₃), 11.8 (CH₃), 8.1 (CH₃). IR (ATR-FTIR), cm⁻¹: 2959 (w), 2830 (w), 2878 (w), 1731 (m), 1457 (w), 1382 (m), 1161 (m), 1114 (w), 1019 (s), 979 (m), 908 (s), 727 (s), 697 (s), 668 (m), 648 (m). HRMS-ESI (m/z): [M+H]⁺ calcd for C₂₈H₄₃O₄, 443.3161; found, 443.3166. [α]_{D}²⁵=+56° (c=0.50, CHCl₃).

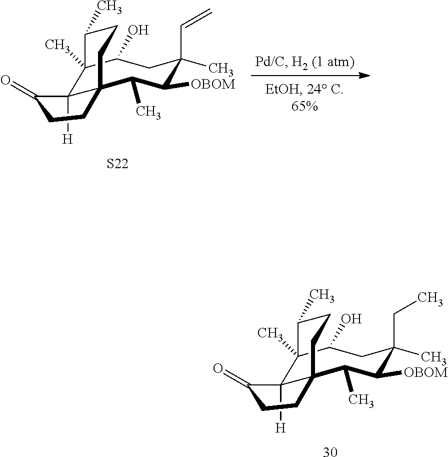

### Synthesis of 11-benzyloxymethylenoxy-19,20-dihydromutilin (30) Via Heterogeneous Hydrogenation

Ethanol (525 μL) was added to a mixture of I-benzyloxymethylenoxymutilin (S22, 50.0 mg, 116 μmol, 1 equiv) and palladium on carbon (5 wt. % loading, 12.2 mg, 0.05 equiv) under argon at 24° C. The reaction vessel was evacuated and refilled using a balloon of hydrogen. This process was repeated four times. An aliquot was taken from the reaction mixture every 30 min and the conversion of S22 was judged by GC-MS analysis. The reaction mixture was stirred for 295 min at 24° C. The hydrogen balloon was replaced with a stream of nitrogen and the product mixture was purged by bubbling nitrogen at 24° C. for 10 min. The resulting mixture was filtered through a pad of celite and the pad was rinsed with dichloromethane (100 mL). The filtrates were combined and the combined filtrates were concentrated to dryness. The residue obtained was purified by automated flash-column chromatography (eluting with hexanes initially, grading to 50% ether-hexanes, linear gradient) to afford 11-benzyloxymethylenoxy-19,20-dihydropleuromutilin (30) as an amorphous white solid (32.5 mg, 65%).

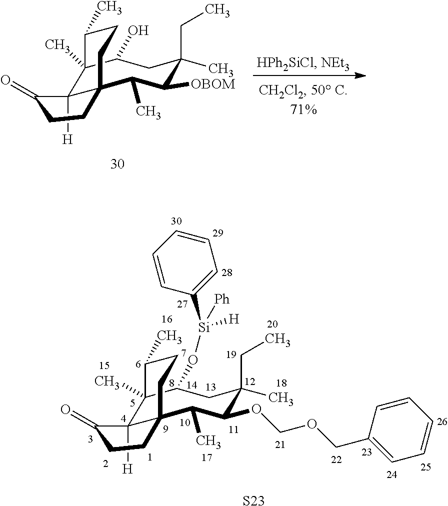

### Synthesis of Silane S23 (FIG. 8, Scheme 8)

A 25-mL round-bottomed flask fused to a Teflon-coated valve was charged with 11-benzyloxymethylenoxy-19,20-dihydropleuromutilin (30, 300 mg, 678 μmol, 1 equiv). Benzene (500 μL) was added and the solution was concentrated to dryness. This process was repeated twice. The reaction vessel was evacuated and refilled using a balloon of argon. This process was repeated two times. Dichloromethane (3.0 mL), triethylamine (378 μL, 2.71 mmol, 4.00 equiv), and (chloro)diphenylsilane (265 μL, 1.36 mmol, 2.00 equiv, 95% purity) were added sequentially to the reaction vessel. The vessel was sealed and the sealed vessel was placed in an oil bath that had been previous heated to 50° C. The reaction was stirred and heated for 90 min at 50° C. The reaction vessel was allowed to cool over 30 min to 24° C. The product mixture was diluted sequentially with pentane (3.0 mL) and an aqueous potassium phosphate buffer solution (pH 7, 0.10 M, 3.0 mL). The diluted mixture was transferred to a separatory funnel and the layers formed were separated. The aqueous layer was extracted with dichloromethane (3×10 mL). The organic layers were combined and the combined organic layers were dried over sodium sulfate. The dried solution was filtered and the filtrate was concentrated to dryness. The residue obtained was purified by automated flash-column chromatography (eluting with hexanes initially, grading to 40% ether-hexanes, linear gradient) to afford silane S23 as an amorphous white solid (300 mg, 71%).

R_{f}=0.59 (40% ether-hexanes; UV, PAA, CAM). ¹H NMR (500 MHz, C₆D₆) δ 7.76-7.26 (m, 4H, 4×H₂₉), 7.29-7.08 (m, 11H, 2×H₂₄, 2×H₂₅, 1×H₂₆, 4×H₂, 2×H₃₀), 5.80 (s, 1H, Si—H), 4.72 (d, J=7.5 Hz, 1H, H₁₁), 4.56-4.48 (m, 4H, 2×H₂₁, 2×H₂₂), 3.02 (d, J=5.5 Hz, 1H, H₁₄), 2.15-2.09 (m, 1H, H₁₀), 1.93 (s, 3H, H₁), 1.87-1.80 (m, 3H, 1×H₁, 2×H₂), 1.80-1.65 (m, 4H, 1×H₄, 1×H₆, 2×H₁₃), 1.43-1.32 (m, 3H, 1×H₁, 1×H₇, 1×H₁₉), 1.29-1.22 (m, 1H, 1×H₈), 1.14-1.09 (m, 1H, 1×H₇), 1.06 (d, J=7.0 Hz, 3H, H₁₇), 1.04-1.00 (m, 1H, 1×H₁₉), 0.97 (t, J=11.5 Hz, 3H, H₂₀), 0.90 (s, 3H, H₁₈), 0.87-0.79 (m, 4H, 1×H₈, 3×H₁₆). ¹³C NMR (150 MHz, C₆D₆) δ 215.4 (C), 138.3 (C), 135.3 (C), 135.0 (C), 135.0 (CH), 134.6 (CH), 130.1 (CH), 130.0 (CH), 128.2 (CH), 128.2 (CH), 127.9 (CH), 127.9 (CH), 127.4 (CH), 96.8 (CH₂), 85.0 (CH), 70.2 (CH₂), 69.5 (CH), 58.6 (CH), 45.1 (CH₂), 45.0 (C), 43.8 (C), 41.2 (C), 37.3 (CH), 35.3 (CH), 34.2 (CH₂), 30.3 (CH₂), 27.1 (CH₂), 26.5 (CH), 24.9 (CH₂), 24.4 (CH₂), 18.9 (CH₃), 14.6 (CH), 11.8 (CH), 9.7 (CH). IR (ATR-FTIR), cm⁻¹: 2933 (w), 1734 (m), 1456 (w), 1428 (w), 1158 (w), 1112 (m), 1024 (s), 994 (w), 812 (m), 731 (s), 697 (s), 497 (s). HRMS-ESI (m/z): [M-Si(C₆H₅)₂+H]⁺ calcd for C₂₈H₄₃O₄, 443.3161; found, 443.3164. [α]_{D}²⁵=+52° (c=0.25, CHCl₃).

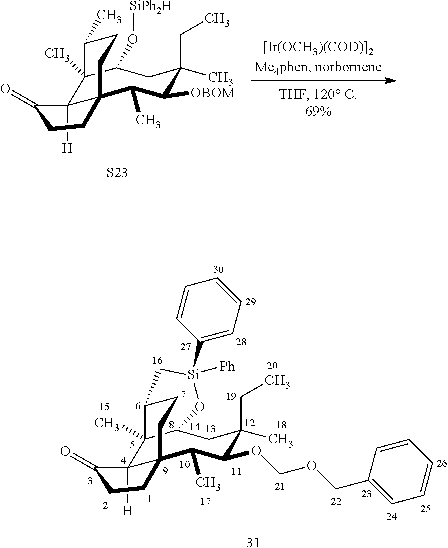

### Synthesis of Silacycle 31 (FIG. 8, Scheme 8)

This experiment was adapted from the work of Hartwig and co-workers.² A 4-mL pressure tube with a Teflon-coated valve was charged with 3,4,7,8-tetramethyl-1,10-phenanthroline (13.7 mg, 58.0 μmol, 12.5 mol %) and norbornene (65.5 mg, 696 μmol, 1.50 equiv) in the glovebox. A 4-mL vial was charged with silane S23 [290 mg, 464 μmol, 1 equiv, dried by azeotropic distillation with benzene (3×1 mL)]. The vessel containing the silane was evacuated and refilled using a balloon of argon. This process was repeated two times. Tetrahydrofuran (350 μL) was transferred into the vessel containing the silane and the resulting solution was added to the vessel containing the ligand and norbornene in the glovebox. The vessel containing the silane was rinsed with tetrahydrofuran (3×50 μL) and the combined rinses were transferred to the reaction vessel.

Methoxy(cyclooctadiene)iridium(I) dimer (15.4 mg, 7.7 μmol, 5.0 mol %) was added to an oven-dried 4-mL vial. Tetrahydrofuran (350 μL) was added into the vial containing the catalyst and the resulting solution was transferred dropwise via syringe to the reaction vessel in the glovebox. The vial containing the catalyst was rinsed with tetrahydrofuran (3×50 μL) and the combined rinses were transferred into the reaction vessel. The reaction vessel was sealed and the reaction mixture was stirred for 1 h at 24° C. in the glovebox. The sealed reaction vessel was then removed from the glovebox and placed in an oil bath that had been preheated to 120° C. The reaction mixture was stirred and heated for 6 h at 120° C. The reaction vessel was allowed to cool over 30 min to 24° C. and the cooled product mixture was concentrated to dryness. The residue obtained was purified by automated flash-column chromatography (eluting with hexanes initially, grading to 15% ether-hexanes, linear gradient) to afford the silacycle 31 as an amorphous white solid (201 mg, 69%).

R_{f}=0.59 (40% ether-hexanes; UV, PAA, CAM). ¹H NMR (500 MHz, C₆D₆) δ 7.71-7.60 (m, 4H, 4×H₂₉), 7.25-7.03 (m, 11H, 2×H₂₄, 2×H₂₄, 1×H₂₆, 4×H₂₈, 2×H₃₀), 4.57 (d, J=7.0 Hz, 1H, H₁₁), 4.52-4.48 (m, 2H, 2×H₂₁), 4.47-4.43 (m, 2H, 2×H₂₂), 2.95 (d, J=6.5 Hz, 1H, H₁₄), 2.24-2.19 (m, 1H, H₁₀), 2.12-2.06 (m, 1H, H₆), 1.95-1.90 (m, 1H, 1×H₂), 1.83-1.72 (m, 5H, 1×H₂, 1×H₃, 3×H₁₅), 1.69-1.61 (m, 3H, 1×H₄, 2×H₁₉), 1.56-1.50 (m, 3H, 1×H₁, 1×H₁₃, 1×H₁₆), 1.26-1.17 (m, 3H, 1×H₁, 1×H₇, 1×H₈), 1.07 (t, J=7.5 Hz, 3H, H₂₀), 1.02-0.89 (m, 4H, 1×H₇, 3×H₁₈), 0.86-0.82 (m, 1H, 1×H₁₆), 0.73 (td, J=14.5, 4.5 Hz, 1H, 1×H₈), 0.53 (d, J=7.0 Hz, 3H, H₇). ¹³C NMR (150 MHz, C₆D₆) δ 215.6 (C), 138.3 (C) 137.1 (C), 136.4 (C), 134.3 (CH), 134.1 (CH), 134.0 (CH), 134.0 (CH), 129.9 (CH), 129.8 (CH), 128.2 (CH), 127.8 (CH), 127.4 (CH), 97.0 (CH₂), 85.3 (CH), 70.3 (CH₂), 66.5 (CH), 58.3 (CH), 44.5 (C), 41.4 (C), 41.0 (C), 41.0 (CH₂), 38.0 (CH), 35.8 (CH), 34.0 (CH₂), 30.1 (CH₂), 27.3 (CH₂) 26.6 (CH₃), 25.5 (CH₂), 21.7 (CH₂), 15.0 (CH₃), 12.8 (CH₂), 12.1 (CH₃), 8.3 (CH₃). IR (ATR-FTIR), cm⁻¹: 2936 (w), 1736 (w), 1457 (w), 1162 (w), 1118 (w), 1021 (s), 957 (w), 736 (w), 697 (s), 496 (s). HRMS-ESI (m/z): [M+H]⁺ calcd for C₄₀H₅₁O₄Si, 623.3557; found, 623.3552. [α]_{D}²⁵=+57° (c=0.50, CHCl₃).

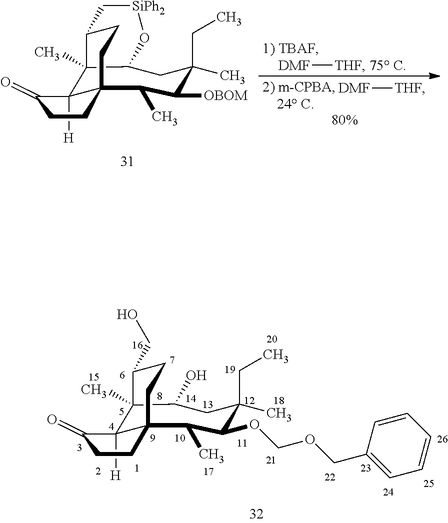

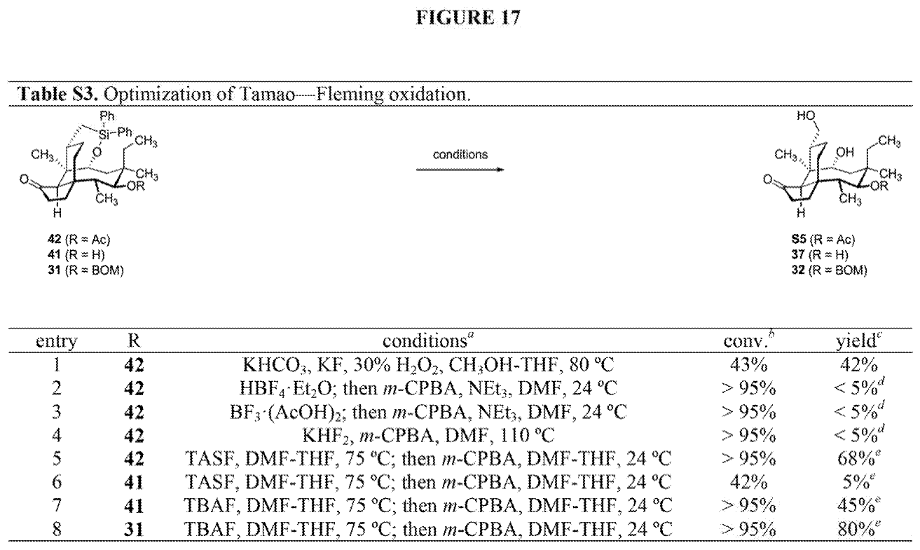

### Synthesis of Diol 32 (FIG. 8, Scheme 8 and FIG. 17, Table 3 Entry 8)

A solution of tetrabutylammonium fluoride (1.0 M, 644 μL, 644 μmol, 2.00 equiv) in tetrahydrofuran was added dropwise via syringe to a solution of the silacycle 31 (201 mg, 322 μmol, 1 equiv) in N,N-dimethylformamide (1.0 mL) at 24° C. The reaction vessel was placed in an oil bath that had been previously heated to 75° C. The reaction mixture was stirred and heated for 5 min at 75° C. The resulting mixture was immediately cooled to 24° C. with an ice bath. Freshly recrystallized m-chloroperbenzoic acid (167 mg, 966 μmol, 3.00 equiv) was added to the reaction mixture at 24° C. The reaction mixture was stirred for 15 min at 24° C. The product mixture was diluted sequentially with ether (5.0 mL) and aqueous potassium phosphate buffer solution (pH 7, 0.10 M, 3.0 mL). The diluted product mixture was transferred to a separatory funnel that had been charged with a mixture of ether and pentane (1:1, v/v, 30 mL). The layers that formed were separated and the organic layer was washed with saturated aqueous sodium bicarbonate solution (3×5 mL). The washed organic layer was dried over sodium sulfate. The dried solution was filtered and the filtrate was concentrated to dryness. The residue obtained was purified by automated flash-column chromatography (eluting with hexanes initially, grading to 50% ethyl acetate-hexanes, linear gradient) to afford the diol 32 as an amorphous white solid (118 mg, 80%).

R_{f}=0.44 (50% ethyl acetate-hexanes; UV, PAA, CAM). ¹H NMR (400 MHz, CDCl₃) δ 7.37-7.27 (m, 5H, 2×H₂₄, 2×H₂₅, 1×H₂₆), 4.79-4.75 (m, 2H, 2×H₂₁), 4.66 (s, 2H, 2×H₂), 4.26 (d, J=11.6 Hz, 1H, H₁), 3.93 (d, J=11.6 Hz, 1H, 1×H₁₆), 3.48 (dd, J=11.6, 4.4 Hz, 1H, 1×H₆), 3.28 (d, J=6.4 Hz, 1H, H₁₄), 2.80 (br s, 2H, 2×OH), 2.48-2.40 (m, 1H, H₁₀), 2.32-2.10 (m, 2H, H₂), 2.07 (s, 1H, H₄), 1.97 (qd, J=14.0, 3.6 Hz, 1H, 1×H₁₉), 1.86 (dt, J=14.4, 3.6 Hz, 1H, 1×H₈), 1.74-1.42 (m, 7H, 2×H₁, 1×H₆, 2×H₇, 2×H₁₃), 1.39-1.33 (m, 4H, 3×H₁₅, 1×H₁₉), 1.17 (td, J=14.0, 4.4 Hz, 1H, 1×H₈), 1.02 (s, 3H, H₁₈), 0.98-0.89 (m, 6H, 3×H₁₇, 3×H₂). ¹³C NMR (100 MHz, CDCl₃) δ 218.0 (C), 137.9 (C), 128.4 (CH), 127.7 (CH×2), 97.0 (CH₂), 85.4 (CH), 70.8 (CH₂), 64.7 (CH), 62.8 (CH₂), 59.7 (CH), 45.3 (C), 43.3 (CH), 42.7 (C), 41.6 (CH₂), 41.3 (C), 35.4 (CH), 34.5 (CH₂), 30.6 (CH₂), 27.1 (CH₃), 25.2 (Cl₂), 22.1 (CH₂), 21.3 (CH₂), 13.7 (CH₃), 12.0 (CH₃), 8.0 (CH₃). IR (ATR-FTIR), cm⁻¹: 3329 (br w), 2935 (w), 2879 (w), 1731 (m), 1457 (w), 1382 (w), 1288 (w), 1162 (w), 1039 (s), 1022 (s), 965 (m), 944 (m), 908 (m), 720 (s), 698 (m). HRMS-ESI (m/z): [M+Na]⁺ calcd for C₂₈H₄₂NaO₅, 481.2930; found, 481.2927. [α]_{D}²⁵=+55° (c=0.50, CHCl₃).

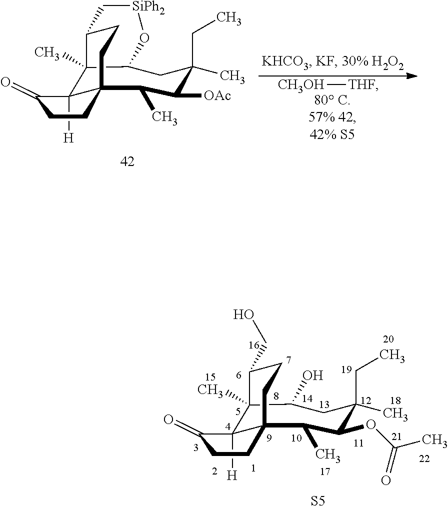

### Synthesis of Diol S5 (FIG. 17, Table S3 Entry 1)

Tetrahydrofuran (100 μL) and an aqueous hydrogen peroxide solution (30% w/w, 33.3 μL, 288 μmol, 20.0 equiv) were added sequentially to a suspension of the silacycle 42 (8.0 mg, 14.4 μmol, 1 equiv), potassium fluoride (5.1 mg, 86.5 μmol, 6.00 equiv), and potassium bicarbonate (8.8 mg, 86.5 μmol, 6.00 equiv) in methanol (100 μL) at 24° C. in a 4-mL pressure tube with a Teflon-coated valve. The tube was sealed and the sealed tube was placed in an oil bat that had been preheated to 80° C. The reaction mixture was stirred and heated at 80° C. for 7 h. The product mixture was diluted sequentially with dichloromethane (2.0 mL), saturated aqueous sodium thiosulfate (1.0 mL), and saturated aqueous sodium bicarbonate (500 μL). The diluted product mixture was transferred to a separatory funnel and the layers that formed were separated. The aqueous layer was extracted with dichloromethane (3×5 mL). The organic layers were combined and the combined organic layers were dried over sodium sulfate. The dried solution was filtered and the filtrate was concentrated to dryness. The residue obtained was purified by automated flash-column chromatography (eluting with hexanes initially, grading to 50% ethyl acetate-hexanes, linear gradient) to afford separately the silacycle 42 as an amorphous white solid (3.4 mg, 42%) and the diol S5 as an amorphous white solid (3.2 mg, 57%).

Diol S5: R_{f}=0.45 (50% ethyl acetate-hexanes; PAA, CAM). ¹H NMR (400 MHz, CDCl₃) δ 4.83 (d, J=6.8 Hz, 1H, H₁₁), 4.23 (d, J=6.4 Hz, 1H, H₁₄), 3.95 (d, J=11.2 Hz, 1H, 1×H₁₆), 3.83 (br s, 1H, C16-OH), 3.51 (dd, J=11.2, 4.0 Hz, 1H, 1×H₆), 2.82 (br s, 1H, C14-OH), 2.51-2.44 (m, 1H, H₁₀), 2.31 (dd, J=19.6, 11.2 Hz, 1H, 1×H₂), 2.21 (s, 1H, H₄), 2.15 (dd, J=19.6, 11.2 Hz, 1H, 1×H₂), 2.07 (s, 3H, H₂₂), 2.01-1.90 (m, 2H, 1×H₁, 1×H₁₉), 1.82 (dt, J=14.8, 2.0 Hz, 1H, 1×H₈), 1.72 (td, J=14.0, 7.2 Hz, 1H, 1×H₁₃), 1.66-1.56 (m, 3H, 1×H₆, 2×H₇), 1.54-1.49 (m, 1H, 1×H₁₃), 1.44-1.34 (m, 5H, 1×H₁, 3×H₁₅, 1×H₁₉), 1.17 (td, J=14.4, 4.0 Hz, 1H, 1×H₈), 0.95 (t, J=7.4 Hz, 3H, H₂₀), 0.85 (s, 3H, H₁₈), 0.77 (d, J=6.8 Hz, 3H, H₁₇). ¹³C NMR (100 MHz, CDCl₃) δ 217.9 (C), 170.6 (C), 78.3 (CH), 64.5 (CH), 62.8 CH₂), 59.7 (CH), 45.2 (C), 43.2 (CH), 42.7 (C), 41.2 (CH₂), 39.9 (C), 34.8 (CH), 34.4 (CH₂), 30.4 (CH₂), 26.0 (CH₃), 25.0 (CH₂), 22.4 (CH₂), 21.3 (CH₃), 20.8 (CH₂), 13.7 (CH), 11.8 (CH₃), 8.0 (CH₃). IR (ATR-FTIR), cm⁻¹: 3192 (br w), 2953 (w), 2863 (w), 1735 (s), 1463 (m), 1385 (w), 1254 (s), 1109 (w), 1024 (w), 974 (m). HRMS-ESI (m/z): [M+Na]⁺ calcd for C₂₂H₃₆NaO₅, 403.2460; found, 403.2462. [α]_{D}²⁵=+53° (c=0.10, CHCl₃).

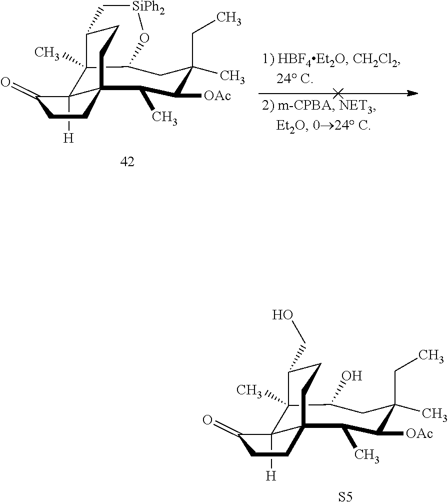

### Attempted Synthesis of Diol S5 (FIG. 17, Table S3 Entry 2)

Tetrafluoroboric acid diethyl ether complex (20.2 μL, 147 μmol, 10.0 equiv) was added dropwise via syringe to a solution of the silacycle 42 [8.0 mg, 14.7 μmol, 1 equiv, dried by azeotropic distillation with benzene (3×200 μL)] in dichloromethane (200 μL) at 24° C. The resulting mixture was stirred for 1 h at 24° C. The product mixture was diluted sequentially with dichloromethane (2.0 mL) and saturated aqueous sodium bicarbonate (500 μL). The diluted product mixture was transferred to a separatory funnel and the layers that formed were separated. The aqueous layer was extracted with dichloromethane (3×5 mL). The organic layers were combined and the combined organic layers were dried over sodium sulfate. The dried solution was filtered and the filtrate was concentrated to dryness. The residue obtained was dissolved in ether (150 μL). A solution of triethylamine (2.5 μL, 17.8 μmol, 1.20 equiv) in ether (50 μL) was added to the reaction mixture and the reaction vessel was cooled to 0° C. with an ice bath. Freshly recrystallized m-chloroperbenzoic acid (10.1 mg, 58.8 μmol, 4.00 equiv) was added to the reaction mixture. The resulting mixture was stirred for 30 min at 0° C., and then the ice bath was removed. The reaction mixture was stirred for 2 h at 24° C. The product mixture was transferred to a separatory funnel that had been charged with ethyl acetate (30 mL). The diluted product mixture was washed with saturated aqueous sodium bicarbonate solution (3×5 mL). The washed organic layer was dried over sodium sulfate. The dried solution was filtered and the filtrate was concentrated to dryness. ¹H NMR analysis of the residue obtained showed complex decompositions.

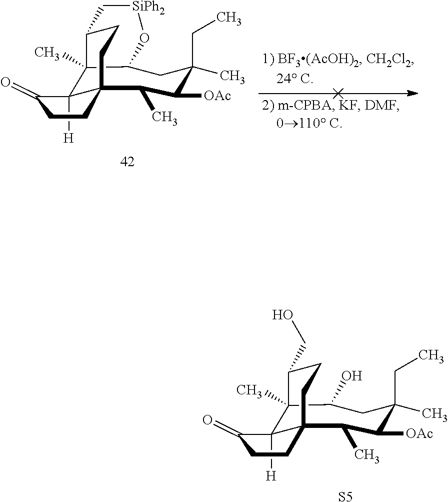

### Attempted Synthesis of Diol 42 (FIG. 17, Table 3 Entry 3)

Boron trifluoride acetic acid complex (20.4 μL, 147 μmol, 10.0 equiv) was added dropwise via syringe to a solution of the silacycle 42 [8.0 mg, 14.7 μmol, 1 equiv, dried by azeotropic distillation with benzene (3×200 μL)] in dichloromethane (200 μL) at 24° C. in a 4-mL vial. The resulting mixture was stirred for 1 h at 24° C. The product mixture was diluted sequentially with dichloromethane (2.0 mL) and saturated aqueous sodium bicarbonate (500 μL). The diluted product mixture was transferred to a separatory funnel and the layers that formed were separated. The aqueous layer was extracted with dichloromethane (3×5 mL). The organic layers were combined and the combined organic layers were dried over sodium sulfate. The dried solution was filtered and the filtrate was concentrated to dryness. The residue obtained was dissolved in ether (150 μL). Potassium fluoride (1.7 mg, 29.4 μmol, 2.00 equiv) was added to the reaction mixture and the reaction vessel was cooled to 0° C. with an ice bath. Freshly recrystallized m-chloroperbenzoic acid (10.1 mg, 58.8 μmol, 4.00 equiv) was added to the reaction mixture. The resulting mixture was stirred for 30 min at 0° C., and then the ice bath was removed. The reaction mixture was stirred for 2 h at 24° C. The product mixture was transferred to a separatory funnel that had been charged with ethyl acetate (30 mL). The diluted product mixture was washed with saturated aqueous sodium bicarbonate solution (3×5 mL). The washed organic layer was dried over sodium sulfate. The dried solution was filtered and the filtrate was concentrated to dryness. ¹H NMR analysis of the residue obtained showed complex decompositions.

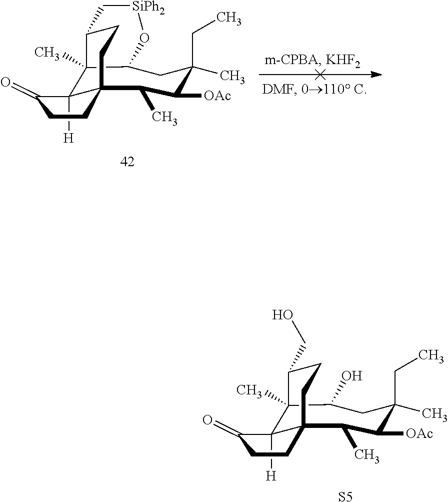

### Attempted Synthesis of Diol S5 (FIG. 17, Table 0.3 Entry 4)

Freshly recrystallized m-chloroperbenzoic acid (7.8 mg, 44.1 μmol, 3.00 equiv) was added to a suspension of the silacycle 42 [8.0 mg, 14.7 μmol, 1 equiv, dried by azeotropic distillation with benzene (3×200 μL)] and potassium bifluoride (2.4 mg, 29.4 μmol, 2.00 equiv) in N,N-dimethylformamide (200 μL) at 0° C. in a 4-mL vial. The reaction vessel was sealed with a Teflon-lined cap. The sealed vial was placed in an oil bath that had been previously heated to 110° C. The reaction mixture was stirred and heated for 2 h at 110° C. The product mixture was transferred to a separatory funnel that had been charged with ethyl acetate (30 mL). The diluted product mixture was washed with saturated aqueous sodium bicarbonate solution (3×5 mL). The washed organic layer was dried over sodium sulfate. The dried solution was filtered and the filtrate was concentrated to dryness. ¹H NMR analysis of the residue obtained showed complex decompositions.

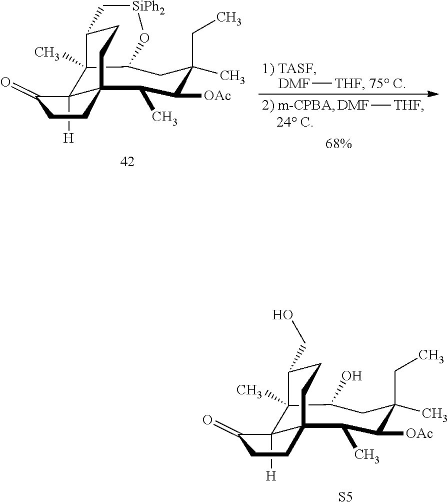

### Synthesis of Diol S5 (FIG. 17, Table S3 Entry 5)

A solution of tris(dimethylamino)sulfonium difluorotrimethylsilicate (3.3 mg, 12.0 μmol, 1.20 equiv) in N,N-dimethylformamide (100 μL) was added dropwise via syringe to a solution of the silacycle 42 (5.4 mg, 10.0 μmol, 1 equiv) in a mixture of tetrahydrofuran and N,N-dimethylformamide (1:1 v/v, 100 μL) at 24° C. The reaction vessel was placed in an oil bath that had been previously heated to 75° C. The reaction mixture was stirred and heated for 2 h at 75° C. The resulting mixture was cooled over 30 min to 24° C. Freshly recrystallized m-chloroperbenzoic acid (5.2 mg, 30.0 μmol, 3.00 equiv) was added to the reaction mixture at 24° C. The reaction mixture was stirred for 75 min at 24° C. The product mixture was diluted sequentially with ether (1.0 mL) and aqueous potassium phosphate buffer solution (pH 7, 0.10 M, 1.0 mL). The diluted product mixture was transferred to a separatory funnel that had been charged with a mixture of ether and pentane (1:1, v/v, 30 mL). The layers that formed were separated and the organic layer was washed with saturated aqueous sodium bicarbonate solution (3×5 mL). The washed organic layer was dried over sodium sulfate. The dried solution was filtered and the filtrate was concentrated to dryness. The residue obtained was purified by automated flash-column chromatography (eluting with hexanes initially, grading to 50% ethyl acetate-hexanes, linear gradient) to afford the diol S5 as an amorphous white solid (2.6 mg, 68%).

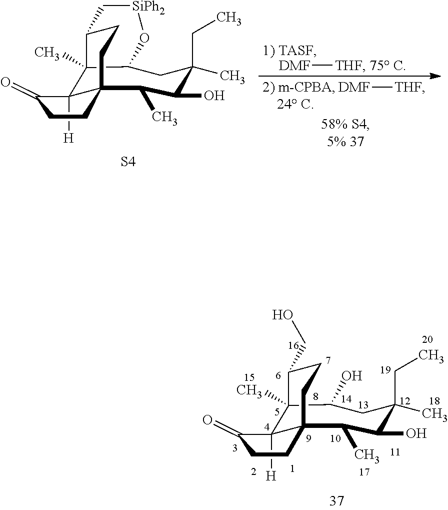

### Synthesis of 16-hydroxy-19,20-dihydromutilin (37, FIG. 17, Table S3 Entry 6)

Tris(dimethylamino)sulfonium difluorotrimethylsilicate (415 mg, 1.15 mmol, 2.00 equiv) was added to a solution of the silacycle S4 (290 mg, 577 μmol, 1 equiv) in a mixture of tetrahydrofuran and N,N-dimethylformamide (1:3 v/v, 12 mL) at 24° C. The reaction vessel was placed in an oil bath that had been previously heated to 75° C. The reaction mixture was stirred and heated for 3 h at 75° C. The resulting mixture was cooled to 24° C. over 30 min. Freshly recrystallized m-chloroperbenzoic acid (299 mg, 1.73 mmol, 3.00 equiv) was added to the reaction mixture at 24° C. The reaction mixture was stirred for 75 min at 24° C. The product mixture was diluted sequentially with ether (10 mL) and aqueous potassium phosphate buffer solution (pH 7, 0.10 M, 10 mL). The diluted product mixture was transferred to a separatory funnel that had been charged with a mixture of ether and pentane (1:1, v/v, 60 mL). The layers that formed were separated and the organic layer was washed with saturated aqueous sodium bicarbonate solution (3×15 mL). The washed organic layer was dried over sodium sulfate. The dried solution was filtered and the filtrate was concentrated to dryness. The residue obtained was purified by automated flash-column chromatography (eluting with hexanes initially, grading to 75% ethyl acetate-hexanes, linear gradient) to afford separately the silacycle S4 as an amorphous white solid (167 mg, 58%) and the triol 37 as an amorphous white solid (10.1 mg, 5%).

Triol 37: R_{f}=0.45 (50% ethyl acetate-hexanes; PAA, CAM). ¹H NMR (500 MHz, CD₃OD) δ 4.24 (d, J=7.0 Hz, 1H, H₁₁), 3.67 (dd, J=11.5, 3.5 Hz, 1H, 1×H₁₆), 3.53 (dd, J=11.5, 3.5 Hz, 1H, 1×H₁₆), 3.41 (d, J=6.5, 1H, H₁₄), 2.38-2.32 (m, 1H, H₁₀), 2.27-2.21 (m, 2H, 1×H₂, 1×H₄), 2.16-2.08 (m, 1H, 1×H₂), 1.89-1.78 (m, 2H, 1×H₇, 1×H₈), 1.71-1.59 (m, 3H, 1×H₁, 1×H₁₃, 1×H₁₉), 1.55-1.42 (m, 5H, 1×H₁, 1×H₆, 1×H₇, 1×H₁₃, 1×H₁₉), 1.33 (s, 3H, H₁₅), 1.16 (td, J=14.0, 4.0 Hz, 1H, 1×H₈), 0.98 (s, 3H, H₁₈), 0.95-0.90 (m, 6H, 3×H₁₇, 3×H₂₀). ¹³C NMR (125 MHz, CD₃OD) δ 219.0 (C), 75 7 (CH), 64.6 (CH), 62.1 (CH₂), 59.2 (CH), 45.3 (C), 44.3 (CH), 42.4 (C), 40.7 (CH₂), 40.3 (C), 34.9 (CH), 33.8 (CH₂), 30.2 (CH₂), 25.9 (CH₃), 24.5 (CH₂), 21.4 (CH₂), 20.6 (CH₂), 13.0 (CH₃), 10.5 (CH₃), 7.1 (CH₃). IR (ATR-FTIR), cm⁻¹: 2991 (w), 1771 (s), 1459 (m), 1383 (w), 1292 (m), 1094 (m), 1059 (s), 1037 (s), 1012 (s), 971 (m), 899 (m), 580 (m). HRMS-ESI (m/z): [M+Na]⁺ calcd for C₂₀H₃₄NaO₄, 361.2355; found, 351.2350.

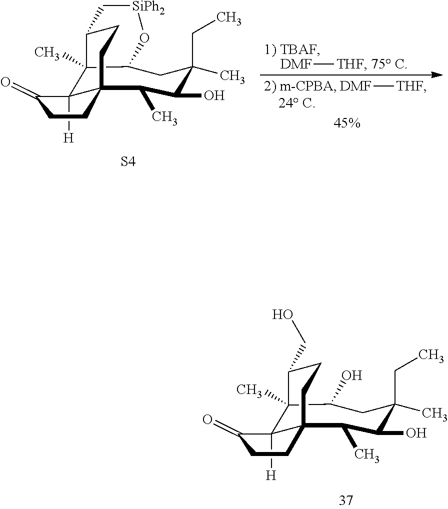

### Synthesis of 16-hydroxy-19,20-dihydromutilin (37, FIG. 17, Table S3 entry 7)

A solution of tetrabutyl ammonium fluoride (1.0 M, 1.52 mL, 1.52 mmol, 1.20 equiv) in tetrahydrofuran was added to a solution of the silacycle S4 (635 mg, 1.26 mmol, 1 equiv) in a mixture of tetrahydrofuran and N,N-dimethylformamide (1:3 v/v, 26 mL) at 24° C. The reaction vessel was placed in an oil bath that had been previously heated to 75° C. The reaction mixture was stirred and heated at 75° C. for 3 h. The resulting mixture was cooled over 30 min to 24° C. Freshly recrystallized m-chloroperbenzoic acid (446 mg, 2.59 mmol, 2.00 equiv) was added to the reaction mixture at 24° C. The reaction mixture was stirred for 75 min at 24° C. The product mixture was diluted sequentially with ether (50 mL) and aqueous potassium phosphate buffer solution (pH 7, 0.10 M, 25 mL). The diluted product mixture was transferred to a separatory funnel that had been charged with a mixture of ether and pentane (1:1, v/v, 150 mL). The layers that formed were separated and the organic layer was washed with saturated aqueous sodium bicarbonate solution (3×25 mL). The washed organic layer was dried over sodium sulfate. The dried solution was filtered and the filtrate was concentrated to dryness. The residue obtained was purified by automated flash-column chromatography (eluting with hexanes initially, grading to 75% ethyl acetate-hexanes, linear gradient) to afford the diol 37 as an amorphous white solid (196 mg, 45%).

A portion of 37 was further purified by recrystallization from ethyl acetate to afford a sample for X-ray analysis.

Triol 37: mp 149-150° C. [α]_{D}²⁵=+57° (c=0.50, CHCl₃).

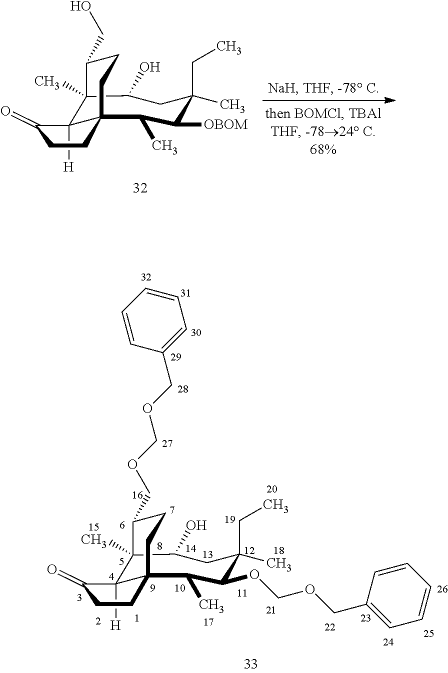

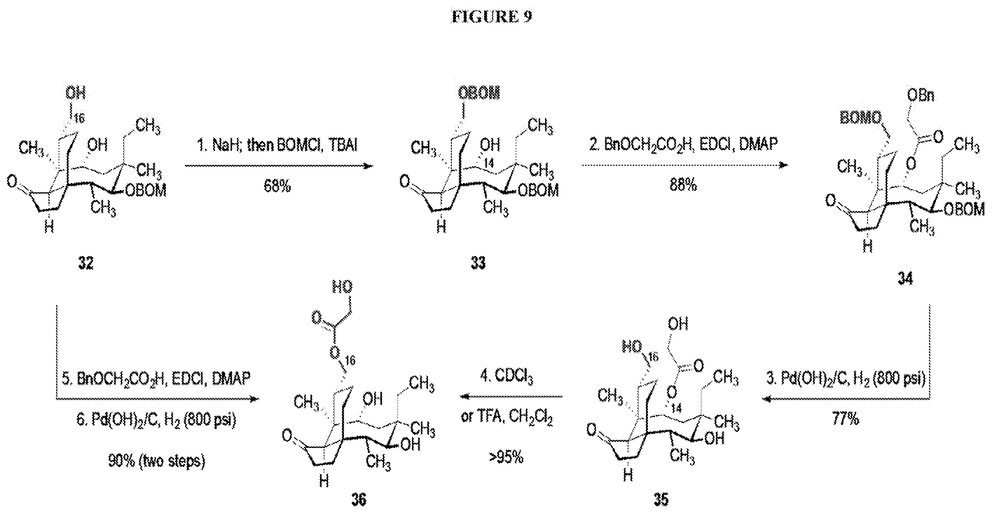

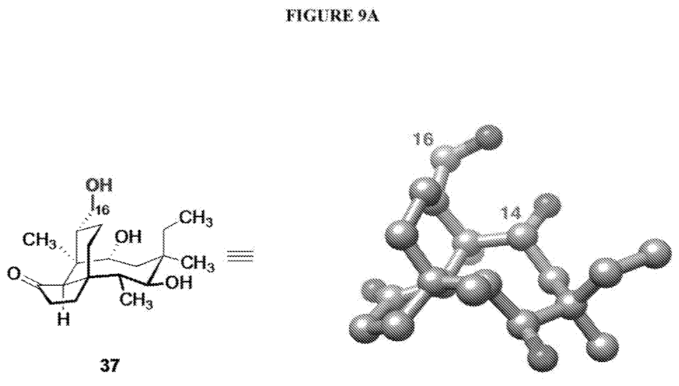

### Synthesis of bis(benzyloxymethyl)ether 33 (FIG. 9, Scheme 9)

Dry sodium hydride (6.8 mg, 283 μmol, 3.30 equiv) was added to a 4-mL vial in the glovebox. The vial was sealed with a septum and the sealed vial was removed out of the glovebox. Tetrahydrofuran (200 μL) was added to the vial containing sodium hydride and the resulting suspension was cooled to −78° C. A separate 4-mL vial was charged with the diol 32 [39.4 mg, 85.8 μmol, 1 equiv, dried by azeotropic distillation with benzene (3×500 μL)] and tetrahydrofuran (400 μL). The resulting diol solution was added dropwise via syringe to the cooled sodium hydride suspension at −78° C. The vial containing starting material was rinsed with tetrahydrofuran (3×50 μL) and the combined rinses were added dropwise via syringe to the reaction vessel at −78° C. The resulting suspension was stirred for 15 min at −78° C. Benzyl chloromethyl ether (14.3 μL, 103 μmol, 1.20 equiv) was added dropwise via syringe to the reaction mixture at −78° C. The resulting mixture was allowed to warm up over 2 h to 24° C. Tetrabutylammonium iodide (3.2 mg, 8.6 μmol, 0.100 equiv) was added to the warmed reaction vessel and the resulting mixture was stirred for 18 h at 24° C. The product mixture was diluted sequentially with ether (5.0 mL) and saturated aqueous ammonium chloride solution (1.0 mL). The diluted product mixture was transferred to a separatory funnel that had been charged with a mixture of ether and pentane (1:1, v/v, 30 mL). The layers that formed were separated and the organic layer was washed with water (3×2.0 mL). The washed organic layer was dried over sodium sulfate. The dried solution was filtered and the filtrate was concentrated to dryness. The residue obtained was purified by automated flash-column chromatography (eluting with hexanes initially, grading to 40% ether-hexanes, linear gradient) to afford the bis(benzyloxymethyl)ether 33 as an amorphous white solid (33.6 mg, 68%).

R_{f}=0.45 (50% ethyl acetate-hexanes; PAA, CAM). ¹H NMR (400 MHz, CDCl₃) δ 7.38-7.27 (m, 10H, 2×H₂₄, 2×H₂₃, 1×H₂₆, 2×H₃₀, 2×H₃₁, 1×H₃₂), 4.80-4.75 (m, 4H, 2×H₂₁, 2×H₂₇), 4.66 (s, 2H, H₂₂), 4.59 (s, 2H, H₂₈), 4.26 (br s, 1H, H₁₁), 3.97 (d, J=3.6 Hz, 1H, OH), 3.88 (dd, J=10.4, 2.4 Hz, 1H, 1×H₁₆), 3.51 (dd, J=10.4, 4.0 Hz, 1H, 1×H₁₆), 3.30 (d, J=6.0 Hz, 1H, H₁₄), 2.51-2.44 (m, 1H, H₁₀), 2.28-2.12 (m, 2H, H₂), 2.09 (s, 1H, H₄), 1.98-1.83 (m, 2H, 1×H₃, 1×H₈), 1.77-1.57 (m, 5H, 2×H₁, 1×H₇, 2×H₁₉), 1.49-1.42 (m, 1H, 1×H₇), 1.41-1.34 (m, 4H, 1×H₁₃, 3×H₁₉), 1.16 (td, J=14.0, 4.4 Hz, 1H, 1×Hₐ), 1.03 (s, 3H, H₁₈), 0.99-0.95 (m, 6H, 3×H₁₇, 3×H₂). ¹³C NMR (125 MHz, CDCl₃) δ 218.0 (C), 137.9 (C), 137.4 (C), 128.4 (CH), 128.4 (CH), 127.8 (CH), 127.8 (CH), 127.6 (CH), 97.1 (CH₂), 94.9 (CH₂), 95.6 (CH), 70.7 (CH₂), 69.9 (CH₂), 69.8 (CH₂), 84.3 (CH), 59.9 (CH), 45.3 (C), 42.8 (C), 42.3 (CH), 41.2 (CH₂), 41.1 (C), 35.5 (CH), 34.5 (CH₂), 30.7 (CH₂), 27.1 (CH₃), 25.3 (CH₂), 22.2 (CH₂), 22.0 (CH₂), 13.9 (CH₃), 12.1 (CH₃), 8.1 (CH₃). IR (ATR-FTIR), cm⁻¹: 3442 (w), 2934 (m), 2879 (m), 1734 (m), 1455 (w), 1381 (w), 1286 (w), 1163 (w), 1107 (m), 1040 (s), 1023 (s), 964 (w), 737 (m), 698 (m). HRMS-ESI (m/z): [M+H]⁺ calcd for C₃₆H₅₁O₆, 579.3686; found, 579.3685. [α]_{D}²⁵=+48° (c=0.50, CHCl₃).

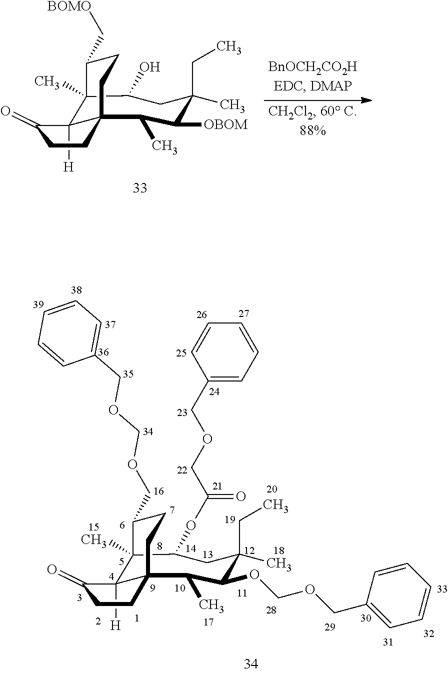

### Synthesis of tris(benzyl)ether 34 (FIG. 9, Scheme 9)

A 4-mL vial was charged with the bis(benzyloxymethylenoxy)ether 33 (33.6 mg, 58.1 μmol, 1 equiv) and benzyloxyacetic acid (20.6 μL, 145 μmol, 2.50 equiv). Benzene (500 μL) was added to the vial. The solution was concentrated to dryness. This process was repeated twice. The reaction vessel was evacuated and refilled using a balloon of argon. This process was repeated twice. Dichloromethane (300 μL), 1-ethyl-3-(3-dimethylaminopropyl)carbodiimide hydrochloride (36.7 mg, 192 μmol, 3.30 equiv), and 4-dimethylaminopyridine (23.4 mg, 192 μmol, 3.30 equiv) were added sequentially to the reaction vessel at 24° C. The vial was sealed and the sealed vial was placed in an oil bath that had been previously heated to 60° C. The reaction mixture was stirred and heated for 1 h at 60° C. The product mixture was allowed to cool to 24° C. over 30 min. The cooled product mixture was concentrated to dryness. The residue obtained was purified by automated flash-column chromatography (eluting with hexanes initially, grading to 40% ether-hexanes, linear gradient) to afford the tris(benzyl)ether 34 as a clear oil (37.2 mg, 88%).

R_{f}=0.55 (40% ether-hexanes; UV, PAA, CAM). ¹H NMR (400 MHz, CDCl₃) δ 7.35-7.28 (m, 15H, 2×H₂₅, 2×H₂₆, 1×H₂₇, 2×H₃₁, 2×H₃₂, 1×H₃₃, 2×H₃₇, 2×H₂₈, 1×H₃₉), 5.79 (d, J=8.0 Hz, 1H, H₁₄), 4.78 (dd, J=12.0, 7.2 Hz, 2H, H₂₃), 4.68-4.60 (m, 5H, 1×H₂₈, 2×H₂₉, 2×H₃₄), 4.60-4.49 (m, 3H, 1×H₂₈, 2×H₃₅), 4.01 (dd, J=26.4, 16.4 Hz, 2H, H₂₂), 3.67 (dd, J=9.2, 1.6 Hz, 1H, 1×H₁₆), 3.31 (d, J=6.0 Hz, 1H, H₁), 2.93 (t, J=9.2 Hz, 1H, 1×H₁₆), 2.59-2.52 (m, 1H, H₁₀), 2.29-2.13 (m, 2H, H₂), 2.09 (s, 1H, H₄), 1.89-1.82 (m, 4H, 1×H₆, 1×H₈, 2×H₁₉), 1.74-1.65 (m, 3H, 1×H₁, 11×H₇, 1×H₁₃), 1.61-1.55 (m, 1H, 1×H₇), 1.50-1.44 (m, 4H, 1×H₁, 3×H₁₅), 1.39-1.30 (m, 1H, 1×H₁₃), 1.15 (td, J=14.8, 4.8 Hz, 1H, 1×H₈), 1.00-0.95 (m, 6H, 3×H₁₇, 3×H₁₈), 0.79 (t, J=7.4 Hz, H₂₀). ¹³C NMR (125 MHz, CDCl₃) δ 216.7 (C), 169.3 (C), 137.9 (C), 137.2 (C), 128.4 (CH), 128.3 (CH), 127.9 (CH), 127.9 (CH), 127.8 (CH), 127.7 (CH), 127.6 (CH), 127.6 (CH), 96.9 (CH₂), 94.6 (CH₂), 84.9 (CH), 73.3 (CH₂), 70.7 (CH₂) 69.2 (CH₂), 68.8 (CH), 68.4 (CH₂), 67.9 (CH₂), 58.6 (CH), 45.1 (C), 43.0 (CH), 41.5 (C), 41.4 (CH₂), 40.5 (C), 35.2 (CH), 34.4 (CH₂), 29.9 (CH₂), 26.7 (CH₃), 25.2 (CH₂), 22.4 (CH₂), 21.6 (CH₂), 15.1 (CH), 12.0 (CH), 8.2 (CH₃). IR (ATR-FTIR), cm⁻¹: 2933 (w), 1774 (w), 1734 (m), 1454 (m), 1111 (s), 1059 (m), 1026 (s), 937 (m), 844 (w), 734 (s), 696 (s), 606 (w). HRMS-ESI (m/z): [M+Na]⁺ calcd for C₄₅H₅₈NaO₈, 747.4029; found, 747.4055. [α]_{D}²⁵+47° (c=0.25, CHCl₃).

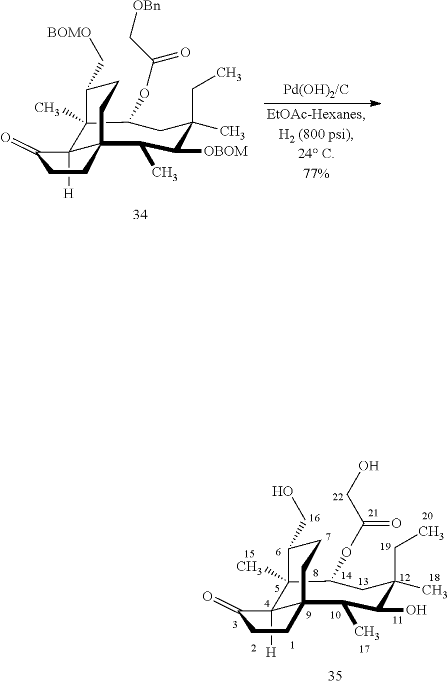

### Synthesis of 16-hydroxy-19,20-dihydropleuromutilin (35, FIG. 9, Scheme 9)

A 4-mL vial was charged with the tris(benzyl)ether 34 (12.4 mg, 17.1 μmol, 1 equiv). Benzene (500 μL) was added to the vial. The solution was concentrated to dryness. This process was repeated twice. The reaction vessel was evacuated and refilled using a balloon of nitrogen. This process was repeated twice. Ethyl acetate (50 μL), hexanes (250 μL), and Pearlman's catalyst (20 wt. % loading, 2.4 mg, 3.4 μmol, 0.200 equiv) were added sequentially to the reaction vessel at 24° C. The vial was placed in a stainless steel hydrogenation apparatus. The apparatus was purged with dihydrogen by pressurizing to 50 psi and venting three times. The vessel was pressurized with dihydrogen (800 psi), sealed, and the reaction mixture was stirred for 18 h at 24° C. The apparatus was depressurized by slowly venting the dihydrogen. The product mixture was filtered through a pad of celite and the pad was rinsed with ether (50 mL). The filtrates were collected and combined and the combined filtrates were concentrated. The residue obtained was purified by automated flash-column chromatography (eluting with hexanes initially, grading to 100% ethyl acetate-hexanes, linear gradient) to afford 16-hydroxy-19,20-dihydropleuromutilin (35) as an amorphous white solid (5.2 mg, 77%).

R_{f}=0.27 (80% ethyl acetate-hexanes; PAA, CAM). ¹H NMR (400 MHz, CDCl₃) δ 5.70 (d, J=8.4 Hz, 1H, H₁₄), 4.08 (s, 2H, H₂₂), 3.68 (dd, J=10.4, 4.8 Hz, 1H, 1×H₁₆), 3.43 (d, J=6.4 Hz, 1H, H₁₁), 3.00 (t, J=9.4 Hz, 1H, 1×H₁₆), 2.46-2.39 (m, 1H, H₁₀), 2.27-2.20 (m, 2H, H₂), 2.11 (s, 1H, H₄), 1.89-1.51 (m, 12H, 2×H₁, 1×H₆, 2×H₇, 1×H₈, 1×H₁₃, 2×H₁₉, 3×OH), 1.47 (s, 3H, H₁), 1.35 (app d, 1H, 1×H₁), 0.13 (td, J=14.0, 4.4 Hz, 1H, 1×H₁), 1.01-0.92 (m, 6H, 3×H₁₇, 3×H₁₈), 0.75 (t, J=7.4 Hz, H₂₀). ¹³C NMR (125 MHz, CDCl₃) δ 216.6 (C), 172.2 (C), 76.4 (CH), 70.0 (CH), 63.2 (CH₂), 61.3 (CH₂), 58.6 (CH), 45.5 (C), 45.2 (CH), 41.6 (C), 41.0 (C), 40.3 (CH₂), 34.4 (CH), 34.3 (CH₂), 29.7 (CH₂) 26.3 (CH₃), 24.9 (CH₂), 21.6 (CH₂) 20.6 (CH₂), 15.3 (CH₃), 11.1 (CH₃), 8.2 (CH₃).

Due to the high instability of this compound, the infra-red spectrum and high-resolution mass was not obtained.

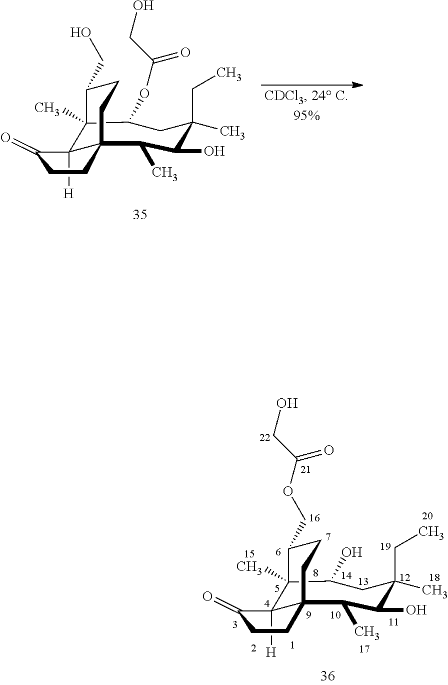

### Acyl Group Migration of 16-hydroxy-9,20-dihydropleuromutilin (35, FIG. 9, Scheme 9)

A solution of 16-hydroxy-19,20-dihydropleuromutilin (35, 2.6 mg, 6.6 μmol, 1 equiv) in chloroform-d (200 μL) was stored in an NMR tube for 5 days at 24° C. The resulting mixture was diluted with chloroform-d (200 μL) and ¹H NMR analysis of the diluted sample showed full conversion (>95%) to the acyl group migrated product 36 as a colorless clear film.

R_{f}=0.32 (80% ethyl acetate-hexanes; PAA, CAM). ¹H NMR (600 MHz, CDCl₃) δ 4.00 (dd, J=10.8, 3.0 Hz, 1H, 1×H₁₆), 3.43 (d, J=7.2 Hz, 1H, H₁₁), 4.18-4.06 (m, 3H, 1×H₁₆, 2×H₂₂), 3.39 (d, J=6.0 Hz, 1H, H₁), 2.55-2.40 (br m, 1H, C22-OH), 2.35-2.28 (m, 1H, H₁₀), 2.27-2.10 (m, 2H, H₂), 2.06 (s, 1H, H₄), 1.86 (td, J=9.0, 3.6 Hz, 1H, H₆), 1.82-1.77 (m, 1H, 1×H₈), 1.70-1.47 (m, 10H, 2×H₁, 2×H₇, 2×H₁₃, 2×H₁₉, 2×OH), 1.37 (s, 3H, H₁₅), 1.10 (td, J=13.8, 5.4 Hz, 1H, 1×H₈), 1.00 (s, 3H, H₁₈), 0.97-0.92 (m, 6H, 3×H₁₇, 3×H₂₀). ¹³C NMR (150 MHz, CDCl₃) δ 217.0 (C), 173.4 (C), 76.6 (CH), 67.9 (CH₂), 65.7 (CH), 60.6 (CH₂), 58.9 (CH), 45.0 (C), 43.4 (CH₂), 41.9 (C), 41.6 (CH), 40.8 (C), 34.6 (CH), 34.2 (CH₂), 29.5 (CH₂), 26.6 (CH₃), 25.0 (CH₂), 22.1 (CH₂), 21.1 (CH₂) 13.4 (CH₃), 11.2 (CH₃), 8.2 (CH₃). IR (ATR-FTIR), cm⁻¹: 3436 (br m), 2932 (m), 1730 (s), 1461 (w), 1383 (w), 1286 (w), 1219 (m), 1095 (m), 1005 (m), 977 (m), 947 (w), 911 (m), 701 (s), 697 (w), 581 (w).

HRMS-ESI (m/z): [M+Na]⁺ calcd for C₂₂H₃₆NaO₆, 419.2410; found, 419.2402.

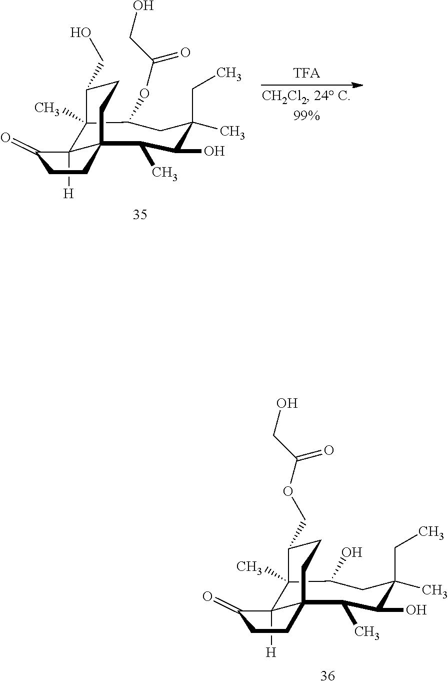

### Acyl Group Migration of 16-hydroxy-19,20-dihydropleuromutilin (35, FIG. 9, Scheme 9)

A 4-mL vial was charged with 16-hydroxy-19,20-dihydropleuromutilin (35, 2.6 mg, 6.6 μmol, 1 equiv). Benzene (200 μL) was added to the reaction vessel and the resulting solution was concentrated to dryness. This process was repeated two times. The reaction vessel was evacuated and refilled using a balloon of argon. This process was repeated two times. Dichloromethane (150 L) was added the reaction vessel. A solution of trifluoroacetic acid (0.0300 μL, 0.390 μmol, 5.00 mol %) in dichloromethane (50 μL) was added dropwise via syringe to the reaction mixture at 24° C. The resulting mixture was stirred for 30 min at 24° C. The product mixture was concentrated to dryness. The residue obtained was dissolved in benzene (200 μL) and the resulting solution was concentrated to dryness. This process was repeated twice to afford 16-hydroxy-9,20-dihydropleuromutilin hydroxyacetate (36) as a colorless clear film (2.6 mg, 99%).

16-Hydroxy-19,20-dihydropleuromutilin hydroxyacetate (36): [α]_{D}²⁵=+22° (c=0.10, CHCl₃).

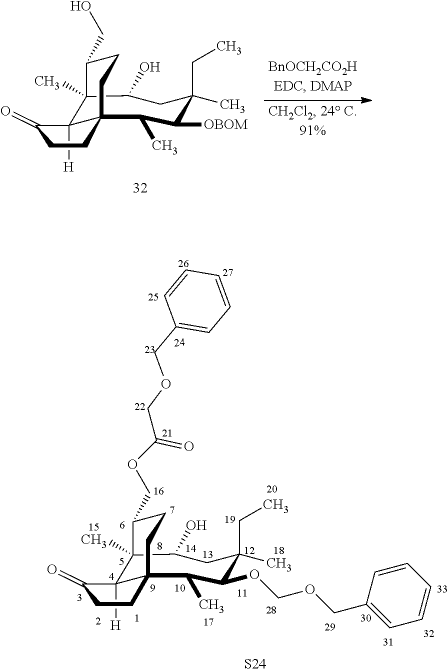

### Synthesis of bis(benzyl)ether S24 (FIG. 9, Scheme 9)

A 4-mL vial was charged with the diol 32 (39.4 mg, 86.0 μmol, 1 equiv) and benzyloxyacetic acid (14.7 t, 103 μmol, 1.20 equiv). Benzene (500 μL) was added to the vial. The solution was concentrated to dryness. This process was repeated twice. The reaction vessel was evacuated and refilled using a balloon of argon. This process was repeated twice. Dichloromethane (400 μL), 1-ethyl-3-(3-dimethylaminopropyl)carbodiimide hydrochloride (24.7 mg, 129 μmol, 1.50 equiv), and 4-dimethylaminopyridine (2.1 mg, 17.2 μmol, 0.200 equiv) were added sequentially to the reaction vessel at 24° C. The reaction mixture was stirred for 90 min at 24° C. The product mixture was concentrated to dryness. The residue obtained was purified by automated flash-column chromatography (eluting with hexanes initially, grading to 40% ether-hexanes, linear gradient) to afford the bis(benzyl)ether S24 as an amorphous white solid (47.2 mg, 91%).

R_{f}=0.52 (33% ethyl acetate-hexanes; UV, PAA, CAM). ¹H NMR (400 MHz, CDCl₃) δ 7.36-7.68 (m, 1H, 2×H₂₅, 2×H₂₆, 1×H₂₇, 2×H₃, 2×H₃₂, 1×H₃₃), 4.78-4.73 (m, 2H, H₂₈), 4.65 (s, 2H, H₂₂), 4.62 (s, 2H, H₂₉), 4.37 (dd, J=11.2, 3.2 Hz, 1H, 1×H₁₆), 4.25 (d, J=7.2 Hz, 1H, H₁), 4.11 (d, J=11.2 Hz, 1H, 1×H₆), 4.07 (s, 2H, H₂₃), 3.27 (d, J=6.0 Hz, 1H, H₁₄), 2.41-2.32 (m, 1H, H₁₀), 2.28-2.12 (m, 2H, H₂), 2.03 (s, 1H, H₄), 1.83-1.40 (m, 10H, 1×H₁, 1×H₆, 2×H₇, 1×H₈, 2×H₁₃, 2×H₁₉, 1×OH), 1.44-1.35 (m, 4H, 1×H₁₇, 3×H₁₅), 1.09 (td, J=14.4, 4.8 Hz, 1H, 1×H₈), 1.01 (s, 3H, H₁₈), 0.94-0.90 (m, 6H, 3×H₁, 3×H₂₀). ¹³C NMR (125 MHz, CDCl₃) δ 217.3 (C), 170.3 (C), 137.8 (C), 137.0 (C), 128.4 (CH), 128.3 (CH), 128.0 (CH), 127.9 (CH), 127.6 (CH), 127.6 (CH), 96.9 (CH₂), 85.1 (CH), 73.2 (CH₂), 70.7 (CH₂), 67.2 (CH₂), 66.8 (CH₂), 65.3 (CH), 59.0 (CH), 44.9 (C), 43.2 (CH₂), 42.0 (CH), 41.6 (C), 41.3 (C), 35.2 (CH), 34.4 (CH₂), 29.8 (CH₂), 27.0 (CH₃), 25.1 (CH₂), 22.0 (CH₂) 21.9 (CH₂), 13.4 (CH), 11.9 (CH₃), 8.1 (CH). IR (ATR-FTIR), cm⁻¹: 3549 (br w), 2930 (m), 2882 (m), 1734 (s), 1497 (w), 1455 (m), 1382 (w), 1285 (w), 1210 (m), 1129 (m), 1040 (s), 1023 (s), 739 (m), 698 (m). HRMS-ESI (m/z): [M+H]⁺ calcd for C₃₇H₅₁O₇, 607.3635; found, 607.3636. [α]_{D}²⁵=+32° (c=0.50, CHCl₃).

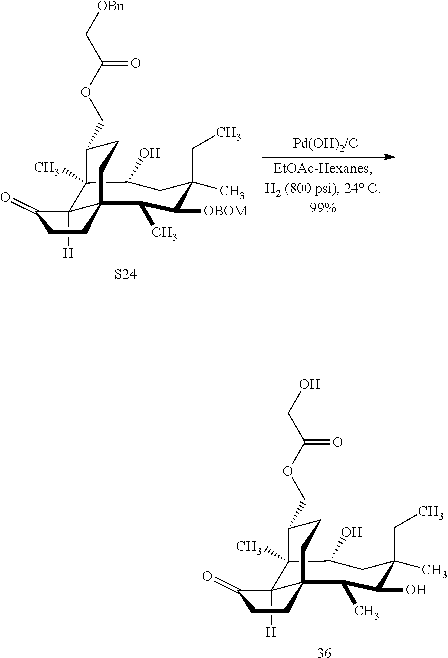

### Synthesis of 16-hydroxy-19,20-dihydropleuromutilin hydroxyacetate (36, FIG. 9, Scheme 9)

A 4-mL vial was charged with the bis(benzyl)ether S24 (11.8 mg, 19.4 μmol, 1 equiv). Benzene (500 μL) was added to the vial. The solution was concentrated to dryness. This process was repeated twice. The reaction vessel was evacuated and refilled using a balloon of nitrogen. This process was repeated twice. Ethyl acetate (50 μL), hexanes (250 μL), and Pearlman's catalyst (20 wt. % loading, 2.7 mg, 3.9 μmol, 0.200 equiv) were added sequentially to the reaction vessel at 24° C. The vial was placed in a stainless steel hydrogenation apparatus. The apparatus was purged with dihydrogen by pressurizing to 50 psi and venting three times. The vessel was pressurized with dihydrogen (800 psi), sealed, and the reaction mixture was stirred for 18 h at 24° C. The apparatus was depressurized by slowly venting the dihydrogen. The product mixture was filtered through a pad of celite and the pad was rinsed with ether (50 mL). The filtrates were collected and combined. The combined filtrates were concentrated to dryness to afford 16-hydroxy-19,20-dihydropleuromutilin hydroxyacetate (36) as colorless clear film (7.8 mg, 99%).

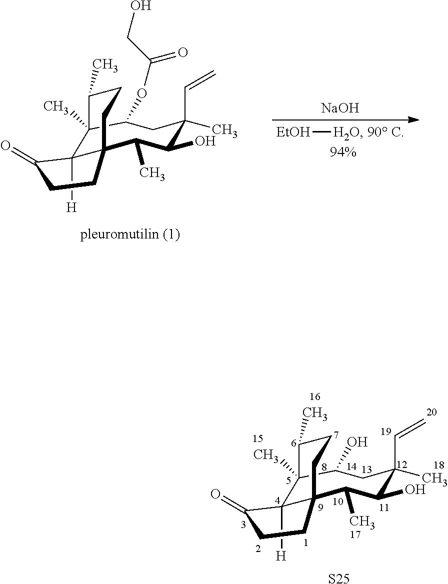

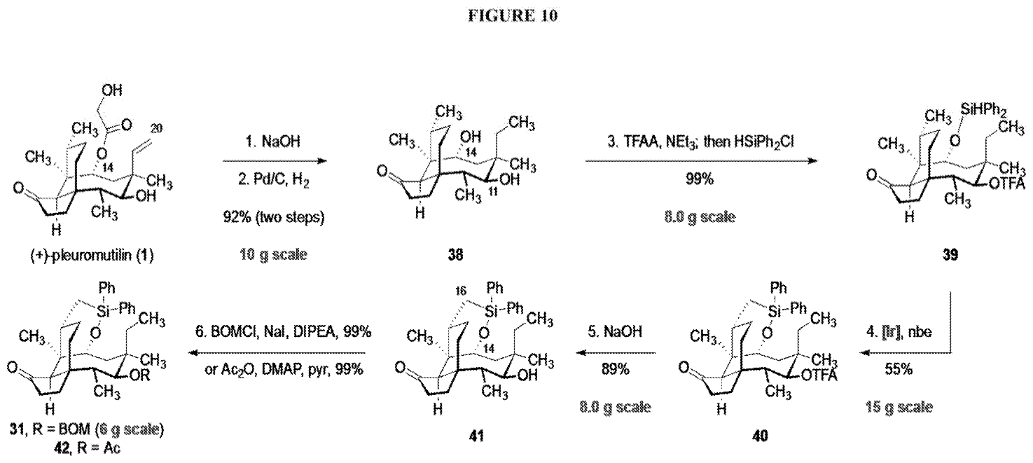

### Synthesis of Mutilin (25, FIG. 10, Scheme 10)

Water (38 mL) and an aqueous solution of sodium hydroxide (50 wt. %, 5.3 mL) were added dropwise sequentially to a solution of pleuromutilin (1, 10.0 g, 26.5 mmol, 1 equiv) in ethanol (90 mL) at 24° C. The reaction mixture was stirred for 12 h at 90° C. The product mixture was transferred to a separatory funnel that had been charged with ether (200 mL). The layers were separated and the aqueous layer was extracted with ether (3×50 mL). The organic layer was dried over sodium sulfate. The dried solution was filtered and the filtrate was concentrated. The residue obtained was purified by automated flash-column chromatography (eluting with hexanes initially, grading to 33% ethyl acetate-hexanes, linear gradient) to afford mutilin (S25) as an amorphous white solid (7.99 g, 94%).

R_{f}=0.65 (50% ethyl acetate-hexanes; PAA, CAM). ¹H NMR (500 MHz, CD₂Cl₂) δ6.16 (dd, J=18.0, 11.0 Hz, 1H, H₁₉), 5.33 (d, J=18.0 Hz, 1H, 1×H₂₀), 5.25 (d, J=11.0 Hz, 1H, 1×H₂₀), 4.31 (t, J=6.8 Hz, 1H, H₁₁), 3.40 (t, J=6.3 Hz, 1H, H₁₄), 2.20-2.11 (m, 3H, 2×H₂, 1×H₁₀), 2.04 (s, 1H, H₄), 1.91 (dd, J=16.0, 7.5 Hz, 1H, 1×H₁₃), 1.73 (dq, J=14.5, 3.5 Hz, 1H, 1×H₈), 1.66-1.54 (m, 4H, 1×H₁, 1×H₆, 1×H₁₃, 1×C14-OH), 1.49-1.42 (m, 2H, 1×H₁, 1×H₇), 1.38-1.30 (m, 4H, 1×H₁₃, 3×H₁₅), 1.29 (d, J=5.5 Hz, 1H, C11-OH), 1.14-1.11 (m, 4H, 1×H₈, 3×H₁₈), 0.93 (d, =7.0 Hz, 3H, H₁₆), 0.90 (d, J=7.0 Hz, 3H, H₁₇). ¹³C NMR (125 MHz, CD₂Cl₂) 8218.0 (C), 140.5 (CH), 115.7 (CH₂), 75.5 (CH), 67.2 (CH), 59.5 (CH), 45.9 (C), 45.9 (CH₂), 45.7 (C), 42.9 (C), 37.5 (CH), 37.1 (CH), 34.9 (CH₂), 30.9 (CH₂), 29.1 (CH₃), 27.7 (CH₂), 25.6 (CH₂), 18.6 (CH₃), 13.9 (CH₃), 11.5 (CH₃). IR (ATR-FTIR), cm⁻¹: 3558 (w), 2956 (w), 2878 (w), 1721 (s), 1459 (w), 1374 (w), 1282 (w), 1117 (m), 1034 (m), 997 (m), 953 (m), 910 (m). HRMS-ESI (m/z): [M+H]⁺ calcd for C₂₀H₃₃O₃, 321.2430; found, 321.2431. [α]_{D}²⁵=+69° (c=1.00, CHCl₃).

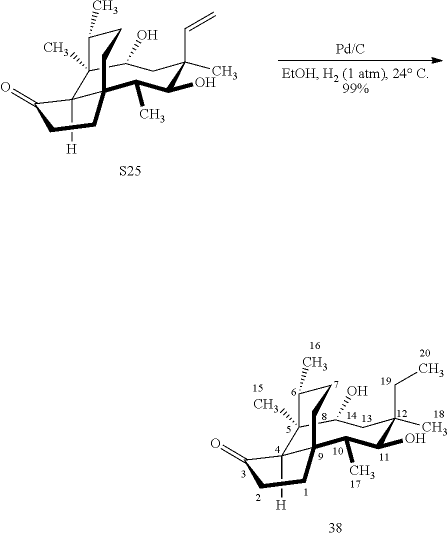

### Synthesis of 19,20-dihydromutilin (38, FIG. 10, Scheme 10)

Palladium on carbon (5 wt. % loading, 2.66 g, 1.25 mmol, 0.05 equiv) was added to a solution of mutilin (S25, 7.99 g, 12.0 mmol, 1 equiv)ethanol (125 mL) at 24° C. The reaction vessel was evacuated and re-filled using a balloon of dihydrogen. This process was repeated four times. The reaction mixture was stirred for 12 h at 24° C. The product mixture was filtered through a short column of celite and the short column was rinsed with dichloromethane (1.0 L). The filtrates were combined and the combined filtrates were concentrated to afford 19,20-dihydromutilin (38) as an amorphous white solid (8.04 g, 99%).

R_{f}=0.61 (50% ethyl acetate-hexanes; PAA, CAM). ¹H NMR (400 MHz, CD₃OD) δ 4.22 (d, J=7.2 Hz, 1H, H₁), 3.39 (d, J=6.0 Hz, 1H, H₁₄), 2.34-2.29 (m, 1H, H₁₀), 2.27-2.17 (m, 2H, 1×H₂, 1×H₄), 2.16-2.06 (m, 1H, 1×H₂), 1.78 (dq, J=14.4, 3.6 Hz, 1H, 1×H₈), 1.72-1.61 (m, 3H, 1×H₁, 1×H₁, 1×H₁₉), 1.60-1.48 (m, 3H, 1×H₆, 1×H₇, 1×H₁₉), 1.46-1.38 (m, 2H, 1×H₁, 1×H₁₃), 1.37-1.33 (m, 1H, 1×H₇), 1.31 (s, 3H, H₁₈), 1.12 (td, J=13.6, 4.0 Hz, 1H, 1×H₈), 0.97 (s, 3H, H₈), 0.95-0.88 (m, 9H, 3×H₆, 3×H₁₇, 3×H₂₀). ¹³C NMR (100 MHz, CD₃OD) δ 219.4 (C), 75.7 (CH), 65.4 (CH), 58.8 (CH), 45.4 (C), 43.1 (CH₂), 42.3 (C), 40.4 (C), 37.2 (CH), 34.6 (CH), 34.0 (CH₂), 30.4 (CH₂), 27.0 (CH₂), 25.9 (CH), 24.4 (CHA 20.5 (CH₂), 17.1 (CH₃), 12.9 (CH₃), 10.4 (CH₃), 7.2 (CH). IR (ATR-FTIR), cm⁻¹: 3495 (br w), 2958 (m), 2928 (m), 2878 (m), 1727 (m), 1461 (w), 1412 (w), 1381 (w), 1285 (w), 117 (m), 1033 (w), 1006 (w), 990 (w), 909 (w), 732 (s). HRMS-ESI (m/z): [M+H]⁴ calcd for C₂₀H₃₅O₃, 323.2580; found, 323.2589. [α]_{D}²⁵=+72° (c=1.00, CH₃OH).

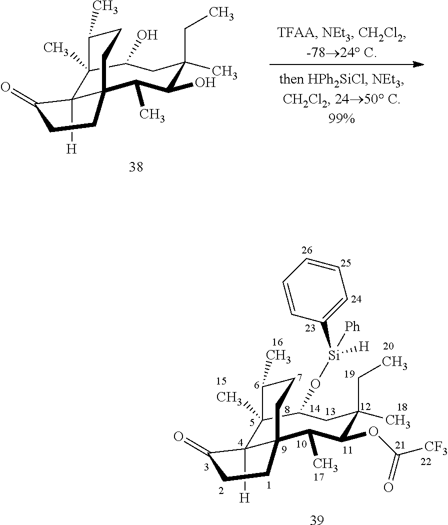

### Synthesis of Silane 39 (FIG. 10, Scheme 10)

Trifluoroacetic anhydride (3.33 mL, 24.2 mmol, 1.00 equiv) was added dropwise via syringe to a solution of 19,20-dihydromutilin [38, 7.80 g, 24.2 mmol, 1 equiv, dried by azeotropic distillation with benzene (50 mL)] and triethylamine (13.5 mL, 96.7 mmol, 4.00 equiv) in dichloromethane (150 mL) at −78° C. The resulting mixture was stirred for 20 min. The reaction mixture was allowed to warm up over 2 h to 24° C. (Chloro)diphenylsilane (10.5 mL, 48.4 mmol, 2.00 equiv) was added dropwise via syringe to the reaction mixture at 24° C. The reaction vessel was placed in an oil bath that had been previously heated to 50° C. The reaction mixture was stirred and heated for 30 min at 50° C. The product mixture was allowed to cool over 1 h to 0° C. with an ice bath. Aqueous potassium phosphate buffer solution (pH 7, 0.10 M, 100 mL) was added dropwise into the reaction vessel at 0° C. The resulting mixture was stirred for 10 min at 0° C. The product mixture was transferred to a separatory funnel. The layers that formed were separated and the aqueous layer was extracted with dichloromethane (3×100 mL). The organic layers were combined and dried over sodium sulfate. The dried solution was filtered and the filtrate was concentrated to dryness. The residue obtained was purified by automated flash-column chromatography (eluting with hexanes initially, trading to 12% ether-hexanes, linear gradient) to afford the silane 39 as an amorphous white solid (14.6 g, 99%).

R_{f}=0.50 (10% ethyl acetate-hexanes; UV, PAA, CAM). ¹H NMR (400 MHz, C₆D₆) δ 7.70-7.65 (m, 4H, 4×H₂₅), 7.18-7.07 (m, 6H, 4×H₂₄, 2×H₂₆), 5.68 (s, 1H, Si—H), 4.80 (d, J=6.8 Hz, 1H, H₁₁), 4.54 (d, J=8.4 Hz, 1H, H₄), 2.18-2.09 (m, 1H, H₁₀), 1.90-1.82 (m, 2H, H₂), 1.78 (s, 3H, H₁₅), 1.75-1.63 (m, 4H, 1×H₁, 1×H₄, 1×H₁, 1×H₁₃), 1.59-1.45 (m, 2H, 1×H₁ 1×H₁₃), 1.28-1.13 (m, 2H, 1×H₈ 1×H₁₉), 1.09-1.04 (m, 2H, 1×H₇ 1×H₁₉), 1.00 (d, J=7.2 Hz, 3H, H₁₆), 0.91-0.85 (m, 1H, 1×H₇), 0.81 (t, J=7.6 Hz, 3H, H₂₀), 0.72 (td, J=14.0, 4.4 Hz, 1H, 1×H₈), 0.68 (s, 3H, H₁₈), 0.51 (d, J 7.2 Hz, 3H, H₁). ¹³C NMR (100 MHz, C₆D₆) δ 214.9 (C), 156.8 (q, J=48.0 Hz, C), 135.4 (CH), 135.0 (CH), 134.9 (CH), 134.8 (CH), 130.8 (C), 130.7 (C), 130.5 (CH), 130.4 (CH), 115.6 (q, J=285 Hz, C), 83.8 (CH), 69.4 (CH), 58.8 (CH), 45.1 (C), 45.1 (C), 44.0 (CH₂), 40.3 (C), 37.4 (CH), 35.0 (CH), 34.3 (CH₂), 30.2 (CH₂), 27.4 (CH₂), 25.2 (CH₂), 25.2 (CH₃, CH₂), 19.2 (CH₃), 14.9 (CH₃), 11.4 (CH₃), 9.7 (CH₃). ¹⁹F NMR (375 MHz, C₆D₆) δ −74.9. IR (ATR-FTIR), cm⁻¹: 3495 (br w), 2958 (m), 2928 (m), 2878 (m), 1727 (m), 1461 (w), 1412 (w), 1381 (w), 1285 (w), 1117 (m), 1033 (w), 1006 (w), 990 (w), 909 (w), 732 (s). HRMS-ESI (m/z): [M-Si(C₆H₅)₂+Na]⁺ calcd for C₂₂H₃₃F₃NaO₄, 441.2229; found, 441.2243. [α]_{D}²⁵=+54° (c=0.50, CHCl₃).

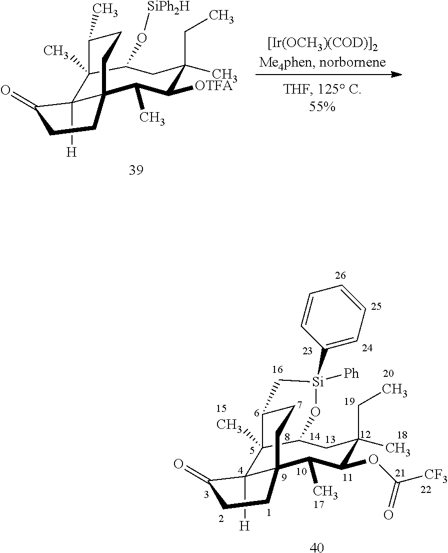

### Synthesis of Silacycle 40 (FIG. 10, Scheme 10)

This experiment was adapted from the work of Hartwig and co-workers.² A 250-mL pressure tube with a Teflon-coated valve was charged with 3,4,7,8-tetramethyl-1,10-phenanthroline (500 mg, 2.12 mmol, 8.75 mol %) and norbornene (3.42 g, 36.3 mmol, 1.50 equiv) in the glovebox. A 200-mL pear-shaped flask was charged with silane 39 [14.6 g, 24.2 mmol, 1 equiv, dried by azeotropic distillation with benzene (3×50 mL)]. The vessel containing the silane was evacuated and refilled using a balloon of argon. This process was repeated two times. Tetrahydrofuran (20 mL) was transferred into the vessel containing the silane and the resulting solution was added to the vessel containing the ligand and norbornene in the glovebox. The vessel containing the silane was rinsed with tetrahydrofuran (3×10 mL) and the combined rinses were transferred to the reaction vessel.

Methoxy(cyclooctadiene)iridium(I) dimer (562 mg, 847 μmol, 3.5 mol %) was added to an oven-dried 20-mL vial. Tetrahydrofuran (4 mL) was added into the vial containing the catalyst and the resulting solution was transferred dropwise via syringe to the reaction vessel in the glovebox. The vial containing the catalyst was rinsed with tetrahydrofuran (3×2 mL) and the combined rinses were transferred into the reaction vessel. The reaction vessel was sealed and the reaction mixture was stirred for 1 h at 24° C. in the glovebox. The sealed reaction vessel was then removed from the glovebox and placed in an oil bath that had been preheated to 125° C. The reaction mixture was stirred and heated for 26 h at 125° C. The reaction vessel was allowed to cool over 30 min to 24° C. and the cooled product mixture was concentrated to dryness. The residue obtained was purified by automated flash-column chromatography (eluting with hexanes initially, grading to 15% ether-hexanes, linear gradient) to afford the silacycle 40 as an amorphous white solid (8.00 g, 55%).

R_{f}=0.54 (15% ethyl acetate-hexanes; UV, PAA, CAM). ¹H NMR (500 MHz, C₆D₆) δ 7.71-7.61 (m, 4H, 4×H₂₅), 7.27-7.12 (m, 6H, 4×H₂₄, 2×H₂₆), 4.75 (d, J=7.0 Hz, 1H, H₁₁), 4.43 (d, J=7.0 Hz, 1H, H₁₄), 2.23-2.19 (m, 1H, H₀), 2.13-2.07 (m, 1H, H₆), 1.93-1.83 (m, 1H, 1×H₂), 1.80-1.73 (m, 4H, 1×H₂, 3×H₁₅), 1.70-1.63 (m, 2H, 1×H₄, 1×H₁₃), 1.61-1.50 (m, 2H, 1×H₁₃, 1×H₁₉), 1.50-1.40 (m, 4H, 1×H₁, 1×H₇, 2×H₈), 1.09-1.05 (m, 1H, 1×H₇), 1.03-0.98 (m, 1H, 1×H₁₆), 0.95 (t, J=7.5 Hz, 3H, H₂₀), 0.85-0.79 (m, 2H, 1×H₁, 1×H₁₉), 0.75 (s, 3H, H₁₈), 0.85-0.62 (m, 1H, 1×H₁₆), 0.27 (d, J=7.0 Hz, 3H, H₁₇). ¹³C NMR (125 MHz, C₆D₆) δ 214.9 (C), 157.0 (q, J=41.2 Hz, C), 137.3 (C), 136.5 (C), 134.7 (CH), 134.4 (CH), 130.4 (CH), 130.4 (CH), 128.3 (CH), 115.6 (q, J=285 Hz, C), 84 2 (CH), 66.6 (CH), 58.5 (CH), 44.5 (C), 41.5 (C), 41.0 (CH₂), 40.3 (C), 38.1 (CH), 35.3 (CH), 34.0 (CH₂), 29.9 (CH₂), 27.5 (CH₂), 25.6 (CH₂), 25.5 (CH₃), 22.4 (CH₂), 15.2 (CH₃), 12.9 (CH₂), 11.6 (CH₃), 8.4 (CH₃). ¹⁹F NMR (470 MHz, C₆D₆) δ −74.8. IR (ATR-FTIR), cm⁻¹: 2942 (w), 1774 (m), 1738 (w), 1463 (w), 1379 (w), 1218 (m), 1161 (s), 1120 (s), 917 (w), 719 (s), 699 (s), 502 (s). HRMS-ESI (m/z): [M+Na]⁺ calcd for C₃₄H₄₁F₃NaO₄Si, 621.2624; found, 621.2625. [α]_{D}²⁵=+55° (c=0.25, CHCl₃).

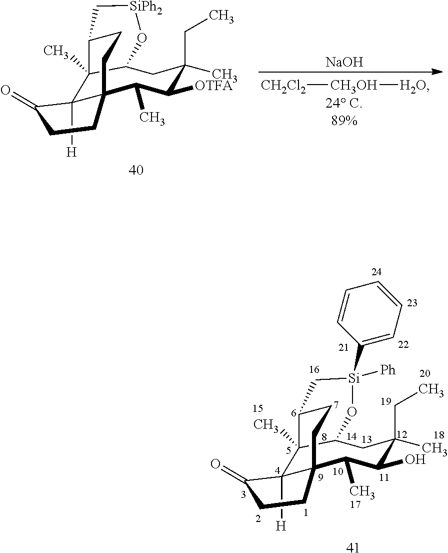

### Synthesis of Silacycle 41 (FIG. 10, Scheme 10)

An aqueous sodium hydroxide solution (1.0 M, 80.2 mL, 80.2 mmol, 6.00 equiv) was added dropwise via syringe to a solution of the silacycle 40 (8.00 g, 13.4 mmol, 1 equiv) in a mixture of dichloromethane and methanol (1:1 v/v, 480 mL) at 24° C. The resulting mixture was stirred for 30 min at 24° C. The resulting mixture was transferred to a separatory funnel. The layers that formed were separated and the aqueous layer was extracted with dichloromethane (3×150 mL). The organic layers were combined and dried over sodium sulfate. The dried solution was filtered and the filtrate was concentrated to dryness. The residue obtained was purified by automated flash-column chromatography (eluting with hexanes initially, grading to 40% ethyl acetate-hexanes, linear gradient) to afford the silacycle 41 as an amorphous white solid (5.97 g, 89%).

R_{f}=0.14 (15% ethyl acetate-hexanes; UV, PAA, CAM). ¹H NMR (400 MHz, C₆D₆) δ 7.74-7.63 (m, 4H, 4×H₂₃), 7.25-7.09 (m, 6H, 4×H₂₂, 2×H₂₄), 4.54 (d, J=7.0 Hz, 1H, H₁₁), 2.94 (br s, 1H, H₁₄), 2.30-2.22 (m, 1H, H₁₀), 2.10-1.90 (m, 1H, H₆), 1.86-1.68 (m, 7H, 2×H₂, 1×H₄, 1×H₁, 3×H₁₅), 1.61-1.44 (m, 5H, 1×H₇, 1×H₁, 1×H₁₆, 2×H₁₉), 1.21-1.10 (m, 2H, 1×H₇, 1×H₈), 1.06 (t, J=7.4 Hz, 3H, H₂), 1.20-0.92 (m, 2H, 1×H₁, 1×OH), 0.90 (s, 3H, H₁₈), 0.88-0.80 (m, 2H, 1×H₁, 1×H₁₆), 0.72 (td, J=13.6, 4.0 Hz, 1H, 1×H₈), 0.45 (d, J=7.4 Hz, 3H, H₁₇). ¹³C NMR (100 MHz, C₆D₆) δ 216.0 (C), 137.4 (C), 136.9 (C), 134.7 (CH), 134.4 (CH), 130.3 (CH), 130.3 (CH), 128.2 (CH), 76.5 (CH), 67.0 (CH), 58.7 (CH), 45.0 (C), 41.8 (C), 41.3 (CH₂), 40.8 (C), 38.4 (CH), 35.6 (CH), 34.3 (CH₂), 30.3 (CH₂), 27.7 (CH₂), 26.6 (CH₃), 25.7 (CH₂), 21.0 (CH₂), 15.5 (CH₃), 13.1 (CH₂), 11.7 (CH₃), 8.7 (CH₃). IR (ATR-FTIR), cm⁻¹: 2922 (w), 1734 (m), 1461 (w), 1428 (s), 1118 (s), 1107 (m), 958 (m), 717 (s), 698 (s), 498 (s). HRMS-ESI (m/z): [M+H]⁺ calcd for C₃₂H₄₃O₄Si, 503.2981; found, 503.2987. [α]_{D}²⁵=+56° (c=0.10, CHCl₃).

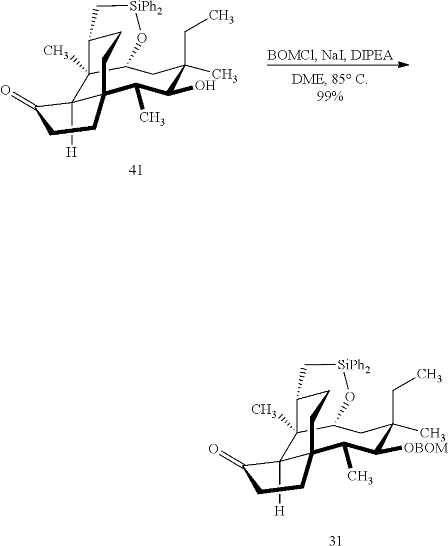

### Synthesis of Silacycle 31 (FIG. 10, Scheme 10)

A 500-mL round-bottomed flask fused to a Teflon-coated valve was charged with silacycle 41 (5.97 g, 11.9 mmol, 1 equiv). Benzene (50.0 mL) was added and the solution was concentrated to dryness. This process was repeated twice. Sodium iodide (5.34 g, 35.6 mmol, 6.00 equiv) was added to the reaction vessel. The reaction vessel was evacuated and refilled using a balloon of argon. This process was repeated twice. 1,2-Dimethoxyethane (205 mL), N,N-diisopropylethylamine (12.4 mL, 71.2 mmol, 6.00 equiv), and benzyl chloromethyl ether (4.95 mL, 35.6 mmol, 3.00 equiv) was added sequentially via syringe to the reaction mixture at 24° C. The reaction vessel was sealed and the sealed vessel was placed in an oil bath that had been previously heated to 85° C. The reaction mixture was stirred and heated for 70 min at 85° C. The product mixture was allowed to cool over 30 min to 0° C. with an ice bath. A saturated aqueous sodium bicarbonate solution (50 mL) was added dropwise via syringe to the product mixture. The resulting mixture was stirred for 10 min at 0° C. The resulting mixture was transferred to a separatory funnel that had been charged with dichloromethane (100 mL). The layers that formed were separated and the aqueous layer was extracted with dichloromethane (3×100 mL). The organic layers were combined and the combined organic layers were dried over sodium sulfate. The dried solution was filtered and the filtrate was concentrated. The residue obtained was purified by automated flash-column chromatography (eluting with hexanes initially, grading to 50% ether-hexanes, linear gradient) to afford the silacycle 31 as an amorphous white solid (7.64 g, 99%).

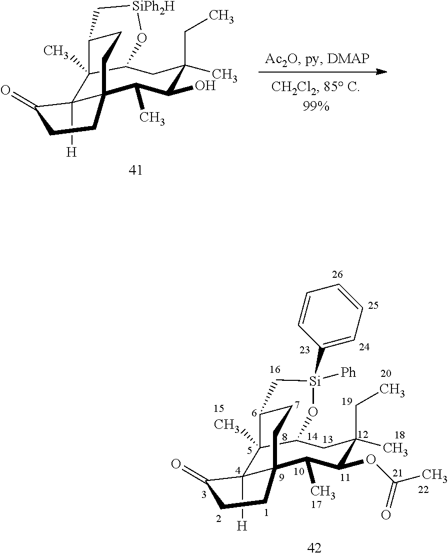

### Synthesis of Silacycle 42 (FIG. 10, Scheme 10)

Pyridine (70.2 μL, 872 μmol, 2.00 equiv) and acetic anhydride (49.5 μL, 523 μmol, 1.20 equiv) were added sequentially dropwise via syringe to a solution of the silacycle 41 (219 mg, 436 μmol, 1 equiv) and 4-dimethylaminopyridine (63.9 mg, 523 μmol, 1.20 equiv) in dichloromethane (2.0 mL) at 24° C. The reaction mixture was stirred for 2 h at 24° C. The product mixture was transferred to a separatory funnel that had been charged with ethyl acetate (50 mL). The organic layer was washed with saturated aqueous sodium bicarbonate solution (3×10 mL). The washed organic layer was dried over sodium sulfate. The dried solution was filtered and the filtrate was concentrated to dryness. The residue obtained was purified by automated flash-column chromatography (eluting with hexanes initially, grading to 33% ether-hexanes, linear gradient) to afford the silacycle 42 as an amorphous white solid (238 mg, 99%).

R_{f}=0.14 (15% ethyl acetate-hexanes; UV, PAA, CAM). ¹H NMR (500 MHz, CD₂Cl₂) δ 7.70-7.68 (m, 2H, 2×H₂₆), 7.49-7.27 (m, 8H, 4×H₄, 4×H₂₃), 4.78 (d, J=7.0 Hz, 1H, H₁₁), 4.50 (d, J=6.0 Hz, 1H, H₄), 2.26-2.15 (m, 4H, 1×H₂, 1×H₄, 1×H₆, 1×H₁₀), 2.12-2.07 (m, 1H, 1×H₂), 2.01 (s, 3H, H₂₂), 1.88-1.81 (m, 1H, 1×H₁), 1.77-1.68 (m, 2H, 2×H₁₃), 1.64-1.50 (m, 2H, 1×H₇, 1×H₈, 3×H₃, 1×H₁₆, 2×H₁₉), 1.35-1.29 (m, 2H, 1×H₁, 1×H₇), 1.11-1.02 (m, 4H, 1×H₈, 3×H₂₀), 0.95 (dd, J=15.5, 2.0 Hz, 1H, 1×H₆), 0.83 (s, 3H, H₁₈), 0.63 (d, J=7.0 Hz, 3H, H₁₇). ¹³C NMR (125 MHz, CD₂Cl₂) δ 218.2 (C), 170.9 (C) 137.3 (C), 136.9 (C), 134.7 (CH), 134.5 (CH), 130.5 (CH), 130.4 (CH), 128.5 (CH), 128.4 (CH), 78.6 (CH), 78.1 (CH), 59.3 (CH), 45.3 (C), 41.6 (C), 41.5 (CH₂), 40.4 (C), 38.7 (CH), 35.7 (CH), 34.9 (CH₂), 30.7 (CH₂), 27.8 (CH₂), 26.0 (CH₂), 26.0 (CH₃), 22.4 (CH₂), 21.0 (CH₃), 15.5 (CH₃), 13.0 (CH₂), 12.5 (CH₃), 8.6 (CH₃). IR (ATR-FTIR), cm⁻¹: 2974 (w), 1728 (s), 1462 (w), 1375 (w), 1245 (s), 1118 (m), 1027 (m), 977 (m), 956 (m), 834 (w), 716 (s), 699 (s), 504 (s). HRMS-ESI (m/z): [M+Na]⁺ calcd for C₃₄H₄₄NaO₄Si, 567.2907; found, 567.2915. [α]_{D}²⁵=+57° (c=0.50, CHCl₃).

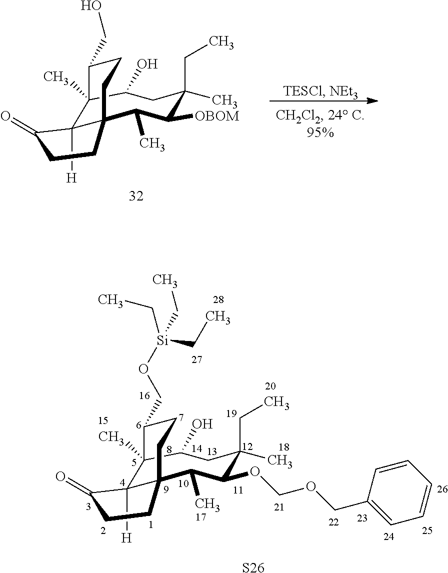

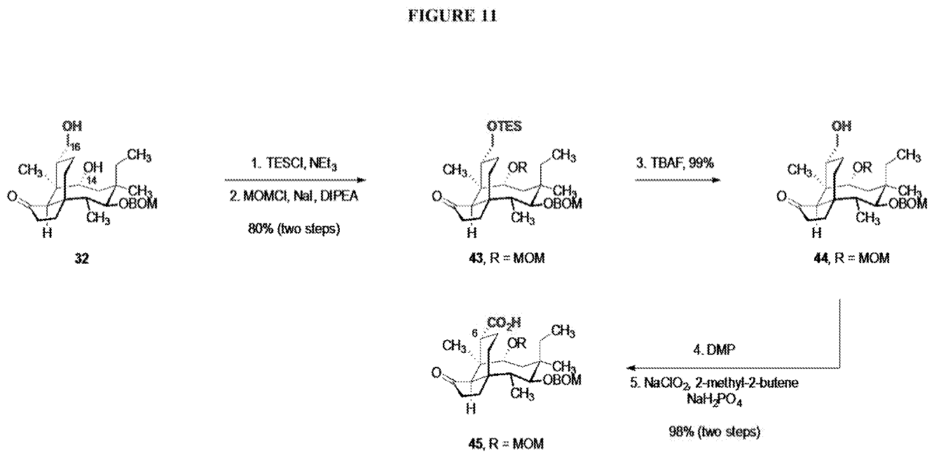

### Synthesis of Alcohol S26 (FIG. 11, Scheme 11)

Chlorotriethylsilane (192 μL, 1.14 mmol, 1.05 equiv) was added dropwise via syringe to a solution of diol 32 [500 mg, 1.09 mmol, 1 equiv, dried by azeotropic distillation with benzene (1.0 mL)] and triethylamine (304 μL, 2.18 mmol, 2.00 equiv) in dichloromethane (4.0 mL) at 24° C. The reaction mixture was stirred at 24° C. for 40 min. The product mixture was transferred to a separatory funnel that had been charged with dichloromethane (25 mL) and aqueous potassium phosphate buffer solution (pH 7, 0.10 M, 10 mL). The layers that formed were separated and the aqueous layer was extracted with dichloromethane (3×25 mL). The organic layers were combine and dried over sodium sulfate. The dried solution was filtered and the filtrate was concentrated to dryness. The residue obtained was purified by automated flash-column chromatography (eluting with hexanes initially, grading to 40% ether-hexanes, linear gradient) to afford the alcohol S26 as a light yellow oil (594 mg, 95%).

R_{f}=0.88 (50% ethyl acetate-hexanes; UV, PAA, CAM). ¹H NMR (400 MHz, CDCl₃) δ 7.36-7.26 (m, 5H, 2×H₂₄, 2×H₂₅, 1×H₂₆), 4.81-4.76 (m, 3H, 2×H₂₁, 1×OH), 4.66 (s, 2H, H₂₂), 4.20 (br s, 1H, H₁), 3.97 (d, J=10.8 Hz, 1H, 1×H₁₆), 3.47 (dd, J=11.2, 4.0 Hz, 1H, 1×H₁₆), 3.28 (d, J=6.0 Hz, 1H, H₁₄), 2.51-2.44 (m, 1H, H₁₀), 2.26-2.10 (m, 2H, H₂), 2.08 (s, 1H, H₄), 1.94 (qd, J=13.6, 3.2 Hz, 1H, 1×H₇), 1.87-1.79 (m, 1H, 1×H₈), 1.77-1.60 (m, 2H, 1×H₁, 1×H₁₉), 1.60-1.47 (m, 4H, 1×H₆, 2×H₁₃, 1×H₁₉), 1.44 (dd, J=9.2, 3.0 Hz, 1H, 1×H₁), 1.36 (s, 3H, H₁), 1.28-1.20 (m, 1H, 1×H₇), 1.15 (td, J=13.6, 4.0 Hz, 1H, 1×H₈), 1.02 (s, 3H, H₁₈), 0.99-0.91 (m, 15H, 3×H₁₇, 3×H₂₀, 9×H₂₈), 0.61 (q, J=8.0 Hz, 6H, H₂₇). ¹³C NMR (100 MHz, CDCl₃) δ 218.1 (C), 138.0 (C), 128.4 (CH), 128.3 (CH), 127.6 (CH), 127.6 (CH), 97.1 (CH₂), 86.7 (CH), 70.7 (CH₂), 63.7 (CH), 63.2 (CH₂), 60.0 (CH), 45.3 (C), 43.4 (CH), 42.8 (C), 41.2 (CH₂), 40.6 (C), 35.4 (CH), 34.5 (CH₂), 30.7 (CH₂), 27.2 (CH₃), 25.2 (CH₂), 22.2 (CH₂), 21.7 (CH₂), 13.9 (CH₃), 12.1 (CH₃), 7.8 (CH₃), 6.6 (CH₃), 4.1 (CH₂). IR (ATR-FTIR) cm⁻¹: 3437 (br w), 2955 (m), 2877 (m), 1736 (m), 1457 (m), 1380 (w), 1232 (w), 1163 (w), 1103 (m), 1045 (s), 1026 (s), 994 (s), 734 (m). HRMS-ESI (m/z): [M+H]⁺ calcd for C₃₄H₅₇O₅Si, 573.3975; found, 573.3963. [α]_{D}²⁵=+48° (c=0.50, CHCl₃).

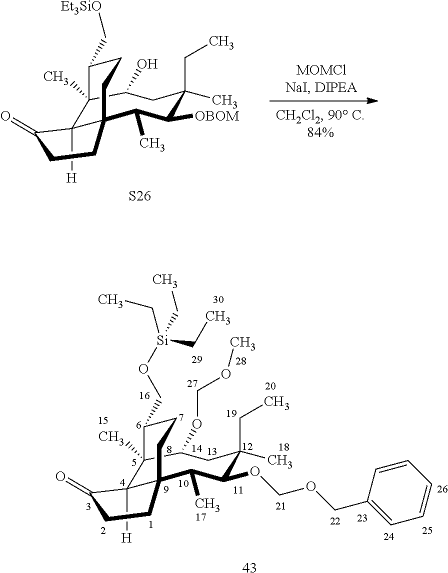

### Synthesis of 16-hydroxy-19,20-dihydromutilin derivative 43 (FIG. 11, Scheme: 11)

A 10-mL pressure tube with a Teflon-coated valve was charged with the alcohol S26 (120 mg, 210 μmol, 1 equiv). Benzene (1.0 mL) was added to the reaction vessel and the solution was concentrated to dryness. This process was repeated twice. Sodium iodide (126 mg, 839 μmol, 4.00 equiv) was added to the tube. The reaction vessel was evacuated and refilled using a balloon of argon. This process was repeated twice. Dichloromethane (2.0 mL), N,N-diisopropylethylamine (438 μL, 2.52 mmol, 12.0 equiv), and chloromethyl methyl ether (95.5 μL, 1.26 mmol, 6.00 equiv) were added sequentially to the reaction vessel at 24° C. The vessel was sealed and the sealed vessel was place in an oil bath that had been previously heated to 90° C. The reaction mixture was stirred and heated for 6 h at 90° C. The product mixture was transferred to a separatory funnel that had been charged with dichloromethane (25 mL) and aqueous potassium phosphate buffer solution (pH 7, 0.10 M, 10 mL). The layers that formed were separated and the aqueous layer was extracted with dichloromethane (3×25 mL). The organic layers were combine and dried over sodium sulfate. The dried solution was filtered and the filtrate was concentrated to dryness. The residue obtained was purified by automated flash-column chromatography (eluting with hexanes initially, grading to 25% ether-hexanes, linear gradient) to afford the 16-hydroxy-19,20-dihydromutilin derivative 43 as an amorphous white solid (108 mg, 84%).

R_{f}=0.30 (10% ethyl acetate-hexanes; UV, PAA, CAM). ¹H NMR (400 MHz, CDCl₃) δ 7.36-7.27 (m, 5H, 2×H₂₄, 2×H₂₅, 1×H₂₆), 4.76 (q, J=6.5 Hz, 2H, H₂₁), 4.67-4.62 (m, 2H, H₂₇), 4.57-4.53 (m, 2H, H₂₂), 4.09 (d, J=7.2 Hz, 1H, H₁₁), 3.84 (dd, J=10.4, 2.0 Hz, 1H, 1×H₁₆), 3.35 (s, 3H, H₂₈), 3.29-3.20 (m, 2H, 1×H₁₄, 1×H₁₆), 2.30-2.24 (m, 1H, H₁₀), 2.22-2.10 (m, 2H, H₂), 2.00 (s, 1H, H₄), 1.93 (qd, J=13.6, 2.0 Hz, 1H, 1×H₈), 1.84-1.42 (m, 9H, 2×H₁, 1×H₆, 2×H₇, 1×H₁₃, 2×H₁₉), 1.40 (s, 3H, H₁₅), 1.34 (dt, J=14.4, 2.4 Hz, 1H, 1×H₁₃), 1.13 (td, J=14.0, 4.0 Hz, 1H, 1×H₈), 1.00 (s, 3H, H₁₈), 0.98-0.88 (m, 15H, 3×H₁, 3×H₂₀, 9×H₃₀), 0.57 (q, J=8.0 Hz, 6H, H₂₉). ¹³C NMR (100 MHz, CDCl₃) δ 217.5 (C), 137.9 (C), 128.4 (CH), 127.6 (CH), 127.6 (CH), 96.9 (CH₂), 95.5 (CH₂), 85.2 (CH), 72.7 (CH), 70.7 (CH₂), 64.1 (CH₂), 58.8 (CH), 55.7 (CH₃), 46.2 (CH), 45.2 (C), 42.4 (C), 41.1 (C), 40.3 (CH₂), 35.3 (CH), 34.6 (CH₂), 30.2 (CH₂), 26.8 (CH₃), 25.3 (CH₂), 22.5 (CH₂), 22.0 (CH₂), 14.8 (CH₃), 12.0 (CH₃), 8.9 (CH₃), 6.8 (CH₃), 4.5 (CH₂). IR (ATR-FTIR), cm⁻¹: 2952 (m), 2876 (m), 1737 (m), 1450 (s), 1153 (w), 1039 (s), 966 (w), 738 (m). HRMS-ESI (m/z): [M+H]⁺ calcd for C₃₆H₆₁O₆Si, 617.4237; found, 617.4215. [α]_{D}²⁵=+51° (c=0.50, CHCl₃).

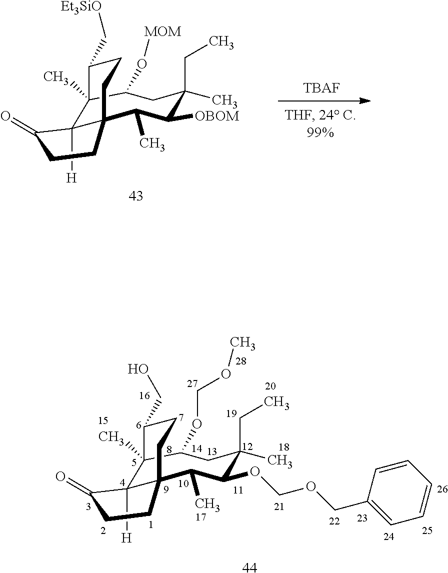

### Synthesis of Alcohol 44 (FIG. 1, Scheme 11)

A solution of tetrabutylammonium fluoride (1.0 M, 81.0 μL, 81.0 μmol, 2.00 equiv) was added dropwise via syringe to a solution of 16-hydroxy-19,20-dihydromutilin derivative 43 (25.0 mg, 40.5 μmol, 1 equiv) in tetrahydrofuran (500 μL) at 24° C. The reaction mixture was stirred for 15 min at 24° C. The product mixture was transferred to a separatory funnel that had been charged with ethyl acetate (25 mL) and saturated aqueous sodium bicarbonate solution (5.0 mL). The layers that formed were separated and the organic layer was washed with saturated aqueous sodium bicarbonate solution (3×5 mL). The washed organic layer was dried over sodium sulfate. The dried solution was filtered and the filtrate was concentrated to dryness. The residue obtained was purified by automated flash-column chromatography (eluting with hexanes initially, grading to 66% ethyl acetate-hexanes, linear gradient) to afford the alcohol 44 as an amorphous white solid (22.6 mg, 99%).

R_{f}=0.27 (33% ethyl acetate-hexanes; UV, PAA, CAM). ¹H NMR (400 MHz, CDCl₃) δ 7.34-7.26 (m, 5H, 2×H₂, 2×H₂₅, 1×H₂₆), 4.76 (q, J=6.7 Hz, 2H, H₂₁), 4.68-4.62 (m, 2H, H₂₇), 4.61-4.58 (m, 2H, H₂₂), 4.19 (d, J=7.6 Hz, 1H, H₁₁), 3.72 (dd, J=10.48, 4.0 Hz, 1H, 1×H₁₆), 3.48 (dd, J=11.6, 6.8 Hz, 1H, 1×H₆), 3.36 (s, 3H, H₂₃), 3.28 (d, J=6.0 Hz, 1H, H₁₄), 2.41 (t, J=7.6 Hz, 1H, OH), 2.26-2.15 (m, 2H, 2×H₂, 1×H₁₀), 2.04 (s, 1H, H₄), 1.83-1.52 (m, 8H, 2×H₁, 1×H₆, 1×H₇, 1×H₈, 2×H₁₃, 1×H₁₉), 1.49-1.38 (m, 4H, 1×H₇, 3×H₁₅), 1.27 (dt, J=18.4, 7.2 Hz, 1H, 1×H₁₉), 1.14 (td, J=13.6, 4.0 Hz, 1H, 1×H₈), 1.01 (s, 3H, H₁₈), 0.97-0.89 (m, 6H, 3×H₁₇, 3×H₂). ¹³C NMR (100 MHz, CDCl₃) δ 217.5 (C), 137.9 (C), 128.4 (CH), 127.7 (CH), 96.9 (CH₂), 94.9 (CH₂), 94.9 (CH₂), 85.2 (CH), 72.4 (CH), 70.7 (CH₂), 63.9 (CH₂), 59.1 (CH), 55.8 (CH₃), 45.5 (CH), 45.2 (C), 42.5 (C), 41.1 (C), 39.7 (CH₂), 35.2 (CH), 34.6 (CH₂), 30.1 (CH₂), 26.8 (CH₃), 25.1 (CH₂), 22.4 (CH₂), 21.7 (CH₂), 20.8 (CH), 15.3 (CH₃), 12.0 (CH₃), 8.9 (CH₃). IR (ATR-FTIR), cm⁻¹: 2937 (w), 2879 (w), 1733 (m), 1458 (w), 1153 (m), 1082 (m), 1024 (s), 966 (m), 907 (s), 727 (s), 697 (s), 646 (m). HRMS-ESI (m/z): [M+Na]⁺ calcd for C₃H₄₆NaO₆, 525.3192; found, 525.3190. [α]_{D}²⁵=+49° (c=0.25, CHCl₃).

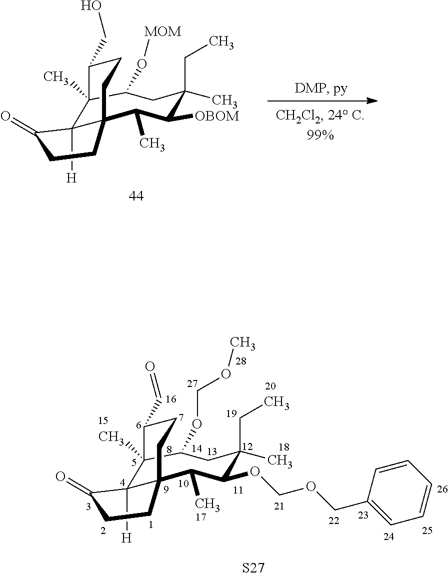

### Synthesis of Aldehyde S27 (FIG. 11, Scheme 11)

Eleven equal portions of Dess-Martin periodinane (233 mg, 550 μmol, 1.10 equiv) was added over 1 h to a solution of the alcohol 44 (251 mg, 500 μmol, 1 equiv) and pyridine (404 μL, 5.00 mmol, 10.0 equiv) in dichloromethane (4.0 mL) at 24° C. The resulting mixture was stirred for 10 min at 24° C. The product mixture was diluted sequentially with ether (5.0 mL), a saturated aqueous sodium bicarbonate solution (2.5 mL) and a saturated aqueous sodium thiosulfate solution (2.5 mL). The resulting mixture was stirred for 10 min at 24° C. The resulting mixture was transferred to a separatory funnel and the layers that formed were separated. The aqueous layer obtained was extracted with dichloromethane (3×25 mL). The organic layers were combined and the combined organic layer was dried over sodium sulfate. The dried solution was filtered and the filtrate was concentrated to dryness. The residue obtained was purified by automated flash-column chromatography (eluting with hexanes initially, grading to 30% ethyl acetate-hexanes, linear gradient) to afford aldehyde S27 as an amorphous white solid (250 mg, 99%).

R_{f}=0.42 (33% ethyl acetate-hexanes; UV, PAA, CAM). ¹H NMR (400 MHz, CD₂Cl₂) δ 9.75 (s, 1H H₁₆), 7.35-7.25 (m, 5H, 2×H₂₄, 2×H₂₅, 1×H₂₄), 4.76 (t, J=6.4 Hz, 2H, H₂₁), 4.63 (dd, J=18.0, 6.0 Hz, 2H, H₂₇), 4.35 (s, 2H, H₂), 3.94 (d, J=7.6 Hz, 1H, H₁₁), 3.31-3.27 (m, 4H, 1×H₁₄, 3×H₂₃), 2.34-2.07 (m, 5H, 2×H₂, 1×H₄, 1×H₆, 1×H₁₀), 1.84-1.61 (m, 9H, 1×H₁, 1×H₇, 1×H₈, 1×H₁₃, 3×H₁₅, 2×H₁₉), 1.54 (dd, J=16.0, 8.0 Hz, 1H, 1×H₁₃), 1.48-1.41 (m, 2H, 1×H₁, 1×H₇), 1.09-1.03 (m, 1H, 1×H₈), 0.99 (s, 3H, H₁₉), 0.95-0.87 (m, 6H, 3×H₁₇, 3×H₂). ¹³C NMR (100 MHz, CD₂Cl₂) δ 217.6 (C), 202.2 (CH), 138.8 (C), 128.8 (CH), 128.1 (CH), 128.1 (CH), 97.9 (CH₂), 96.5 (CH₂), 85.8 (CH), 73.1 (CH), 71.2 (CH₂), 58.3 (CH), 56.5 (CH₃), 53.7 (CH), 45.2 (C), 44.6 (C), 41.8 (C), 38.4 (CH₂), 36.1 (CH), 34.7 (CH₂), 26.1 (CH₂), 27.1 (CH₃), 25.8 (CH₂), 22.9 (CH₂), 18.0 (CH₂), 15.7 (CH₃), 12.5 (CH₃), 9.0 (CH₃). IR (ATR-FTIR), cm⁻¹: 2959 (w), 2879 (w), 1735 (s), 1464 (m), 1241 (w), 1162 (m), 1106 9w), 1041 (s), 1023 (s), 937 (w). HRMS-ESI (m/z): [M+H]⁺ calcd for C₃₀H₄₅O₆, 501.3216; found, 501.3198. [α]_{D}²⁵=+46° (c=0.10, CHCl₃).

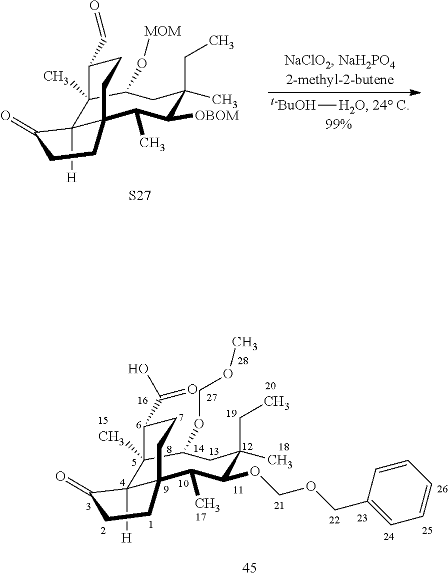

### Synthesis of Carboxylic Acid 45 (FIG. 11, Scheme 11)

2-Methyl-2-butene (636 μL, 6.00 mmol, 12.0 equiv) and a solution of sodium chlorite (301 mg, 3.33 mmol, 6.65 equiv) and sodium phosphate monobasic (368 mg, 2.67 mmol, 5.34 equiv) in water (2.3 mL) were added to a solution of the aldehyde S27 (250 mg, 500 μmol, 1 equiv) in tert-butanol (7.1 mL) at 24° C. The reaction mixture was stirred for 2 h at 24° C. The product mixture was transferred to a separatory funnel that had been charged with ethyl acetate (25 mL) and an aqueous hydrochloric acid solution (1 M, 10 mL). The layers that formed were separated and the aqueous layer was extracted with ethyl acetate (3×25 mL). The organic layers were combined and dried over sodium sulfate. The dried solution was filtered and the filtrate was concentrated to dryness. The residue obtained was purified by flash-column chromatography (eluting with 25% ethyl acetate-hexanes-0.5% acetic acid, isocratic gradient) to afford carboxylic acid 45 as an amorphous white solid (253 mg, 99%).

R_{f}=0.42 (33% ethyl acetate-hexanes; UV, PAA, CAM). ¹H NMR (400 MHz, CD₂Cl₂) δ 11.1 (br s, OH), 7.36-7.27 (m, 5H, 2×H₂₄, 2×H₂, 1×H₂₆), 4.78 (q, J=6.7 Hz, 2H, H₂), 4.65 (q, J=10.6 Hz, 2H, H₂₂), 4.53 (d, J=6.4 Hz, 1H, 1×H₂₇), 4.41 (d, J=6.4 Hz, 1H, 1×H₂₇), 4.07 (d, J=8.0 Hz, 1H₁₁), 3.31-3.27 (m, 4H, 1×H₁₄, 3×H₂₈), 2.47 (dd, J=13.2, 8.0 Hz, 1H, H₆), 2.32-2.07 (m, 4H, 2×H₂, 1×H₄, 1×H₁₀), 1.97 (qd, J=13.2, 2.8 Hz, 1H, 1×H₁₉), 1.82-1.70 (m, 4H, 1×H₁, 1×H₇, 1×H₈, 1×H₁₃), 1.63-1.47 (m, 7H, 1×H₁, 1×H₇, 1×H₁₃, 3×H₁₃, 1×H₁₉), 1.06 (td, J=14.4, 4.0 Hz, 1H, 1×H₈), 1.00 (s, 3H, H₁₈), 0.99-0.91 (m, 6H, 3×H₁₇, 3×H₂₀). ¹³C NMR (100 MHz, CD₂Cl₂) δ 217.1 (C), 181.1 (C), 138.8 (C), 128.8 (CH), 128.1 (CH), 128.1 (CH), 98.3 (CH₂), 97.8 (CH₂), 85.8 (CH), 75.8 (CH), 71.1 (CH₂), 58.2 (CH), 55.7 (CH₃), 45.9 (CH), 45.1 (C), 44.3 (C), 42.0 (CH₂), 40.6 (C), 35.6 (CH), 34.7 (CH₂), 28.1 (CH₂), 27.2 (CH₃), 25.4 (CH₂), 23.1 (CH₂), 21.4 (CH₂), 16.1 (CH₃), 12.3 (CH₃), 9.1 (CH₃). IR (ATR-FTIR), cm⁻¹: 2837 (w), 1706 (s), 1410 (m), 1289 (s), 1234 (s), 1162 (w), 1038 (m), 1020 (s), 935 (m), 744 (w), 700 (w), 627 (m), 480 (w). HRMS-ESI (m/z): [M+H]⁺ calcd for C₃₀H₄₅O₇, 517.3165; found, 517.3174. [α]_{D}²⁵=+52° (c=0.50, CHCl₃).

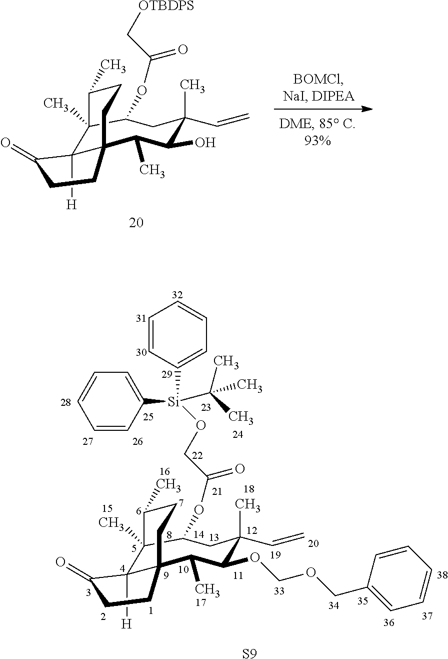

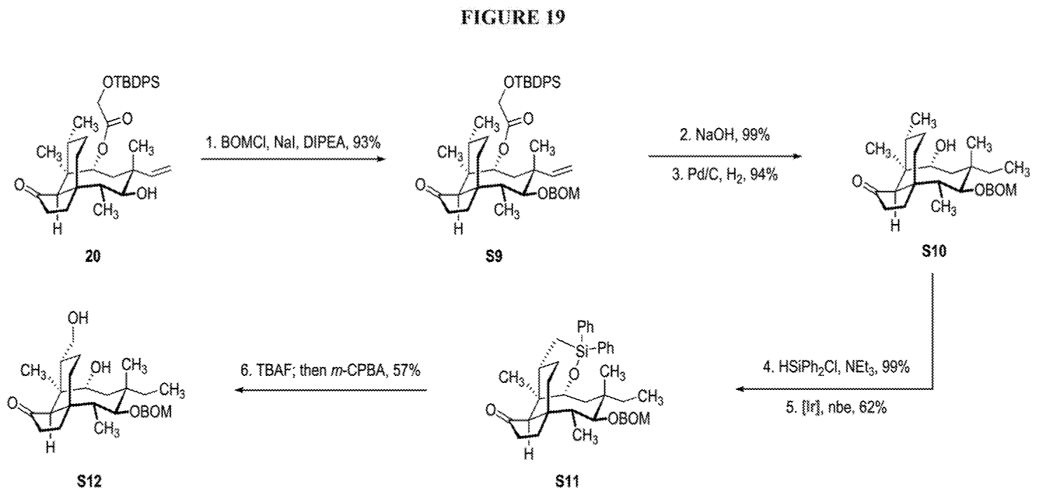

### Synthesis of O-tert-butyldiphenylsilyl-11-benzyloxymethylenoxy-12-epi-pleuromutilin (S9, FIG. 19, Scheme 0.2)

A 100-mL round-bottomed flask fused to a Teflon-coated valve was charged with O-tert-butyldiphenylsilyl-12-epi-pleuromutilin (20, 617 mg, 1.00 mmol, 1 equiv). Benzene (2.0 mL) was added and the solution was concentrated to dryness. This process was repeated twice. Sodium iodide (600 mg, 4.00 mmol, 4.00 equiv) was added to the reaction vessel. The reaction vessel was evacuated and refilled using a balloon of argon. This process was repeated twice. 1,2-Dimethoxyethane (10 mL), N,N-diisopropylethylamine (1.05 mL, 6.00 mmol, 6.00 equiv), and benzyl chloromethyl ether (556 μL, 4.00 mmol, 4.00 equiv) was added sequentially via syringe to the reaction mixture at 24° C. The reaction vessel was sealed and the sealed vessel was placed in an oil bath that had been previously heated to 85° C. The reaction mixture was stirred and heated for 1.5 h at 85° C. The product mixture was allowed to cool to over 30 min 0° C. with an ice bath. A saturated aqueous sodium bicarbonate solution (5.0 mL) was added dropwise via syringe to the product mixture. The resulting mixture was stirred for 10 min at 0° C. The resulting mixture was transferred to a separatory funnel that had been charged with dichloromethane (50 mL). The layers that formed were separated and the aqueous layer was extracted with dichloromethane (3×20 mL). The organic layers were combined and the combined organic layers were dried over sodium sulfate. The dried solution was filtered and the filtrate was concentrated. The residue obtained was purified by automated flash-column chromatography (eluting with hexanes initially, grading to 25% ether-hexanes, linear gradient) to afford O-tert-butyldiphenylsilyl-11-benzyloxymethylenoxy-12-epi-pleuromutilin (S9) as an amorphous white solid (683 mg, 93%).

R_{f}=0.52 (20% ethyl acetate-hexanes; UV, PAA, CAM). ¹H NMR (400 MHz, CD₂Cl₂) δ 7.72-7.66 (m, 4H, 2×H₂₇, 2×H₃₁), 7.47-7.28 (m, 11H, 2×H₂₆, 1×H₂₈, 2×H₃₀, 1×H₃₂, 2×H₃₆, 2×H₃₇, 1×H₃₈), 5.92 (dd, J=17.6, 10.8 Hz, 1H, H₁₉), 5.68 (d, J=8.4 Hz, 1H, H₁₄), 5.07 (d, J=17.6 Hz, 1H, 1×H₂₀), 5.01 (d, J=10.8 Hz, 1H, 1×H₂₀), 4.71 (s, 2H, H₃₃), 4.68-4.61 (m, 2H, H₃₄), 4.17 (dd, J=22.8, 6.0 Hz, 2H, H₂₂), 3.45 (d, J=6.0 Hz, 1H, H₁), 2.56-2.49 (m, 1H, H₁₀), 2.23-2.16 (m, 2H, H₂), 2.13-2.06 (m, 2H, 1×H₄, 1×H₁₃), 1.83-1.75 (m, 2H, 1×H₁, 1×H₈), 1.64-1.55 (m, 2H, 1×H₆, 1×H₇), 1.48 (td, J=9.6, 3.6 Hz, 1H, 1×H₁), 1.42-1.35 (m, 4H, 1×H₇, 3×H₁₅), 1.30 (s, 3H, H₁₈), 1.20-1.13 (m, 1H, 1×H₈), 1.10 (s, 9H, H₂₄), 1.00 (d, J=7.2 Hz, 3H, H₁₆), 0.98-0.92 (m, 1H, 1×H₁₃), 0.63 (d, J=6.0 Hz, 3H, H₁₇). ¹³C NMR (100 MHz, CD₂Cl₂) δ 216.8 (C), 169.7 (C), 148.4 (CH), 138.3 (C), 135.5 (CH), 132.8 (C), 129.8 (CH), 128.2 (CH), 127.8 (CH), 127.5 (CH), 127.4 (CH), 111.2 (CH₂), 96.7 (CH₂), 82.0 (CH), 70.4 (CH₂), 68.6 (CH), 62.8 (CH₂), 58.1 (CH), 45.2 (C), 44.5 (C), 43.5 (CH) 41.9 (C), 36.8 (CH), 35.6 (CH), 34.5 (CH₂), 30.4 (CH₂), 26.9 (CH₂), 26.4 (CH), 25.1 (CH₂), 19.0 (C), 16.5 (CH₃), 15.4 (CH₃), 14.6 (CH), 11.5 (CH₃). IR (ATR-FTR), cm⁻¹: 2932 (w), 2859 (w) 1734 (m), 1454 (w) 1428 (w), 1382 (w), 1287 (w), 1210 (w), 1137 (s), 1113 (s), 1025 (s), 966 (m), 914 (w), 824 (m), 740 (w), 701 (s), 613 (w), 505 (m). HRMS-ESI (m/z): [M+Na]⁺ calcd for C₄₀H₆₀NaO₆Si, 759.4057; found, 759.4054. [α]_{D}²⁵=+28° (c=1.00, CHCl₃).

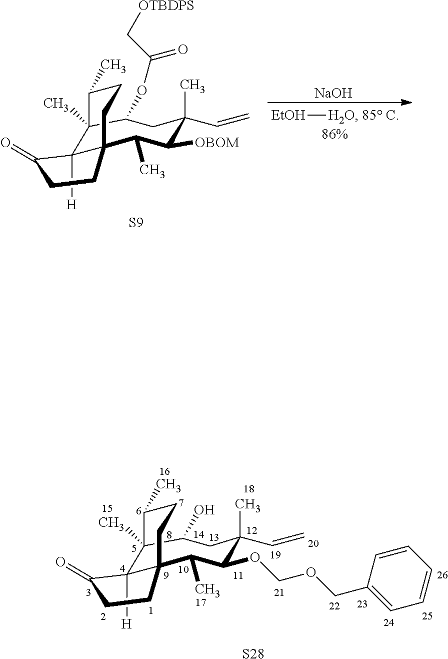

### Synthesis of 1-benzyloxymethylenoxy-2-epi-mutilin (S28, FIG. 19, Scheme S2)

Water (1.32 mL) and an aqueous sodium hydroxide solution (50% w/w, 184 μL) were added dropwise via syringe to a solution of the O-tert-butyldiphenylsilyl-11-benzyloxymethylenoxy-2-epi-pleuromutilin (S9, 683 mg, 1.00 mmol, 1 equiv) in ethanol (2.1 mL) in a 25-mL round-bottomed flask fitted with a reflux condenser at 24° C. The reaction vessel was placed in an oil bath that had been previously heated to 85° C. The reaction mixture was stirred and heated for 4 h at 85° C. The resulting mixture was allowed to cool over 30 min to 24° C. The product mixture was transferred to a separatory funnel that had been charged with dichloromethane (50 mL). The layers that formed were separated and the aqueous layer was extracted with dichloromethane (3×20 mL). The organic layers were combined and the combined organic layers were dried over sodium sulfate. The dried solution was filtered and the filtrate was concentrated. The residue obtained was purified by automated flash-column chromatography (eluting with hexanes initially, grading to 33% ethyl acetate-hexanes, linear gradient) to afford 11-benzyloxymethylenoxy-12-epi-pleuromutilin (S28) as an amorphous white solid (352 mg, 86%).

R_{f}=0.52 (20% ethyl acetate-hexanes; UV, PAA, CAM). ¹H NMR (400 MHz, CDCl₃) δ 7.35-7.25 (m, 5H, 2×H₂₄, 2×H₂₅, 1×H₂₆), 5.90 (dd, J=17.6, 10.8 Hz, 1H, H₁₉), 5.04 (d, J=17.6 Hz, 1H, 1×H₂₀), 4.97 (d, J=10.8 Hz, 1H, 1×H₂₀), 4.67 (s, 2H, H₂₁), 4.61 (dd, J=16.8, 4.8 Hz, 2H, H₂₂), 4.35 (br s, 1H, H₁), 3.40 (d, J=6.0 Hz, 1H, H₁), 2.39-2.42 (m, 1H, H₁₀), 2.26-2.09 (m, 2H, H₂), 2.05-1.96 (m, 2H, 1×H₄, 1×H₁₃), 1.77-1.68 (m, 2H, 1×H₁, 1×H₈), 1.67-1.60 (m, 1H, 1×H₆), 1.53 (qd, J=14.0, 3.6 Hz, 1H, 1×H₇), 1.45-1.35 (m, 2H, 1×H₁, 1×H₇), 1.34 (s, 3H, H₁₅), 1.23 (s, 3H, H₁₈), 1.17-1.09 (m, 2H, 1×H₈, 1×H₁₃), 0.95 (app t, 6H, 3×H₁₆, 1×H₁₇). ¹³C NMR (100 MHz, CDCl₃) δ 217.6 (C), 148.4 (CH), 137.9 (C), 128.3 (CH), 127.5 (CH), 127.5 (CH), 111.1 (CH₂), 96.5 (CH₂), 82.1 (CH), 70.5 (CH₂), 66.2 (CH), 58.9 (CH), 46.0 (CH₂), 45.2 (C), 44.3 (C), 42.5 (C), 36.9 (CH), 35.5 (CH), 34.5 (CH₂), 30.5 (CH₂), 27.0 (CH₂), 25.0 (CH₂), 18.9 (CH₃), 14.9 (C₃), 13.3 (CH₃), 11.7 (CH₃). IR (ATR-FTIR), cm⁻¹: 3504 (br w), 2981 (w), 2930 (m), 2876 (w), 1732 (m), 1497 (w), 1454 (m), 1411 (w), 1378 (m), 1287 (w), 1165 (w), 1115 (w), 1025 (s), 970 (w), 910 (w), 735 (m), 698 (m). HRMS-ESI (m/z): [M+H]⁺ calcd for C₂₈H₄₁O₄, 441.3005; found, 441.3003. [α]_{D}²⁵=+66° (c=0.50, CHCl₃).

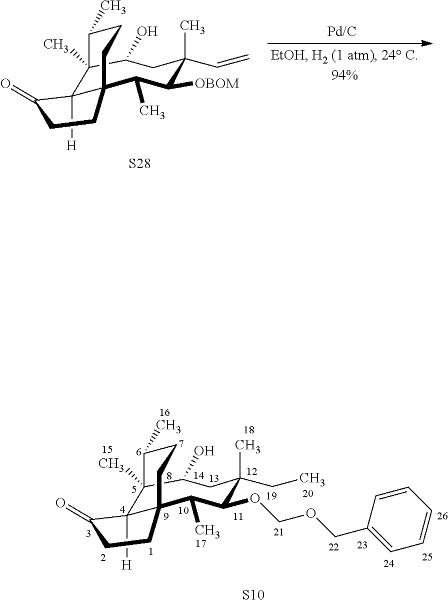

### Synthesis of 1-benzyloxymethylenoxy-12-epi-19,20-mutilin (S10, FIG. 19, Scheme S2)

Palladium on carbon (5 wt. % loading, 67.4 mg, 31.0 μmol, 0.05 equiv) was added to a solution of 11-benzyloxymethylenoxy-12-epi-pleuromutilin (S28.278 mg, 619 μmol, 1 equiv) in ethanol (4.0 mL) at 24° C. The reaction vessel was evacuated and re-filled using a balloon of dihydrogen. This process was repeated four times. The reaction mixture was stirred for 12 h at 24° C. The product mixture was filtered through a short column of celite and the short column was rinsed with dichloromethane (250 mL). The filtrates were combined and the combined filtrates were concentrated to dryness. The residue obtained was purified by automated flash-column chromatography (eluting with hexanes initially, grading to 25% ether-hexanes, linear gradient) to afford 11-benzyloxymethylenoxy-2-epi-19,20-dihydromutilin (S10) as an amorphous white solid (262 mg, 94%).

R_{f}=0.39 (20% ethyl acetate-hexanes; UV, PAA, CAM). ¹H NMR (500 MHz, CD₂Cl₂) δ 7.35-7.28 (m, 5H, 2×H₂₄, 2×H₂₅, 1×H₂₆), 4.77 (s, 2H, H₂₁), 4.68-4.62 (m, 2H, H₂₂), 4.43 (d, J=8.0 Hz, 1H, H₁₁), 3.33 (d, J=6.5 Hz, 1H, H₁₄), 2.40-2.32 (m, 1H, H₁₀), 2.25-2.11 (m, 2H, H₂), 1.96 (s, 1H, H₄), 1.89 (dd, J=12.4, 6.4 Hz, 1H, 1×H₁₃), 1.79-1.67 (m, 2H, 1×H₁, 1×H₈), 1.63-1.44 (m, 5H, 1×H₁, 1×H₆, 1×H₇, 2×H₁₉), 1.42-1.35 (m, 2H, 1×H₇, 1×OH), 1.33 (s, 3H, H₁₉), 1.17-1.10 (m, 2H, 1×H₈, 1×H₁₃), 1.02 (s, 3H, H₁₈), 0.96 (d, J=5.5 Hz, 3H, H₆), 0.94 (d, J=5.5 Hz, 3H, H₇), 0.89 (t, J=7.3 Hz, 3H, H₂). ¹³C NMR (125 MHz, CD₂Cl₂) δ 218.2 (C), 138.9 (C), 128.8 (CH), 128.2 (CH), 128.1 (CH), 97.5 (CH₂), 82.7 (CH), 71.2 (CH₂), 66.9 (CH), 59.5 (CH), 45.8 (CH₂), 44.5 (C), 43.2 (C), 41.3 (C), 37.7 (CH₂), 36.2 (CH), 36.2 (CH), 34.8 (CH₂), 31.2 (CH₂), 27.7 (CH₂), 25.8 (CH₂), 18.6 (CH₃), 17.2 (CH₃), 13.8 (CH₃), 12.2 (CH₃), 8.4 (CH₃). IR (ATR-FTIR), cm⁻¹: 3502 (br w), 2957 (m), 2881 (w), 1833 (m), 1455 (w), 1381 (w), 1162 (w), 1114 (w), 1084 (w), 1026 (s), 968 (w), 736 (w), 698 (w). HRMS-ESI (m/z): [M+H]⁺ calcd for C₂₈H₄O₄, 443.3161; found, 443.3159. [α]_{D}²⁵=+62° (c=0.50, CHCl₃).

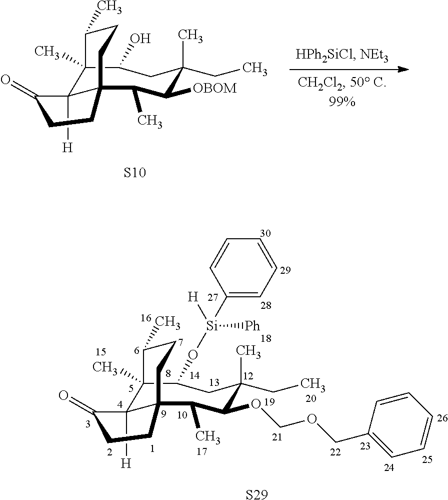

### Synthesis of Silane S29 (FIG. 19, Scheme S2)

A 10-mL round-bottomed flask fused to a Teflon-coated valve was charged with 11-benzyloxymethylenoxy-12-epi-19,20-dihydromutilin (S10, 262 mg, 593 μmol, 1 equiv). Benzene (1.0 mL) was added and the solution was concentrated to dryness. This process was repeated twice. The reaction vessel was evacuated and refilled using a balloon of argon. This process was repeated two times. Dichloromethane (1.5 mL), triethylamine (330 μL, 2.37 mmol, 4.00 equiv), and (chloro)diphenylsilane (232 μL, 1.19 μmol, 2.00 equiv, 95% purity) were added sequentially to the reaction vessel. The vessel was sealed and the sealed vessel was placed in an oil bath that had been previous heated to 50° C. The reaction was stirred and heated for 90 min at 50° C. The reaction vessel was allowed to cool over 30 min to 24° C. The product mixture was diluted sequentially with pentane (3.0 mL) and an aqueous potassium phosphate buffer solution (pH 7, 0.10 M, 1.0 mL). The diluted mixture was transferred to a separatory funnel and the layers formed were separated. The aqueous layer was extracted with dichloromethane (3×5.0 mL). The organic layers were combined and the combined organic layers were dried over sodium sulfate. The dried solution was filtered and the filtrate was concentrated to dryness. The residue obtained was purified by automated flash-column chromatography (eluting with hexanes initially, grading to 25% ether-hexanes, linear gradient) to afford silane S29 as an amorphous white solid (372 mg, 99%).

R_{f}=0.52 (20% ethyl acetate-hexanes; UV, PAA, CAM). ¹H NMR (400 MHz, C₆D₆) δ 7.70-7.62 (m, 4H, 4×H₂₉), 7.22-7.00 (m, 11H, 2×H₂₄, 2×H₂₅, 1×H₂₆, 4×H₂₈, 2×H₃₀), 5.71 (s, 1H, Si—H), 4.68 (d, J=10.5 Hz, 1H, H₁₁), 4.49 (dd, J=13.5, 7.5 Hz, 2H, H₂₁), 4.44 (s, 2H, H₂₂), 3.02 (d, J=7.5 Hz, 1H, H₁₄), 2.20-2.13 (m, 1H, H₁₀), 1.88 (s, 3H, H₁₅), 1.85-1.78 (m, 2H, H₂), 1.76-1.67 (m, 3H, 1×H₄, 1×H₆, 1×H₁₃), 1.62-1.54 (m, 1H, 1×H₁₃), 1.51-1.41 (m, 1H, 1×H₁₉), 1.39-1.28 (m, 3H, 1×H₁, 1×H₇, 1×H₈), 1.20-1.11 (m, 1H, 1×H₁₉), 1.11-1.03 (m, 4H, 1×H₇, 3×H₁₆), 1.00-0.89 (m, 1H, 1×H₁), 0.84-0.77 (m, 4H, 1×H₈, 3×H₁₈), 0.74 (d, J=7.2 Hz, 3H, H₁₇), 0.61 (t, J=9.5 Hz, 3H, H₂₀). ¹³C NMR (100 MHz, C₆D₆) δ 215.3 (C), 138.2 (C), 135.5 (C), 134.8 (CH), 134.8 (CH), 130.1 (CH), 130.0 (CH), 128.2 (CH), 128.2 (CH), 96.5 (CH₂), 82.9 (CH), 70.3 (CH₂), 70.1 (CH), 58.6 (CH), 44.9 (C), 44.0 (C), 42.0 (CH₂), 41.0 (C), 37.4 (CH), 35.6 (CH), 34.2 (CH₂), 34.0 (CH₂), 30.4 (CH₂), 27.1 (CH₂), 25.1 (CH₂), 18.8 (CH₃), 15.9 (CH₃), 14.7 (CH₃), 11.6 (CH₃), 7.8 (CH₃). IR (ATR-FTIR), cm⁻¹: 2957 (w), 2879 (w), 2113 (w), 1734 (m), 1455 (w), 1429 (w), 1379 (w), 1161 (w), 1113 (m), 1035 (s), 1025 (s), 968 (w), 850 (m), 809 (m), 732 (s), 698 (s), 497 (w). HRMS-ESI (m/z): [M+Na]⁺ calcd for C₄₀H₅₂NaO₄Si, 647.3533; found, 647.3528. [α]_{D}²⁵=+58° (c=0.10, CHCl₃).

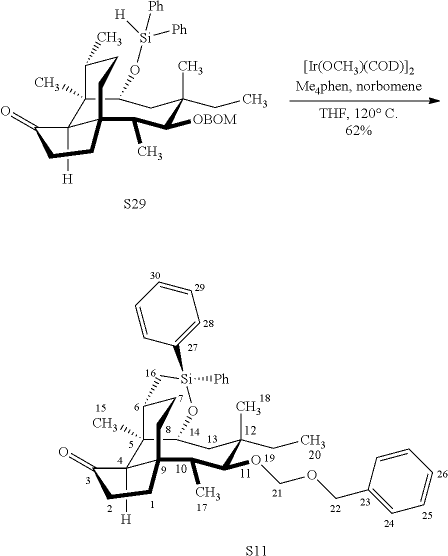

### Synthesis of Silacycle S11 (FIG. 19, Scheme S2)

This experiment was adapted from the work of Hartwig and co-workers.² A 4-mL pressure tube with a Teflon-coated valve was charged with 3,4,7,8-tetramethyl-1,10-phenanthroline (17.5 mg, 74.4 μmol, 12.5 mol %) and norbornene (83.7 mg, 893 mmol, 1.50 equiv) in the glovebox. A 4-mL vial was charged with silane S29 [372 mg, 595 μmol, 1 equiv, dried by azeotropic distillation with benzene (3×1.0 mL)]. The vessel containing the silane was evacuated and refilled using a balloon of argon. This process was repeated two times. Tetrahydrofuran (500 μL) was transferred into the vessel containing the silane and the resulting solution was added to the vessel containing the ligand and norbornene in the glovebox. The vessel containing the silane was rinsed with tetrahydrofuran (3×100 μL) and the combined rinses were transferred to the reaction vessel.

Methoxy(cyclooctadiene)iridium(I) dimer (19.6 mg, 29.8 μmol, 5 mol %) was added to an oven-dried 20-mL vial. Tetrahydrofuran (500 μL) was added into the vial containing the catalyst and the resulting solution was transferred dropwise via syringe to the reaction vessel in the glovebox. The vial containing the catalyst was rinsed with tetrahydrofuran (3×100 μL) and the combined rinses were transferred into the reaction vessel. The reaction vessel was sealed and the reaction mixture was stirred for 1 h at 24° C. in the glovebox. The sealed reaction vessel was then removed from the glovebox and placed in an oil bath that had been preheated to 120° C. The reaction mixture was stirred and heated for 7 h at 120° C. The reaction vessel was allowed to cool over 30 min to 24° C. and the cooled product mixture was concentrated to dryness. The residue obtained was purified by automated flash-column chromatography (eluting with hexanes initially, grading to 15% ether-hexanes, linear gradient) to afford the silacycle S11 as an amorphous white solid (231 mg, 62%).

R_{f}=0.50 (20% ethyl acetate-hexanes; UV, PAA, CAM). ¹H NMR (400 MHz, C₆D₆) δ 7.78-7.65 (m, 4H, 4×H₂₉), 7.36-7.06 (m, 11H, 2×H₂₄, 2×H₂₅, 1×H₂₆, 4×H₂₈, 2×H₃₀), 4.77 (d, J=7.2 Hz, 1H, H₁₁), 4.64-4.45 (m, 4H, 2×H₂₁, 2×H₂₂), 3.09 (d, J=6.8 Hz, 1H, H₁₄), 2.32-2.26 (m, 1H, H₆), 2.24-2.13 (m, 1H, H₁₀), 1.91 (s, 3H, H₁₅), 1.88-1.78 (m, 3H, 2×H₂, 1×H₁₉), 1.78-1.72 (m, 2H, 1×H₄, 1×H₁₉), 1.72-1.56 (m, 2H, 1×H₁, 1×H₁₆), 1.42-1.19 (m, 6H, 1×H₁, 2×H₈, 1×H₁₃, 3×H₁₈), 1.18-1.10 (m, 1H, 1×H₇), 1.03-0.96 (m, 1H, 1×H₁), 0.93 (t, J=7.4 Hz, 3H, H₂₀), 0.89-0.82 (m, 1H, 1×H₁₃), 0.78 (td, J=14.0, 4.4 Hz, 1H, 1×H₁₆), 0.60 (d, J=7.2 Hz, 3H, H₁₇). ¹³C NMR (100 MHz, C₆D₆) δ 215.4 (C), 138.3 (C), 136.9 (C), 136.3 (C), 134.4 (CH), 130.0 (CH), 129.9 (CH), 129.9 (CH), 128.2 (CH), 128.2 (CH), 128.0 (CH), 127.8 (CH), 96.9 (CH₂), 83.3 (CH), 70.4 (CH₂), 66.8 (CH), 58.2 (CH), 44.4 (C), 42.0 (C), 41.2 (CH₂), 40.8 (C), 37.2 (CH), 36.6 (CH), 34.1 (CH₂), 34.0 (CH₂), 30.2 (CH₂), 27.2 (CH₂), 25.7 (CH₂), 16.3 (CH₃), 15.3 (CH₃), 12.9 (CH₂), 12.0 (CH), 8.1 (CH₃). IR (ATR-FTIR), cm⁻¹: 2957 (w), 1736 (m), 1457 (w), 1429 (w), 1380 (w), 1160 (w), 1118 (m), 1023 (s), 990 (m), 737 (s), 717 (s), 698 (s), 500 (s). HRMS-ESI (m/z): [M+Na]⁺ calcd for C₄₀H₅₀NaO₄Si, 645.3376; found, 645.3382. [α]_{D}²⁵=+46° (c=0.25, CHCl₃).

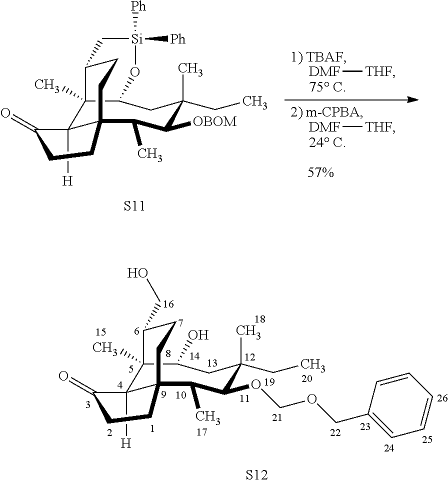

### Synthesis of diol S12 (FIG. 19, Scheme S2)

A solution of tetrabutyl ammonium fluoride (1.0 M, 740 μL, 740 μmol, 2.00 equiv) in tetrahydrofuran was added to a solution of the silacycle S11 (230 mg, 370 μmol, 1 equiv) in a mixture of tetrahydrofuran and N,N-dimethylformamide (1:3 v/v, 3.0 mL) at 24° C. The reaction vessel was placed in an oil bath that had been previously heated to 75° C. The reaction mixture was stirred and heated for 10 min at 75° C. The resulting mixture was immediately cooled to 24° C. using an ice bath. Freshly recrystallized m-chloroperbenzoic acid (192 mg, 1.11 mmol, 3.00 equiv) was added to the reaction mixture at 24° C. The reaction mixture was stirred for 90 min at 24° C. The product mixture was diluted sequentially with ether (5.0 mL) and aqueous potassium phosphate buffer solution (pH 7, 0.10 M, 2.0 mL). The diluted product mixture was transferred to a separatory funnel that had been charged with a mixture of ether and pentane (1:1, v/v, 50 mL). The layers that formed were separated and the organic layer was washed with saturated aqueous sodium bicarbonate solution (3×5 mL). The washed organic layer was dried over sodium sulfate. The dried solution was filtered and the filtrate was concentrated to dryness. The residue obtained was purified by automated flash-column chromatography (eluting with hexanes initially, grading to 100% ethyl acetate-hexanes, linear gradient) to afford the diol S12 as an amorphous white solid (97.2 mg, 57%).

R_{f}=0.50 (20% ethyl acetate-hexanes; UV, PAA, CAM). ¹H NMR (400 MHz, CDCl₃) δ 7.37-7.27 (m, 5H, 2×H₂₄, 2×H₂₅, 1×H₂₆), 4.78-4.74 (m, 2H, H₂₁), 4.69-4.62 (m, 2H, H₂₂), 4.35 (d, J=7.6 Hz, 1H, H₁₁), 3.94 (d, J=7.2 Hz, 1H, 1×H₆), 3.49 (dd, J=11.6, 4.0 Hz, 1H, 1×H₁₆), 3.33 (d, J=6.4 Hz, 1H, H₁₄), 2.47-2.40 (m, 1H, H₁₀), 2.28-2.12 (m, 2H, H₂), 2.04-1.92 (m, 2H, 1×H₄, 1×H₉), 1.87 (dq, J=14.4, 2.8 Hz, 1H, 1×H₈), 1.80 (dd, J=15.6, 7.6 Hz, 1H, 1×H₁₃), 1.74-1.60 (m, 2H, 1×H₁, 1×OH), 1.59-1.52 (m, 3H, 1×H₆, 1×H₇, 1×OH), 1.51-1.42 (m, 2H, 1×H₁, 1×H₇), 1.42-1.32 (m, 4H, 3×H₁₅, 1×H₉), 1.30-1.23 (m, 1H, 1×H₁₃), 1.18 (td, J=14.0, 4.4 Hz, 1H, 1×H₈), 1.02 (s, 3H, H₁₈), 0.95 (d, J=7.2 Hz, 3H, H₁₇), 0.89 (t, J=7.6 Hz, 3H, H₂₀). ¹³C NMR (100 MHz, CDCl₃) δ 217.9 (C), 137.9 (C), 128.4 (CH), 127.8 (2×CH), 96.9 (CH₂), 82.6 (CH), 70.8 (CH₂), 64.9 (CH), 62.8 (CH₂), 59.6 (CH), 45.2 (C), 43.5 (CH), 42.8 (C), 41.8 (CH₂), 40.7 (C), 36.0 (CH), 34.6 (CH₂), 34.2 (CH₂), 30.7 (CH₂), 25.3 (CH₂), 21.2 (CH₂) 16.7 (CH₃), 13.9 (CH₃), 12.0 (CH₃), 8.1 (CH₃). IR (ATR-FTIR), cm⁻¹: 3274 (br w), 2952 (m), 2878 (m), 1733 (m), 1458 (w), 1384 (w), 1161 (w), 1082 (w), 1025 (s), 966 (m), 736 (w), 698 (w). HRMS-ESI (m/z): [M+H]⁺ calcd for C₂₈H₄₃O₅, 459.3110; found, 459.3109. [α]_{D}²⁵=+51° (c=0.25, CHCl₃).

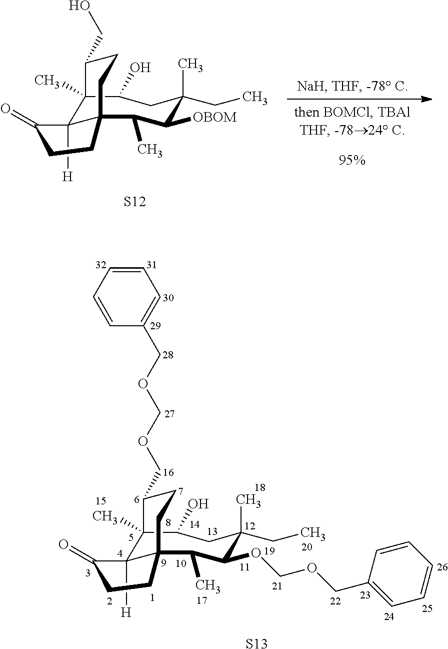

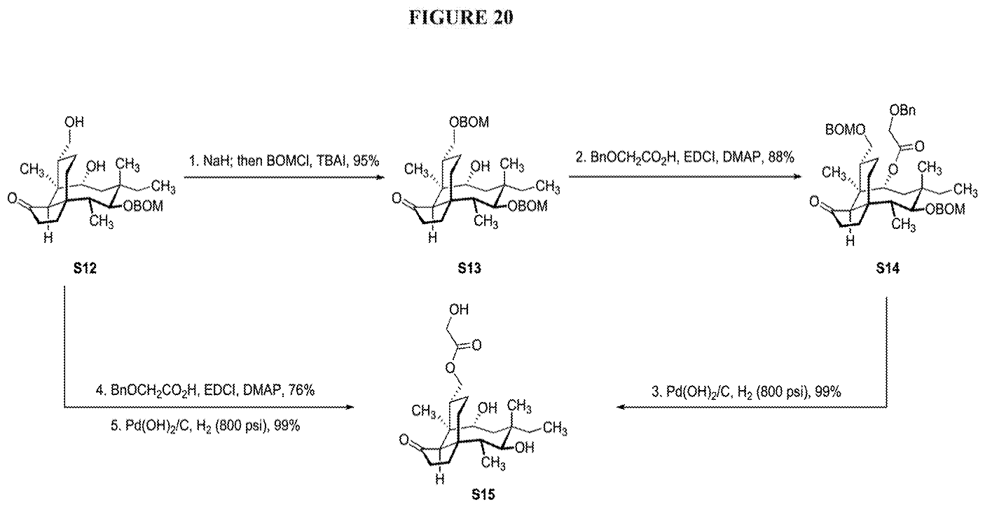

### Synthesis of bis(benzyloxymethyl)ether S13 (FIG. 20, Scheme S3)

Dry sodium hydride (8.4 mg, 350 μmol, 3.30 equiv) was added to a 4-mL vial in the glovebox. The vial was sealed with a septum and the sealed vial was removed out of the glovebox. Tetrahydrofuran (300 μL) was added to the vial containing sodium hydride and the resulting suspension was cooled to −78° C. A separate 4-mL vial was charged with the diol S12 [48.6 mg, 106 μmol, 1 equiv, dried by azeotropic distillation with benzene (3×500 μL)] and tetrahydrofuran (400 μL). The resulting diol solution was added dropwise via syringe to the cooled sodium hydride suspension at −78° C. The vial containing starting material was rinsed with tetrahydrofuran (3×100 μL) and the combined rinses were added dropwise via syringe to the reaction vessel at −78° C. The resulting suspension was stirred for 15 min at −78° C. Benzyl chloromethyl ether (17.7 μL, 127 μmol, 1.20 equiv) was added dropwise via syringe to the reaction mixture at −78° C. The resulting mixture was allowed to warm up over 2 h to 24° C. Tetrabutylammonium iodide (3.9 mg, 10.6 μmol, 0.100 equiv) was added to the warmed reaction vessel and the resulting mixture was stirred for 18 h at 24° C. The product mixture was diluted sequentially with ether (5.0 mL) and saturated aqueous ammonium chloride solution (1.0 mL). The diluted product mixture was transferred to a separatory funnel that had been charged with a mixture of ether and pentane (1:1, v/v, 30 mL). The layers that formed were separated and the organic layer was washed with water (3×2.0 mL). The washed organic layer was dried over sodium sulfate. The dried solution was filtered and the filtrate was concentrated to dryness. The residue obtained was purified by automated flash-column chromatography (eluting with hexanes initially, grading to 40% ether-hexanes, linear gradient) to afford the bis(benzyloxymethyl)ether S13 as an amorphous white solid (58.2 mg, 95%).

R_{f}=0.50 (20% ethyl acetate-hexanes; UV, PAA, CAM). ¹H NMR (400 MHz, CDCl₃) δ 7.38-7.30 (m, 10H, 2×H₂₄, 2×H₂₅, 1×H₂₆, 2×H₃₀, 2×H₃₁, 1×H₃₂), 4.80-4.74 (m, 4H, 2×H₂₁, 2×H₂₇), 4.67 (s, 2H, H₂₂), 4.59 (s, 2H, H₂₈), 4.35 (d, J=7.2 Hz, 1H, H₁₁), 4.06 (br s, 1H, OH), 3.90 (d, J=9.6 Hz, 1H, 1×H₁₆), 3.52 (dd, J=10.4, 4.0 Hz, 1H, 1×H₁₆), 3.33 (d, J=6.4 Hz, 1H, H₁₄), 2.47-2.40 (m, 1H, H₁₀), 2.29-2.12 (m, 2H, H₂), 2.02 (s, 1H, H₄), 1.99-1.85 (m, 2H, 1×H₈, 1×H₁₉), 1.78-1.68 (m, 3H, 1×H₁, 1×H₆, 1×H₁₃), 1.62-1.55 (m, 1H, 1×H₇), 1.51-1.45 (m, 2H, 1×H₁, 1×H₇), 1.42-1.32 (m, 4H, 3×HIS, 1×H₁₉), 1.32 (app d, 1H, 1×H₁₃), 1.17 (td, J=13.6, 3.6 Hz, 1H, 1×H₈), 1.05 (s, 3H, H₁₈), 0.96 (d, J=7.2 Hz, 3H, H₁₇), 0.91 (t, J=7.2 Hz, 3H, H₂₀). ¹³C NMR (100 MHz, CDCl₃) δ 217.9 (C), 137.9 (C), 137.3 (C), 128.4 (CH), 128.3 (CH), 127.8 (CH), 127.7 (CH), 127.6 (CH), 127.6 (CH), 96.8 (CH₂), 94.8 (CH₂), 82.8 (CH), 70.7 (CH₂), 69.9 (CH₂), 68.8 (CH₂), 64.5 (CH), 59.7 (CH), 45.1 (C), 42.8 (C), 42.4 (CH), 41.2 (CH₂), 40.6 (C), 36.0 (CH), 34.5 (CH), 34.2 (CH₂) 30.7 (CH₂), 25.3 (CH₂), 21.9 (CH₂), 16.6 (CH₃), 14.0 (CH₃), 12.0 (CH₃), 8.1 (CH₃). IR (ATR-FTR), cm⁻¹: 2932 (w), 2878 (w), 1735 (m), 1454 (m), 1384 (w), 1202 (w), 1110 (s), 1025 (s), 959 (m), 734 (s), 695 (s). HRMS-ESI (m/z): [M+H]⁺ calcd for C₃₆H₅₁O₆, 579.3686; found, 579.3688. [α]_{D}²⁵=+32° (c=0.10, CHCl₃).

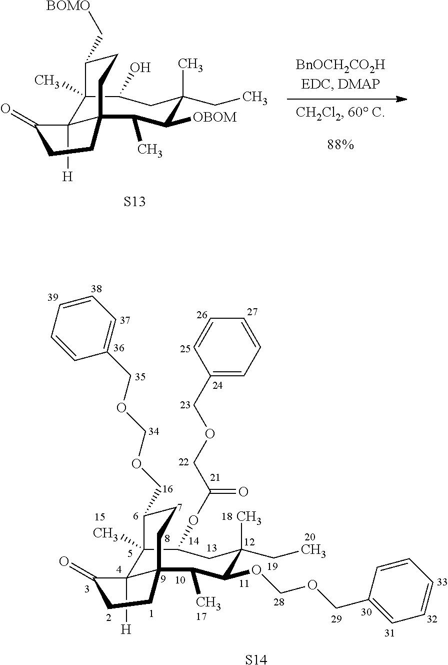

### Synthesis of tris(benzyl)ether S14 (FIG. 20. Scheme S3)

A 4-mL vial was charged with the bis(benzyloxymethylenoxy)ether S13 (29.3 mg, 50.6 μmol, 1 equiv) and benzyloxyacetic acid (18.0 μL, 127 μmol, 2.50 equiv). Benzene (500 μL) was added to the vial. The solution was concentrated to dryness. This process was repeated twice. The reaction vessel was evacuated and refilled using a balloon of argon. This process was repeated twice. Dichloromethane (300 μL), 1-ethyl-3-(3-dimethylaminopropyl)carbodiimide hydrochloride (32.0 mg, 167 μmol, 3.30 equiv), and 4-dimethylaminopyridine (20.4 mg, 167 μmol, 3.30 equiv) were added sequentially to the reaction vessel at 24° C. The vial was sealed and the sealed vial was placed in an oil bath that had been previously heated to 60° C. The reaction mixture was stirred and heated for 1 h at 60° C. The product mixture was allowed to cool over 30 min to 24° C. The cooled product mixture was concentrated to dryness. The residue obtained was purified by automated flash-column chromatography (eluting with hexanes initially, grading to 40% ether-hexanes, linear gradient) to afford the tris(benzyl)ether S14 as a clear oil (32.4 mg, 88%).

R_{f}=0.55 (40% ether-hexanes; UV, PAA, CAM). ¹H NMR (400 MHz, CDCl₃) δ 7.33-7.18 (m, 15H, 2×H₂₅, 2×H₂₆, 1×H₂₇, 2×H₃₁, 2×H₃₂, 1×H₃₃, 2×H₃₇, 2×H₃₈, 1×H₃₉), 5.73 (d, J=8.2 Hz, 1H, H₁₄), 4.74 (dd, J=11.6, 7.4 Hz, 2H, H₂₂), 4.63-4.61 (m, 2H, H₂), 4.61-4.57 (m, 2H, H₃₄), 4.57-4.53 (m, 2H, H₂₉), 4.47 (br s, 2H, H₃₅), 3.95 (dd, J=24.0, 16.0 Hz, 2H, H₂), 3.62 (d, J=9.2 Hz, 1H, 1×H₁₆), 3.66 (d, J=6.0 Hz, 1H, H₁), 2.87 (t, J=9.2 Hz, 1H, 1×H₁₆), 2.53-2.46 (m, 1H, H₁₀), 2.24-2.08 (m, 2H, H₂), 2.03 (s, 1H, H₄), 1.85-1.48 (m, 9H, 2×H₁, 1×H₆, 2×H₇, 1×H₈, 1×H₁₃, 2×H₁₉), 1.42 (s, 3H, H₁₅), 1.30 (d, J=16.8 Hz, 1H, 1×H₁₃), 1.13-1.06 (m, 1H, 1×H₈), 0.96-0.89 (m, 6H, 3×H₁₇, 3×H₁₈), 0.74 (t, J=7.4 Hz, H₂₀). ¹³C NMR (100 MHz, CDCl₃) δ 217.0 (C), 169.6 (C), 138.1 (C), 137.5 (C), 128.7 (CH), 128.7 (CH), 128.2 (CH), 128.2 (CH), 128.2 (C), 128.0 (CH), 127.9 (CH), 127.8 (CH), 97.2 (CH₂), 94.9 (CH₂), 85.3 (CH), 73.6 (CH₂), 71.0 (CH₂), 69.5 (CH₂), 69.2 (CH), 68.7 (CH₂), 68.2 (CH₂) 59.0 (CH), 45.4 (C), 43.4 (CH), 41.8 (C), 41.7 (C), 40.8 (CH₂), 35.5 (CH), 34.8 (CH₂), 30.2 (CH₂), 27.0 (CH), 26.6 (CH₃), 25.5 (CH₂), 22.8 (CH), 21.9 (CH₂) 15.4 (CH₃), 12.3 (CH₃), 8.5 (CH₃). IR (ATR-FTIR), cm⁻¹: 2933 (w), 1774 (w), 1734 (m), 1454 (m), 1111 (s), 1059 (m), 1026 (s), 937 (m), 844 (w), 734 (s), 696 (w), 606 (w). HRMS-ESI (m/z): [M+H]⁺ calcd for C₄₅H₅₉O₈, 727.4210; found, 727.4204. [α]_{D}⁵=+29° (c=0.10, CHCl₃).

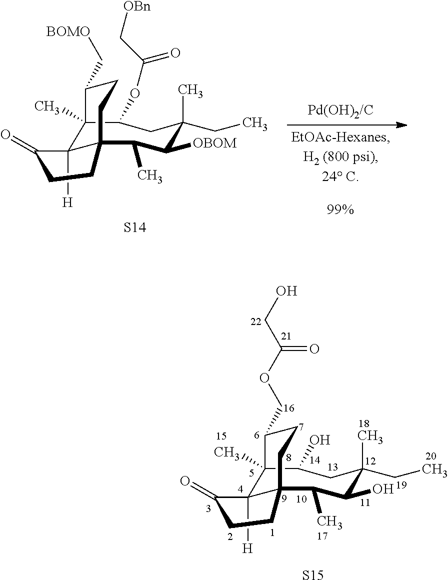

### Global Deprotection of the tris(benzyl)ether S14 with Concomitant Acyl Migration (FIG. 20, Scheme S3)

A 4-mL vial was charged with the tris(benzyl)ether S14 (4.7 mg, 6.5 μmol, 1 equiv). Benzene (200 μL) was added to the vial. The solution was concentrated to dryness. This process was repeated twice. The reaction vessel was evacuated and refilled using a balloon of nitrogen. This process was repeated twice. Ethyl acetate (50 μL), hexanes (250 μL), and Pearlman's catalyst (20 wt. % loading, 1.8 mg, 3.6 μmol, 0.400 equiv) were added sequentially to the reaction vessel at 24° C. The vial was placed in a stainless steel hydrogenation apparatus. The apparatus was purged with dihydrogen by pressurizing to 50 psi and venting three times. The vessel was pressurized with dihydrogen (800 psi), sealed, and the reaction mixture was stirred for 18 h at 24° C. The apparatus was depressurized by slowly venting the dihydrogen. The product mixture was filtered through a pad of celite and the pad was rinsed with ether (50 mL). The filtrates were collected and combined and the combined filtrates were concentrated to afford 12-epi-16-hydroxy-19,20-dihydropleuromutilin hydroxyacetate (S15) as a colorless clear film (2.8 mg, 99%).

R_{f}=0.50 (20% ethyl acetate-hexanes; PAA, CAM). ¹H NMR (600 MHz, CD₂Cl₂) δ 4.39 (dd, J=11.4, 3.0 Hz, 1H, 1×H₁₆), 4.30 (t, J=7.5 Hz, 1H, H₁₁), 4.09 (d, J=4.8 Hz, 2H, H₂₂), 4.04 (t, J=10.5 Hz, 1H, 1×H₁₆), 3.49 (t, J=6.0 Hz, 1H, H₁), 2.36 (t, J=5.4 Hz, 1H, C11-OH), 2.31-2.21 (m, 2H, 1×H₂, 1×H₁₀), 2.18-2.10 (m, 1H, 1×H₂), 2.04 (dd, J=13.8, 7.8 Hz, 1H, 1×H₁₃), 1.99 (s, 1H, H₄), 1.84-1.77 (m, 2H, 1×H₆, 1×H₈), 1.67-1.59 (m, 2H, 1×H₁, 1×H₁₉), 1.58-1.55 (m, 2H, 1×H₇, 1×C14-OH), 1.53-1.46 (m, 2H, 1×H₇, 1×H₁₉), 1.40-1.32 (m, 5H, 1×H₁, 3×H₁₅, 1×C22-OH), 1.10 (td, J=13.8, 4.2 Hz, 1H, 1×H₈), 1.06 (app d, 1H, 1×H₁₃), 0.98 (s, 3H, H₁₈), 0.91 (d, J=7.2 Hz, 3H, H₁₇), 0.88 (t, J=7.5 Hz, 3H, H₂A). ¹³C NMR (150 MHz, CD₂Cl₂) δ 217.2 (C), 174.0 (C), 72.3 (CH), 68.5 (CH₂), 66.2 (CH), 61.1 (CH₂), 59.1 (CH), 45.6 (C), 44.7 (CH₂), 42.5 (C), 42.5 (CH), 40.7 (C), 35.7 (CH), 35.3 (CH₂), 34.8 (CH₂), 30.2 (CH₂), 25.7 (CH₂), 22.7 (CH₂), 17.6 (CH₃), 13.9 (CH₃), 11.6 (CH₃), 8.2 (CH₃). IR (ATR-FTIR), cm⁻¹: 3434 (br m), 2957 (m), 2879 (m), 1731 (s), 1462 (w), 1381 (w), 1284 (w), 1221 (m), 1095 (m), 1030 (w), 1000 (m), 954 (w), 711 (w). HRMS-ESI (m/z): [M+H]⁺ calcd for C₂₂H₃₇O₈, 397.2590; found, 397.2599.

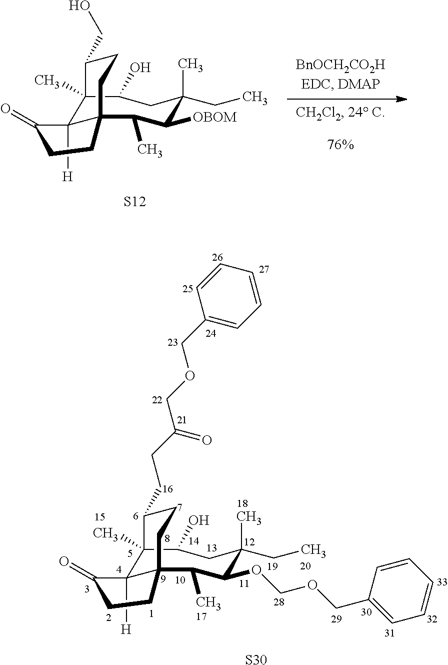

### Synthesis of bis(benzyl)ether S30 (FIG. 20, Scheme 53)

A 4-mL vial was charged with the diol S12 (9.3 mg, 20.3 μmol, 1 equiv) and benzyloxyacetic acid (3.5 μL, 24.3 μmol, 1.20 equiv). Benzene (200 μL) was added to the vial. The solution was concentrated to dryness. This process was repeated twice. The reaction vessel was evacuated and refilled using a balloon of argon. This process was repeated twice. Dichloromethane (200 μL), 1-ethyl-3-(3-dimethylaminopropyl)carbodiimide hydrochloride (5.8 mg, 30.4 μmol, 1.50 equiv), and 4-dimethylaminopyridine (0.5 mg, 4.1 μmol, 0.200 equiv) were added sequentially to the reaction vessel at 24° C. The reaction mixture was stirred for 90 min at 24° C. The product mixture was concentrated to dryness. The residue obtained was purified by automated flash-column chromatography (eluting with hexanes initially, grading to 40% ether-hexanes, linear gradient) to afford the bis(benzyl)ether S30 as a colorless clear film (9.4 mg, 76%).

R_{f}=0.55 (40% ether-hexanes; UV, PAA, CAM). ¹H NMR (400 MHz, CDCl₃) δ 7.37-7.29 (m, 10H, 2×H₂₅, 2×H₂₆, 1×H₂₇, 2×H₃₁, 2×H₃₂, 1×H₃₃), 4.76 (s, 2H, H₂₂), 4.68-4.60 (m, 4H, 2×H₂₃, 2×H₂₈), 4.39 (dd, J=11.2, 2.8 Hz, 1H, 1×H₁₆), 4.33 (d, J=7.6 Hz, 1H, H₁₁), 4.15-4.00 (m, 3H, 1×H₁₆, 2×H₂₉), 3.31 (d, J=6.0 Hz, 1H, H₄), 2.42-2.29 (m, 1H, H₁₀), 2.26-2.11 (m, 2H, H₂), 1.94 (s, 1H, H₄), 1.89-1.75 (m, 3H, 1×H₆, 1×H₈, 1×H₁₃), 1.74-1.66 (m, 1H, 1×H₁), 1.64-1.59 (m, 3H, 2×H₁₉, 1×OH), 1.56-1.42 (m, 3H, 1×H₁, 2×H₇), 1.39 (s, 3H, H₁₅), 1.20-1.06 (m, 2H, 1×H₈, 1×H₁₃), 0.99 (s, 3H, H₁₈), 0.94 (d, J=7.2 Hz, 3H, H₁₇), 0.87 (t, J=7.6 Hz, 3H, H₂₀). ¹³C NMR (100 MHz, CDCl₃) δ 217.2 (C), 170.3 (C), 137.8 (C), 137.1 (C), 128.5 (CH), 128.4 (CH), 128.0 (CH), 128.0 (CH), 127.7 (CH), 127.7 (CH), 96.8 (CH₂), 82.3 (CH), 73.3 (CH₂), 70.8 (CH₂), 67.3 (CH₂), 66.9 (CH₂), 65.6 (CH), 58.9 (CH), 44.8 (C), 43.7 (CH₂), 42.2 (C), 41.8 (CH), 40.9 (C), 35.8 (CH), 34.4 (CH₂), 34.1 (CH₂), 29.9 (CH₂), 25.3 (CH₂), 22.0 (CH₂), 16.7 (CH₃), 13.6 (CH₃), 11.9 (CH₂), 8.1 (CH₃). IR (ATR-FTIR), cm⁻¹: 3514 (br w), 2955 (m), 2880 (m), 1734 (s), 1497 (w), 1455 (m), 1383 (w), 1282 (w), 1209 (m), 111 (m), 1025 (s), 736 (m), 698 (m). HRMS-ESI (m/z): [M+H]⁺ calcd for C₃₇H₅₁O₇, 607.3635; found, 607.3630. [α]_{D}²⁵=+33° (c=0.10, CHCl₃).

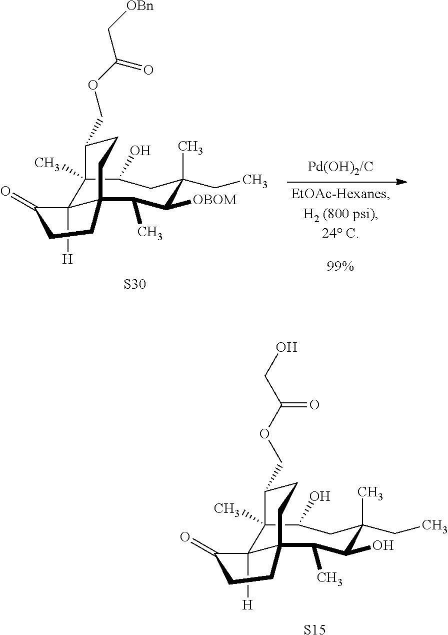

### Synthesis of 12-epi-16-hydroxy-19,20-dihydropleuromutilin hydroxyacetate (S15, FIG. 20, Scheme S3)

A 4-mL vial was charged with the bis(benzyl)ether S30 (4.0 mg, 6.7 μmol, 1 equiv). Benzene (200 μL) was added to the vial. The solution was concentrated to dryness. This process was repeated twice. The reaction vessel was evacuated and refilled using a balloon of nitrogen. This process was repeated twice. Ethyl acetate (50 μL), hexanes (250 μL), and Pearlman's catalyst (20 wt. % loading, 1.8 mg, 3.6 μmol, 0.400 equiv) were added sequentially to the reaction vessel at 24° C. The vial was placed in a stainless steel hydrogenation apparatus. The apparatus was purged with dihydrogen by pressurizing to 50 psi and venting three times. The vessel was pressurized with dihydrogen (800 psi), sealed, and the reaction mixture was stirred for 18 h at 24° C. The apparatus was depressurized by slowly venting the dihydrogen. The product mixture was filtered through a pad of celite and the pad was rinsed with ether (50 mL). The filtrates were collected and combined and the combined filtrates were concentrated to afford 12-epi-16-hydroxy-19,20-dihydropleuromutilin hydroxyacetate (S15) as a colorless clear film (2.7 mg, 99%).

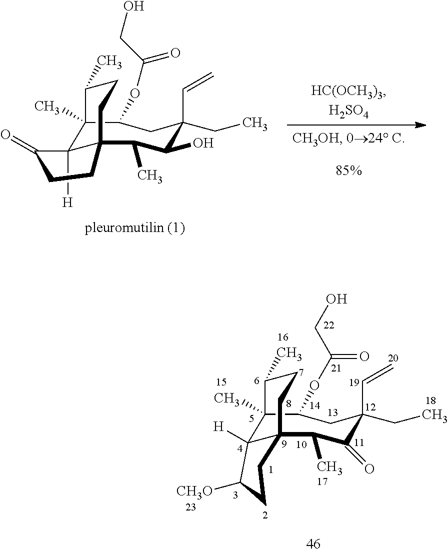

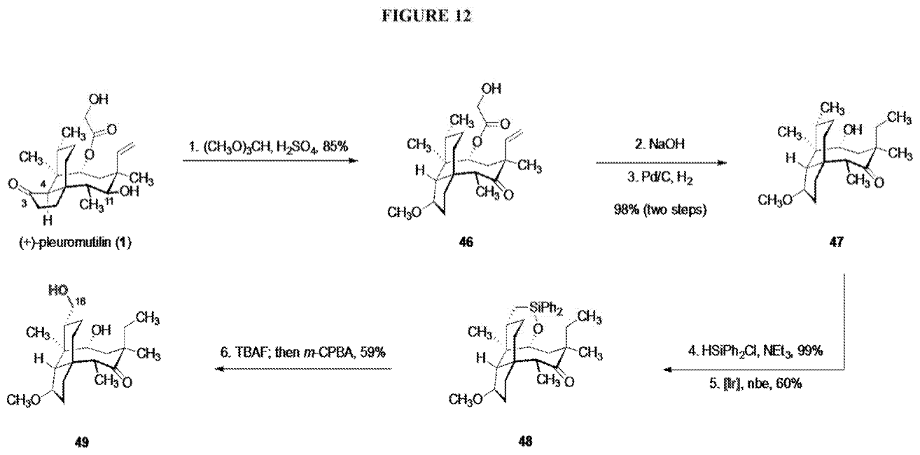

### Synthesis of 4-epi-pleuromutilin (46, FIG. 12. Scheme 12)

This experiment was adapted from the work of Berner and co-woerks.⁴ Sulfuric acid (264 μL) was added slowly dropwise into a solution of pleuromutilin (1, 1.00 g, 2.64 mmol, 1 equiv) and trimethyl orthoformate (1.59 mL) in methanol (16 mL) at 0° C. using an ice bath. The reaction mixture was stirred for 15 min at 0° C. then the ice bath was removed. The reaction mixture was allowed to warm up over 30 min to 24° C. The resulting mixture was stirred for 24 h at 24° C. A saturated aqueous sodium carbonate solution (30 mL) was added dropwise via syringe to the product mixture. The resulting mixture was transferred to a separatory funnel that had been charged with dichloromethane (50 mL). The layers were separated and the aqueous layer was extracted with dichloromethane (3×50 mL). The organic layers were combined and dried over sodium sulfate. The dried solution was filtered and the filtrate was concentrated. The residue obtained was purified by automated flash-column chromatography (eluting with hexanes initially, grading to 30% ethyl acetate-hexanes, linear gradient) to afford 4-epi-pleuromutilin (46) as an amorphous white solid (879 mg, 85%).

R_{f}=0.48 (25% ethyl acetate-hexanes; PAA, CAM). ¹H NMR (400 MHz, CDCl₃) δ 6.64 (dd, J=17.6, 10.8 Hz, 1H, H₁₉), 5.87 (d, J=6.4 Hz, 1H, H₄), 5.32 (d, J=10.8 Hz, 1H, 1×H₂₀), 5.03 (d, J=17.6 Hz, 1H, 1×H₂₀), 4.11 (ddd, J=15.0, 11.2, 3.6 Hz, 2H, H₂₂), 3.45 (ddd, J=8.8, 5.4, 3.6 Hz, 1H, H₃), 3.22 (s, 3H, H₂₃), 2.91 (q, J=6.4 Hz, 1H, H₁₀), 2.49 (dd, J=15.6, 10.4 Hz, 1H, 1×H₁₃), 2.40 (t, J=5.4 Hz, 1H, OH), 2.20 (td, J=9.2, 2.4 Hz, 1H, 1×H₈), 2.04-1.98 (m, 2H, 1×H₂, 1×H₇), 1.73 (d, J=11.2 Hz, H₄), 1.60-1.52 (m, 2H, 1×H₁, 1×H₁₃), 1.47 (td, J=11.2, 3.6 Hz, 1H, 1×H₁), 1.37-1.28 (m, 1H, H₆), 1.26-1.41 (m, 8H, 1×H₂, 1×H₈, 3×H₁₅, 3×H₁₈), 1.08 (td, J=13.6, 4.8 Hz, 1H, 1×H₇), 0.99 (d, J=6.4 Hz, 3H, H₁₆), 0.79 (d, J=6.8 Hz, 3H, H₁₇). ¹³C NMR (100 MHz, CDCl₃) δ 215.1 (C), 172.5 (C), 140.0 (CH), 118.4 (CH₂), 83.0 (CH), 73.5 (CH), 64.1 (CH), 61.3 (CH₂), 56.8 (CH₃), 53.8 (C), 47.5 (C), 45.1 (CH), 44.9 (CH), 44.3 (CH₂), 43.2 (C), 40.2 (CH₂), 30.6 (CH₂), 29.4 (CH₂), 28.6 (CH₂), 25.5 (CH₃), 20.2 (CH₃), 16.4 (CH₃), 15.7 (CH₃). IR (ATR-FTIR), cm⁻¹: 3432 (br w), 2978 (m), 2928 (m), 2865 (w), 1735 (m), 1699 (m), 1456 (m), 1373 (w), 1282 (w), 1230 (m), 1098 (s), 1065 (w), 992 (m), 971 (m), 733 (m). HRMS-ESI (m/z): [M+H]⁺ calcd for C₂₃H₃₇O₅, 393.2642; found, 393.2643. [α]_{D}²⁵=−47° (c=1.00, CHCl₃).

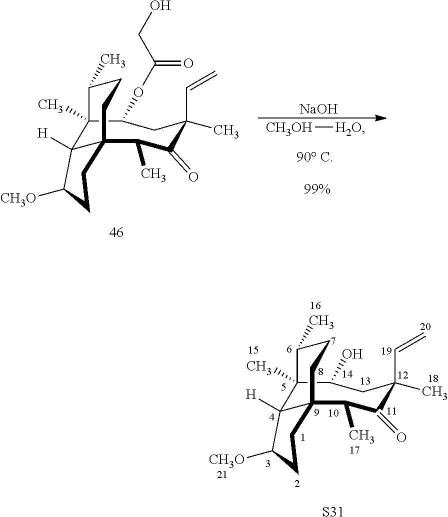

### Synthesis of 4-epi-mutilin (31 FIG. 12. Scheme 12)

Water (3.2 mL) and an aqueous sodium hydroxide solution (50% w/w, 445 μL) were added dropwise via syringe to a solution of 4-epi-pleuromutilin (46, 879 mg, 2.24 mmol, 1 equiv) in ethanol (5.1 mL) in a 25-mL round-bottomed flask fitted with a reflux condenser at 24° C. The reaction vessel was placed in an oil bath that had been previously heated to 90° C. The reaction mixture was stirred and heated for 4 h at 90° C. The resulting mixture was allowed to cool over 30 min to 24° C. The product mixture was transferred to a separatory funnel that had been charged with dichloromethane (50 mL). The layers that formed were separated and the aqueous layer was extracted with dichloromethane (3×20 mL). The organic layers were combined and the combined organic layers were dried over sodium sulfate. The dried solution was filtered and the filtrate was concentrated. The residue obtained was purified by automated flash-column chromatography (eluting with hexanes initially, grading to 33% ethyl acetate-hexanes, linear gradient) to afford 4-epi-mutilin (S31) as an amorphous white solid (751 mg, 99%).

R_{f}=0.48 (25% ethyl acetate-hexanes; PAA, CAM). ¹H NMR (400 MHz, CDCl₃) δ 6.00 (dd, J=17.6, 10.8 Hz, 1H, H₁₉), 5.26 (d, J=10.8 Hz, 1H, 1×H₂₀), 5.24 (d, J=17.6 Hz, 1H, 1×H₂₀), 4.63 (dd, J=9.2, 5.6 Hz, 1H, H₁₄), 3.47 (ddd, J=13.6, 8.0, 5.2 Hz, 1H, H₃), 2.94 (s, 3H, H₂₁), 2.92 (q, J=6.5 Hz, 1H, H₁₀), 2.42 (dd, J=15.2, 9.2 Hz, 1H, 1×H₁₃), 2.18 (td, J=9.2, 2.4 Hz, 1H, 1×H₈), 2.01-1.96 (m, 2H, 1×H₂, 1×H₇), 1.81 (d, J=15.2 Hz, 1H, 1×H₁₃), 1.71 (d, J=11.6 Hz, 1H, H₄), 1.60-1.50 (m, 1H, 1×H₁₃), 1.49-1.42 (m, 1H, 1×H₁), 1.39-1.29 (m, 1H, H₆), 1.27-1.18 (m, 1H, 1×H₈), 1.17-1.13 (m, 7H, 1×H₂, 3×H₁₅, 3×H₁₈), 1.09-1.03 (m, 4H, 1×H₇, 3×H₁₆), 0.97 (d, J=6.8 Hz, 3H, H₁₇). ¹³C NMR (100 MHz, CDCl₃) δ 216.8 (C), 140.6 (CH), 117.0 (CH₂), 83.2 (CH₃), 69.1 (CH), 64.2 (C), 56.8 (CH), 54.5 (C), 47.7 (C), 45.4 (CH), 44.8 (CH₂), 44.2 (CH), 44.1 (CH), 40.5 (CH₂), 30.6 (CH₂), 29.4 (CH₂), 28.8 (CH₂), 25.8 (CH₃), 18.8 (CH₃), 17.9 (CH₄), 15.2 (CH₃). IR (ATR-FTIR), cm⁻¹: 3534 (br w), 2974 (m), 2924 (m), 2662 (m), 1696 (m), 1456 (m), 1373 (w), 1130 (w), 1111 (w), 1098 (m), 986 (m), 911 (m), 730 (s), 961 (w), 647 (w). HRMS-ESI (m/z): [M+H]⁺ calcd for C₂₁H₃₅O₃, 335.2586; found, 335.2590. [α]_{D}²⁵=−78° (c=1.00, CHCl₃).

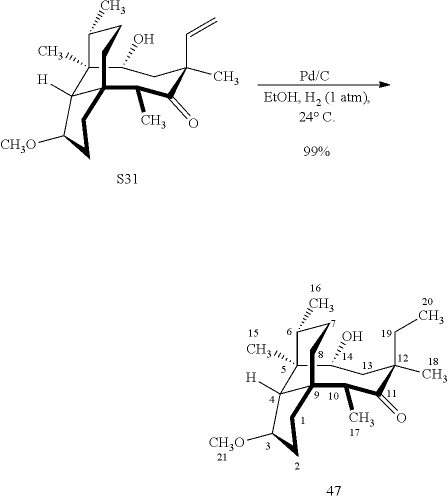

### Synthesis of 4-epi-mutilin (47, FIG. 12, Scheme 12)

Palladium on carbon (5 wt. % loading, 239 mg, 112 μmol, 0.05 equiv) was added to a solution of 4-epi-mutilin (S31, 749 mg, 2.24 mmol, 1 equiv) in ethanol (10 mL) at 24° C. The reaction vessel was evacuated and re-filled using a balloon of dihydrogen. This process was repeated four times. The reaction mixture was stirred for 12 h at 24° C. The product mixture was filtered through a short column of celite and the short column was rinsed with dichloromethane (250 mL). The filtrates were combined and the combined filtrates were concentrated to afford 4-epi-19,20-dihydromutilin (47) as an amorphous white solid (751 mg, 99%).

R_{f}=0.46 (25% ethyl acetate-hexanes; PAA, CAM). ¹H NMR (400 MHz, CDCl₃) δ 4.59 (dd, J=10.0, 5.6 Hz, 1H, H₁₄), 3.49-3.42 (m, 1H, H₁₃), 3.21 (s, 3H, H₂₁), 3.05 (q, J=6.8 Hz, 1H, H₁₀), 2.35 (dd, J=15.2, 9.6 Hz, 1H, 1×H₁₃), 2.18 (td, J=10.8, 3.6 Hz, 1H, 1×H₈), 2.03-1.85 (m, 3H, 1×H₂, 1×H₇, 1×H₁₉), 1.68 (d, J=11.6 Hz, 1H, 1×H₁₃), 1.66-1.52 (m, 3H, 1×H₁, 1×H₄, 1×H₁₉, 1×OH), 1.51-1.43 (m, 2H, 1×H₁, 1×H₈), 1.38-1.28 (m, 1H, H₆), 1.26-1.16 (m, 1H, 1×H₇), 1.14-1.10 (m, 4H, 1×H₂, 3×H₁₉), 1.07 (d, J=6.8 Hz, 3H, H₁₆), 1.02 (s, 3H, H₁₈), 0.82 (t, J=7.6 Hz, 3H, H₂). ¹³C NMR (100 MHz, CDCl₃) δ 219.5 (C), 83.1 (CH₁), 68.4 (CH), 64.1 (C), 56.8 (CH), 51.5 (C), 47.7 (CH), 45.6 (CH), 45.3 (CH₂), 44.3 (C), 41.8 (CH), 40.6 (CH₂), 30.4 (CH₂), 30.2 (CH), 29.4 (CH₂), 28.9 (CH₂) 22.7 (CH₃), 18.9 (CH₃), 17.9 (CH₃), 14.0 (CH₃), 8.7 (CH₃). IR (ATR-FTIR), cm⁻¹: 3520 (br w), 2973 (m), 2929 (m), 2862 (m), 1689 (m), 1456 (m), 1375 (w), 1246 (w), 1099 (s), 1018 (m), 986 (s), 911 (m), 733 (s), 668 (w). HRMS-ESI (m/z): [M+H]⁺ calcd for C₂₁H₃₇O₃, 337.2743; found, 337.2739. [α]_{D}²⁵=−80° (c=0.50, CHCl₃).

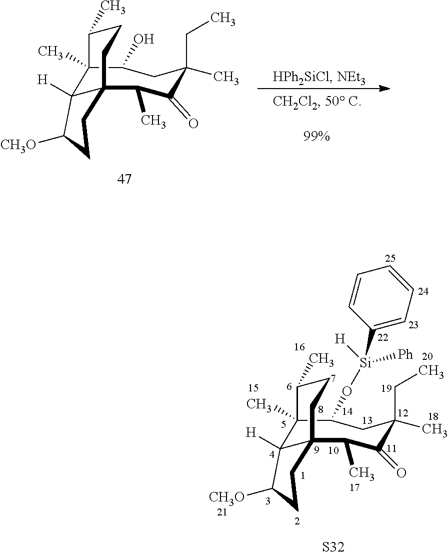

### Synthesis of Silane 532 (FIG. 12, Scheme 12)

A 25-mL round-bottomed flask fused to a Teflon-coated valve was charged with 4-epi-19,20-mutilin (47, 751 mg, 2.24 μmol, 1 equiv). Benzene (2.5 mL) was added and the solution was concentrated to dryness. This process was repeated twice. The reaction vessel was evacuated and refilled using a balloon of argon. This process was repeated two times. Dichloromethane (8.0 mL), triethylamine (1.25 mL, 8.96 mmol, 4.00 equiv), and (chloro)diphenylsilane (877 μL, 4.48 mmol, 2.00 equiv, 95% purity) were added sequentially to the reaction vessel. The vessel was sealed and the sealed vessel was placed in an oil bath that had been previous heated to 50° C. The reaction was stirred and heated at 50° C. for 20 min. The reaction vessel was allowed to immediately cool to 24° C. with an ice bath. The product mixture was diluted sequentially with pentane (5.0 mL) and an aqueous potassium phosphate buffer solution (pH 7, 0.10 M, 2.5 mL). The diluted mixture was transferred to a separatory funnel and the layers formed were separated. The aqueous layer was extracted with dichloromethane (3×10 mL). The organic layers were combined and the combined organic layers were dried over sodium sulfate. The dried solution was filtered and the filtrate was concentrated to dryness. The residue obtained was purified by automated flash-column chromatography (eluting with hexanes initially, grading to 20% ether-hexanes, linear gradient) to afford silane S32 as an amorphous white solid (1.16 g, 99%).

R_{f}=0.50 (20% ether-hexanes; UV, PAA, CAM). ¹H NMR (600 MHz, C₆D₆) δ 7.79-7.72 (m, 4H, 4×H₂₄), 7.21-7.14 (m, 6H, 4×H₂₃, 2×H₂), 5.81 (s, 1H, Si—H), 4.98 (d, J=9.6 Hz, 1H, H₁₄), 3.62 (dt, J=13.8, 6.0 Hz, 1H, H₃), 3.08 (s, 3H, H₂₁), 2.86 (t, J=6.6 Hz, 1H, H₁₀), 2.63 (dd, J=15.6, 9.6 Hz, 1H, 1×H₁₃), 2.31 (td, J=10.2, 4.2 Hz, 1H, 1×H₂), 2.15-1.85 (m, 1H, 1×H₇), 1.84 (d, J=15.6 Hz, 1H, 1×H₆), 1.82-1.75 (m, 3H, 1×H₄, 1×H₇, 1×H₈), 1.68 (s, 3H, H₁₅), 1.45-1.36 (m, 1H, 1×H₁), 1.31-1.18 (m, 3H, 1×H₁, 2×H₁₉), 1.14 (d, J=6.6 Hz, 3H, H₁₆), 1.05 (dd, J=13.2, 6.6 Hz, 1H, 1×H₂), 1.02 (s, 3H, HIS), 0.89 (d, J=6.0 Hz, 3H, H₁), 0.86-0.79 (m, 2H, 1×H₈, 1×H₁₃), 0.58 (t, J=7.5 Hz, 3H, H₂₀). ¹³C NMR (150 MHz, C₆D₆) δ 217.2 (C), 135.4 (C), 135.0 (CH), 135.0 (CH), 134.9 (C), 134.7 (CH), 130.2 (CH), 83.1 (CH₃), 71.7 (CH), 64.1 (CH), 56.2 (CH₃), 51.7 (C), 47.4 (C), 46.4 (CH), 45.8 (C), 45.7 (C), 41.9 (CH), 40.5 (CH₂), 30.9 (CH₂), 30.2 (CH₂), 29.4 (CH₂), 28.9 (CH₂), 22.8 (CH), 20.5 (CH₃), 18.6 (CH), 13.5 (CH₃), 8.8 (CH₃). IR (ATR-FTIR), cm⁻¹: 2926 (w), 1689 (m), 1452 (m), 1429 (m), 1373 (w), 1113 (s), 1099 (s), 1048 (s), 1026 (s), 990 (m), 864 (s), 810 (s), 729 (s), 697 (s), 678 (s), 492 (m), 473 (m), 443 (m). HRMS-ESI (m/z): [M+Na]⁺ calcd for C₃₃H₄₆NaO₃Si, 541.3114; found, 541.3110. [α]_{D}²⁵=−67° (c=0.25, CHCl₃).

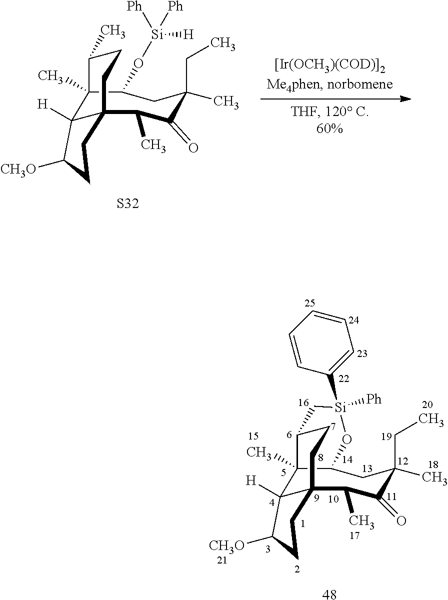

### Synthesis of Silacycle 48 (FIG. 12, Scheme 12)

This experiment was adapted from the work of Hartwig and co-workers.² A 25-mL pressure tube with a Teflon-coated valve was charged with 3,4,7,8-tetramethyl-1,10-phenanthroline (66.2 mg, 280 μmol, 12.5 mol %) and norbornene (316 mg, 3.36 mmol, 1.50 equiv) in the glovebox. A 4-mL vial was charged with silane S32 [1.16 g, 2.24 mmol, 1 equiv, dried by azeotropic distillation with benzene (3×5.0 mL)]. The vessel containing the silane was evacuated and refilled using a balloon of argon. This process was repeated two times. Tetrahydrofuran (1.5 mL) was transferred into the vessel containing the silane and the resulting solution was added to the vessel containing the ligand and norbornene in the glovebox. The vessel containing the silane was rinsed with tetrahydrofuran (3×500 μL) and the combined rinses were transferred to the reaction vessel.

Methoxy(cyclooctadiene)iridium(I) dimer (74.2 mg, 112 μmol, 5 mol %) was added to an oven-dried 4-mL vial. Tetrahydrofuran (500 μL) was added into the vial containing the catalyst and the resulting solution was transferred dropwise via syringe to the reaction vessel in the glovebox. The vial containing the catalyst was rinsed with tetrahydrofuran (3×500 μL) and the combined rinses were transferred into the reaction vessel. The reaction vessel was sealed and the reaction mixture was stirred for 1 h at 24° C. in the glovebox. The sealed reaction vessel was then removed from the glovebox and placed in an oil bath that had been preheated to 120° C. The reaction mixture was stirred and heated for 7 h at 120° C. The reaction vessel was allowed to cool over 30 min to 24° C. and the cooled product mixture was concentrated to dryness. The residue obtained was purified by automated flash-column chromatography (eluting with hexanes initially, grading to 15% ether-hexanes, linear gradient) to afford the silacycle 48 as an amorphous white solid (695 mg, 60%).

R_{f}=0.41 (20% ether-hexanes; UV, PAA, CAM). ¹H NMR (600 MHz, C₆D₆) δ 7.78-7.69 (m, 4H, 4×H₂₄), 7.27-7.17 (m, 6H, 4×H₂₃, 2×H₂₅), 5.09 (d, J=8.4 Hz, 1H, H₁₄), 4.32-4.30 (m, 1H, H₁), 3.62-3.58 (m, 1H, H₄), 2.80 (t, J=6.4 Hz, 1H, H₁₀), 2.54 (dd, J=15.0, 8.4 Hz, 1H, 1×H₁₃), 2.22-2.15 (m, 2H, 1×H₇, 1×H₈), 1.97 (d, J=15.0 Hz, 1H, 1×H₁₃), 1.81-1.63 (m, 5H, 1×H₁, 1×H₂, 1×H₆, 1×H₁₆, 1×H₁₉), 1.62 (s, 3H, H₁₅), 1.51-1.44 (m, 1H, 1×H₂), 1.35-1.22 (m, 2H, 1×H₇, 1×H₈), 1.16 (s, 3H, H₁₈), 1.03-0.97 (m, 1H, 1×H₁), 0.95-0.89 (m, 1H, 1×H₁₆), 0.80 (td, J=12.6, 5.4 Hz, 1H, 1×H₁₉), 0.74 (t, J=7.5 Hz, 3H, H₂₀), 0.70 (d, J=6.6 Hz, 3H, H₁₇). ¹³C NMR (150 MHz, C₆D₆) δ 217.5 (C), 136.8 (C), 136.3 (C), 134.3 (CH), 134.2 (CH), 130.0 (CH), 129.9 (CH), 82.7 (CH), 68.5 (CH), 63.5 (CH), 56.1 (CH₃), 51.4 (C), 48.0 (C), 47.5 (CH), 44.2 (CH₂), 42.9 (C), 42.7 (CH), 40.7 (CH₂), 32.1 (CH₂), 30.0 (CH₂), 29.4 (CH₂), 29.1 (CH₂), 22.9 (CH₃), 18.7 (CH₃), 14.4 (CH₃), 12.9 (CH₂), 8.6 (CH). IR (ATR-FTIR), cm⁻¹: 2926 (w), 1689 (m), 1452 (m), 1429 (m), 1373 (w), 1113 (s), 1099 (s), 1048 (s), 1026 (s), 990 (m), 864 (s), 810 (s), 729 (s), 697 (s), 678 (s), 492 (m), 473 (m), 443 (m). HRMS-ESI (m/z): [M+Na]⁺ calcd for C₃₃H₄₄NaO₃Si, 539.2957; found, 539.2952. [α]_{D}²⁵=−65° (c=0.25, CHCl₃).

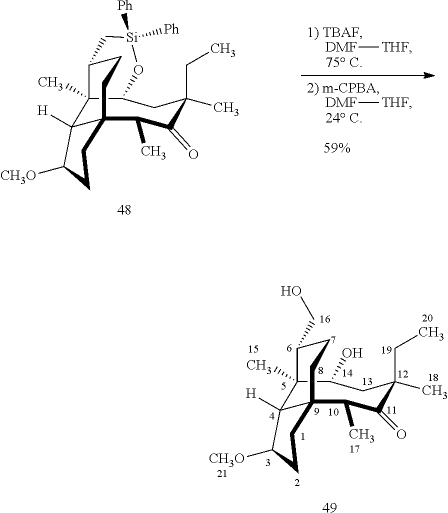

### Synthesis of Diol 49 (FIG. 12, Scheme 12)

A solution of tetrabutylammonium fluoride (1.0 M, 2.68 mL, 2.68 mmol, 2.00 equiv) in tetrahydrofuran was added dropwise via syringe to a solution of the silacycle 48 (695 mg, 1.34 μmol, 1 equiv) in N,N-dimethylformamide (8.0 mL) at 24° C. The reaction vessel was placed in an oil bath that had been previously heated to 75° C. The reaction mixture was stirred and heated for 5 min at 75° C. The resulting mixture was immediately cooled to 24° C. with an ice bath. Freshly recrystallized m-chloroperbenzoic acid (694 mg, 4.03 mmol, 3.00 equiv) was added to the reaction mixture at 24° C. The reaction mixture was stirred for 15 min at 24° C. The product mixture was diluted sequentially with ether (5.0 mL) and aqueous potassium phosphate buffer solution (pH 7, 0.10 M, 3.0 mL). The diluted product mixture was transferred to a separatory funnel that had been charged with a mixture of ether and pentane (1:1, v/v, 50 mL). The layers that formed were separated and the organic layer was washed with saturated aqueous sodium bicarbonate solution (3×10 mL). The washed organic layer was dried over sodium sulfate. The dried solution was filtered and the filtrate was concentrated to dryness. The residue obtained was purified by automated flash-column chromatography (eluting with hexanes initially, grading to 80% ethyl acetate-hexanes, linear gradient) to afford the diol 49 as an amorphous white solid (278 mg, 59%).

R_{f}=0.42 (75% ethyl acetate-hexanes; PAA, CAM). ¹H NMR (600 MHz, CDCl₃) δ 4.70 (d, J=9.0 Hz, 1H, H₁₄), 4.36 (br s, 1H, C16-OH), 4.06 (d, J=12.0 Hz, 1H, 1×H₁₆), 4.00 (br s, 1H, C14-OH), 3.48 (dd, J=12.0, 4.2 Hz, 1H, 1×H₁₆), 3.41 (ddd, J=13.8, 8.4, 5.4 Hz, 1H, H₃), 3.16 (s, 3H, H₂₁), 3.10 (q, J=6.6 Hz, 1H, H₁₀), 2.28 (dd, J=15.6, 9.0 Hz, 1H, 1×H₁₃), 2.14 (dd, J=13.8, 3.0 Hz, 1H, 1×H₂), 2.10-1.90 (m, 2H, 1×H₁, 1×H₁₉), 1.94-1.83 (m, 2H, 1×H₇, 1×H₈), 1.67-1.54 (m, 3H, 1×H₄, 1×H₈, 1×H₁₃), 1.45-1.39 (m, 1H, 1×H₁), 1.22-1.02 (m, 6H, 1×H₂, 1×H₆, 1×H₇, 3×H₁₅, 1×H₁₉), 0.99 (s, 3H, H₁₈), 0.94 (d, J=6.6 Hz, 3H, H₁₇), 0.77 (t, J=7.5 Hz, 3H, H₂₀). ¹³C NMR (150 MHz, CDCl₃) δ 219.6 (C), 83.1 (CH), 66.5 (CH), 64.5 (CH), 62.3 (CH₂), 56.7 (CH₃), 52.3 (CH), 51.5 (C), 47.9 (C), 44.6 (C), 44.01 (CH₂), 42.2 (CH), 40.5 (CH₂), 30.6 (CH₂), 30.3 (CH₂), 29.4 (CH₂), 23.0 (CH₂), 22.8 (CH₃), 18.5 (CH₃), 14.1 (CH₃), 8.6 (CH₃). R. (ATR-FTIR), cm⁻¹: 3161 (br w), 2942 (w), 2932 (w), 2864 (w), 1693 (m), 1454 (w), 1384 (m), 1241 (w), 1088 (s), 1045 (m), 1020 (w), 999 (w), 979 (m), 908 (m), 733 (m). HRMS-ESI (m/z): [M+H]⁺ calcd for C₂₁H₃₇O₄, 353.2692; found, 353.2702. [α]_{D}²⁵=−67° (c=0.25, CHCl₃).

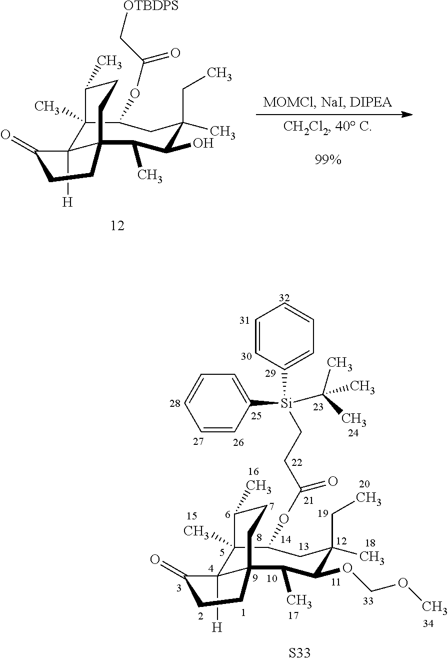

### Synthesis of O-tert-butyldiphenylsilyl-11-methoxymethylenoxy-19,20-dihydropleuromutilin S33 (Adaptation of Scheme 13)

A 4-mL vial was charged with O-tert-butyldiphenylsilyl-19,20-dihydropleuromutilin 12 [50.0 mg, 80.8 μmol, 1 equiv, dried by azeotropic distillation from benzene (500 μL)]. Sodium iodide (48.5 mg, 385 μmol, 4.00 equiv) was added to the tube. The reaction vessel was evacuated and refilled using a balloon of argon. This process was repeated twice. Dichloromethane (300 μL), N,N-diisopropylethylamine (28.5 μL, 98.5 μmol, 12.0 equiv), and chloromethyl methyl ether (18.4 μL, 146 μmol, 3.00 equiv) were added sequentially to the reaction vessel at 24° C. The vial was sealed with a Teflon-lined cap and the sealed vial was place in an oil bath that had been previously heated to 40° C. The reaction mixture was stirred and heated for 12 h at 40° C. The product mixture was transferred to a separatory funnel that had been charged with dichloromethane (25 mL) and aqueous potassium phosphate buffer solution (pH 7, 0.10 M, 5 mL). The layers that formed were separated and the aqueous layer was extracted with dichloromethane (3×5 mL). The organic layers were combine and dried over sodium sulfate. The dried solution was filtered and the filtrate was concentrated to dryness. The residue obtained was purified by automated flash-column chromatography (eluting with hexanes initially, grading to 20% ethyl acetate-hexanes, linear gradient) to afford the O-tert-butyldiphenylsilyl-11-methoxymethylenoxy-19,20-dihydropleuromutilin (S33) as an amorphous white solid (55.3 mg, 99%).

R_{f}=0.63 (20% ethyl acetate-hexanes; UV, PAA, CAM). ¹H NMR (400 MHz, CDCl₃) δ 7.70-7.66 (m, 4H, 2×H₂₇, 2×H₃₁), 7.44-7.26 (m, 6H, 2×H₂₆, 1×H₂₈, 2×H₃₀, 1×H₃₂), 5.71 (d, =8.0 Hz, 1H, 1H₁₄), 4.63 (t, J=6.5 Hz, 2H, H₃₃), 4.15 (dd, J=22.7, 6.4 Hz, 2H, H₂₂), 3.40 (s, 3H, H₄), 3.22 (d, J=6.0 Hz, 1H, H₁₁), 2.53-2.46 (m, 1H, 1×H₁₀), 2.30-2.13 (m, 2H, H₂) 2.06 (s, 1H, H₄), 1.85-1.44 (m, 8H, 2×H₁, 1×H₆, 1×H₇, 1×H₈, 1×H₁₃, 2×H₁₉), 1.39 (s, 3H, H₁₅), 1.35-1.26 (m, 1H, 1×H₇), 1.26-1.17 (m, 1H, 1×H₁₃), 1.16-1.10 (m, 1H, 1×H₈), 1.08 (s, 9H, H₂₄), 0.95-0.89 (m, 6H, 3×H₁₆, 3×H₁₈), 0.75 (t, J=7.4 Hz, 3H, H₂₀), 0.63 (d, J=5.6 Hz, 3H, H₁₇). ¹³C NMR (100 MHz, CDCl₃) δ 217.4 (C), 169.9 (C), 135.5 (CH), 132.9 (C), 132.8 (C), 129.9 (CH), 127.8 (CH), 127.8 (CH), 98.8 (CH₂), 84.6 (CH₃), 68.6 (CH), 62.9 (CH₂), 58.2 (CH), 56.7 (CH), 45.4 (C), 41.9 (C), 41.4 (C), 41.2 (CH₂), 36.8 (CH), 34.9 (CH), 34.7 (CH₂), 30.5 (CH₂), 26.9 (CH₂), 26.7 (CH₃), 26.6 (CH₃), 25.0 (CH₂), 21.7 (CH₂), 19.2 (C), 16.4 (CH₃), 14.9 (CH₃), 1.7 (CH₃), 8.2 (CH₃). IR (ATR-FTIR), cm⁻¹: 2933 (w), 2862 (w), 1735 (m), 1461 (w), 1428 (w), 1383 (w), 1285 (w), 1214 (w), 1144 (s), 1113 (s), 1087 (m), 1047 (s), 1018 (s), 970 (m), 824 (m), 741 (m), 701 (s), 613 (m), 580 (w), 504 (s), 490 (s). HRMS-ESI (m/z): [M+Na]⁺ calcd for C₄₀H₅₈NaO₆Si, 685.3900; found, 685.3894. [α]_{D}²⁵=+21° (c=0.10, CHCl₃).

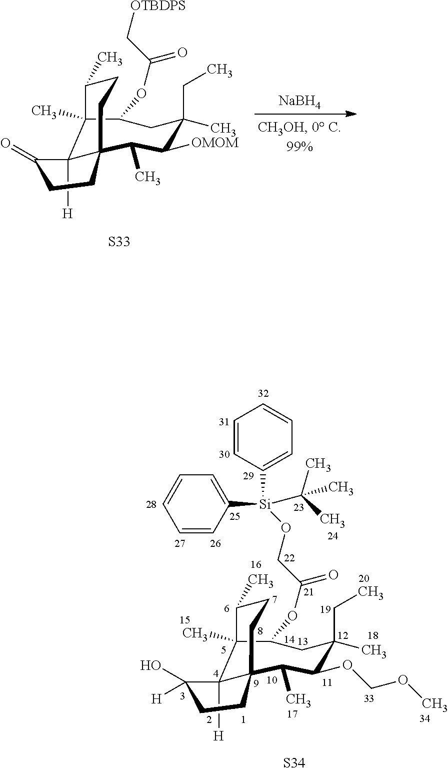

### Sodium Borohydride Reduction of O-tert-butyldiphenylsilyl-11-methoxymethylenoxy-19,20-dihydropleuromutilin S34 (Adaptation of Scheme 13)

Three equal portions of sodium borohydride (2.9 mg, 75.4 μmol, 5.00 equiv) were added over 1 h to a solution of O-tert-butyldiphenylsilyl-11-methoxymethylenoxy-19,20-dihydropleuromutilin (S33, 10.0 mg, 15.1 μmol, 1 equiv) in methanol (200 μL) at 0° C. The reaction mixture was stirred for 3 h at 0° C. The product mixture was transferred to a separatory funnel that had been charged with dichloromethane (10 mL) and aqueous potassium phosphate buffer solution (pH 7, 0.10 M, 1.0 mL). The layers that formed were separated and the aqueous layer was extracted with dichloromethane (3×5 mL). The organic layers were combine and dried over sodium sulfate. The dried solution was filtered and the filtrate was concentrated to dryness. The residue obtained was purified by automated flash-column chromatography (eluting with hexanes initially, grading to 20% ethyl acetate-hexanes, linear gradient) to afford the axial alcohol S34 as an amorphous white solid (10.2 mg, 99%). Relative stereochemistry at the C3 position was determined by 2D NOESY analysis.

R_{f}=0.57 (20% ethyl acetate-hexanes; UV, PAA, CAM). ¹H NMR (400 MHz, CDCl₃) δ 7.70-7.65 (m, 4H, 2×H₂₇, 2×H₃₁), 7.44-7.35 (m, 6H, 2×H₂₆, 1×H₂₈, 2×H₃₀, 1×H₃₂), 5.55 (d, J=9.2 Hz, 1H, H₁₄), 4.60 (d, J=6.8, 1H, 1×H₃₃), 4.56 (d, J=6.8, 1H, 1×H₃₃), 4.51 (t, J=3.2 Hz, 1H, H₃), 4.14 (dd, J=11.2, 2.8 Hz, 2H, H₂₂), 3.39 (s, 3H, H₃₄), 3.40 (d, J=6.0 Hz, 1H, H₁₁), 2.30-2.20 (m, 1H, H₁₀), 2.19-2.10 (m, 1H, H₆), 2.01-1.93 (m, 1H, 1×H₂), 0.83-1.59 (m, 7H, 2×H₁, 1×H₂, 1×H₄, 1×H₁₃, 2×H₁₉), 1.51-1.43 (m, 3H, 1×H₇, 1×H₈, 1×OH), 1.37-1.32 (m, 1H, 1×H₈), 1.27-1.21 (m, 1H, 1×H₇), 1.17-1.11 (m, 4H, 1×H₁₃, 3×H₁₅), 1.08 (s, 9H, H₂₄), 0.89 (s, 3H, H₁₉), 0.86 (d, J=7.2 Hz, 3H, H₁₆), 0.77 (t, J=7.4 Hz, 3H, H₂₀), 0.63 (d, J=7.2 Hz, 3H, H₁₇). ¹³C NMR (100 MHz, CDCl₃) δ 169.9 (C), 135.5 (CH), 132.9 (C), 129.8 (CH), 127.7 (CH), 98.8 (CH₂), 85.3 (CH), 77.2 (CH), 70.6 (CH), 62.9 (CH₂), 56.6 (CH₃), 51.2 (CH), 45.7 (C), 42.1 (C), 41.6 (CH₂), 41.3 (C), 36.6 (CH), 34.6 (CH), 34.3 (CH₂), 32.8 (CH₂), 31.9 (CH₂), 27.6 (CH₂), 26.7 (CH₃), 26.6 (CH₃), 21.8 (CH₂), 19.2 (C), 17.6 (CH₃), 16.7 (CH₃), 12.5 (CH₃), 8.2 (CH₃). IR (ATR-FTIR), cm⁻¹: 3524 (br w), 2935 (m), 2858 (m), 1752 (m), 1463 (m), 1428 (m), 1371 (w), 1295 (w), 1214 (w), 1144 (s), 1113 (s), 1089 (m), 1039 (s), 1020 (s), 969 (w), 916 (w), 824 (m), 714 (w), 702 (s), 678 (s), 613 (m), 504 (m). HRMS-ESI (m/z): [M+Na]⁺ calcd for C₄₀H₆₀NaO₆Si, 687.4057; found, 687.4049. [α]_{D}²⁵=+22° (c=0.10, CHCl₃).

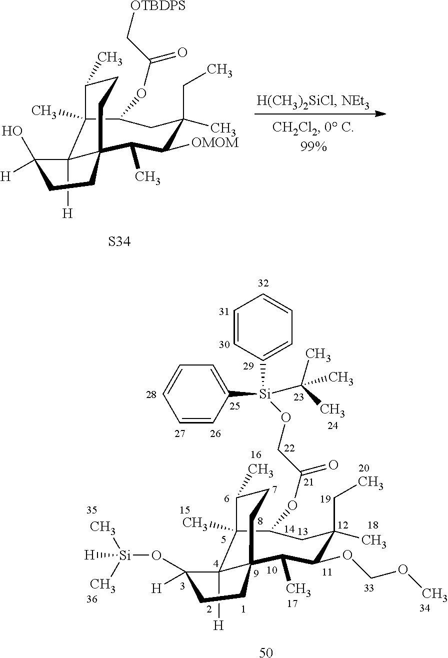

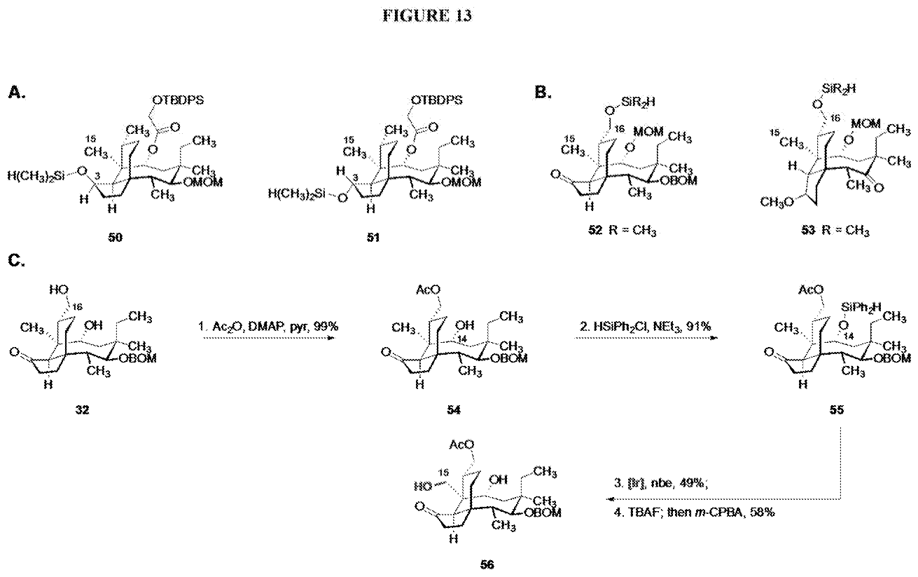

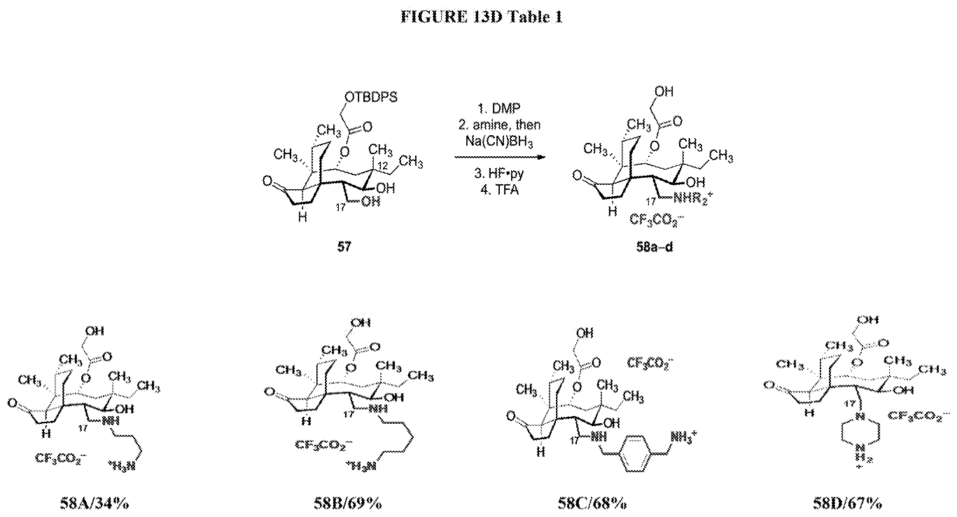

### Synthesis of Silane 50 (FIG. 13, Scheme 13)

Dimethylchlorosilane (15.4 μL, 139 μmol, 2.00 equiv) was added dropwise via syringe to a solution of the axial alcohol S34 [46.1 mg, 69.3 μmol, 1 equiv, dried by azeotropic distillation with benzene (500 μL)] and triethylamine (38.6 μL, 277 μmol, 4.00 equiv) in dichloromethane (500 μL) at 0° C. The reaction mixture was stirred for 30 min at 0° C. The product mixture was diluted sequentially with pentane (2.5 mL) and aqueous potassium phosphate buffer solution (pH 7, 0.10 M, 1.0 mL). The diluted mixture was transferred to a separatory funnel and the layers formed were separated. The aqueous layer was extracted with dichloromethane (3×10 mL). The organic layers were combined and the combined organic layers were dried over sodium sulfate. The dried solution was filtered and the filtrate was concentrated to dryness to afford the silane 50 as a colorless oil (51.1 mg, 99%).

R_{f}=0.75 (20% ether-hexanes; UV, PAA, CAM). ¹H NMR (400 MHz, C₆D₆) δ 7.82-7.79 (m, 4H, 2×H₂₇, 2×H₃₁), 7.24-7.22 (m, 6H, 2×H₂₆, 1×H₂₈, 2×H₃₀, 1×H₃₂), 5.80 (d, J=9.2 Hz, 1H, H₄), 4.83 (sep, J=2.8 Hz, 1H, Si—H), 4.54 (d, J=6.8, 1H, 1×H₃₃), 4.48 (d, J=6.8, 1H, 1×H₃₃), 4.26-4.21 (m, 3H, 1×H₁, 2×H₂₂), 3.21 (s, 3H, H₃₄), 3.08 (d, J=5.6 Hz, 1H, H₁₁), 2.43-2.39 (m, 2H, 1×H₆, 1×H₁₀), 2.12-2.03 (m, 1H, 1×H₁₃, 1×H₁₉), 1.92-1.88 (m, 1H, 1×H₁₉), 1.78-1.68 (m, 3H, 1×H₂, 1×H₇, 1×H₁₃), 1.63-1.57 (m, 3H, 1×H₁, 1×H₇, 1×H₈), 1.78-1.68 (m, 4H, 1×H₁, 1×H₂, 1×H₄, 1×H₈), 1.25-1.17 (m, 12H, 3×H₁₅, 9×H₂₄), 1.03-0.94 (m, 9H, 3×H₁₆, 3×H₁₈, 3×H₂₀), 0.78 (d, J=7.2 Hz, 3H, H₁₇), 0.13 (d, J=2.8 Hz, 3H, H₃₅), 0.11 (d, J=2.8 Hz, 3H, H₃₆). C NMR (100 MHz, C₆D₆) δ 169.7 (C), 136.1 (CH), 136.1 (CH), 133.6 (C), 133.5 (C), 130.2 (CH), 128.2 (CH), 128.2 (CH), 99.1 (CH₂), 85.5 (CH), 79.3 (CH), 70.7 (CH), 63.4 (CH₂), 56.4 (CH₃), 51.7 (CH), 46.3 (C), 42.5 (C), 42.3 (CH₂), 41.8 (C), 36.5 (CH), 35.0 (CH), 33.5 (CH₂), 32.9 (CH₂), 32.4 (CH₂), 28.3 (CH₂), 27.1 (CH₃), 27.0 (CH₃), 22.4 (CH₂), 19.6 (C), 17.5 (CH₃), 17.1 (CH₃), 13.0 (CH₃), 8.8 (CH₃), −0.73 (CH₃), −1.3 (CH₃). IR (ATR-FTIR), cm⁻¹: 2958 (m), 1754 (w), 1727 (w), 1463 (w), 1428 (w), 1370 (w), 1290 (w), 1252 (m), 1212 (w), 1145 (s), 1113 (s), 1070 (m), 1039 (s), 1022 (s), 942 (m), 911 (s), 824 (m), 740 (m), 701 (s), 613 (m), 498 (s). HRMS-ESI (m/z): [M-Si(CH₃)₂+Na]⁺ calcd for C₄₀H₆₀NaO₆Si, 687.4057; found, 687.4048. [α]_{D}²⁵=+24° (c=0.25, CHCl₃).

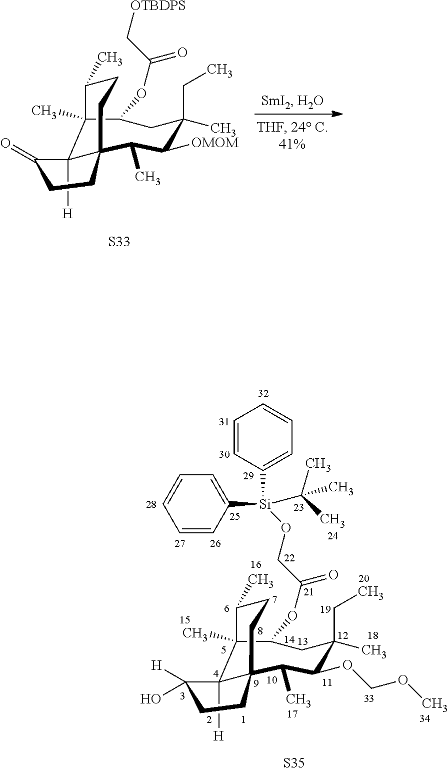

### Samarium(II) Iodide Reduction of O-tert-butyldiphenylsilyl-11-methoxymethylenoxy-19,20-dihydropleuromutilin S35 (Scheme 13)

Water (219 μL, 12.2 mmol, 800 equiv) was added dropwise into a solution of samarium(II) iodide in tetrahydrofuran (0.10 M, 1.22 mL, 30.2 μmol, 8.00 equiv). A solution of O-tert-butyldiphenylsilyl-11-methoxymethylenoxy-19,20-dihydropleuromutilin (S33, 10.1 mg, 15.1 μmol, 1 equiv) in tetrahydrofuran (800 μL). The resulting mixture was stirred for 5 h at 24° C. The product mixture was transferred to a separatory funnel that had been charged with dichloromethane (10 mL). The layers that formed were separated and the aqueous layer was extracted with dichloromethane (3×10 mL). The organic layers were combine and dried over sodium sulfate. The dried solution was filtered and the filtrate was concentrated to dryness. The residue obtained was purified by automated flash-column chromatography (eluting with hexanes initially, grading to 70% ethyl acetate-hexanes, linear gradient) to afford the equatorial alcohol S35 as a colorless clear film (4.1 mg, 41%).

R_{f}=0.57 (66% ethyl acetate-hexanes; UV, PAA, CAM). ¹H NMR (400 MHz, CDCl₃) δ 7.70-7.64 (m, 4H, 2×H₂₇, 2×H₃₁), 7.45-7.32 (m, 6H, 2×H₂, 1×H₂, 2×H₃₀, 1×H₃₂), 5.63 (d, J=8.8 Hz, 1H, H₁₄), 4.59 (dd, J=11.2, 4.8 Hz, 2H, H₃₃), 4.39 (t, J=6.6 Hz, 1H, H₃), 4.14 (dd, J=23.2, 6.8 Hz, 2H, H₂₂), 3.39 (s, 3H, H₃₄), 3.23 (d, J=6.0 Hz, 1H, H₁₁), 2.29-2.18 (m, 2H, 1×H₂, 1×H₁₀), 1.83-1.77 (m, 2H, 1×H₁₃, 1×H₁₉), 1.72-1.49 (m, 8H, 2×H₁, 1×H₂, 1×H₄, 1×H₆, 1×H₈, 1×H₁₉, 1×OH), 1.31-1.14 (m, 4H, 2×H₇, 1×H₈, 1×H₁₃), 1.07 (s, 9H, H₂₄), 1.05 (s, 3H, H₁₅), 0.93 (s, 3H, H₁₈), 0.82 (d, J=7.2 Hz, 3H, H₁₆), 0.75 (t, J=7.4 Hz, 3H, H₂₀), 0.68 (d, J=6.0 Hz, 3H, H₁₇). ¹³C NMR (100 MHz, CDCl₃) δ 169.9 (C), 135.5 (CH), 132.8 (C), 129.8 (CH), 127.7 (CH), 98.5 (CH₂), 83.9 (CH₃), 74.8 (CH), 70.0 (CH), 62.9 (CH₂), 56.6 (CH₃), 56.5 (CH), 47.1 (C), 41.7 (C), 41.1 (CH₂), 40.7 (C), 36.8 (CH), 34.3 (CH), 32.0 (CH₂), 31.1 (CH₂), 29.6 (CH₂), 26.9 (CH₂), 26.6 (CH₃), 26.6 (CH), 21.7 (CH₂), 19.2 (C), 18.1 (CH₃), 16.4 (CH₃), 12.3 (CH₃), 8.2 (CH₃). IR (ATR-FTIR), cm⁻¹: 2935 (m), 1753 (m), 1462 (w), 1428 (w), 1373 (w), 1292 (w), 1212 (w), 1140 (s), 1113 (s), 1036 (s), 968 (m), 944 (m), 917 (w), 824 (m), 740 (m), 701 (s), 678 (s), 613 (m), 503 (s), 489 (s). HRMS-ESI (m/z): [M+Na]⁺ calcd for C₄₀H₆₀NaO₆Si, 687.4057; found, 687.4057.

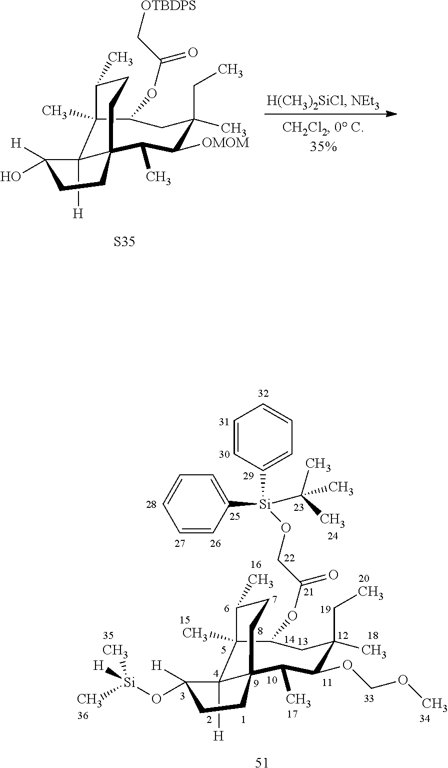

### Synthesis of Silane 51 (FIG. 3, Scheme 13)

Dimethylchlorosilane (6.4 μL, 57.1 μmol, 2.00 equiv) was added dropwise via syringe to a solution of the equatorial alcohol S35 [19.0 mg, 28.6 μmol, 1 equiv, dried by azeotropic distillation with benzene (500 μL)] and triethylamine (15.9 μL, 114 μmol, 4.00 equiv) in dichloromethane (200 μL) at 0° C. The reaction mixture was stirred for 30 min at 0° C. The product mixture was diluted sequentially with pentane (2.5 mL) and aqueous potassium phosphate buffer solution (pH 7, 0.10 M, 1.0 mL). The diluted mixture was transferred to a separatory funnel and the layers formed were separated. The aqueous layer was extracted with dichloromethane (3×10 mL). The organic layers were combined and the combined organic layers were dried over sodium sulfate. The dried solution was filtered and the filtrate was concentrated to dryness. The residue obtained was purified by flash-column chromatography on neutral alumina (eluting with 20% ether-hexanes) to afford the silane 51 as a colorless clear film (7.2 mg, 35%).

R_{f}=0.77 (20% ether-hexanes; UV, PAA, CAM). ¹H NMR (400 MHz, C₆D₆) δ 7.83-7.79 (m, 4H, 2×H₂₇, 2×H₃₁), 7.26-7.23 (m, 6H, 2×H₂₆, 1×H₂₈, 2×H₃₀, 1×H₃₂), 5.87 (d, J=8.8 Hz, 1H, H₁₄), 4.88 (sep, J=2.9 Hz, 1H, Si—H), 4.44-4.41 (m, 2H, H₃₃), 4.30 (td, J=7.8, 2.4 Hz, 1H, H₃), 4.24 (s, 2H, H₃₃), 3.27 (d, J=6.0 Hz, 1H, H₁₁), 3.18 (s, 3H, H₃₄), 2.43-2.33 (m, 1H, 1×H₁₀), 2.11-1.82 (m, 6H, 1×H₁, 1×H₂, 1×H₄, 1×H₇, 1×H₁₃, 1×H₁₉), 1.63-1.54 (m, 2H, 1×H₇, 1×H₈), 1.47-1.31 (m, 2H, 1×H₂, 1×H₁₉), 1.25 (s, 3H, H₁₈), 1.22-1.15 (m, 10H, 1×H₁, 9×H₂₄), 0.99-0.85 (m, 8H, 1×H₈, 1×H₁₃, 3×H₁₆, 3×H₂₀), 0.83-0.75 (m, 6H, 3×H₁₇, 3×H₁₈), 0.17 (d, J=2.8 Hz, 3H, H₃₅), 0.14 (d, J=2.8 Hz, 3H, H₃₆). ¹³C NMR (100 MHz, C₆D₆) δ 169.8 (C), 136.1 (CH), 136.1 (CH), 133.5 (C), 133.5 (C), 130.2 (CH), 128.2 (CH), 128.2 (CH), 99.2 (CH₂), 84.7 (CH), 77.2 (CH), 70.2 (CH), 63.4 (CH₂), 56.5 (CH), 56.4 (CH₃), 46.7 (C), 42.2 (C), 41.8 (C), 41.5 (CH₂), 37.4 (CH), 34.8 (CH), 32.3 (CH₂), 31.6 (CH₂), 30.1 (CH₂), 27.4 (CH₂), 27.0 (CH₃), 26.6 (CH₃), 22.3 (CH₂), 19.6 (C), 18.5 (CH), 16.9 (CH₃), 12.8 (CH), 8.7 (CH₃), −0.35 (CH₃), −1.0 (CH₃). IR (ATR-FTIR), cm⁻¹: 2961 (w), 1753 (w), 1460 (w), 1428 (w), 1260 (m), 1211 (w), 1139 (m), 1094 (s), 1037 (s), 1019 (s), 903 (m), 799 (s), 740 (w), 701 (m), 613 (m), 501 (m). HRMS-ESI (m/z): [M-Si(CH₃)₂+Na]⁺ calcd for C₄₀H₆₀NaO₆Si, 687.4057; found, 687.4064.

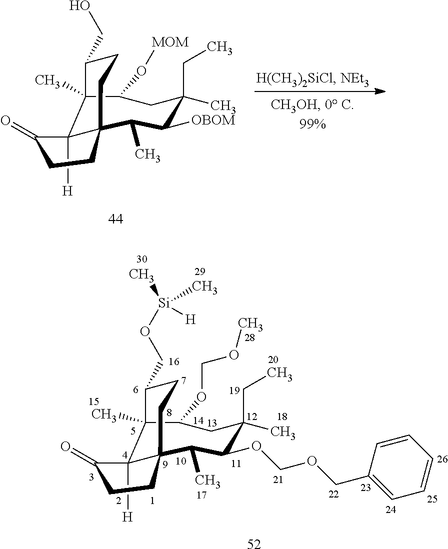

### Synthesis of Silane 52 (Scheme 13)

Dimethylchorosilane (8.8 μL, 79.6 μmol, 2.00 equiv) was added dropwise via syringe to a solution of the alcohol 44 [20.0 mg, 39.8 μmol, 1 equiv, dried by azeotropic distillation with benzene (500 μL)] and triethylamine (22.2 μL, 159 μmol, 4.00 equiv) in dichloromethane (500 μL) at 0° C. The reaction mixture was stirred for 30 min at 0° C. The product mixture was diluted sequentially with pentane (2.5 mL) and aqueous potassium phosphate buffer solution (pH 7, 0.10 M, 1.0 mL). The diluted mixture was transferred to a separatory funnel and the layers formed were separated. The aqueous layer was extracted with dichloromethane (3×10 mL). The organic layers were combined and the combined organic layers were dried over sodium sulfate. The dried solution was filtered and the filtrate was concentrated to dryness. The residue obtained was purified by flash-column chromatography on neutral alumina (eluting with 20% ether-hexanes) to afford the silane 52 as an amorphous white solid (4.4 mg, 20%).

R_{f}=0.77 (20% ether-hexanes; UV, PAA, CAM). ¹H NMR (500 MHz, C₆D₆) δ 7.31-7.08 (m, 5H, 2×H₂₄, 2×H₂₅, 1×H₂₆), 5.03-5.00 (m, 1H, Si—H), 4.59-4.51 (m, 4H, 2×H₂₁, 2×H₂₂), 4.44-4.39 (m, 2H, H₂₇), 4.24 (d, J=13.0 Hz, 1H, H₁₁), 4.08 (d, J=8.0 Hz, 1H, 1×H₁₆), 3.59 (t, J=8.2 Hz, 1H, 1×H₁₆), 3.13 (s, 3H, H₂), 3.01 (d, J=5.5 Hz, 1H, H₁₄), 2.16-2.09 (m, 3H, 2×H₂, 1×H₁₀), 1.90-1.67 (m, 7H, 2×H₁, 1×H₄, 1×H₇, 3×H₁₅), 1.55-1.30 (m, 6H, 1×H₆, 1×H₇, 1×H₈, 2×H₁₃, 1×H₁₉), 1.05-0.97 (m, 4H, 1×H₁₉, 3×H₂₀), 0.96-0.88 (m, 4H, 1×H₈, 3×H₁₈), 0.84 (d, J=7.0 Hz, 3H, H₁₇), 0.26 (s, 6H, 3×H₂₉, 3×H₃₀). ¹³C NMR (125 MHz, C₆D₆) δ 215.6 (C), 138.7 (C), 97.2 (CH), 95.8 (CH₂), 85.5 (CH₃), 73.1 (CH₂), 70.7 (CH), 66.0 (CH₂), 58.8 (CH), 55.7 (C), 46.4 (CH), 45.3 (C), 41.4 (CH₂), 40.5 (C), 35.7 (C), 34.4 (CH₂), 30.2 (CH₂), 26.9 (CH₃), 25.5 (CH₂), 23.1 (CH₂), 22.6 (CH₂), 15.3 (CH₃), 12.4 (CH₃), 9.3 (CH₃), 1.4 (CH₃), −1.2 (CH₃). IR (ATR-FTIR), cm⁻¹: 2985 (w), 2930 (w), 2870 (w), 1733 (m), 1457 (w), 1381 (w), 1153 (m), 1079 (w), 1036 (s), 1000 (m), 917 (m), 733 (s), 699 (w). HRMS-ESI (m/z): [M-Si(CH₃)₂+Na]⁺ calcd for C₃₀H₄₆NaO₆, 525.3192; found, 525.3177.

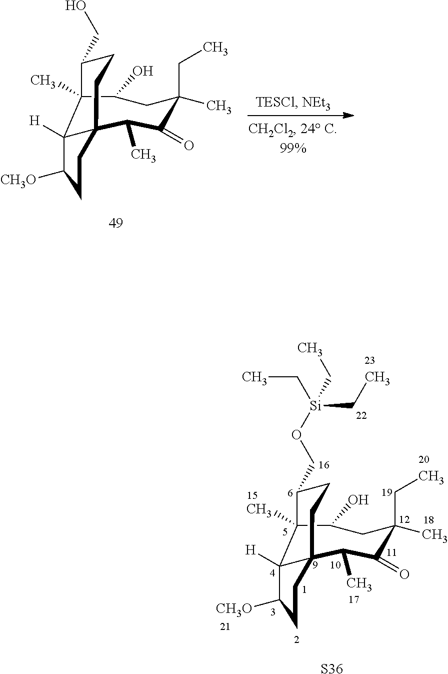

### Synthesis of Alcohol S36 (Scheme 13)

Chlorotriethylsilane (105 μL, 624 μmol, 1.05 equiv) was added dropwise via syringe to a solution of diol 49 [200 mg, 567 μmol, 1 equiv, dried by azeotropic distillation with benzene (1.0 mL)] and triethylamine (158 μL, 1.13 mmol, 2.00 equiv) in dichloromethane (6.5 mL) at 24° C. The reaction mixture was stirred for 35 min at 24° C. The product mixture was transferred to a separatory funnel that had been charged with dichloromethane (25 mL) and aqueous potassium phosphate buffer solution (pH 7, 0.10 M, 10 mL). The layers that formed were separated and the aqueous layer was extracted with dichloromethane (3×25 mL). The organic layers were combine and dried over sodium sulfate. The dried solution was filtered and the filtrate was concentrated to dryness. The residue obtained was purified by automated flash-column chromatography (eluting with hexanes initially, grading to 40% ether-hexanes, linear gradient) to afford the alcohol S36 as an amorphous white solid (199 mg, 99%).

R_{f}=0.69 (30% ethyl acetate-hexanes; PAA, CAM). ¹H NMR (400 MHz, CDCl₃) δ 4.87 (d, J=4.0 Hz, 1H, OH), 4.68 (dd, J=9.2, 4.0 Hz, 1H, H₁₄), 4.15 (dd, J=11.2, 1.6 Hz, 1H, 1×H₁₉), 3.54 (dd, J=11.2, 4.0 Hz, 1H, 1×H₁₆), 3.51-3.45 (m, 1H, H₃), 3.20 (s, 3H, H₂₁), 3.15 (q, J=6.5 Hz, 1H, H₁₀), 2.28-1.94 (m, 5H, 1×H₁, 1×H₂, 1×H₈, 1×H₁₃, 1×H₁₉), 1.83-1.78 (m, 1H, 1×H₇), 1.39-1.33 (m, 3H, 1×H₄, 1×H₆, 1×H₁₉), 1.36 (dq, J=15.2, 3.6 Hz, 1H, 11×H₈), 1.27-1.05 (m, 6H, 1×H₁, 1×H₂, 1×H₇, 2×H₁₅), 1.03-0.94 (m, 15H, 3×H₁₇, 3×H₁₈, 9×H₂₃), 0.82 (t, J=7.4 Hz, 3H, H₂₀), 0.70-0.48 (m, 6H, H₂₂). ¹³C NMR (100 MHz, CDCl₃) δ 219.8 (C), 83.2 (CH), 66.1 (CH), 64.8 (CH), 63.1 (CH₂), 56.7 (CH₃), 52.4 (CH), 51.4 (C), 48.0 (C), 45.0 (C), 43.0 (CH₂), 42.2 (CH), 40.6 (CH₂), 30.9 (CH₂), 30.1 (CH₂), 29.6 (CH₂), 23.4 (CH₃), 22.8 (CH₂), 18.8 (CH₃), 14.2 (CH₃), 8.7 (CH₃), 6.6 (CH₃), 4.2 (CH₂). IR (ATR-FTIR), cm⁻¹: 2935 (m), 2876 (m), 1693 (m), 1458 (m), 1145 (w), 1099 (s), 1070 (s), 1034 (s), 1004 (m), 973 (w), 742 (m). HRMS-ESI (m/z): [M+Na]⁺ calcd for C₂₇H₅₀NaO₄Si, 489.3376; found, 489.3379. [α]_{D}²⁵=−65° (c=0.10, CHCl₃).

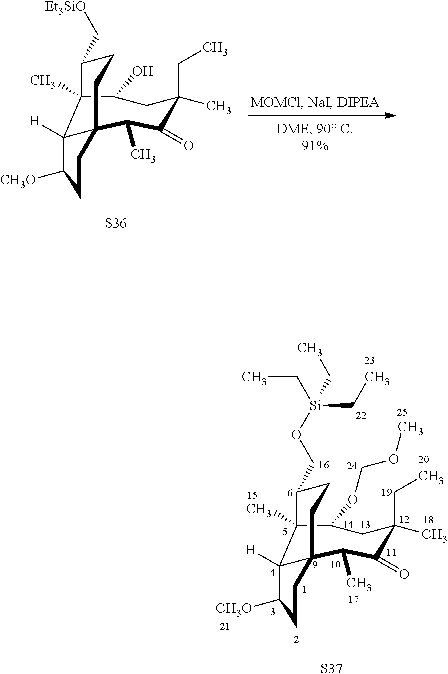

### Synthesis of 4-epi-16-hydroxy-19,20-dihydromutilin Derivative S37 (Scheme 13)

A 4-mL vial was charged with the alcohol S36 (60.0 mg, 129 μmol, 1 equiv). Benzene (500 μL) was added to the reaction vessel and the solution was concentrated to dryness. This process was repeated twice. Sodium iodide (77.1 mg, 514 μmol, 4.00 equiv) was added to the tube. The reaction vessel was evacuated and refilled using a balloon of argon. This process was repeated twice. 1,2-Dimethoxyethane (1.0 mL), N,N-diisopropylethylamine (269 μL, 1.54 mmol, 12.0 equiv), and chloromethyl methyl ether (58.6 μL, 711 μmol, 6.00 equiv) were added sequentially to the reaction vessel at 24° C. The vessel was sealed and the sealed vessel was place in an oil bath that had been previously heated to 90° C. The reaction mixture was stirred and heated for 6 h at 90° C. The product mixture was transferred to a separatory funnel that had been charged with dichloromethane (25 mL) and aqueous potassium phosphate buffer solution (pH 7, 0.10 M, 10 mL). The layers that formed were separated and the aqueous layer was extracted with dichloromethane (3×25 mL). The organic layers were combine and dried over sodium sulfate. The dried solution was filtered and the filtrate was concentrated to dryness. The residue obtained was purified by automated flash-column chromatography (eluting with hexanes initially, grading to 25% ether-hexanes, linear gradient) to afford the 4-epi-16-hydroxy-19,20-dihydromutilin derivative S37 as a colorless oil (59.5 mg, 91%).

R_{f}=0.69 (30% ethyl acetate-hexanes; PAA, CAM). ¹H NMR (400 MHz, CDCl₃) δ 4.65 (t, J=6.7 Hz, 2H, H₂₄), 4.40 (d, J=9.6 Hz, 1H, 1×H₁₆), 4.08 (dd, J=10.4, 2.4 Hz, 1H, 1×H₁₆), 3.48-3.42 (m, 1H, H₃), 3.39 (s, 3H, H₂₁), 3.25-3.15 (m, 4H, 1×H₁₄, 3×H₂₅), 3.06 (q, J=6.5 Hz, 1H, H₁₀), 2.28 (dd, J=15.6, 9.6 Hz, 1H, 1×H₁₃), 2.22-2.12 (m 1H, 1×H₈), 2.10-1.88 (m, 4H, 1×H₁, 1×H₂, 1×H₇, 1×H₁₉), 1.78 (d, J=16.0 Hz, 1H, 1×H₇), 1.67 (d, J=11.2 Hz, 1H, 1×H₁₃), 1.55-1.46 (m, 1H, 1×H₁), 1.41-1.29 (m, 2H, 1×H₄, 1×H₆), 1.26-1.06 (m, 6H, 1×H₂, 1×H₈, 3×H₁₅, 1×H₁₉), 1.03 (s, 3H, H₁₈), 1.02-0.88 (m, 12H, 3×H₁₇, 9×H₂₃), 0.77 (t, J=6.8, 3H, H₂), 0.59 (q, J=8.2 Hz, 6H, H₂₂). ¹³C NMR (100 MHz, CDCl₃) δ 219.1 (C), 96.3 (CH₂), 83.1 (CH₃), 76.3 (CH), 64.1 (CH), 63.8 (CH₂), 56.7 (CH), 55.8 (CH), 54.5 (CH), 51.6 (C), 47.8 (C), 44.3 (C), 42.3 (CH), 42.1 (CH₂), 40.4 (CH₂), 30.6 (CH₂), 30.5 (CH₂), 29.4 (CH₂), 23.6 (CH₂), 22.9 (CH₃), 19.0 (CH₃), 14.0 (CH₃), 8.9 (CH₃), 6.8 (CH₃), 4.5 (CH₂). IR (ATR-FTIR), cm⁻¹: 3447 (br w), 2935 (m), 2876 (m), 2810 (w), 1693 (m), 1458 (m), 1414 (w), 1383 (w), 1242 (w), 1149 (w), 1099 (s), 1061 (s), 1001 (s), 982 (s), 908 (w), 861 (w), 766 (m), 730 (s). HRMS-ESI (m/z): [M+H]⁺ calcd for C₂₉H₅₀O₅Si, 511.3819; found, 511.3856. [α]_{D}²⁵=49° (c=0.10, CHCl₃).

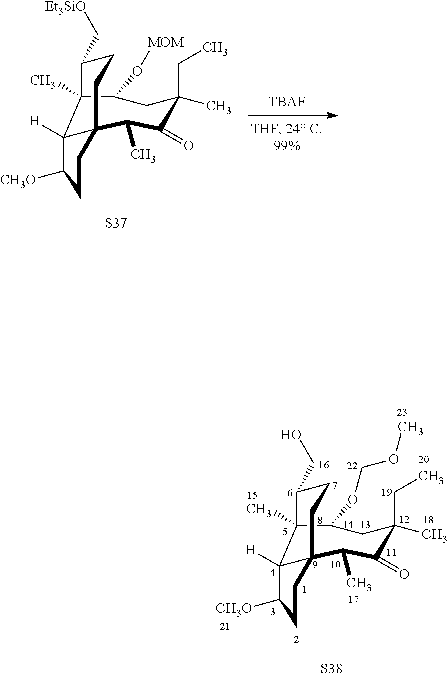

### Synthesis of Primary Alcohol S38 (Scheme 13)

A solution of tetrabutylammonium fluoride in tetrahydrofuran (1.0 M, 196 μL, 196 μmol, 2.00 equiv) was added dropwise via syringe to a solution of the 4-epi-16-hydroxy-19,20-dihydromutilin derivative S37 (50.0 mg, 97.9 μmol, 1 equiv) in tetrahydrofuran (1.0 mL) at 24° C. The reaction mixture was stirred for 2.5 h at 24° C. The product mixture was diluted sequentially with ether (5.0 mL) and aqueous potassium phosphate buffer solution (pH 7, 0.10 M, 3.0 mL). The diluted product mixture was transferred to a separatory funnel that had been charged with a mixture of ether and pentane (1:1, v/v, 50 mL). The layers that formed were separated and the organic layer was washed with saturated aqueous sodium bicarbonate solution (3×10 mL). The washed organic layer was dried over sodium sulfate. The dried solution was filtered and the filtrate was concentrated to dryness. The residue obtained was purified by automated flash-column chromatography (eluting with hexanes initially, grading to 50% ethyl acetate-hexanes, linear gradient) to afford the diol S38 as a light yellow oil (44.3 mg, 99%).

R_{f}=0.36 (50% ethyl acetate-hexanes; PAA, CAM). ¹H NMR (400 MHz, CDCl₃) δ 4.68 (q, J=6.7 Hz, 21, H₂), 4.40 (d, J=9.6 Hz, 1H, H₁₄), 3.86 (dd, J=11.6, 4.8 Hz, 1H, 1×H₁₆), 3.54 (dd, J=11.6, 7.2 Hz, 1H, 1×H₁₆), 3.49-3.42 (m, 1H, H₃), 3.39 (s, 31, H₂₁), 3.20 (s, 3H, H₂₃), 3.04 (q, J=9.2 Hz, 1H, H₁₀), 2.33-1.92 (m, 6H, 1×H₁, 2×H₂, 1×H₇, 1×H₈, 1×H₁₃), 1.81-1.74 (m, 1H, 1×H₁₉), 1.71-1.60 (m, 3H, 1×H₄, 1×H₁₃, 1×H₁₉), 1.52-1.42 (m, 1H, 1×H₁), 1.34-1.06 (m, 7H, 1×H₆, 1×H₇, 1×H₈, 3×H₁₅, 1×OH), 1.03 (s, 3H, H₁₈), 0.96 (d, J=8.0 Hz, 3H, H₁₇), 0.76 (t, J=7.6 Hz, 3H, H₂₁). ¹³C NMR (100 MHz, CDCl₃) δ 218.9 (C), 95.3 (CH₂), 83.1 (CH), 75.6 (CH), 64.3 (CH), 63.5 (CH), 56.7 (CH₃), 55.8 (CH), 53.7 (CH), 51.5 (C), 47.6 (C), 44.5 (C), 42.7 (CH), 41.5 (CH₂), 40.3 (CH₂), 30.6 (CH₂), 30.3 (CH₂), 29.4 (CH₂), 23.2 (CH₂), 22.9 (CH), 19.7 (CH₃), 13.9 (CH₃), 8.9 (CH₃). IR (ATR-FTIR), cm⁻¹: 3447 (br w), 2935 (m), 2876 (m), 2810 (w), 1693 (m), 1458 (m), 1414 (w), 1383 (w), 1242 (w), 1149 (w), 1099 (s), 1061 (s), 1001 (s), 982 (s), 908 (w), 861 (w), 766 (m), 730 (s). HRMS-ESI (m/z): [M+Na]⁺ calcd for C₂₃H₄₀NaO₅, 419.2773; found, 419.2765. [α]_{D}²⁵=−52° (c=0.10, CHCl₃).

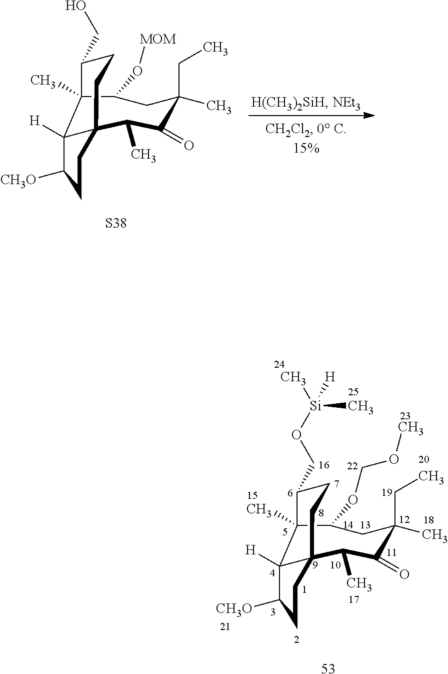

### Synthesis of Silane 53 (Scheme 13)

Dimethylchlorosilane (11.1 μL, 99.9 μmol, 2.00 equiv) was added dropwise via syringe to a solution of the alcohol S38 [19.8 mg, 49.9 μmol, 1 equiv, dried by azeotropic distillation with benzene (500 μL)] and triethylamine (27.8 μL, 200 μmol, 4.00 equiv) in dichloromethane (500 μL) at 0° C. The reaction mixture was stirred for 30 min at 0° C. The product mixture was diluted sequentially with pentane (2.5 mL) and aqueous potassium phosphate buffer solution (pH 7, 0.10 M, 1.0 mL). The diluted mixture was transferred to a separatory funnel and the layers formed were separated. The aqueous layer was extracted with dichloromethane (3×10 mL). The organic layers were combined and the combined organic layers were dried over sodium sulfate. The dried solution was filtered and the filtrate was concentrated to dryness. The residue obtained was purified by flash-column chromatography on neutral alumina (eluting with 20/a ether-hexanes) to afford the silane 53 as a colorless clear film (3.4 mg, 15%).

R_{f}=0.88 (20% ether-hexanes; PAA, CAM). ¹H NMR (600 MHz, C₆D₆) δ 5.01 (br s, 1H, Si—H), 4.49 (s, 2H, H₂₂), 4.40 (dd, J=10.2, 2.4 Hz, 1H, H₁₄), 4.37 (d, J=9.6 Hz, 1H, 1×H₁₆), 3.64-3.58 (m, 1H, H₃), 3.48 (t, J=10.2 Hz, 1H, 1×H₁₆), 3.17 (s, 3H, H₂₁), 3.04 (s, 3H, H₂₃), 3.06 (q, J=6.6 Hz, 1H, H₁₀), 2.39 (dd, J=15.6, 9.6 Hz, 1H, 1×H₁₃), 2.30 (td, J=10.2, 3.6 Hz, 1H, 1×H₂), 2.19-2.08 (m, 3H, 1×H₁, 1×H₇, 1×H₁₉), 1.92 (dt, J=13.2, 4.2 Hz, 1H, 1×H₈), 1.84-1.78 (m, 1H, 1×H₁), 1.78-1.73 (m, 3H, 1×H₄, 1×He, 1×H₁₃), 1.60 (s, 3H, H₁₅), 1.58-1.53 (m, 1H, 1×H₇), 1.40 (td, J=12.6, 3.6 Hz, 1H, 1×H₈), 1.13 (s, 3H, H₁₈), 1.05-0.99 (m, 1H, 1×H₂), 0.95 (d, J=6.6 Hz, 3H, H₁₇), 0.90 (td, J=13.8, 4.8 Hz, 1H, 1×H₁₈), 0.68 (t, J=7.5 Hz, 3H, H₂₀), 0.27-0.23 (m, 6H, 3×H₂₄, 3×H₂₅). ¹³C NMR (150 MHz, C₆D₆) δ 217.7 (C), 96.5 (CH₂), 83.4 (CH₃), 76.5 (CH), 65.7 (CH₂), 64.4 (CH), 56.5 (CH), 55.8 (CH), 54.8 (CH), 51.8 (C), 47.9 (C), 44.8 (C), 42.6 (CH₂), 42.4 (CH₃), 40.6 (CH₂), 30.9 (CH₂), 30.8 (CH₂), 29.7 (CH₂), 24.1 (CH₃), 23.4 (CH₃), 19.8 (CH₃), 14.3 (CH₃), 9.2 (CH₃), 1.44 (CH₃), −1.20 (C13). IR (ATR-FTIR), cm⁻¹: 2933 (m), 1690 (m), 1458 (m), 1145 (m), 1095 (m), 1029 (s), 958 (w), 915 (m), 730 (s), 647 (w). HRMS-ESI (m/z): [M-Si(CH₃)₂+Na]⁺ calcd for C₂₃H₄₀NaO₅, 419.2773; found, 419.2780.

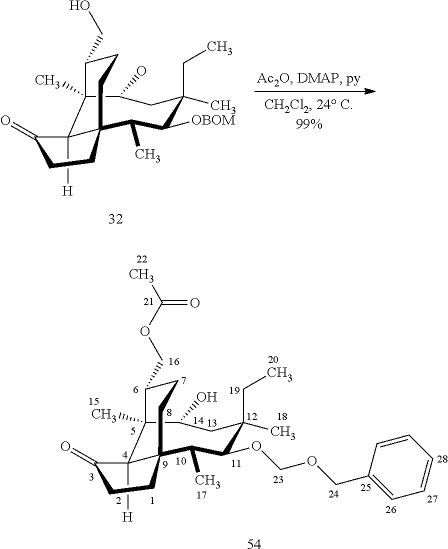

### Synthesis of Acetate 54 (FIG. 13, Scheme 13)

A 4-mL vial was charged with the diol 32 (30.0 mg, 65.4 μmol, 1 equiv). Benzene (200 μL) was added to the vial. The solution was concentrated to dryness. This process was repeated twice. The reaction vessel was evacuated and refilled using a balloon of argon. This process was repeated twice. Dichloromethane (1.0 mL), pyridine (15.8 μL, 196 μmol, 3.00 equiv), 4-dimethylaminopyridine (9.6 mg, 78.5 μmol, 1.20 equiv), and acetic anhydride (7.5 μL, 78.5 μmol, 1.20 equiv) were added sequentially to the reaction vessel at 24° C. The reaction mixture was stirred for 1 h at 24° C. The product mixture was concentrated to dryness. The residue obtained was purified by automated flash-column chromatography (eluting with hexanes initially, grading to 33% ethyl acetate-hexanes, linear gradient) to afford the acetate 54 as an amorphous white solid (32.7 mg, 99%).

R_{f}=0.55 (40% ether-hexanes; UV, PAA, CAM). ¹H NMR (400 MHz, CD₂Cl₂) δ 7.36-7.26 (m, 5H, 2×H₂₆, 2×H₂₇, 1×H₂₈), 4.76 (s, 2H, H₂₃), 4.65 (2, 2H, H₂₄), 4.28-4.22 (m, 2H, 1×H₁₁, 11×H₁₆), 3.89 (td, J=11.2, 2.0 Hz, 1H, 1×H₁₆), 3.27 (dd, J=6.4, 2.4 Hz, 1H, H₁₄), 2.42-2.35 (m, 1H, H₁₀), 2.27-2.08 (m, 2H, H₂), 2.05 (s, 1H, H₄), 1.99 (s, 3H, H₂₂), 1.83-1.43 (m, 10H, 2×H₁, 1×H₆, 2×H₇, 1×H₈, 1×H₁₃, 2×H₁₉, 1×OH), 1.42-1.36 (m, 1H, 1×H₁₃), 1.35 (s, 3H, H₁₅), 1.12 (tt, J=14.4, 3.6 Hz, 1H, 1×H_{R}), 1.01 (s, 3H, H₁₈), 0.97-0.88 (m, 6H, 3×H₁₇, 3×H₂₀). ¹³C NMR (100 MHz, CD₂Cl₂) δ 217.7 (C), 171.5 (C), 138.9 (C), 128.9 (CH), 128.1 (CH), 128.1 (CH), 97.3 (CH₂), 85.7 (CH), 71.2 (CH₂), 67.1 (CH₂), 66.1 (CH), 59.5 (CH), 45.6 (C), 44.0 (CH₂), 42.6 (C), 42.4 (CH), 41.9 (C), 35.9 (CH), 34.9 (CH₂), 30.4 (CH₂), 27.4 (CH₃), 25.8 (CH₂), 22.7 (CH₂), 22.6 (CH₂), 21.4 (CH₃), 13.8 (CH₃), 12.3 (CH₃), 8.5 (CH₃). IR (ATR-FTIR), cm⁻¹: 3494 (br w), 2933 (w), 1730 (m), 1461 (w), 1368 (w), 1244 (m), 1086 (w), 1019 (s), 977 (s), 940 (m), 734 (s), 698 (m). HRMS-ESI (m/z): [M+H]⁺ calcd for C₃₀H₄₅O₆, 501.3216; found, 501.3211. [α]_{D}²⁵=+57° (c=0.10, CHCl₃).

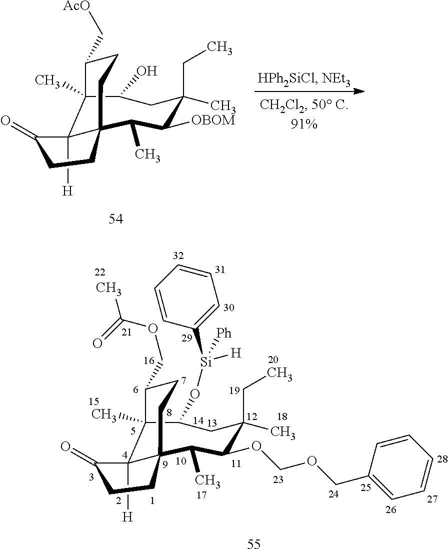

### Synthesis of Silane 55 (FIG. 13, Scheme 13)

A 10-mL round-bottomed flask fused to a Teflon-coated valve was charged with the diol 54 (180 mg, 360 μmol, 1 equiv). Benzene (5001 μL) was added to the vial. The solution was concentrated to dryness. This process was repeated twice. The reaction vessel was evacuated and refilled using a balloon of argon. This process was repeated twice. Dichloromethane (2.0 mL), triethylamine (200 μL, 1.44 mmol, 4.00 equiv), and chlorodiphenylsilane (141 μL, 719 μmol, 2.00 equiv) were added sequentially to the reaction vessel at 24° C. The reaction vessel was sealed and the sealed vessel was placed in an oil bath that had been previously heated to 50° C. The reaction mixture was stirred and heated for 1 h at 50° C. The product mixture was diluted sequentially with pentane (2.0 mL) and aqueous potassium phosphate buffer solution (pH 7, 0.10 M, 15 mL). The diluted product mixture was transferred to a separatory funnel. The layers that formed were separated and the aqueous layer was extracted with dichloromethane (3×20 mL). The organic layers were combined and dried over sodium sulfate. The dried solution was filtered and the filtrate was concentrated to dryness. The residue obtained was purified by automated flash-column chromatography (eluting with hexanes initially, grading to 20% ethyl acetate-hexanes, linear gradient) to afford the silane 55 as an amorphous white solid (221 mg, 91%).

R_{f}=0.47 (20% ether-hexanes; UV, PAA, CAM). ¹H NMR (600 MHz, C4) δ 7.97-7.85 (m, 4H, 4×H₃₁), 7.30-7.08 (m, 11H, 2×H₂₆, 2×H₂₇, 1×H₂₈, 4×H₃₀, 2×H₃₂), 5.84 (s, 1H, Si—H), 4.74 (d, J=7.8 Hz, 1H, H₁₁), 4.60 (dd, J=11.4, 3.0 Hz, 1H, 1×H₁₆), 4.55-4.47 (m, 4H, 2×H₂₃, 2×H₂₄), 4.34 (t, J=10.8 Hz, 1H, 1×H₁₆), 2.99 (d, J=6.0 Hz, 1H, H₁₄), 2.15-2.08 (m, 2H, 1×H₆, 1×H₁₀), 1.88-1.76 (m, 7H, 1×H₂, 1×H₇, 1×H₈, 1×H₁₃, 3×H₂₂), 1.71 (s, 1H, 1×H₄), 1.67-1.60 (m, 5H, 1×H₁, 1×H₁₃, 3×H₁₅), 1.41-1.30 (m, 4H, 1×H₁, 1×H₂, 1×H₇, 1×H₁₉), 1.03 (t, J=7.8 Hz, 3H, H₂₀), 0.97-0.94 (m, 1H, 1×H₁₉), 0.93 (s, 3H, H₁₈), 0.79 (td, J=14.4, 4.2 Hz, 1H, 1×H₁₁), 0.75 (d, J=7.2 Hz, 3H, H₁₇). ¹³C NMR (150 MHz, C₆D₆) 215.4 (C), 170.3 (C), 138.6 (CH), 135.7 (C), 135.1 (C), 135.1 (C), 134.9 (CH), 134.8 (CH), 134.7 (CH), 130.8 (CH), 130.6 (CH), 128.7 (CH), 128.6 (CH), 128.5 (CH), 97.2 (CH₂), 85.4 (CH), 70.7 (CH₂), 69.5 (CH), 67.5 (CH₂), 58.8 (CH), 45.1 (C), 43.9 (CH₂), 43.6 (C), 42.9 (CH), 41.7 (C), 35.8 (CH), 34.3 (CH₂), 29.9 (CH), 26.8 (CH), 25.3 (CH₂), 24.8 (CH₂), 22.8 (CH₂), 20.7 (CH₃), 15.1 (CH₃), 12.3 (CH₃), 10.2 (CH₃). IR (ATR-FTIR), cm⁻¹: 2931 (w), 1734 (m), 1455 (w), 1429 (w), 1368 (w), 1241 (m), 1158 (w), 1113 (m), 1024 (s), 941 (w), 847 (m), 823 (m), 734 (s), 698 (s), 499 (w). HRMS-ESI (m/z): [M-Si(C₆H₅)₂+Na]⁺ calcd for C₃₀H₄₄NaO₆, 523.3036; found, 523.3022. [α]_{D}²⁵=+42° (c=0.10, CHCl₃).

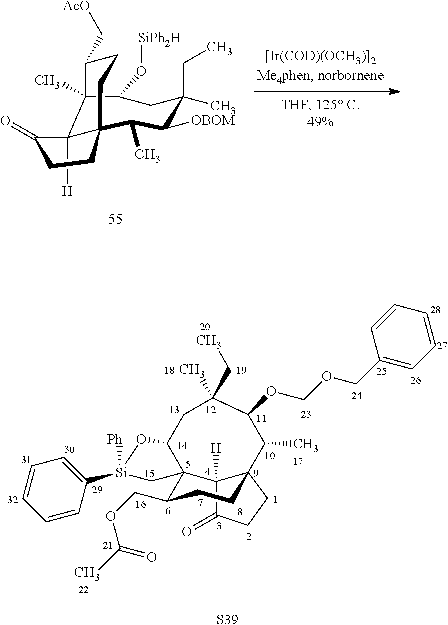

### Synthesis of Silacycle S39 (FIG. 13. Scheme 13)

This experiment was adapted from the work of Hartwig and co-workers.² A 4-mL pressure tube with a Teflon-coated valve was charged with 3,4,7,8-tetramethyl-1,10-phenanthroline (7.7 mg, 32.8 μmol, 12.5 mol %) and norbornene (37.0 mg, 393 μmol, 1.50 equiv) in the glovebox. A 4-mL vial was charged with silane 55 [210 mg, 262 μmol, 1 equiv, dried by azeotropic distillation with benzene (3×1.0 mL)]. The vessel containing the silane was evacuated and refilled using a balloon of argon. This process was repeated two times. Tetrahydrofuran (200 μL) was transferred into the vessel containing the silane and the resulting solution was added to the vessel containing the ligand and norbornene in the glovebox. The vessel containing the silane was rinsed with tetrahydrofuran (3×100 μL) and the combined rinses were transferred to the reaction vessel.

Methoxy(cyclooctadiene)iridium(I) dimer (8.7 mg, 13.1 μmol, 5.0 mol %) was added to an oven-dried 4-mL vial. Tetrahydrofuran (200 μL) was added into the vial containing the catalyst and the resulting solution was transferred dropwise via syringe to the reaction vessel in the glovebox. The vial containing the catalyst was rinsed with tetrahydrofuran (3×100 μL) and the combined rinses were transferred into the reaction vessel. The reaction vessel was sealed and the reaction mixture was stirred for 1 h at 24° C. in the glovebox. The sealed reaction vessel was then removed from the glovebox and placed in an oil bath that had been preheated to 120° C. The reaction mixture was stirred and heated for 2 h at 120° C. The reaction vessel was allowed to cool over 30 min to 24° C. and the cooled product mixture was concentrated to dryness. The residue obtained was filtered through a pad of silica gel (2.5×4.5 cm). The filter cake was washed with a mixture of ether and hexanes (1:1, v/v, 250 mL). The filtrate were combined and the combined filtrates were concentrated to dryness. The residue obtained purified by automated flash-column chromatography (eluting with hexanes initially, grading to 40% ether-hexanes, linear gradient) to afford the silacycle S39 as an amorphous white solid (102 mg, 49%).

R_{f}=0.45 (33% ether-hexanes; UV, PAA, CAM). ¹H NMR (500 MHz, CD₂Cl₂) δ 7.65-7.53 (m, 4H, 4×H₃₁), 7.35-7.24 (m, 11H, 2×H₂, 2×H₂₇, 1×H₂₈, 4×H₃₀, 2×H₃₂), 4.72 (dd, J=10.5, 3.5 Hz, 2H, H₂₃), 4.61 (s, 2H, H₂₄), 4.57 (d, J=9.5 Hz, 1H, H₁₁), 3.90-3.78 (m, 2H, H₁₆), 3.27 (d, J=5.5 Hz, 1H, H₁₄), 2.35 (s, 1H, H₄), 2.29 (dd, J=19.5, 11.0 Hz, 1H, 1×H₂), 2.24-2.15 (m, 2H, 1×H₂, 1×H₁₀), 2.04 (d, J=13.2 Hz, 1H, 1×H₁₅), 1.64 (s, 3H, H₂₂), 1.81-1.63 (m, 6H, 2×H₁, 1×H₆, 1×H₇, 1×H₈, 11×H₁₅), 1.61-1.36 (m, 4H, 1×H₇, 1×H₁₃, 2×H₁₉), 1.28 (dd, J=16.0, 10.0 Hz, 1H, 1×H₁₃), 1.09 (td, J=14.0, 3.5 Hz, 1H, 1×H₈), 0.94-0.89 (m, 3H, 3×H₁₇, 3×H₂₀), 0.76 (s, 3H, H₁₈). ¹³C NMR (150 MHz, C₆D₆) 216.2 (C), 170.3 (C), 138.3 (C), 138.2 (C), 134.5 (CH), 134.1 (CH), 133.9 (CH), 129.6 (CH), 129.5 (CH), 128.3 (C), 127.8 (CH), 127.6 (CH), 127.5 (CH), 127.5 (CH), 96.6 (CH₂), 83.8 (CH), 78.3 (CH), 70.5 (CH₂), 67.0 (CH₂), 61.4 (CH), 48.0 (C), 45.2 (CH₂), 43.2 (CH), 42.1 (C), 34.2 (CH₂), 33.4 (CH), 29.4 (CH₂), 26.7 (CH₃), 24.5 (CH₂), 21.9 (CH₂), 21.8 (CH₂), 20.6 (CH₃), 19.8 (CH₂), 10.8 (CH₃), 7.7 (CH₃). IR (ATR-FTR), cm⁻¹: 2931 (w), 1734 (m), 1455 (w), 1429 (w), 1368 (w), 1241 (m), 1158 (w), 1113 (m), 1024 (s), 941 (w), 847 (m), 823 (m), 734 (s), 698 (s), 499 (w). HRMS-ESI (m/z): [M+H]⁺ calcd for C₄₂H₅₃O₆Si, 681.3611; found, 681.3615. [α]_{D}²⁵=+39° (c=0.10, CHCl₃).

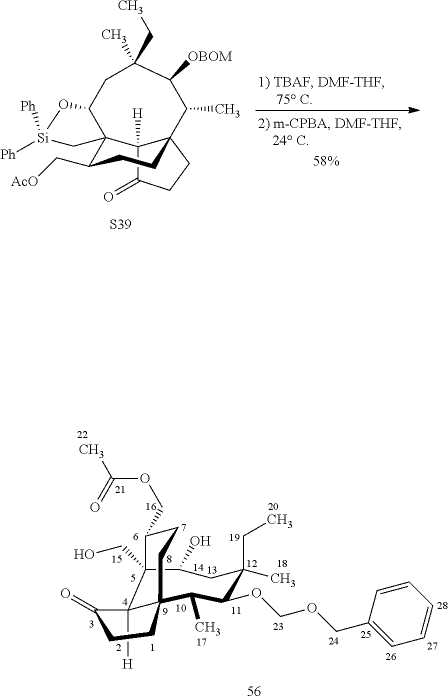

### Synthesis of Diol 56 (FIG. 13. Scheme 13)

A solution of tetrabutylammonium fluoride (1.0 M, 200 μL, 200 μmol, 2.00 equiv) in tetrahydrofuran was added dropwise via syringe to a solution of the silacycle S39 (68.1 mg, 100 μmol, 1 equiv) in a mixture of N,N-dimethylformamide (600 μL) and tetrahydrofuran (200 μL) at 24° C. The reaction vessel was placed in an oil bath that had been previously heated to 75° C. The reaction mixture was stirred and heated for 5 min at 75° C. The resulting mixture was immediately cooled to 24° C. with an ice bath. Freshly recrystallized m-chloroperbenzoic acid (34.5 mg, 200 μmol, 2.00 equiv) was added to the reaction mixture at 24° C. The reaction mixture was stirred for 15 min at 24° C. The product mixture was diluted sequentially with ether (5.0 mL) and aqueous potassium phosphate buffer solution (pH 7, 0.10 M, 3.0 mL). The diluted product mixture was transferred to a separatory funnel that had been charged with a mixture of ether and pentane (1:1, v/v, 30 mL). The layers that formed were separated and the organic layer was washed with saturated aqueous sodium bicarbonate solution (3×5 mL). The washed organic layer was dried over sodium sulfate. The dried solution was filtered and the filtrate was concentrated to dryness. The residue obtained was purified by automated flash-column chromatography (eluting with hexanes initially, grading to 50% ethyl acetate-hexanes, linear gradient) to afford the diol 56 as an amorphous white solid (29.8 mg, 58%).

R_{f}=0.45 (33% ether-hexanes; UV, PAA, CAM). ¹H NMR (500 MHz, CD₂Cl₂) δ 7.35-7.27 (m, 5H, 2×H₂₆, 2×H₂₇, 1×H₂₈), 4.84-4.74 (m, 2H, H₂₃), 4.67-4.60 (m, 2H, H₂₄), 4.30-4.24 (m, 1H, H₁₁), 4.24-4.12 (m, 3H, 1×H₁, 2×H₁₆), 3.90 (dd, J=11.5, 8.5 Hz, 1H, 1×H₁₉), 3.28 (d, J=6.0 Hz, 1H, H₁₄), 3.03 (br s, 1H, C15-OH), 2.40 (s, 1H, H₄), 2.38-2.32 (m, 1H, H₁₀), 2.27 (dd, J=10.5, 4.5 Hz, 2H, H₂), 2.17 (br s, 1H, C14-OH), 2.70 (dd, J=16.5, 8.0 Hz, 1H, 1×H₁₃), 1.99 (s, 31, H₂₂), 1.85 (dq, J=18.5, 3.5 Hz, 1H, 1×H₈), 1.81-1.76 (m, 1H, 1×H₁), 1.70-1.64 (m, 3H, 1×H₇, 2×H₁₉), 1.63-1.52 (m, 3H, 1×H₁, 1×H₆, 1×H₇), 1.51-1.45 (m, 1H, 1×H₁), 1.17 (td, J=14.5, 4.5 Hz, 1H, 1×H₈), 1.00 (s, 3H, H₁₅), 0.95 (d, J=7.0 Hz, 3H, H₁₇), 0.92 (t, J=7.5 Hz, 3H, H₂₀). ¹³C NMR (150 MHz, CD₂Cl₂) 221.7 (C), 171.5 (C), 138.8 (C), 128.9 (CH), 128.1 (CH), 128.1 (CH), 97.9 (CH₂), 85.6 (CH), 71.2 (CH₂), 66.8 (CH₂) 66.0 (CH), 62.8 (CH₂), 57.7 (CH), 46.1 (C), 45.4 (C), 44.4 (CH₂), 41.9 (C), 41.1 (CH), 35.9 (CH), 35.3 (CH₂), 30.4 (CH₂), 27.3 (CH₃), 26.7 (CH₂), 22.3 (CH₂), 22.2 (CH₂), 21.4 (CH₃), 12.4 (CH₃), 8.6 (CH₃). IR (ATR-FTIR), cm⁻¹: 3344 (br w), 2951 (m), 1740 (m), 1459 (w), 1365 (w), 1248 (s), 1172 (w), 1104 (w), 1041 (s), 1018 (s), 968 (m) 939 (m), 736 (m), 697 (m). HRMS-ESI (m/z): [M+K]⁺ calcd for C₃₀H₄₄KO₇, 555.2724; found, 555.2737. [α]_{D}²⁵=+44° (c=0.10, CHCl₃).

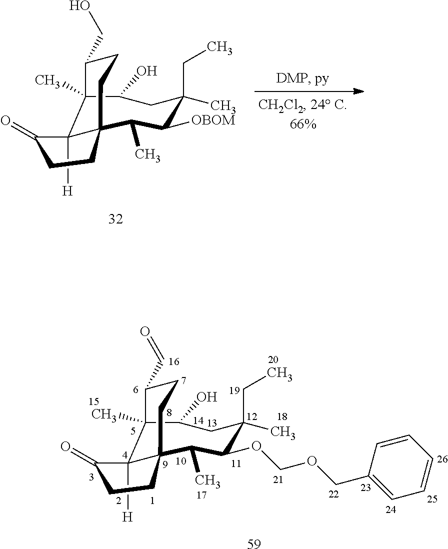

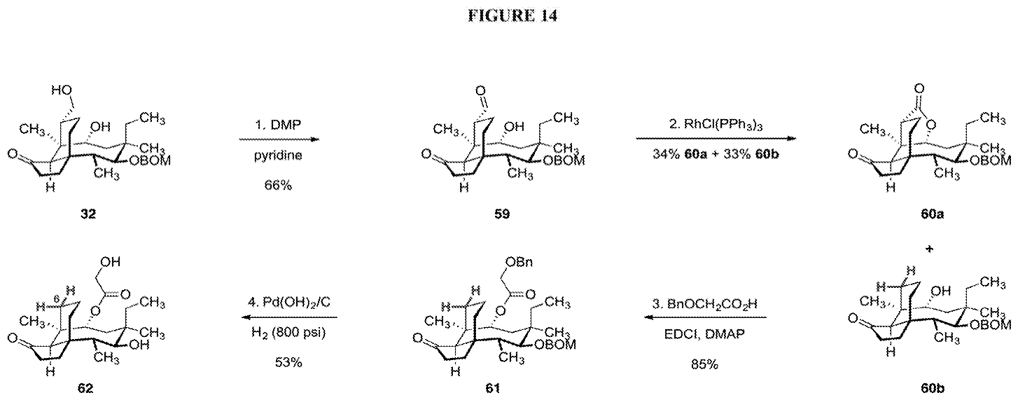

### Synthesis of Aldehyde 59 (FIG. 14, Scheme 14)

Six equal portions of Dess-Martin periodinane (30.5 mg, 72.0 μmol, 1.10 equiv) was added over 1 h to a solution of the diol 32 (30.0 mg, 65.4 μmol, 1 equiv) and pyridine (52.9 μL, 654 mmol, 10.0 equiv) in dichloromethane (500 μL) at 24° C. The resulting mixture was stirred for 10 min at 24° C. The product mixture was diluted sequentially with ether (1.0 mL), a saturated aqueous sodium bicarbonate solution (500 μL) and a saturated aqueous sodium thiosulfate solution (500 μL). The resulting mixture was stirred for 10 min at 24° C. The resulting mixture was transferred to a separatory funnel and the layers that formed were separated. The aqueous layer obtained was extracted with dichloromethane (3×10 mL). The organic layers were combined and the combined organic layer was dried over sodium sulfate. The dried solution was filtered and the filtrate was concentrated to dryness. The residue obtained was purified by automated flash-column chromatography (eluting with hexanes initially, grading to 20% ethyl acetate-hexanes, linear gradient) to afford aldehyde 59 as a clear oil (20.1 mg, 66%).

R_{f}=0.59 (30% ethyl acetate-hexanes; UV, PAA, CAM). ¹H NMR (400 MHz, CDCl₃) δ 9.84 (s, 1H, H₁₆), 7.32-7.26 (m, 2H, H₂₅), 7.22-7.17 (m, 2H, H₂₄), 7.13-7.06 (m, 1H, H₆), 4.58-4.50 (m, 4H, 2×H₂₁, 2×H₂₂), 4.10 (br s, 1H, H₁), 2.96 (d, J=6.0 Hz, 1H, H₄), 2.28-2.20 (m, 3H, 1×H₆, 1×H₁₀, 1×OH), 1.82-1.77 (m, 2H, H₂), 1.73 (s, 3H, H₁₉), 1.69-1.59 (m, 2H, 1×H₇, 1×H₁₉), 1.59-1.54 (m, 2H, 1×H₄, 1×H₁₉), 1.53-1.49 (m, 1H, 1×H₇), 1.46-1.40 (m, 1H, 1×H₁), 1.40-1.35 (m, 1H, 1×H₈), 1.35-1.30 (m, 2H, H₁₃), 1.05-0.95 (m, 1H, 1×H₁), 0.93 (s, 3H, H₁₈), 0.89 (t, J=11.4 Hz, 3H, H₂₀), 0.81 (d, J=10.8 Hz, 3H, H₁₇), 0.64 (td, J=21.6, 6.6 Hz, 1H, 1×H₈). ¹³C NMR (100 MHz, CDCl₃) δ 215.1 (CH), 202.6 (C), 138.3 (C), 128.3 (CH), 128.2 (CH), 127.5 (CH), 96.8 (CH₂), 85.1 (CH), 70.3 (CH₂), 64.4 (CH), 58.0 (CH), 53.3 (CH), 44.2 (C), 41.9 (CH₂), 41.1 (C), 35.3 (1×CH, 1×C), 33.6 (CH₂), 28.4 (CH₂), 26.7 (CH₃), 25.1 (CH₂), 22.0 (CH₂), 17.6 (CH₂), 13.9 (CH₃), 11.9 (CH₃), 8.1 (CH). IR (ATR-FTIR), cm⁻¹: 2949 (w), 2882 (w), 1735 (s), 1707 (s), 1464. (w), 1382 (w), 1242 (w), 1162 (w), 1105 (w), 1040 (s), 1024 (s), 935 (w), 740 (w). HRMS-ESI (m/z): [M+H]⁺ calcd for C₂₈H₄₁O₅, 457.2854; found, 457.2955. [α]_{D}²⁵=+47° (c=0.10, CHCl₃).

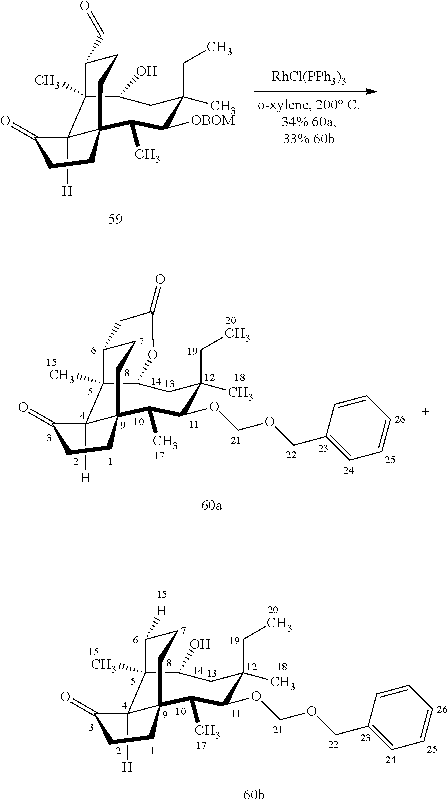

### Tsuji-Wilkinson Decarboxylation of Aldehyde 59 (FIG. 14, Scheme 14)

A 4-mL pressure tube with a Teflon-coated valve was charged with the aldehyde 59 (20.1 mg, 44.0 μmol, 1 equiv). Benzene (500 μL) was added and the solution was concentrated to dryness. This process was repeated twice. Wilkinson's catalyst (204 mg, 220 μmol, 5.00 equiv) was added to the reaction vessel. The reaction vessel was evacuated and refilled using a balloon of argon. This process was repeated twice. o-Xylene (2.0 mL) was added to the reaction vessel and the resulting mixture was degassed by bubbling argon through the solution for 5 min. The reaction vessel was transferred into the glovebox. The reaction vessel was sealed and the sealed vessel was removed out of the glovebox. The sealed reaction vessel was placed in a sand bath that had been previously heated to 200° C. The resulting mixture was stirred and heated for 24 h at 200° C. The product mixture was cooled over 2 h to 24° C. The cooled product mixture was diluted sequentially with ether (5.0 mL). The diluted product mixture was filtered through a pad of silica gel and the pad was rinsed with a mixture of ethyl acetate and hexanes (1:4 v/v, 100 mL). The filtrates were combined and the combined filtrates were concentrated to dryness. The residue obtained was purified by automated flash-column chromatography (eluting with hexanes initially, grading to 66% ether-hexanes, linear gradient) to afford separately the lactone 60a as an amorphous white solid (6.9 mg, 34%) and 11-benzyloxymethylenoxy-6-desmethyl-19,20-dihydromutilin (60b) as a colorless clear film (6.2 mg, 33%).

Lactone 60a: R_{f}=0.18 (40% ethyl acetate-hexanes; UV, PAA, CAM). ¹H NMR (400 MHz, CD₂Cl₂) δ 7.36-7.26 (m, 5H, 2×H₂₄, 2×H₂₅, 1×H₂₆), 4.99 (d, J=7.6 Hz, 1H, H₄), 4.76 (dd, J=8.0, 0.80 Hz, 2H, H₂₁), 4.64 (dd, J=15.2, 3.2 Hz, 2H, H₂₂), 3.33 (d, J=6.8 Hz, 1H, H₁₁), 2.62-2.55 (M, 1H, 1×H₁₀), 2.37 (dd, J=10.4, 8.0 Hz, 1H, H_{b}), 2.22 (dd, J=19.2, 10.8 Hz, 1H, 1×H₂), 2.15-2.05 (m, 2H, 1×H₂, 1×H₄), 1.93-1.65 (m, 5H, 2×H₁, 1×H₇, 2×H₁₉), 1.65-1.52 (m, 3H, 1×H₈, 2×H₁₃), 1.48 (dd, J=16.0, 7.6 Hz, 1H, 1×H₇), 1.25 (s, 3H, H₁₅), 1.14 (td, J=13.6, 6.0 Hz, 1H, 1×H₈), 1.05 (s, 3H, H₁₈), 0.97 (d, J=7.2 Hz, 3H, H₁₇), 0.91 (t, J=7.6 Hz, 3H, H₂). ¹³C NMR (100 MHz, CD₂Cl₂) δ 216.2 (C), 178.4 (C), 138.7 (C), 12.9 (CH), 128.2 (2×CH), 98.4 (CH₂), 85.5 (CH), 77.2 (CH), 71.3 (CH), 53.7 (CH), 45.4 (CH), 44.0 (C), 43.4 (C), 42.4 (C), 38.6 (CH), 34.2 (CH₂), 32.2 (CH₂), 27.9 (CH₂), 27.5 (CH₂), 26.5 (CH₃), 23.1 (CH₂), 19.3 (CH₂) 16.6 (CH₃), 14.0 (CH₃), 8.3 (CH₃). IR (ATR-FTIR), cm⁻¹: 2036 (w), 2879 (w), 1770 (s), 1742 (s), 1454 (w), 1385 (w), 1316 (w), 1305 (w), 1272 (w), 1198 (m), 1166 (m), 1096 (m), 1034 (s), 1020 (s), 955 (m), 924 (s), 738 (s), 698 (m), 675 (m). HRMS-ESI (m/z): [M+H]⁺ calcd for C₂H₃₉O₅, 455.2797; found, 455.2799.

11-Benzyloxymethylenoxy-16-desmethyl-19,20-dihydromutilin (60b): R_{f}=0.25 (40% ethyl acetate-hexanes; UV, PAA, CAM). ¹H NMR (600 MHz, CDCl₃) δ 7.37-7.28 (m, 5H, 2×H₂₄, 2×H₂₃, 1×H₂₆), 4.76 (dd, J=11.4, 4.2 Hz, 2H, H₂₁), 4.66 (s, 2H, H₂₂), 4.19 (t, J=7.2 Hz, 1H, H₁₁), 3.28 (d, J=6.6 Hz, 11H, H₄), 2.44-2.40 (m, 1H, H₁₀), 2.25-2.12 (m, 2H, H₂), 1.99 (s, 1H, H₄), 1.73-1.53 (m, 8H, 2×H₁, 2×H₆, 1×H₇, 2×H₁₃, 1×H₁₉), 1.48-1.42 (m, 2H, 1×H₈, 1×H₁₉), 1.33-1.26 (m, 2H, 1×H₇, 1×OH), 1.25 (s, 3H, H₁₈), 1.04-1.01 (m, 4H, 1×H₈, 3×H₁₈), 0.96-0.91 (m, 6H, 3×H₁₇, 3×H₂₀). ¹³C NMR (150 MHz, CDCl₃) δ 217.7 (C), 137.9 (C), 128.4 (CH), 127.7 (CH), 127.6 (CH), 97.1 (CH₂), 85.4 (CH), 70.7 (CH₂), 66.4 (CH), 56.8 (CH), 45.0 (C), 41.3 (CH₂), 41.3 (C), 39.4 (C), 35.1 (CH), 34.6 (CH₂), 29.7 (CH₂), 29.2 (CH₂), 27.1 (CH₃), 25.6 (CH₂), 22.1 (CH₂), 17.8 (CH₂), 15.1 (CH₃), 12.2 (CH₃), 8.0 (CH₃). IR (ATR-FTIR), cm⁻¹: 2976 (w), 2924 (m), 1736 (m), 1461 (w), 1380 (w), 1287 (w), 1147 (m), 1067 (m), 1039 (s), 970 (w), 944 (w), 917 (w). HRMS-ESI (m/z): [M+H]⁺ calcd for C₂₇H₄₁O₄, 429.3005; found, 429.3007.

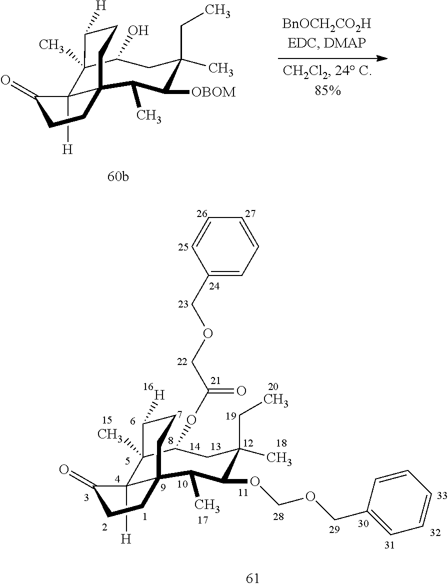

### Synthesis of bis(benzyl)ether 61 (FIG. 14, Scheme 14)

A 4-mL vial was charged with 11-benzyloxymethylenoxy-6-desmethyl-19,20-dihydromutilin (60b, 6.2 mg, 14.5 μmol, 1 equiv) and benzyloxyacetic acid (6.2 μL, 43.4 μmol, 3.00 equiv). Benzene (500 μL) was added to the vial. The solution was concentrated to dryness. This process was repeated twice. The reaction vessel was evacuated and refilled using a balloon of argon. This process was repeated twice. Dichloromethane (300 μL), I-ethyl-3-(3-dimethylaminopropyl)carbodiimide hydrochloride (8.3 mg, 43.4 μmol, 3.00 equiv), and 4-dimethylaminopyridine (5.3 mg, 43.4 μmol, 3.00 equiv) were added sequentially to the reaction vessel at 24° C. The reaction mixture was stirred for 1 h at 24° C. The product mixture was concentrated to dryness. The residue obtained was purified by flash-column chromatography (eluting with hexanes initially, grading to 12% ether-hexanes, linear gradient) to afford the bis(benzyl)ether 61 as a clear oil (7.1 mg, 85%).

R_{f}=0.23 (30% ethyl acetate-hexanes; UV, PAA, CAM). ¹H NMR (600 MHz, CDCl₃) δ 7.38-7.28 (m, 10H, 2×H₂₅, 2×H₂₆, 1×H₂₇, 2×H₃₁, 2×H₃₂, 1×H₃₃), 5.79 (d, J=7.8 Hz, 1H H₁₄) 4.77 (dd, J=12.6, 4.8 Hz, 2H, H₂₂), 4.68-4.60 (m, 4H, 2×H₂₃, 2×H₂₉), 4.08 (dd, J=22.2, 6.0 Hz, 2H, H₂₈), 3.30 (d, J=6.6 Hz, 1H, H₁), 2.63-2.58 (m, 1H, H₁₀), 2.26-2.13 (m, 2H, H₂), 2.03 (s, 1H, H₄), 1.88 (q, J=14.0 Hz, 1H, 1×H₉), 1.81-1.76 (m, 1H, 1×H₇), 1.75-1.59 (m, 4H, 1×H₁, 1×H₇, 1×H₈, 1×H₁₃), 1.50-1.38 (m, 3H, 1×H₁, 1×H₁₃, 1×H₁₉), 1.31-1.26 (m, 4H, 3×H₁₃, 1×H₁₆), 1.14 (d, J=13.8 Hz, 1H, 1×H₆), 1.03-0.98 (m, 4H, 1×H₈, 3×H₁₈), 0.96 (d, J=7.2 Hz, 3H, H₁₃), 0.77 (t, J=7.5 Hz, 3H, H₂₀). ¹³C NMR (150 MHz, CDCl₃) δ 217.1 (C), 169.6 (C), 137.9 (C), 137.1 (C), 128.5 (CH), 128.4 (CH), 128.1 (CH), 128.0 (CH), 127.7 (CH), 127.7 (CH), 67.0 (CH₂), 85.2 (CH), 73.3 (CH₂), 70.8 (CH₂), 69.3 (CH), 67.1 (CH₂), 56.3 (CH), 45.1 (C), 41.3 (C), 39.2 (C), 38.5 (CH₂), 35.4 (CH), 34.5 (CH₂), 29.8 (CH₂), 29.1 (CH₃), 26.7 (CH₃), 25.6 (CH₂), 21.8 (CH₂), 17.7 (CH₂), 16.5 (CH₃), 12.3 (CH₃), 8.0 (CH₃). IR (ATR-FTIR), cm⁻¹: 2957 (w), 2878 (w), 1755 (m), 1734 (m), 1460 (m), 1428 (w), 1286 (w), 1239 (w), 1214 (w), 1113 (s), 1071 (m), 1007 (m), 952 (m), 916 (w), 839 (m), 738 (m), 701 (s), 613 (m), 499 (s). HRMS-ESI (m/z): [M+H]⁺ calcd for C₃₆H₄₉O₆, 577.3529; found, 577.3538.

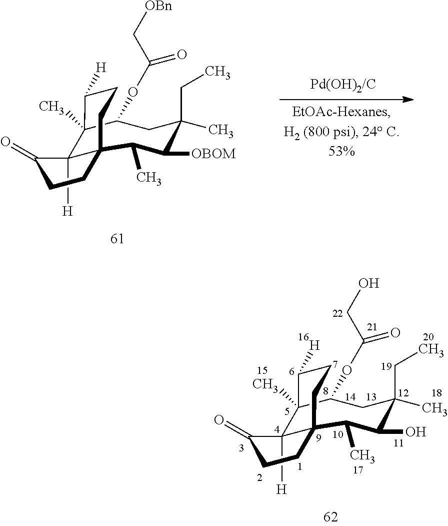

### Synthesis of 16-desmethyl-19,20-dihydropleuromutilin (62, FIG. 14, Scheme 14)

A 4-mL vial was charged with the bis(benzyl)ether 61 (7.1 mg, 12.3 μmol, 1 equiv). Benzene (500 μL) was added to the vial. The solution was concentrated to dryness. This process was repeated twice. The reaction vessel was evacuated and refilled using a balloon of nitrogen. This process was repeated twice. Ethyl acetate (50 μL), hexanes (250 μL), and Pearlman's catalyst (20 wt. % loading, 4.3 mg, 6.2 μmol, 0.500 equiv) were added sequentially to the reaction vessel at 24° C. The vial was placed in a stainless steel hydrogenation apparatus. The apparatus was purged with dihydrogen by pressurizing to 50 psi and venting three times. The vessel was pressurized with dihydrogen (800 psi), sealed, and the reaction mixture was stirred for 12 h at 24° C. The apparatus was depressurized by slowly venting the dihydrogen. The product mixture was filtered through a pad of celite and the pad was rinsed with ether (50 mL). The filtrates were collected and combined and the combined filtrates were concentrated. The residue obtained was purified by automated flash-column chromatography (eluting with hexanes initially, grading to 50% ethyl acetate-hexanes, linear gradient) to afford 16-desmethyl-19,20-dihydropleuromutilin (62) as an amorphous white solid (2.2 mg, 53%).

R_{f}=0.23 (30% ethyl acetate-hexanes; PAA, CAM). ¹H NMR (600 MHz, CDCl₃) δ 5.76 (d, J=7.8 Hz, 1H, H₄), 4.13 (d, J=5.4 Hz, 2H, H₂), 3.43 (t, J=6.0 Hz, 1H, H₁₁), 2.53-2.48 (m, 1H, H₁₀), 2.35 (td, J=5.4, 1.2 Hz, 1H, C22-OH), 2.28-2.15 (m, 2H, H₂), 2.07 (s, 1H, H₄), 1.87-1.79 (m, 1H, 1×H_{1Y}), 1.76-1.65 (m, 3H, 1×H₁, 1×H₇, 1×H₈), 1.63-1.59 (m, 1H, 1×H₁₃), 1.53-1.50 (m, 2H, 1×H₁, 1×C11-OH), 1.49-1.45 (m, 1H, 1×H₇), 1.45-1.41 (m, 1H, 1×H₁₃), 1.41-1.37 (m, 1H, 1×H₉), 1.33 (td, J=13.8, 4.8 Hz, 1H, 1×H₆), 1.28 (s, 3H, H₁₅), 1.15-1.10 (m, 1H, 1×H₁₆), 1.04-0.99 (m, 4H, 1×H₈, 3×H₁₈), 0.97 (d, J=7.2 Hz, 3H, H₁₇), 0.76 (t, J=7.5 Hz, 3H, H₂₀). ¹³C NMR (150 MHz, CDCl₃) δ 216.7 (C), 172.6 (C), 76.5 (CH), 70.6 (CH), 60.5 (CH₂), 56.2 (C), 45.2 (C), 40.8 (CH₂), 38.1 (CH₂), 38.3 (C), 34.7 (CH), 34.4 (CH₂), 29.7 (CH₂), 28.8 (CH₂), 26.3 (CH₂), 25.4 (CH₃), 20.8 (CH), 17.6 (CH₂), 16.4 (CH₃), 11.4 (CH₃), 8.0 (CH₃). IR (ATR-FTIR), cm⁻¹: 3369 (br w), 2964 (m), 2940 (m), 2914 (m), 1725 (s), 1456 (m), 1420 (w), 1383 (w), 1373 (w), 1214 (m), 1081 (w), 1160 (w), 1103 (s), 1043 (w), 1010 (m), 998 (w), 951 (m), 933 (w), 661 (w), 562 (w), 511 (w). HRMS-ESI (m/z): [M+H]⁺ calcd for C₂H₃₅O₅, 367.2484; found, 367.2487.

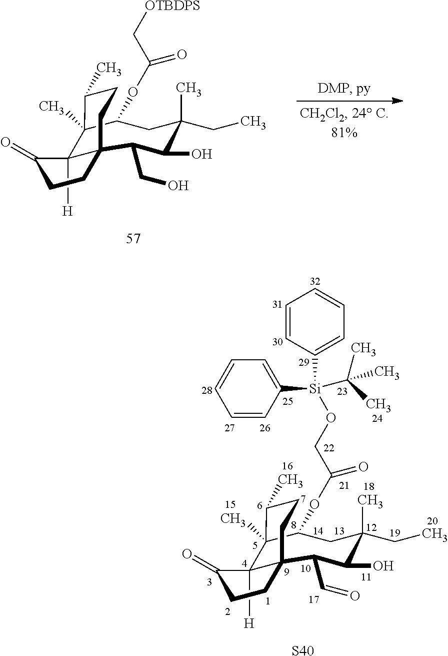

### Synthesis of O-tert-butyldiphenylsilyl-12-epi-17-oxo-19,20-dihydropleuromutilin (S40, FIG. 13D, Table 1)

Five equal portions of Dess-Martin periodinane (26.9 mg, 63.4 μmol, 1.10 equiv) was added over 1 h to a solution of O-tert-butyldiphenylsilyl-12-epi-17-hydroxy-19,20-dihydropleuromutilin 57 (36.6 mg, 57.6 μmol, 1 equiv) and pyridine (46.6 μL, 576 μmol, 10.0 equiv) in dichloromethane (500 μL) at 24° C. The resulting mixture was stirred for 2 h at 24° C. The product mixture was diluted sequentially with ether (1.0 mL), a saturated aqueous sodium bicarbonate solution (500 μL) and a saturated aqueous sodium thiosulfate solution (500 μL). The resulting mixture was stirred for 5 min at 24° C. The resulting mixture was transferred to a separatory funnel and the layers that formed were separated. The aqueous layer obtained was extracted with dichloromethane (3×5 mL). The organic layers were combined and the combined organic layer was dried over sodium sulfate. The dried solution was filtered and the filtrate was concentrated to dryness. The residue obtained was purified by automated flash-column chromatography (eluting with hexanes initially, grading to 20% ether-hexanes, linear gradient) to afford O-tert-butyldiphenylsilyl-2-epi-17-oxo-19,20-dihydropleuromutilin (S40) as an amorphous white solid (29.7 mg, 81%).

R_{f}=0.25 (33% ether-dichloromethane; UV, PAA, CAM). ¹H NMR (400 MHz, CDCl₃) δ 9.76 (d, J=4.4 Hz, 1H, H₇), 7.73-7.67 (m, 4H, 2×H₂₇, 2×H₃₁), 7.46-7.41 (m, 6H, 2×H₂₆, 1×H₂₈, 2×H₃₀, 1×H₃₂), 5.32 (d, J=8.0 Hz, 1H, H₁₄), 4.18 (dd, J=25.6, 9.2 Hz, 2H, H₂₂), 3.97 (d, J=6.8 Hz, 1H, H₁₁), 3.07 (t, J=5.6 Hz, 11H, H₁₀), 2.45-2.14 (m, 3H, 2×H₂, 1×H₁₉), 2.04 (s, 1H, H₄), 1.98 (dd, J=16.0, 8.4 Hz, 1H, 1×H₇), 1.88-1.81 (m, 1H, 1×H₈), 1.73-1.32 (m, 10H, 1×H₁, 1×H₆, 1×H₇, 1×H₁, 3×H₁₅, 1×H₁₉, 1×OH), 1.28-1.18 (m, 1H, 1×H₈), 1.15-1.08 (m, 12H, 3×H₁₈, 9×H₂₄), 0.98 (d, J=16.0 Hz, 1H, 1×H₁₃), 0.90 (t, J=7.4 Hz, 3H, H₂₀), 0.63 (d, J=6.8 Hz, 3H, H₁₆). ¹³C NMR (100 MHz, CDCl₃) δ 214.8 (CH), 202.5 (C), 169.6 (C), 135.6 (CH), 135.6 (CH), 132.8 (C), 132.7 (C), 129.9 (CH), 127.8 (CH), 72.8 (CH), 68.3 (CH), 62.8 (CH₂), 57.8 (CH), 54.8 (CH), 43.4 (C), 41.8 (C), 41.3 (CH₂), 40.3 (C), 36.4 (CH), 34.2 (CH₂), 33.3 (CH₂), 31.0 (CH₂), 26.7 (CH₃), 26.6 (CH₂), 26.4 (CH₂), 19.2 (C), 17.9 (CH₃), 16.5 (CH₃), 14.7 (CH₃), 7.8 (CH₃). IR (ATR-FTIR), cm⁻¹: 2942 (w), 2881 (w), 1737 (s), 1454 (m), 1383 (w), 1267 (w), 1243 (w), 1191 (w), 1161 (w), 1119 (s), 1036 (s), 1019 (s), 988 (s), 959 (s), 925 (s), 735 (s), 697 (s), 564 (w). HRMS-ESI (m/z): [M+H]⁺ calcd for C₃₈H₅₂O₆Si, 633.3611; found, 633.3608. [α]_{D}²⁵=+20° (c=0.10, CHCl₃).

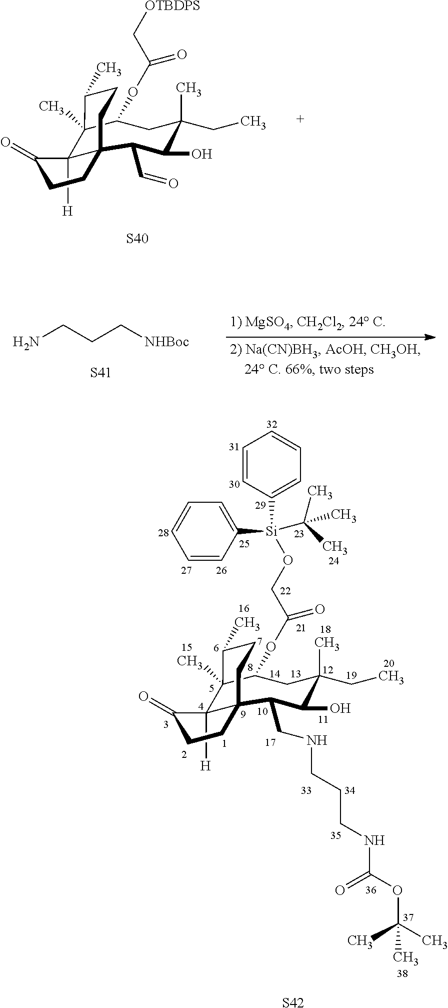

### Synthesis of Secondary Amine S40 (FIG. 13D, Table 1)

N-(tert-Butylcarbonyl)-1,3-diaminopropane (S41, 16.5 mg, 93.8 μmol, 2.00 equiv) was added to a suspension of O-tert-butyldiphenylsilyl-12-epi-17-oxo-19,20-dihydropleuromutilin S40 [29.7 mg, 46.9 μmol, 1 equiv, dried by azeotropic distillation with benzene (200 μL)] and anhydrous magnesium sulfate (28.5 mg, 235 mmol, 5.00 equiv) in dichloromethane (300 μL). The reaction was stirred for 4 h at 24° C. The resulting mixture was filtered through a small column of powdered sodium sulfate (0.5 cm×0.5 cm). The column was rinsed with dichloromethane (5.0 mL). The filtrates were combined and the combined filtrates were concentrated to dryness. The residue obtained was transferred to a 4-mL vial with benzene (1.5 mL) and the resulting solution was concentrated to dryness. The reaction vessel was evacuated and refilled using a balloon of argon. This process was repeated twice. The residue obtained was dissolved in methanol (200 μL). Sodium cyanoborohydride (6.0 mg, 93.8 μmol, 2.00 equiv) and a solution of acetic acid (2.9 μL, 49.2 μmol, 1.05 equiv) in methanol (100 μL) were added to the reaction vessel at 24° C. The reaction mixture was stirred for 2 h at 24° C. The product mixture was transferred to a separatory funnel that had been charged with dichloromethane (10 mL) and saturated aqueous sodium bicarbonate solution (2.0 mL). The layers that formed were separated and the aqueous layer was extracted with dichloromethane (3×5 mL). The organic layers were combined and dried over sodium sulfate. The dried solution was filtered and the filtrate was concentrated to dryness. The residue obtained was purified by automated flash-column chromatography (eluting with dichloromethane initially, grading to 10% methanol-dichloromethane, linear gradient) to afford the secondary amine S42 as a colorless clear film (24.6 mg, 66%).

R_{f}=0.75 (10% methanol-dichloromethane; UV, PAA, CAM). ¹H NMR (400 MHz, CDCl₃) δ 7.70-7.65 (m, 4H, 2×H₂₇, 2×H₃₁), 7.46-7.35 (m, 6H, 2×H₂₆, 1×H₂₈, 2×H₃₀, 1×H₃₂), 5.54 (d, J=8.0 Hz, 1H, H₁₄), 4.90 (br s, 1H, NH), 4.14 (dd, J=22.4, 5.6 Hz, 2H, H₂₂), 3.64 (d, J=5.6 Hz, 1H, H₁₁), 3.27-3.12 (m, 2H, H₁₇), 3.06-2.94 (m, 1H, 1×H₃₃), 2.90-2.78 (m, 1H, 1×H₃₃, 1×H₃₅), 2.78-2.60 (m, 1H, 1×H₃₅), 2.31-2.15 (m, 3H, 2×H₂, 1×H₁₀), 2.05-1.96 (m, 2H, 1×H₄, 1×H₁₃), 1.95-1.85 (m, 1H, 1×H₁), 1.84-1.70 (m, 3H, 1×H₆, 1×H₈, 1×OH), 1.67-1.50 (m, 4H, 1×H₁, 1×H₇, 1×H₁₉, 1×H₃₅), 1.49-1.40 (m, 10H, 1×H₃₅, 9×H₃₈), 1.39-1.33 (m, 4H, 3×H₁₅, 1×H₁₉), 1.32-1.23 (m, 1H, 1×H₁₇), 1.19-1.12 (m, 1H, 1×H), 1.07 (s, 9H, H₂₄), 1.02 (s, 3H, H₁₈), 0.89-0.86 (m, 1H, 1×H₁₃), 0.83 (t, J=7.2 Hz, 3H, H₂₀), 0.61 (d, J=6.4 Hz, 3H, H₁₆). ¹³C NMR (100 MHz, CDCl₃) δ 216.7 (C), 169.8 (C), 156.3 (C), 135.5 (CH), 132.7 (C), 132.6 (C), 129.9 (CH), 127.8 (CH), 79.5 (C), 773 (C), 72.2 (CH), 68.8 (CH), 62.8 (CH₂), 58.1 (CH), 48.2 (CH₂) 46.3 (CH₂), 44.5 (CH₂), 41.9 (C), 41.5 (CH₂), 39.9 (CH), 39.5 (C), 38.0 (CH₂), 36.6 (CH), 34.5 (CH₂), 34.4 (CH₂), 30.7 (CH₂), 28.4 (CH₃), 27.0 (CH₂), 26.7 (CH), 25.6 (CH₂), 19.2 (CH₃), 19.1 (C), 16.7 (CH₃), 14.9 (CH₃), 7.9 (CH₃). IR (ATR-FTIR), cm⁻¹: 2925 (s), 1722 (s), 1650 (s), 1540 (w), 1494 (m), 1456 (m), 1409 (w), 1375 (w), 1276 (s), 1152 (m), 1117 (s), 1017 (s), 980 (m), 954 (m), 939 (m), 917 (m), 956 (w), 809 (w), 734 (s), 699 (m). HRMS-ESI (m/z): [M+H]⁺ calcd for C₄₇H₇₀N₂O₇Si, 791.5031; found, 791.5017.

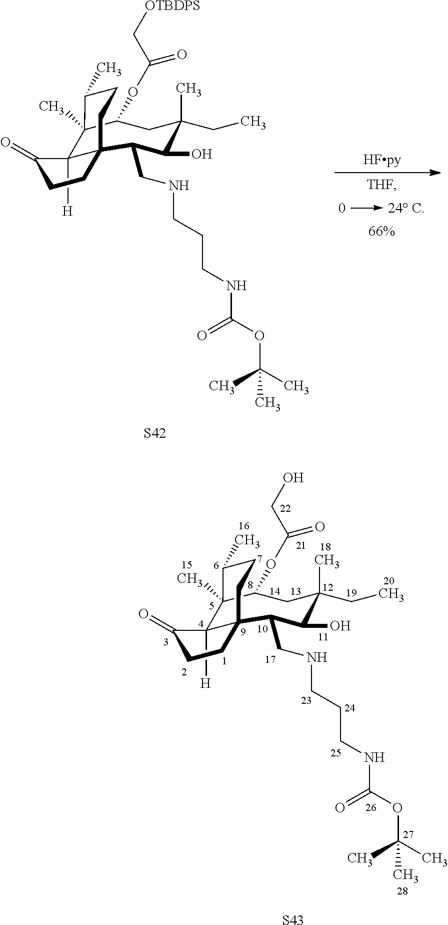

### Synthesis of Amino Alcohol S43

Olah's reagent (4.0 μL, 155 μmol, 5.00 equiv) was added dropwise via syringe to a solution of the secondary amine S42 (24.6 mg, 31.1 μmol, 1 equiv) in tetrahydrofuran (300 μL) at 0° C. The reaction mixture was allowed to warm up over 3.5 h to 24° C. The product mixture was transferred to a separatory funnel that had been charged with dichloromethane (10 mL) and saturated aqueous sodium bicarbonate solution (2.0 mL). The layers that formed were separated and the aqueous layer was extracted with dichloromethane (3×5 mL). The organic layers were combined and dried over sodium sulfate. The dried solution was filtered and the filtrate was concentrated to dryness. The residue obtained was purified by automated flash-column chromatography (eluting with dichloromethane-1% ammonium hydroxide initially, grading to 10% methanol-dichloromethane-1% ammonium hydroxide, linear gradient) to afford the amino alcohol S43 as a colorless clear film (11.4 mg, 66%).

R_{f}=0.15 (10% methanol-dichloromethane; UV, CAM). ¹H NMR (500 MHz, CDCl₃) δ 5.63 (d, J=8.0 Hz, 1H, H₁₄), 4.87 (br s, 1H, NH), 4.09 (d, J=10.0 Hz, 1H, 1×H₂₂), 4.01 (d, J=10.0 Hz, 1H, 1×H₂₂), 3.60 (d, J=6.5 Hz, 1H, H₁₁), 3.23-3.13 (m, 2H, H₁₇), 2.84-2.68 (m, 3H, 1×H₂₃, 2×H₂₅), 2.64-2.54 (m, 1H, 1×H₂₅), 2.27 (dd, J=19.5, 11.0 Hz, 1H, 1×H₂), 2.21-2.13 (m, 2H, 1×H₂, 1×H₁₀), 2.09-2.03 (m, 2H, 1×H₄, 1×H₁₃), 1.85 (t, J=11.2 Hz, 1H, 1×H₈), 1.80-1.74 (m, 1H, 1×H₇), 1.74-1.67 (m, 2H, 1×H₁, 1×H₁₉), 1.66-1.52 (m, 3H, 1×H₆, 1×H₇, 1×OH), 1.48-1.29 (m, 16H, 1×H₁, 3×H₁₅, 1×H₁₉, 2×H₂₄, 9×H₂₈) 1.16 (td, J=14.0, 4.5 Hz, 1H, 1×H₈), 1.05-0.95 (m, 4H, 1×H₁₃, 3×H₁₈), 0.85 (t, J=7.5 Hz, 3H, H₂₀), 0.69 (d, J=6.0 Hz, 3H, H₁₆). ¹³C NMR (125 MHz, CDCl₃) δ 216.6 (C), 172.1 (C), 156.1 (C), 79.3 (C), 72.7 (CH), 70.3 (CH), 61.3 (CH₂), 58.0 (CH), 48.3 (CH₂), 46.6 (CH₂), 44.5 (C), 41.9 (C), 41.4 (CH₂), 40.1 (CH), 40.0 (C), 38.2 (CH₂), 36.5 (CH), 34.5 (CH₂), 34.4 (CH₂), 30.6 (CH₂), 30.0 (CH₂), 28.4 (CH₃), 27.0 (CH₂), 25.6 (CH₂), 18.8 (CH₃), 16.7 (CH₃), 14.9 (CH₃), 7.9 (CH₃). IR (ATR-FTIR), cm⁻¹: 2931 (s), 1731 (m), 1647 (m), 1495 (w), 1462 (m), 1284 (m), 1155 (w). HRMS-ESI (m/z): [M+H]⁺ calcd for C₃₀H₅₃N₂O₇, 553.3853; found, 553.3825.

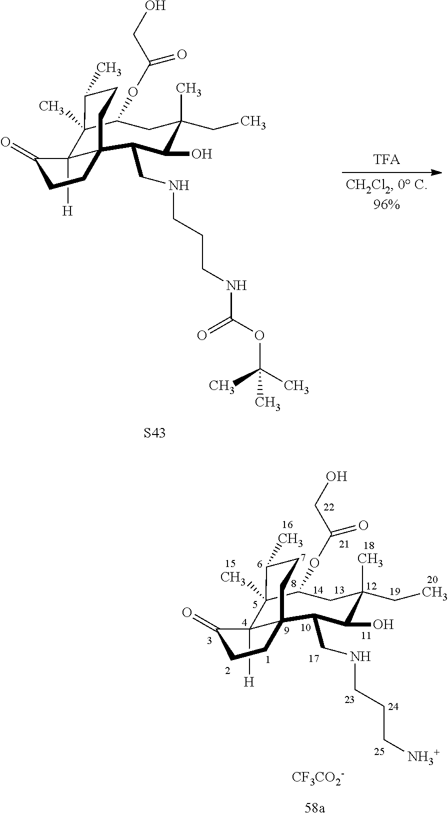

### Synthesis of Diamine 58a (FIG. 13D, Table 1)

Trifluoroacetic acid (47.7 μL, 619 μmol, 30.0 equiv) was added dropwise via syringe to a solution of the amino alcohol S43 (11.4 mg, 20.6 μmol, 1 equiv) in dichloromethane (200 μL) at 0° C. The reaction was stirred for 2 h at 0°. The product mixture was concentrated to dryness at 0° C. The residue obtained was dissolved in anhydrous dichloromethane (500 μL) at 0° C. and the solution was concentrated to dryness. This process was repeated three times. The residue obtained was dissolved in anhydrous methanol (500 μL) at 0° C. and the solution was concentrated to dryness to afford the diamine trifluoroacetic acid salt 58 as a colorless clear film (11.2 mg, 96%).

¹H NMR (500 MHz, CD₃OD) δ 5.58 (d, J=8.0 Hz, 1H, H₁₄), 4.03 (t, J=17.7 Hz, 2, H₂₂), 3.76 (d, J=7.5 Hz, 1H, H₁₁), 3.24-3.08 (m, 4H, 2×H₂₃, 2×H₂₅), 3.04 (t, J=7.8 Hz, 2H, H₁₇), 2.56 (t, J=8.0 Hz, 1H, H₁₀), 2.32 (dd, J=20.0, 11.2 Hz, 1H, 1×H₂), 2.23 (s, 1H, H₄), 2.21-2.08 (m, 4H, 1×H₂, 1×H₁₃, 2×H₂₄), 1.84-1.76 (m, 2H, 1×H₁, 1×H₁₉) 1.70-1.54 (m, 3H, 1×H₁, 1×H₆, 1×H₈), 1.49-1.39 (m, 6H, 2×H₂, 3×H₁₅, 1×H₁₉), 1.32-1.21 (m, 1H, 1×H₈), 1.13-1.06 (m, 4H, 1×H₁₃, 3×H₁₈), 0.88 (t, J=7.5 Hz, 3H, H₂₀), 0.75 (d, J=6.0 Hz, 3H, H₁₆). ¹³C NMR (125 MHz, CD₃OD) δ 216. (C), 171.7 (C), 160.9 (q, J=34.6 Hz, C), 116.4 (q, J=289 Hz, C), 71.1 (CH), 68.6 (CH), 60.4 (CH₂), 57.5 (CH), 47.4 (CH₂), 44.8 (CH₂), 44.1 (C), 41.7 (C), 40.9 (CH₂), 39.5 (CH), 39.3 (C), 36.5 (CH₂), 36.5 (CH), 33.7 (CH₂), 33.5 (CH₂), 30.0 (CH₂), 26.6 (CH₂), 24.7 (CH₂), 23.2 (CH₂), 17.2 (CH₃), 15.7 (CH₃), 13.9 (CH₃), 6.6 (CH). ¹⁹F NMR (470 MHz, CD₃OD) δ −77.1. IR (ATR-FTIR), cm⁻¹: 2931 (s), 1731 (m), 1647 (m), 1495 (w), 1462 (m), 1284 (m), 1155 (w). HRMS-ESI (m/z): [M−CF₃CO₂]⁺ calcd for C₂₅H₄₅N₂O₅, 452.3328; found, 452.3358. [α]_{D}²⁵=+48° (c=1.00, CH₃OH).

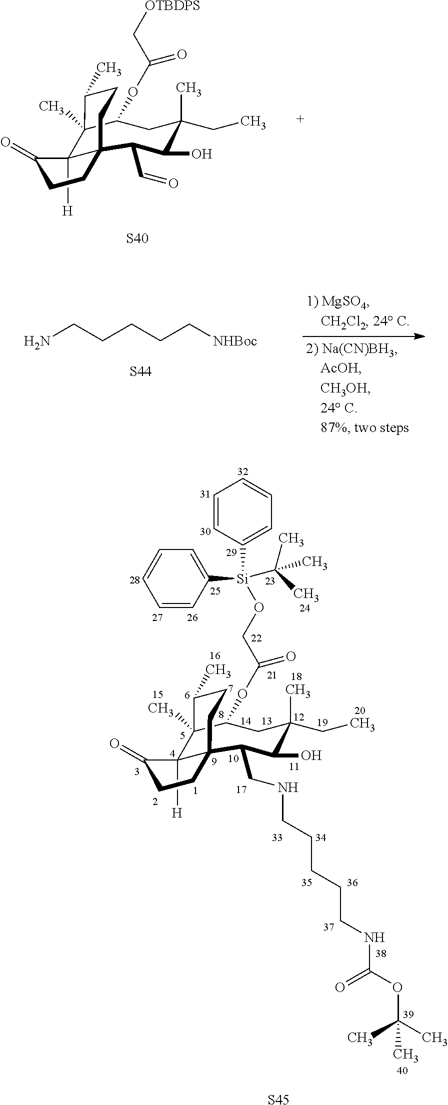

### Synthesis of Secondary Amine S45 (Table 1)

N-(tert-Butylcarbonyl)-1,5-diaminopentane (S44, 14.5 mg, 71.7 μmol, 2.00 equiv) was added to a suspension of O-tert-butyldiphenylsilyl-12-epi-17-oxo-19,20-dihydropleuromutilin 940 [22.7 mg, 35.9 μmol, 1 equiv, dried by azeotropic distillation with benzene (200 μL)] and anhydrous magnesium sulfate (21.5 mg, 179 mmol, 5.00 equiv) in dichloromethane (300 μL). The reaction was stirred for 4 h at 24° C. The resulting mixture was filtered through a small column of powdered sodium sulfate (0.5 cm×0.5 cm). The column was rinsed with dichloromethane (5.0 mL). The filtrates were combined and the combined filtrates were concentrated to dryness. The residue obtained was transferred to a 4-mL vial with benzene (1.5 mL) and the resulting solution was concentrated to dryness. The reaction vessel was evacuated and refilled using a balloon of argon. This process was repeated twice. The residue obtained was dissolved in methanol (200 μL). Sodium cyanoborohydride (4.5 mg, 71.7 μmol, 2.00 equiv) and a solution of acetic acid (2.2 μL, 37.7 μmol, 1.05 equiv) in methanol (100 μL) were added to the reaction vessel at 24° C. The reaction mixture was stirred for 4 h at 24° C. The product mixture was transferred to a separatory funnel that had been charged with dichloromethane (10 mL) and saturated aqueous sodium bicarbonate solution (2.0 mL). The layers that formed were separated and the aqueous layer was extracted with dichloromethane (3×5 mL). The organic layers were combined and dried over sodium sulfate. The dried solution was filtered and the filtrate was concentrated to dryness. The residue obtained was purified by automated flash-column chromatography (eluting with dichloromethane-1% ammonium hydroxide initially, grading to 10% methanol-dichloromethane-1% ammonium hydroxide, linear gradient) to afford the secondary amine S45 as a colorless clear film (25.5 mg, 87%).

R_{f}=0.77 (10% methanol-dichloromethane; UV, PAA, CAM). ¹H NMR (400 MHz, CDCl₃) δ 7.68-7.62 (m, 4H, 2×H₂₇, 2×H₃₁), 7.46-7.35 (m, 6H, 2×H₂₆, 1×H₂₈, 2×H₃₀, 1×H₃₂), 5.53 (d, J=8.0 Hz, 1H, H₁₄), 4.71 (br s, 1H, NH), 4.14 (dd, J=19.2, 2.4 Hz, 2H, H₂₂), 3.65 (d, J=5.6 Hz, 1H, H₁₁), 3.14-2.70 (m, 6H, 2×H₁₇, 2×H₃₃, 2×H₃₇), 2.44 (br s, 1H, H₁₀), 2.28 (dd, J=19.2, 10.8 Hz, 1H, 1×H₂), 2.20-2.11 (m, 1H, 1×H₂), 2.04-1.94 (m, 2H, 1×H₄, 1×H₁₃), 1.78 (d, J=14.4 Hz, 1H, 1×H₈), 1.73-1.61 (m, 3H, 2×H₁, 1×H₇), 1.61-1.46 (m, 6H, 1×H₆, 1×H₇, 2×H₁₉, 2×H₃₄), 1.42 (s, 9H, H₄₀), 1.39-1.31 (m, 7H, 3×H₁₅, 2×H₃₆, 2×H₃₅), 1.14 (td, J=13.6, 2.8 Hz, 1H, 1×H₈), 1.07 (s, 9H, H₂₄), 1.01 (s, 3H, H₁₈), 0.88-0.77 (m, 4H, 1×H₁₃, 3×H₂₀), 0.61 (d, J=6.8 Hz, 3H, H₁₆). ¹³C NMR (100 MHz, CDCl₃) δ 216.3 (C), 170.0 (C), 156.1 (C), 135.5 (CH), 132.7 (C), 132.6 (C), 130.0 (CH), 127.8 (CH), 127.8 (CH), 79.2 (C), 72.0 (CH), 68.8 (CH), 62.9 (CH₂), 58.1 (CH), 48.4 (CH₂), 48.2 (CH₂), 44.4 (C), 41.9 (C), 41.5 (CH₂), 40.2 (C), 39.8 (CH₂), 39.1 (CH), 36.6 (CH), 34.4 (CH₂), 33.3 (CH₂), 30.6 (CH₂), 29.5 (CH₂), 28.4 (CH₃), 26.9 (CH₂), 26.7 (1×CH₃, 1×CH₂), 25.5 (CH₂), 23.9 (CH₂), 19.2 (C), 19.1 (CH₃), 16.7 (CH₃), 14.9 (CH₃), 7.9 (CH₃). IR (ATR-FTIR), cm⁻¹: 3381 (br w), 2947 (w), 1733 (m), 1673 (s), 1465 (w), 1429 (w), 1199 (s), 1134 (s), 1098 (m), 1023 (s), 966 (w), 837 (m), 799 (m), 722 (s). HRMS-ESI (m/z): [M+11]⁺ calcd for C₄₈H₇₅N₂O₇Si, 819.5344; found, 819.5352.

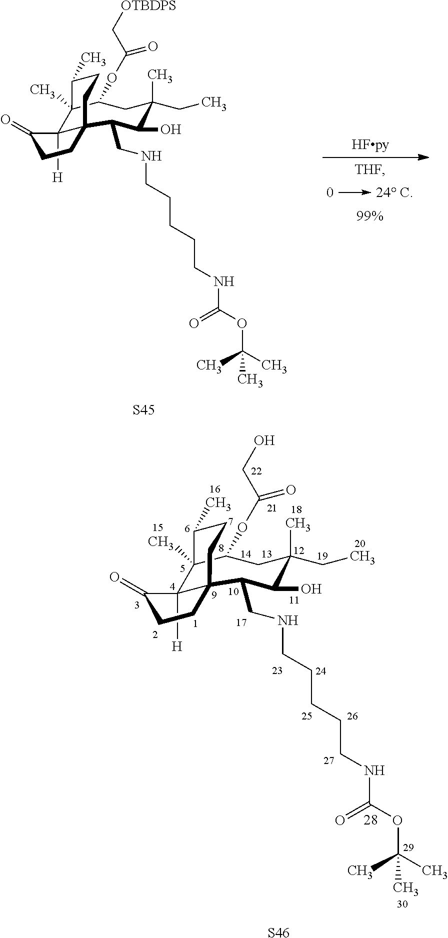

### Synthesis of Amino alcohol S46 (Table 1)

Olah's reagent (4.0 μL, 155 μmol, 5.00 equiv) was added dropwise via syringe to a solution of the secondary amine S45 (25.5 mg, 31.1 μmol, 1 equiv) in tetrahydrofuran (300 μL) at 0° C. The reaction mixture was allowed to warm up over 3.5 h to 24° C. The product mixture was transferred to a separatory funnel that had been charged with dichloromethane (10 mL) and saturated aqueous sodium bicarbonate solution (2.0 mL). The layers that formed were separated and the aqueous layer was extracted with dichloromethane (3×5 mL). The organic layers were combined and dried over sodium sulfate. The dried solution was filtered and the filtrate was concentrated to dryness. The residue obtained was purified by automated flash-column chromatography (eluting with dichloromethane-1% ammonium hydroxide initially, grading to 10% methanol-dichloromethane-1% ammonium hydroxide, linear gradient) to afford the amino alcohol S46 as a colorless clear film (18.9 mg, 99%).

R_{f}=0.15 (10% methanol-dichloromethane; UV, CAM). ¹H NMR (400 MHz, CDCl₃) δ 5.62 (d, J=8.0 Hz, 1H, H₄), 4.64 (br s, 1H, NH), 4.04 (t, J=16.5 Hz, 2H, H₂₂), 3.58 (d, J=6.4 Hz, 1H, H₁₁), 3.15-3.06 (m, 2H, H₁₇), 2.83-2.68 (m, 3H, 2×H₂₃, 1×H₂₇), 2.55-2.50 (m, 1H, 1×H₂₇), 2.30-2.08 (m, 3H, 2×H₂, 1×H₁₀), 2.07-2.02 (m, 2H, 1×H₄, 1×H₁), 1.84 (t, J=11.3 Hz, 1H, 1×H₇), 1.76 (d, J=14.0 Hz, 1H, 1×H₈), 1.68-1.44 (m, 8H, 1×H₁, 1×H₆, 1×H₇, 1×H₁₉, 2×H₂₄, 2×H₂₆), 1.44-1.40 (m, 12H, 3×H₁₅, 9×H₁₃), 1.38-1.29 (m, 4H, 1×H₁, 1×H₁₉, 2×H₂₅), 1.17-1.10 (m, 1H, 1×H₈), 0.99 (d, J=16.0 Hz, 1H, 1×H₁₃), 0.96 (s, 3H, H₁₈), 0.84 (t, J=7.4 Hz, 3H, H₂₀), 0.69 (d, J=6.4 Hz, 3H, H₁₆). ¹³C NMR (100 MHz, CDCl₃) δ 216.6 (C), 172.2 (C), 156.0 (C), 79.1 (C), 72.8 (CH), 70.3 (CH), 61.4 (CH₂), 58.0 (CH), 49.1 (CH₂), 48.1 (CH₂), 44.5 (C), 41.9 (C), 41.4 (CH₂), 40.4 (CH₂), 40.1 (CH₂), 40.0 (CH), 36.5 (CH), 34.5 (CH₂), 34.4 (CH₂), 30.6 (CH₂), 29.7 (CH₂), 29.1 (C), 28.4 (CH₃), 27.0 (CH₂), 25.6 (CH₂), 24.3 (CH₂), 18.7 (CH), 16.7 (CH), 14.9 (CH₃), 7.9 (CH₃). IR (ATR-FTIR), cm⁻¹: 2931 (s), 1731 (m), 1647 (m), 1495 (w), 1462 (m), 1283 (m), 1155 (w). HRMS-ESI (m/z): [M+H]⁺ calcd for C₃₂H₅₇N₂O₇, 581.4166; found, 581.4160.

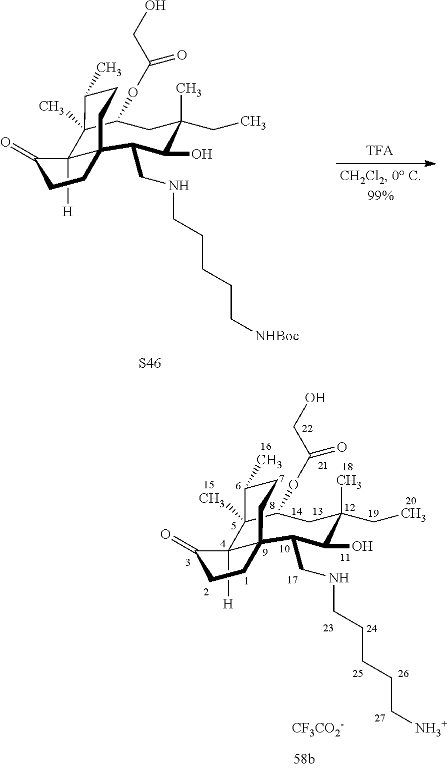

### Synthesis of Diamine 58b (FIG. 131), Table 1)

Trifluoroacetic acid (75.3 μL, 976 μmol, 30.0 equiv) was added dropwise via syringe to a solution of the amino alcohol S46 (18.9 mg, 32.5 μmol, 1 equiv) in dichloromethane (300 μL) at 0° C. The reaction was stirred for 3 h at 0°. The product mixture was concentrated to dryness at 0° C. The residue obtained was dissolved in anhydrous dichloromethane (500 μL) at 0° C. and the solution was concentrated to dryness. This process was repeated three times. The residue obtained was dissolved in anhydrous methanol (500 μL) at 0° C. and the solution was concentrated to dryness to afford the diamine trifluoroacetic acid salt 58b as a colorless clear film (19.2 mg, 99%).

¹H NMR (400 MHz, CD₃OD) 5.59 (d, J=8.0 Hz, 1H, H₁), 4.03 (t, J=16.0 Hz, 2H, H₂), 3.77 (d, J=7.2 Hz, 1H, H₁₁), 3.22-3.01 (m, 4H, 2×H₂₃, 2×H₂₇), 2.96 (t, J=7.6 Hz, 2H, H₁₇), 2.56 (t, J=8.0 Hz, 1H, H₁₀), 2.32 (dd, J=20.0, 11.2 Hz, 1H, 1×H₂), 2.23 (s, 1H, H₄), 2.21-2.11 (m, 2H, 1×H₂, 1×H₁₃), 1.84-1.64 (m, 6H, 1×H₇, 1×H₈, 2×H₁₉, 2×H₂₄), 1.67-1.54 (m, 3H, 1×H₁, 2×H₂₆), 1.49-1.40 (m, 8H, 1×H₁, 1×H₆, 1×H₇, 3×H₁₅, 2×H₂₅), 1.30-1.22 (m, 1H, 1×H₈), 1.12-1.06 (m, 4H, 1×H₁₃, 3×H₁₈), 0.88 (t, J=7.6 Hz, 3H, H₂₀), 0.75 (d, J=6.4 Hz, 3H, H₁₆). ¹³C NMR (100 MHz, CD₃OD) δ 217.7 (C), 173.1 (C), 161.9 (q, J=27.7 Hz, C), 116.4 (q, J=289 Hz, C), 72.5 (CH), 70.1 (CH), 61.7 (CH₂), 58.9 (CH), 49.2 (CH₂), 48.8 (2×CH₂), 45.5 (C), 43.1 (C), 42.3 (CH), 41.0 (C), 40.7 (CH), 40.3 (CH₂), 38.0 (CH), 35.2 (CH₂), 34.9 (CH₂), 31.4 (CH₂), 28.0 (C), 28.0 (CH₂), 26.2 (CH₂), 26.1 (CH₂), 24.5 (CH₂), 18.7 (CH₃), 17.1 (CH₃), 15.3 (CH₃), 8.0 (CH₃). ¹⁹F NMR (375 MHz, CD₃OD) δ −77.2. IR (ATR-FTIR), cm⁻¹: 3375 (br w), 2958 (w), 1733 (w), 1674 (s), 1464. (w), 1200 (s), 1135 (s), 1099 (m), 1023 (s), 967 (w), 837 (m), 800 (s), 722 (s). HRMS-ESI (m/z): [M−CF₃CO₂⁻]⁺ calcd for C₂₇H₄₉N₂O₅, 481.3636; found, 481.3634. [α]_{D}²⁵=+42° (c=1.00, CH₃OH).

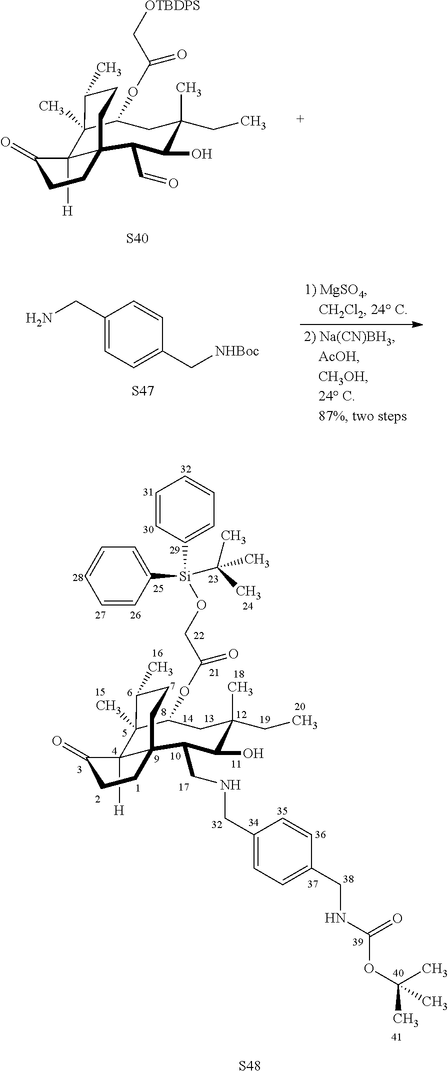

### Synthesis of Secondary Amine S48 (Table 1)

tert-Butyl (4-(aminomethyl)benzyl)carbamate (S47, 12.7 mg, 53.8 μmol, 1.50 equiv) was added to a suspension of O-tert-butyldiphenylsilyl-12-epi-17-oxo-19,20-dihydropleuromutilin S40 [22.7 mg, 35.9 μmol, 1 equiv. dried by azeotropic distillation with benzene (200 μL)] and anhydrous magnesium sulfate (21.6 mg, 180 mmol, 5.00 equiv) in dichloromethane (300 μL). The reaction was stirred for 3 h at 24° C. The resulting mixture was filtered through a small column of powdered sodium sulfate (0.5 cm×0.5 cm). The column was rinsed with dichloromethane (5.0 mL). The filtrates were combined and the combined filtrates were concentrated to dryness. The residue obtained was transferred to a 4-mL vial with benzene (1.5 mL) and the resulting solution was concentrated to dryness. The reaction vessel was evacuated and refilled using a balloon of argon. This process was repeated twice. The residue obtained was dissolved in methanol (200 μL). Sodium cyanoborohydride (4.5 mg, 71.7 μmol, 2.00 equiv) and a solution of acetic acid (2.2 μL, 37.7 μmol, 1.05 equiv) in methanol (100 μL) were added to the reaction vessel at 24° C. The reaction mixture was stirred for 4 h at 24° C. The product: mixture was transferred to a separatory funnel that had been charged with dichloromethane (10 mL) and saturated aqueous sodium bicarbonate solution (2.0 mL). The layers that formed were separated and the aqueous layer was extracted with dichloromethane (3×5 mL). The organic layers were combined and dried over sodium sulfate. The dried solution was filtered and the filtrate was concentrated to dryness. The residue obtained was purified by automated flash-column chromatography (eluting with dichloromethane-1% ammonium hydroxide initially, grading to 10% methanol-dichloromethane-1% ammonium hydroxide, linear gradient) to afford the secondary amine S48 as a colorless clear film (26.5 mg, 87%).

R_{f}=0.63 (10% methanol-dichloromethane; UV, PAA, CAM). ¹H NMR (400 MHz, CD₂Cl₂) δ 7.73-7.64 (m, 4H, 2×H₂₇, 2×H₃₁), 7.49-7.35 (m, 6H, 2×H₂₆, 1×H₂₈, 2×H₃₀, 1×H₃₂), 7.34-7.19 (m, 4H, 2×H₃₅, 2×H₃₆), 5.58 (d, J=8.0 Hz, 1H, H₁₄), 5.04 (br s, 1H, NH), 4.36-4.25 (m, 2H, H₃₈), 4.23-4.09 (m, 2H, H₂₂), 3.87-3.71 (m, 2H, H₃₃), 3.57 (d, J=6.0 Hz, 1H, H₁₁), 2.89 (d, J=9.2 Hz, 1H, 1×H₁₇), 2.81 (t, J=11.2 Hz, 1H, 1×H₁₇), 2.30-2.09 (m, 3H, 2×H₂, 1×H₁₀), 2.08-1.98 (m, 2H, 1×H₄, 1×H₁₃), 1.85-1.72 (m, 2H, 1×H₁, 1×H₇), 1.65-1.50 (m, 5H, 1×H₁, 1×H₆, 1×H₁₉, 1×OH, 1×NH), 1.46 (s, 9H, H₄₁), 1.41-1.31 (m, 6H, 1×H₇, 1×H₈, 3×H₁₅, 1×H₁₉), 1.12-1.04 (m, 10H, 1×H₈, 9×H₂₄), 0.98 (s, 3H, H₁₈), 0.91-0.80 (m, 4H, 1×H₁₃, 3×H₂₀), 0.62 (d, J=6.4 Hz, 3H, H₁₆). ¹³C NMR (100 MHz, CD₂Cl₂) δ 216.6 (C), 169.7 (C), 155.8 (C), 138.4 (C), 138.1 (C), 135.5 (CH), 132.9 (C), 132.8 (C), 129.9 (CH), 128.3 (CH), 127.9 (CH), 127.7 (CH), 127.4 (CH), 79.1 (C), 72.6 (CH), 68.9 (CH), 62.9 (CH₂), 58.0 (CH), 53.3 (CH₂), 47.7 (CH₂), 44.6 (C), 44.2 (CH₂), 41.9 (CH₂), 41.4 (C), 40.2 (CH), 40.0 (C), 36.7 (CH), 34.6 (CH₂), 34.4 (CH₂), 30.6 (CH₂), 28.1 (CH₃), 27.1 (CH₂), 26.4 (CH₃), 25.5 (CH₂), 19.0 (C), 18.7 (CH₃), 16.5 (CH₃), 14.7 (CH₃), 7.7 (CH₃). IR (ATR-FTIR), cm⁻¹: 2935 (w), 1750 (w), 1463 (w), 1428 (w), 1371 (w), 1296 (w), 1265 (w), 1215 (w), 1143 (s), 1113 (s), 1019 (s), 999 (m), 970 (w), 915 (w), 823 (m), 738 (s), 701 (s), 613 (m), 504 (s), 489 (s). HRMS-ESI (m/z): [M+H]⁺ calcd for C₅₁H₇₃N₂O₇Si, 853.5187; found, 853.5192.

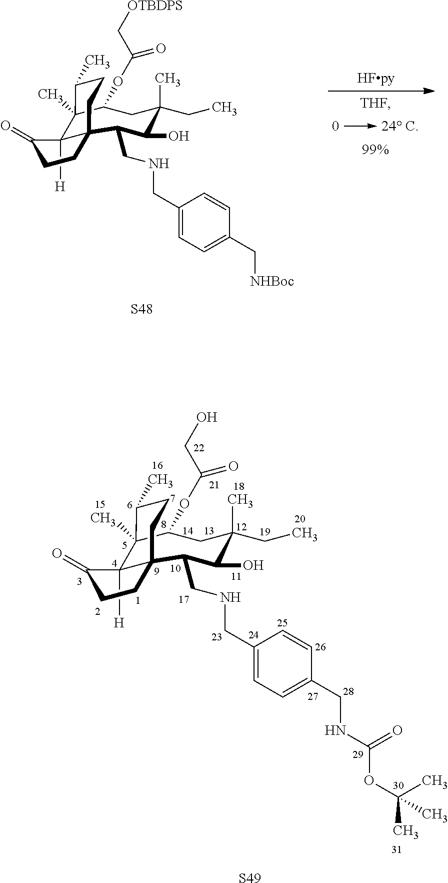

### Synthesis of Amino Alcohol S49 (Table 1)

Olah's reagent (4.0 μL, 155 μmol, 5.00 equiv) was added dropwise via syringe to a solution of the secondary amine S48 (26.5 mg, 31.1 μmol, 1 equiv) in tetrahydrofuran (300 μL) at 0° C. The reaction mixture was allowed to warm up over 3.5 h to 24° C. The product mixture was transferred to a separatory funnel that had been charged with dichloromethane (10 mL) and saturated aqueous sodium bicarbonate solution (2.0 mL). The layers that formed were separated and the aqueous layer was extracted with dichloromethane (3×5 mL). The organic layers were combined and dried over sodium sulfate. The dried solution was filtered and the filtrate was concentrated to dryness. The residue obtained was purified by automated flash-column chromatography (eluting with dichloromethane-1% ammonium hydroxide initially, grading to 10% methanol-dichloromethane-1% ammonium hydroxide, linear gradient) to afford the amino alcohol S49 as a colorless clear film (19.1 mg, 99%).

R_{f}=0.33 (10% methanol-dichloromethane-1% ammonium hydroxide; UV, PAA, CAM). ¹H NMR (500 MHz, CD₂Cl₂) δ 7.36-7.20 (m, 4H, 2×H₂₅, 2×H₂₆), 5.70-5.54 (m, 1H, H₁₄), 5.05 (br s, NH), 4.36-4.19 (m, 2H, H₂₈), 4.10-3.96 (m, 2H, H₂₂), 3.86-3.69 (m, 2H, H₂₃), 3.64-3.51 (m, 1H, H₁₁), 2.89 (d, J=11.0 Hz, 1H, 1×H₁₇), 2.81 (t, J=11.2 Hz, 1H, 1×H₁₇₃), 2.34-2.01 (m, 6H, 2×H₂, 1×H₄, 1×H₈, 1×H₁₀, 1×H₁₃), 1.85-1.72 (m, 2H, 1×H₁, 1×H₇), 1.66-1.55 (m, 3H, 1×H₆, 1×H₇, 1×H₁₃), 1.48-1.31 (m, 14H, 3×H₁₅, 2×H₁₉, 9×H₃₁), 1.21-1.12 (m, 1H, 1×H₁), 1.10-1.03 (m, 1H, 1×H₈), 0.97 (s, 3H, H₁₈), 0.91-0.81 (m, 3H, H₂₀), 0.76-0.65 (m, 3H, H₁₆). ¹³C NMR (100 MHz, CD₂Cl₂) δ 216.5 (C), 172.1 (C), 155.8 (C), 138.4 (C), 138.1 (C), 128.3 (CH), 127.4 (CH), 72.6 (CH), 70.1 (CH), 61.3 (CH₂), 57.8 (CH), 53.3 (CH₂), 47.8 (CH₂), 44.6 (CH), 44.1 (CH₂), 41.9 (C), 41.3 (CH₂), 40.1 (CH), 40.1 (C), 36.6 (CH₂), 34.5 (CH₂), 34.3 (CH₂), 30.5 (CH₂), 28.1 (CH₃), 27.1 (CH₂), 25.5 (CH₂) 18.6 (CH₃), 16.5 (CH₃), 14.6 (CH₃), 7.6 (CH₃). IR (ATR-FTIR), cm⁻¹: 3354 (br w), 2928 (w), 1725 (w), 1647 (w), 1464. (w), 1408 (w), 1282 (w), 1154 (w), 1102 (w), 1019 (s), 969 (w). HRMS-ESI (m/z): [M+H]⁺ calcd for C₃₅H₅₅N₂₇, 615.4009; found, 615.4003.

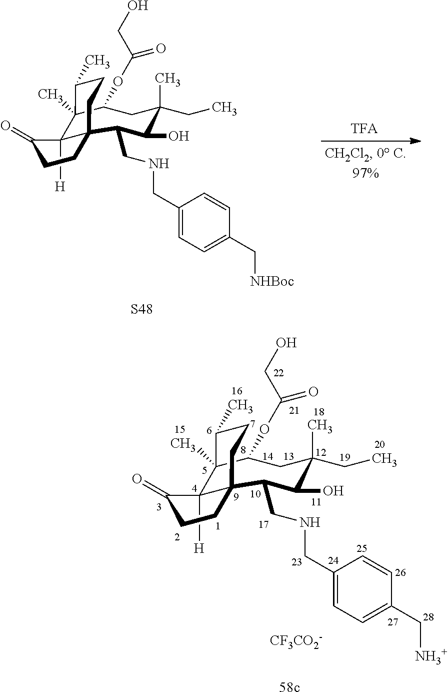

### Synthesis of Diamine 58c (FIG. 13D, Table 1)

Trifluoroacetic acid (72.0 μL, 932 μmol, 30.0 equiv) was added dropwise via syringe to a solution of the amino alcohol S49 (19.1 mg, 31.1 μmol, 1 equiv) in dichloromethane (300 μL) at 0° C. The reaction was stirred for 2.5 h at 0° C. The product mixture was concentrated to dryness at 0° C. The residue obtained was dissolved in anhydrous dichloromethane (500 μL) at 0° C. and the solution was concentrated to dryness. This process was repeated three times. The residue obtained was dissolved in anhydrous methanol (500 μL) at 0° C. and the solution was concentrated to dryness to afford the diamine trifluoroacetic acid salt 58c as a colorless clear film (18.9 mg, 97%).

¹H NMR (400 MHz, CD₃OD) δ 7.67-7.50 (m, 4H, 2×H₂₅, 2×H₂₆), 5.56 (d, J=8.0 Hz, 1H, H₁₄), 4.39 (d, J=13.2 Hz, 1H, 1×H₂₃), 4.24 (t, J=16.8 Hz, 1H, 1×H₂₃), 4.17 (s, 2H, H₂₈), 4.03 (t, J=16.0 Hz, 2H, H₂₂), 3.71 (d, J=7.2 Hz, 1H, H₁₁), 3.14 (d, J=12.0 Hz, 1H, 1×H₁₇), 3.07 (d, J=11.2 Hz, 1H, 1×H₁₇), 2.57 (t, J=8.2 Hz, 1H, H₁₀), 2.34-2.22 (m, 1H, 1×H₂), 2.21-2.01 (m, 3H, 1×H₂, 1×H₄, 1×H₁₃), 1.78-1.67 (m, 2H, 1×H₁, 1×H₈), 1.66-1.58 (m, 2H, 1×H₆, 1×H₁₉), 1.52 (dd, J=14.0, 7.2 Hz, 1H, 1×H₇), 1.48-1.38 (m, 5H, 1×H₇, 3×H₁₅, 1×H₁₉), 1.37-1.32 (m, 1H, 1×H₁), 1.27-1.20 (m, 1H, 1×H₈), 1.12-1.02 (m, 4H, 1×H₁₃, 3×H₁₈), 0.85 (t, J=7.2 Hz, 3H, H₂₀), 0.74 (d, J=6.0 Hz, 3H, H₁₆). ¹³C NMR (100 MHz, CD₃OD) δ 217.6 (C), 173.1 (C), 162.0 (q, J=39.5 Hz, C), 136.1 (C), 133.2 (C), 131.6 (CH), 130.9 (CH), 117.7 (q, J=289 Hz, C), 72.7 (CH), 70.1 (CH), 61.8 (CH₂), 58.9 (CH), 51.8 (CH₂), 45.5 (C), 43.8 (CH₂), 43.1 (C), 43.0 (CH₂), 42.3 (CH₂), 40.9 (C), 40.6 (CH), 37.9 (CH), 35.1 (CH₂), 34.8 (CH₂), 31.4 (CH₂), 28.0 (CH₂), 26.2 (CH₂), 18.7 (CH₃), 17.1 (CH₃), 15.3 (CH₃), 8.0 (CH₃). ¹⁹F NMR (375 MHz, CD₃OD) δ −77.1. IR (ATR-FTIR), cm⁻¹: 2944 (w), 1732 (m), 1671 (s), 1460 (w), 1429 (w), 1385 (w), 1199 (s), 1178 (s), 1132 (s), 1096 (s), 1025 (s), 966 (w), 836 (m), 799 (s), 722 (s). HRMS-ESI (m/z): [M−CF₃CO₂⁻]⁺ calcd for C₃₀H₄₇N₂O₅, 515.3479; found, 515.3475. [α]_{D}²⁵=+41° (c=1.00. CH₃OH).

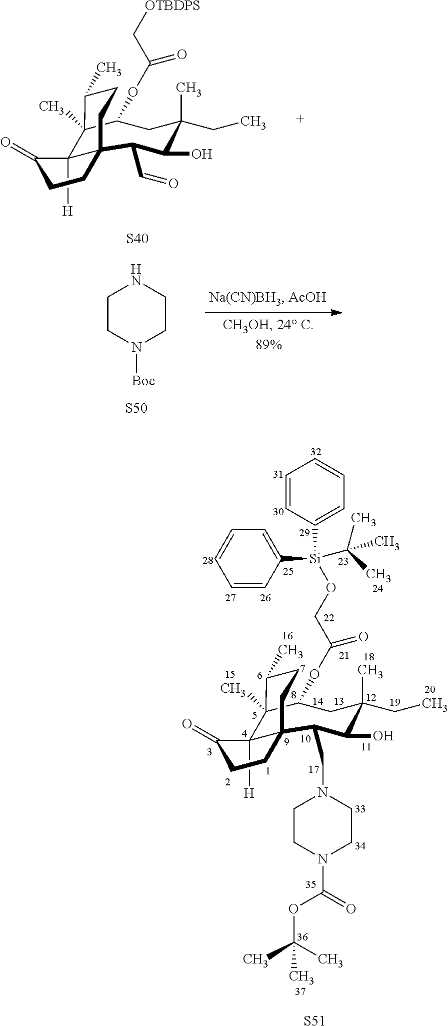

### Synthesis of Secondary Amine S51 (Table 1)

tert-Butyl piperazine-1-carboxylate (S50, 13.3 mg, 53.8 μmol, 2.00 equiv) was added to a solution of O-tert-butyldiphenylsilyl-12-epi-17-oxo-19,20-dihydropleuromutilin S40 [22.7 mg, 35.9 μmol, 1 equiv, dried by azeotropic distillation with benzene (200 μL)]methanol (200 μL). The reaction was stirred for 2 h at 24° C. Sodium cyanoborohydride (4.5 mg, 71.7 μmol, 2.00 equiv) and a solution of acetic acid (2.2 μL, 37.7 μmol, 1.05 equiv) in methanol (100 μL) were added to the reaction vessel at 24° C. The reaction mixture was stirred for 4 h at 24° C. The product mixture was transferred to a separatory funnel that had been charged with dichloromethane (10 mL) and saturated aqueous sodium bicarbonate solution (2.0 mL). The layers that formed were separated and the aqueous layer was extracted with dichloromethane (3×5 mL). The organic layers were combined and dried over sodium sulfate. The dried solution was filtered and the filtrate was concentrated to dryness. The residue obtained was purified by automated flash-column chromatography (eluting with hexanes initially, grading to 33% acetone-hexanes, linear gradient) to afford the secondary amine S51 as a colorless clear film (25.9 mg, 89%).

R_{f}=0.43 (33% acetone-hexanes; UV, CAM). ¹H NMR (400 MHz, CD₂Cl₂) δ 7.73-7.64 (m, 4H, 2×H₂₇, 2×H₃₁), 7.46-7.34 (m, 6H, 2×H₂₆, 1×H₂₉, 2×H₃₀, 1×H₃₂), 6.03 (br s, 1H, OH), 5.58 (d, J=8.0 Hz, 1H, H₁₄), 4.13 (dd, J=19.2, 2.4 Hz, 2H, H₂), 3.57 (d, J=4.0 Hz, 1H, H₁₁), 2.81 (t, J=11.8 Hz, 1H, 1×H₁₇), 2.42-2.30 (m, 2H, 1×H₁₀, 1×H₁₇), 2.29-2.08 (m, 3H, 2×H₂, 1×H₃₃), 2.04-1.95 (m, 2H, 1×H₄, 1×H₁₃), 1.86-1.70 (m, 3H, 1×H₁, 1×H₆, 1×H₈), 1.68-1.53 (m, 4H, 2×H₇, 1×H₁₉, 1×H₃₃), 1.52-1.44 (m, 10H, 1×H₃₄, 9×H₃₇), 1.42-1.34 (m, 6H, 1×H₁, 3×H₁₅, 2×H₃₄), 1.32-1.21 (m, 2H, 1×H₁₉, 1×H₃₃), 1.18-1.10 (m 1H, 1×H₈, 1×H₃₃), 1.18-1.03 (m 10H, 9×H₂₄, 1×H₃₄), 0.97 (s, 3H H₁₈), 0.94-0.89 (m, 1H, 1×H₁₃), 0.84 (t, J=7.6 Hz, 3H, H₂), 0.65 (d, J=6.0 Hz, 3H, H₁₆). ¹³C NMR (100 MHz, CD₂Cl₂) δ 216.6 (C), 169.8 (C), 154.6 (C), 135.5 (CH), 132.7 (C), 129.9 (C), 127.8 (CH), 79.9 (C), 73.2 (CH), 68.7 (CH), 62.9 (CH₂), 58.0 (CH), 57.6 (CH₂), 44.3 (CH), 41.9 (C), 41.4 (CH₂), 40.4 (C), 36.7 (CH), 35.2 (CH), 34.7 (CH₂), 34.3 (CH₂), 30.6 (CH₂), 28.4 (CH₃), 26.9 (CH₂), 26.7 (CH₃), 25.5 (CH₂), 19.2 (C), 18.7 (CH₃), 16.6 (CH₃), 15.0 (CH₃), 7.9 (CH₃). IR (ATR-FTR), cm⁻¹: 2954 (w), 1731 (m), 1459 (w), 1428 (w), 1374 (w), 1285 (w), 1113 (s), 1058 (m), 1008 (m), 951 (w), 916 (w), 837 (m), 824 (m), 737 (m), 700 (s), 613 (m), 497 (s). HRMS-ESI (m/z): [M+H]⁺ calcd for C₄₇H₇₁N₂O₇Si, 803.5031; found, 803.5009.

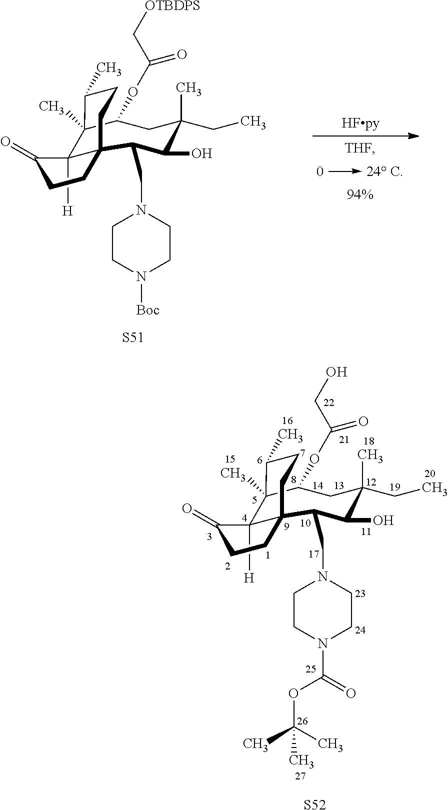

### Synthesis of Amino Alcohol S52 (Table 1)

Olah's reagent (4.0 μL, 155 μmol, 5.00 equiv) was added dropwise via syringe to a solution of the secondary amine S51 (25.9 mg, 31.1 μmol, 1 equiv) in tetrahydrofuran (300 μL) at 0° C. The reaction mixture was allowed to warm up over 3.5 h to 24° C. The product mixture was transferred to a separatory funnel that had been charged with dichloromethane (10 mL) and saturated aqueous sodium bicarbonate solution (2.0 mL). The layers that formed were separated and the aqueous layer was extracted with dichloromethane (3×5 mL). The organic layers were combined and dried over sodium sulfate. The dried solution was filtered and the filtrate was concentrated to dryness. The residue obtained was purified by automated flash-column chromatography (eluting with dichloromethane-1% ammonium hydroxide initially, grading to 10% methanol-dichloromethane-1% ammonium hydroxide, linear gradient) to afford the amino alcohol S52 as a colorless clear film (17.6 mg, 94%).

R_{f}=0.53 (10% methanol-dichloromethane-1% ammonium hydroxide; PAA, CAM). ¹H NMR (400 MHz, CDCl₃) δ 6.00 (br s, 1H, C11-OH), 5.65 (d, J=8.0 Hz, 1H, H₁₄), 4.05 (td, J=16.8.4.8 Hz, 2H, H₂₂), 3.58 (d, J=6.4 Hz, 1H, H₁), 2.82 (t, J=11.6 Hz, 1H, 1×H₁₇), 2.45 (t, J=5.2 Hz, 1H, C22-OH), 2.42-2.29 (m, 3H, 1×H₆, 1×H₁₇, 1×H₂₃), 2.27-2.1 (m, 3H, 2×H₂, 1×H₂₃), 2.10-2.05 (m, 1H, 1×H₁₃), 2.03 (s, 1H, H₄), 1.88-1.67 (m, 3H, 1×H₁, 1×H₈, 1×H₂₃), 1.66-1.51 (m, 4H, 1×H₁, 2×H₇, 1×H₁₉), 1.50-1.41 (m, 14H, 3×H₁₅, 9×H₂₇, 2×H₂₄), 1.40-1.23 (m, 3H, 1×H₁₉, 2×H₂₄), 1.23-1.08 (m, 2H, 1×H₈, 1×H₂₃), 1.05-0.98 (m, 1H, 1×H₁₃), 0.96 (s, 3H, H₁₈), 0.85 (t, J=7.4 Hz, 3H, H₂₀), 0.70 (d, J=6.8 Hz, 3H, H₁₆). ¹³C NMR (100 MHz, CDCl₃) δ 216.3 (C), 172.2 (C), 154.6 (C), 79.9 (C), 73.2 (CH), 70.2 (CH), 61.3 (CH₂), 57.9 (CH), 57.5 (CH₂), 44.3 (C), 41.9 (C), 41.3 (CH₂), 40.5 (C), 36.6 (CH), 35.3 (CH), 34.6 (CH₂), 34.2 (CH₂), 30.5 (2×CH), 28.4 (1×CH₂, 1×CH₃), 26.9 (CH₂), 25.5 (CH₂), 18.5 (CH₃), 16.6 (CH₃), 14.9 (CH₃), 7.9 (CH₃). IR (ATR-FTIR), cm⁻¹: 3364 (m), 2932 (s), 1721 (s), 1648 (s), 1549 (m), 1495 (m), 1452 (m), 1409 (w), 1396 (w), 1277 (s), 1154 (m), 1088 (m), 1016 (s), 969 (m), 923 (w), 867 (2), 807 (w), 774 (w). HRMS-ESI (m/z): [M+H]⁺ calcd for C₃H₅₃N₂O₇, 565.3853; found, 565.3845.

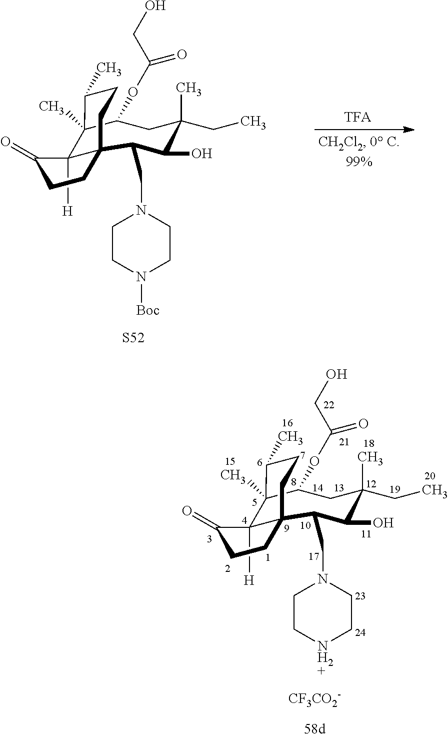

### Synthesis of Diamine 58d (Table 1)

Trifluoroacetic acid (72.0 μL, 935 μmol, 30.0 equiv) was added dropwise via syringe to a solution of the amino alcohol S52 (17.6 mg, 31.2 μmol, 1 equiv) in dichloromethane (300 μL) at 0° C. The reaction was stirred for 2.5 h at 0° C. The product mixture was concentrated to dryness at 0° C. The residue obtained was dissolved in anhydrous dichloromethane (500 μL) at 0° C. and the solution was concentrated to dryness. This process was repeated three times. The residue obtained was dissolved in anhydrous methanol (500 μL) at 0° C. and the solution was concentrated to dryness to afford the diamine trifluoroacetic acid salt 58d as a colorless clear film (18.6 mg, 99%).

¹H NMR (400 MHz, CD₃OD) 5.57 (d, J=8.0 Hz, 1H, H₁₄), 4.05 (t, J=15.9 Hz, 2H, H₂₂), 3.79 (d, J=7.2 Hz, 1H, H₁₁), 3.76-3.63 (m, 4H, 2×H₂₃, 2×H₂₄), 3.62-3.6483 (m, 4H, 2×H₂₃, 2×H₂₄), 3.41 (t, J=11.6 Hz, 1H, 1×H₁₇), 3.30-3.26 (m, 1H, 1×H₁₇), 2.55 (t, J=9.0 Hz, 1H, H₁₀), 2.38-2.12 (m, 4H, 1×H₁, 2×H₂, 1×H₄), 1.88-1.74 (m, 2H, 1×H₇, 1×H₈), 1.70-1.54 (m, 3H, 1×H₁, 1×H₆, 1×H₁₉), 1.52-1.39 (m, 6H, 1×H₇, 1×H₁₃, 3×H₁₅, 1×H₁₉), 1.27-1.19 (m, 1H, 1×H₈), 1.16-1.05 (m, 4H, 1×H₃, 3×H₁₈), 0.89 (t, J=9.6 Hz, 3H, H₂₀), 0.76 (d, J=6.0 Hz, 3H, H₁₆). ¹³C NMR (100 MHz, CD₃OD) δ 216.0 (C), 172.1 (C), 160.1 (q, J=42.6 Hz, C), 115.9 (q, J=285 Hz, C), 71.8 (CH), 68.8 (CH), 60.4 (CH₂), 58.2 (CH₂), 57.4 (CH), 48.6 (CH₂), 44.1 (C), 41.7 (C), 40.7 (CH₂), 40.3 (CH₂), 39.8 (C), 36.5 (CH), 36.4 (CH), 33.7 (CH₂), 33.4 (CH₂), 29.9 (CH₂), 26.4 (CH₂), 24.8 (CH₂), 17.3 (CH₃), 15.6 (CH₃), 13.9 (CH₃), 6.6 (CH₃). ¹⁹F NMR (375 MHz, CD₃OD) δ −77.4. IR (ATR-FTIR), cm⁻¹: 2926 (w), 1732 (m), 1671 (s), 1463 (w), 1382 (w), 1175 (s), 1129 (s), 1092 (s), 1026 (m), 956 (w), 836 (m), 798 (m), 722 (s). HRMS-ESI (m/z): [M+H]⁺ calcd for C₂₆H₄₅N₂O₅, 465.3323; found, 465.3322. [α]_{D}²⁵=+49° (c=1.00, CH₃OH).

### BIBLIOGRAPHY FOR SPECIFIC EXAMPLES

1. Pangborn, A. B.; Giardello, M. A.; Grubbs, R. H.; Rosen, R. K.; Timmers, F. J. Organometallics 1996, 15, 1518.
2. Simmons, E. M.; Hartwig, J. F. Nature 2012, 483, 70.
3. Iwasaki, K.; Wan, K. K.; Oppedisano, A.; Crossley, S. W. M.; Shenvi, R. A. J. Am. Chem. Soc. 2014, 136, 1300.
4. Berner, H.; Schulz, G.; Schneider, H. Tetrahedron 1980, 36, 1807.

### REFERENCES

1. Isolation: (a) Kavanagh, F.; Hervey, A.; Robbins, W. J. Proc. Natl. Acad. Sci. U.S.A. 1951, 37, 570. (b) Kavanagh, F.; Hervey, A.; Robbins, W. J. Proc. Natl. Acad. Sci. U.S.A. 1952, 38, 555. Structure determination: (c) Anchel, M. J. Biol. Chem. 1952, 199, 133. (d) Arigoni, D. Gazz. Chim. Ital. 1962, 92, 884. (e) Birch, A. J.; Holzapfel, C. W.; Rickards, R. W. Tetrahedron 1966, 22, 359. (f) Arigoni, D. Pure Appl. Chem. 1968, 17, 331. For the biosynthesis of (+)-pleuromutilin (1), see refs. 1e, 1f, and the following: (g) Bailey, A. M.; Alberti, F.; Kilaru, S.; Collins, C. M.; de Mattos-Shipley, K.; Hartley, A. J.; Hayes, P.; Griffin, A.; Lazarus, C. M.; Cox, R. J.; Willis, C. L.; O'Dwyer, K.; Spence, D. W.; Foster, G. D. Sci. Rep. 2016, 6, 25202. (h) Alberti, F.; Khairudin, K.; Venegas, E. R.; Davies, J. A.; Hayes. P. M.: Willis, C. L.; Bailey, A. M.; Foster, G. D. Nat. Commun. 2017, 8, 1831.
2. (a) Schlunzen, F.; Pyetan, E.; Fucini, P.; Yonath, A.; Harms, J. M. Mol. Microbiol. 2004, 54, 1287. (b) Davidovich, C.; Bashan, A.; Auerbach-Nevo, T.; Yaggie, R. D.; Gontarek, R. R.; Yonath, A. Proc. Natl. Acad. Sci. U.S.A. 2007, 104, 4291.
3. For reviews, see: (a) Phillips, O. A.; Sharaf, L. H. Expert Opin. Ther. Pat. 2007, 17, 429. (b) Changhua, H.; Yi, Z. Mini-Rev. Med. Chem. 2009, 9, 1397. (c) Novak, R. Ann. N. Y. Acad. Sci. 2011, 1241, 71. (d) Tang, Y. Z.; Liu, Y. H.; Chen, J. X. Mini-Rev. Med. Chem. 2012, 12, 53. (e) Ruofeng, S.; Jiatu, W.; Wenzhu, G.; Jianping, L. Curr. Top. Med. Chem. 2013, 13, 3013. (f) Fazakerley, N. J.; Procter, D. J. Tetrahedron 2014, 70, 6911. (g) Paukner, S.; Riedl, R. Cold Spring Harbor Perspect. Med. 2017, 7, a027110.
4. Daum, R. S.; Kar, S.; Kirkpatrick, P. Nat. Rev. Drug Discovery 2007, 6, 865.
5. website: globenewswire.com/news-release/2017/09/18/1124000/0/en/Nabriva-Therapeutics-Announces-Positive-Topline-Results-from-Global-Phase-3-Clinical-Trial-Evaluating-IV-and-Oral-Lefamulin-for-the-Treatment-of-Community-Acquired-Bacterial-Pneumo.html accessed Jan. 4, 2018
6. Berner, H.; Vyplel, H.; Schulz, G.; Schneider, H. Monatsh. Chem. 1986, 117, 1073.
7. (a) Paukner, S.; Gruss, A.; Fritsche, T. R.; Ivezic-Schoenfeld, Z.; Jones, R. N., In Vitro Activity of the Novel Pleuromutilin BC-3781 Tested Against Bacterial Pathogens Causing Sexually Transmitted Diseases (STD). In 53rd Interscience Conference on Antimicrobial Agents and Chemotherapy, Denver, Colo., 2013. (b) Paukner, S.; Strickmann, D. B.; Ivezic-Schoenfeld, Z., Extended Spectrum Pleuromutilins: Mode-of-action Studies. In 24th European Congress of Clinical Microbiology and Infectious Diseases (ECCMID), Barcelona, Spain, 2014. (c) Wicha, W. W.; Ivezic-Schoenfeld, Z., In Vivo Activity of Extended Spectrum Pleuromutilins in Murine Sepsis Model. In 24th European Congress of Clinical Microbiology and Infectious Diseases (ECCMID), Barcelona, Spain, 2014. (d) Paukner, S.; Kollmann, H.; Riedl, R.; Ivezic-Schoenfeld, Z., Kill curves of the Novel Extended-Spectrum Pleuromutilin Antibiotic BC-9529. In 25th European Congress of Clinical Microbiology and Infectious Diseases (ECCMID), Copenhagen, Denmark, 2015. (e) Paukner, S.; Wicha, W. W.; Heilmayer, W.; Thirring, K.; Riedl, R., Extended Spectrum Pleuromutilins: Potent Translation Inhibitors with Broad-Spectrum Antibacterial Activity In Vitro an In Vivo. In Interscience Conference of Antimicrobial Agents and Chemotherapy/International Congress of Chemotherapy and Infection (ICAAC/ICC), San Diego, Calif., 2015. (f) Paukner, S.; Wicha, W. W.; Thirring, K.; Kollmann, H.; Ivezic-Schoenfeld, Z., In Vitro and In Vivo Efficacy of Novel Extended Spectrum Pleuromutilins Against S. aureus and S. pneumoniae. In 25th European Congress of Clinical Microbiology and Infectious Diseases (ECCMID), Copenhagen, Denmark, 2015. (g) Thirring, K.; Heilmayer, W.; Riedl, R.; Kollmann, H.; Ivezic-Schoenfeld, Z.; Wicha, W.; Paukner, S.; Strickmann, D. Preparation of 12-epi-pleuromutilin derivatives as antimicrobial agents. Patent WO2015110481A1, 2015. (h) Wicha, W. W.; Paukner, S.; Strickmann, D. B.; Thirring, K.; Kollmann, H.; Heilmayer, W.; Ivezic-Schoenfeld, Z., Efficacy of Novel Extended Spectrum Pleuromutilins Against E. coli In Vitro and In Vivo. In 25th European Congress of Clinical Microbiology and Infectious Diseases (ECCMID), Copenhagen, Denmark, 2015.
8. (a) Murphy, S. K.; Zeng, M.; Herzon, S. B. Org Lett. 2016, 18, 4880. (b) Murphy, S. K.; Zeng, M.; Herzon, S. B. Science 2017, 356, 956. (c) Murphy, S. K.; Zeng, M.; Herzon, S. B. Org. Lett. 2017, 19, 4980. (d) Zeng, M.; Murphy, S. K.; Herzon, S. B. J. Am. Chem. Soc. 2017, 139, 16377. For a review of prior syntheses and synthetic studies of (+)-pleuromutilin (1), see ref. 3f. For a recent synthesis of (+)-pleuromutilin (1), see: (e) Farney, E. P.; Feng, S. S.; Schäfers, F.; Reisman, S. E. J. Am. Chem. Soc. 2018, Article ASAP, DOI: 10.1021/jacs.7b13260.
9. For reviews, see: (a) Shugrue, C. R.; Miller, S. J. Chem. Rev. 2017, 117, 11894. (b) Karimov, R. R.; Hartwig, J. F. Angew. Chem., Int. Ed. Engl. Early View, DOI 10.1002/anie.201710330.
10. Hanson, R. L.; Matson, J. A.; Brzozowski, D. B.; LaPorte, T. L.; Springer, D. M.; Patel, R. N. Org. Process Res. Dev. 2002, 6, 482.
11. Zhou, J.; Hartwig, J. F. Angew. Chem., Int. Ed. 2008, 47, 5783.
12. Uccello, D. P.; Miller, S. M.; Dieterich, N. A.; Stepan, A. F.; Chung, S.; Farley, K. A.; Samas, B.; Chen, J.; Montgomery, J. I. Tetrahedron Lett. 2011, 52, 4247.
13. Chen, M. S.; White, M. C. Science 2010, 327, 566.
14. Paradine, S. M.; Griffin, J. R.; Zhao, J.; Petronico, A. L.; Miller, S. M.; White, M. C. Nat. Chem. 2015, 7, 987.
15. (a) Simmons, E. M.; Hartwig, J. F. Nature 2012, 483, 70. (b) Li, B.; Driess, M.; Hartwig, J. F. J. Am. Chem. Soc. 2014, 136, 6586.
16. Egger, H.; Reinshagen, H. J. Antibiot. 1976, 29, 923.
17. Karmel, C.; Li, B.; Hartwig, J. F. J. Am. Chem. Soc. 2018, 140, 1460.
18. Dess, D. B.; Martin, J. C. J. Am. Chem. Soc. 1991, 113, 7277.
19. Iwasaki, K.; Wan, K. K.; Oppedisano, A.; Crossley, S. W.; Shenvi, R. A. J. Am. Chem. Soc. 2014, 136, 1300.
20. Richter, M. F.; Drown, B. S.; Riley, A. P.; Garcia, A.; Shirai, T.; Svec, R. L.; Hergenrother, P. J. Nature 2017, 545, 299.
21. Tsuji, J.; Ohno, K. Tetrahedron Lett. 1%5, 6, 3969.

1. A method of introducing a hydroxyl group onto an unsubstituted methyl group of pleuromutilin which is adjacent to a free hydroxyl group in said compound wherein the vinyl group at C₁₉-C₂₀ of the compound has been reduced to an ethyl group and the remaining functional groups in said compound other than said free hydroxyl group are optionally protected, the method comprising introducing a C₁-C₄ dialkyl- or diphenylhydrosilyl group onto the free hydroxyl group to form a hydrosilane group, conducting a dehydrogenative C—H silylation reaction catalyzed by an iridium or ruthenium catalyst on the hydrosilane group to form a cyclosilane group with the adjacent methyl group and thereafter conducting a Tamao-Fleming oxidation on the cyclosilane group to provide a compound which contains an alcohol group on each of the two carbon atoms which formed the cyclosilane.

2. The method according to claim 1 wherein said hydrosilyl group is a dialkylhydrosilyl group.

3. The method according to claim 1 wherein said dialkylhydrosilyl group is a dimethyl or diethylhydrosilyl group.

4. The method according to claim 1 wherein said hydrosilyl group is a diphenylhydrosilyl group.

5. The method according to claim 1 wherein said iridium catalyst is methoxy(cyclooctadiene)iridium(I) dimer.

6. The method according to claim 5 wherein the dehydrogenative C—H silylation reaction catalyzed by methoxy(cyclooctadiene)iridium(I) dimer is conducted in the presence of norbornene and 3,4,7,8-tetramethyl-1,10-phenantholine (Me₄phen) in solvent at elevated temperature.

7. The method according to claim 1 wherein said Tamao-Fleming oxidation is conducted using a fluoride desilylating agent in combination with an oxidizing agent.

8. The method according to claim 7 wherein said fluoride desilylating agent is hydrogen fluoride, potassium fluoride, sodium fluoride or tetra-n-butyl ammonium fluoride.

9. The method according to claim 7 wherein said oxidizing agent is hydrogen peroxide, meta-chloroperbenzoic acid or a mixture thereof.

10. The method according to claim 1 wherein said pleuromutilin compound is compound 12 of FIG. 3, compound S18 (Scheme 5), compound 25 of FIG. 7, compound 30 of FIG. 8, compound 38 or its trifluoroacetylated analog of FIG. 10, compound 47 of FIG. 12 and compound 54 of FIG. 13.#

11-36. (canceled)

37. A compound according to the chemical structure: 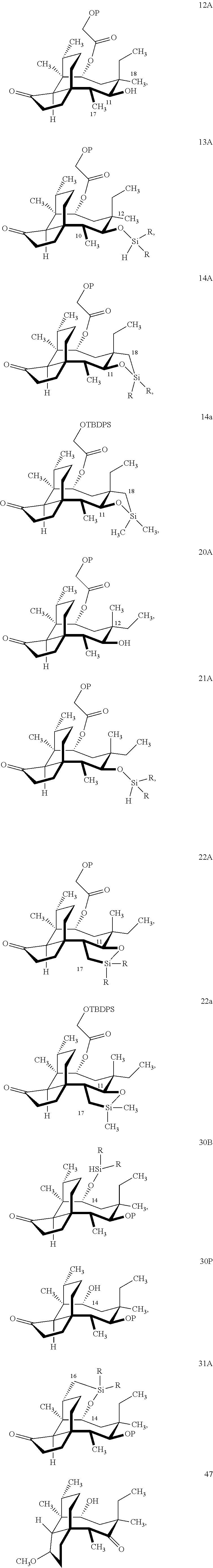 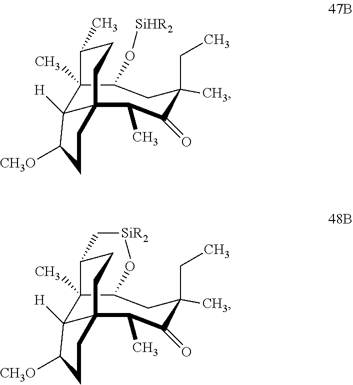
Where R is a C₁-C₃ alkyl group or a phenyl group and P is a protecting group, or
a pharmaceutically acceptable salt, stereoisomer, solvate or polymorph thereof.

38-41. (canceled)

42. A compound according to claim 37 wherein R is methyl or phenyl.

43. (canceled)

44. (canceled)

45. A compound according to claim 37 wherein P is a silyl protecting group.

46. A compound according to claim 45 wherein P is a tert.-butyldiphenylsilyl group.

47. A compound according to claim 37 wherein P is a butyloxymethylacetal (BOM) group.

48. (canceled)

49. (canceled)

50. (canceled)

51. A compound selected from the group consisting of compounds 12, 13, 14a, 14b, 15a and 15b of FIG. 3; compounds 20, S18, 21, 22a, 22b, 23a and 23b of FIG. 5; compound 24 of FIG. 6; compounds 25, 26, 27 and 28 of FIG. 7; compounds 30, 31 and 32 of FIG. 8; compounds 33, 34, 35 and 36 of FIG. 9, compound 37 of FIG. 9A; compounds 38 (or its trifluoracetylated analog), 39, 40, 41 and 42 of FIG. 10; compounds 43, 44 and 45 of FIG. 11; compounds 46, 47 and 49 of FIG. 12; compounds 50, 51, 52 and 53 of FIG. 13A; compounds 54, 55 and 56 of FIG. 13B; compounds 57, 58A, 58B, 58C and 58D of FIG. 13D, Table 1, compounds 59, 60a, 60b, 61 and 62 of FIG. 14, compound S3a of FIG. 16, compound S5 and 37 of FIG. 17, compounds S6, S7 and S8 of FIG. 18, compounds S9, S10, S1 and S12 of FIG. 19, compounds S13, S14 and S15 of FIG. 20, and their pharmaceutically acceptable salts, non-salts, alternative salts, stereoisomers, solvates and polymorphs thereof.

52. (canceled)

53. (canceled)

54. A pharmaceutical composition comprising an effective amount of at least one compound according to claim 37 in combination with a pharmaceutically acceptable carrier, additive or excipient.

55. A pharmaceutical composition comprising an effective amount of at least one compound according to claim 51 in combination with a pharmaceutically acceptable carrier, additive or excipient.

56. The composition according to claim 54 which further includes at least one additional bioactive agent.

57. The composition according to claim 54 which further includes at least one additional antibiotic.
